(19) United States
(12) Patent Application Publication
(10) Pub. No.: US 2004/0133008 A1
(43) Pub. Date: Jul. 8, 2004

(54) Amide compounds

(75) Inventors: Yoshikazu Inoue, Osaka (JP); Takeshi Terasawa, Osaka (JP); Hisashi Takasugi, Osaka (JP); Akira Nagayoshi, Osaka (JP); Koji Ueshima, Osaka (JP); Masae Sawada, Osaka (JP); Yoshiro Furukawa, Osaka (JP); Masafumi Mikami, Osaka (JP); Kazumasa Hinoue, Osaka (JP); Daisuke Fukumoto, Osaka (JP)

Correspondence Address: OBLON, SPIVAK, MCCLELLAND, MAIER & NEUSTADT, P.C., 1940 DUKE STREET, ALEXANDRIA, VA, 22314, US

(73) Assignees: FUJISAWA PHARMACEUTICAL CO., LTD., Osaka-shi (JP); DAISO CO., LTD., Osaka-shi (JP)

(21) Appl. No.: 10/694,091

(22) Filed: Oct. 28, 2003

(30) Foreign Application Priority Data
Oct. 29, 2002 (AU) 2002952331
May 27, 2003 (AU) 2003902622

(51) Int. Cl. C07D 211/06

(52) U.S. Cl. 546/226; 548/530

(57) ABSTRACT

A compound of the formula (I)

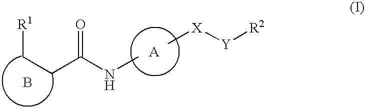

wherein

R¹ is hydrogen, lower alkyl, lower alkenyl, halo(lower)alkyl, cyclo(lower)alkyl, lower alkoxy, lower alkylthio, acyl, optionally substituted aryl or NR³R⁴;

R² is hydrogen; or aryl or heteroaryl, each of which may be substituted;

X is direct bond or bivalent residue derived from piperazine;

Y is -(A¹)ₙ-(A²)ₘ-, wherein n and m are independently 0 or 1);

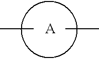

is bivalent residue derived from arene or heteroarene; and

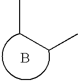

is bivalent residue derived from arene or heteroarene, or a salt thereof. The compound of the present invention and a salt thereof inhibit apolipoprotein B (Apo B) secretion and are useful as a medicament for prophylactic and treatment of diseases or conditions resulting from elevated circulating levels of Apo B.

### TECHNICAL FIELD

This invention relates to new amide compounds and salts thereof which inhibit apolipoprotein B (Apo B) secretion and are useful as a medicament.

### BACKGROUND ART

Apo B is the main component of lipoprotein such as VLDL (very low density lipoprotein), IDL (intermediate density lipoprotein) and LDL (low density lipoprotein). Compounds that inhibit Apo B secretion are useful for the treatment of diseases or conditions resulting from elevated circulating levels of Apo B, such as hyperlipemia, hyperlipidemia, hyperlipoproteinemia, hypercholesterolemia, hypertriglyceridemia, atherosclerosis, pancreatitis, non-insulin dependent diabetes mellitus (NIDDM), obesity and coronary heart diseases. Compounds that inhibit Apo B secretion have been described in WO96/40640, WO98/23593, WO98/56790 and WO00/32582. Compounds that inhibit Apo B secretion are also useful in reducing intestinal fat absorption, reducing food intake and treating obesity in combination with a known anti-obesity agent (EP 1 099 438, EP 1 099 439 and EP 1 099 441).

### DISCLOSURE OF INVENTION

This invention relates to new amide compounds.

One object of this invention is to provide new and useful amide compounds and salts thereof that inhibit Apo B secretion.

A further object of this invention is to provide a pharmaceutical composition comprising said amide compound or a pharmaceutically acceptable salt thereof.

Still further object of this invention is to provide a use of said amide compounds or pharmaceutically acceptable salts thereof as a medicament for prophylactic and therapeutic treatment of diseases or conditions resulting from elevated circulating levels of Apo B, such as hyperlipemia, hyperlipidemia, hyperlipoproteinemia, hypercholesterolemia, hypertriglyceridemia, atherosclerosis, pancreatitis, non-insulin dependent diabetes mellitus (NIDDM), obesity, coronary heart diseases, myocardial infarction, stroke, restenosis and Syndrome X.

Another object of this invention is to provide a method for inhibiting or decreasing Apo B secretion in a mammal, which comprises administering an Apo B secretion inhibiting or decreasing amount of said amide compound or a pharmaceutically acceptable salt thereof to the mammal.

Still further object of this invention is to provide a method for preventing or treating a disease or condition resulting from elevated circulating levels of Apo B in a mammal, such as hyperlipemia, hyperlipidemia, hyperlipoproteinemia, hypercholesterolemia, hypertriglyceridemia, atherosclerosis, pancreatitis, NIDDM, obesity, coronary heart diseases, myocardial infarction, stroke, restenosis and Syndrome X, which method comprises administering an effective amount of said amide compound or a pharmaceutically acceptable salt thereof to the mammal.

The object amide compounds of the present invention are novel and can be represented by the following general formula (I)

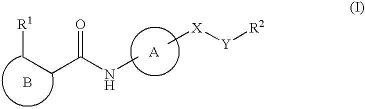

wherein

R¹ is hydrogen, lower alkyl, lower alkenyl, halo(lower)alkyl, cyclo(lower)alkyl, lower alkoxy, lower alkylthio, acyl, optionally substituted aryl or NR³R⁴, wherein

R³ and R⁴ are each independently hydrogen, lower alkyl, cyclo(lower)alkyl or acyl; or

R³, R⁴ and nitrogen atom to which they are attached form an optionally substituted, saturated or partially saturated N-containing heterocyclic group optionally having one or more oxygen or sulfur atom(s) and optionally having one or two lower alkyl(s);

R² is hydrogen; or aryl or heteroaryl in which imino group is optionally protected by amino protective group, each of which is optionally substituted by cyano, optionally protected amino, lower alkyl or heteroaryl substituted by one or more lower alkyl(s);

X is direct bond or bivalent residue derived from piperazine;

Y is -(A¹)ₙ-(A²)ₘ-

wherein

A¹ is —O—, —NH—, —N(R⁵)—, —CO—, —CH(OH)—, —NH—CO—, —CO—NH—, —CH₂—NH—CO—, —CH₂—CO—NH— or —(CH₂)₂—NH—CO—, wherein R⁵ is amino protective group,

A² is lower alkylene optionally substituted with lower alkyl or heteroaryl, and

n and m are independently 0 or 1;

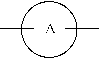

is bivalent residue derived from arene or heteroarene; and

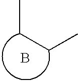

is bivalent residue derived from arene or heteroarene selected from

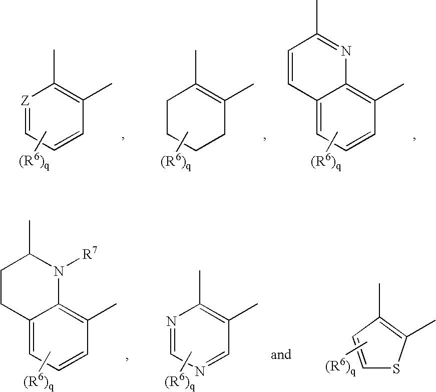

wherein

Z is N or C(R¹⁰),

R⁶ is hydrogen, halogen, lower alkyl, lower alkoxy, halo(lower)alkyl, lower alkanoyl, lower alkylthio or —NR⁸R⁹, wherein R⁸ and R⁹ are each independently lower alkyl, or R⁸, R⁹ and nitrogen atom to which they are attached form an optionally substituted, saturated or partially saturated N-containing heterocyclic group optionally having one or two lower alkyl(s);

R⁷ is lower alkyl;

R¹⁰ is the same as R⁶ defined above; and

q is 1 or 2,

or a salt thereof.

The preferred embodiments of the amide compound of the present invention is represented by the general formula (I), wherein

R¹ is hydrogen, lower alkyl, lower alkenyl, halo(lower)alkyl, cyclo(lower)alkyl, lower alkoxy, lower alkylthio, lower alkylsulfonyl or NR³R⁴,

wherein R³ and R⁴ are each independently hydrogen, lower alkyl, cyclo(lower)alkyl, lower alkanoyl; or

R³, R⁴ and nitrogen atom to which they are attached form an optionally substituted, saturated or partially saturated N-containing heterocyclic group selected from

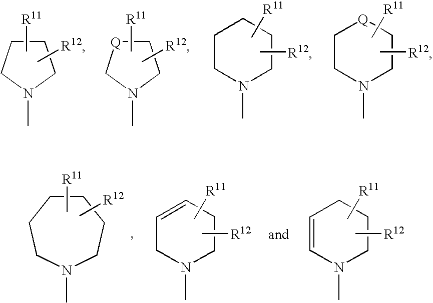

wherein R¹¹ and R¹² are each independently hydrogen or lower alkyl, and Q is —N(R¹³)—, —O—, —S—, —SO— or —SO₂—, wherein R¹³ is hydrogen or lower alkyl;

R² is hydrogen, phenyl, pyridinyl, pyrimidinyl, pyrazolyl, thiazolyl, pyrrolyl, triazolyl in which imino group is optionally protected by amino protective group, tetrazolyl, furanyl or thienyl, each of which is optionally substituted by cyano, optionally protected amino, lower alkyl or pyrrolyl substituted by one or more lower alkyl (s);

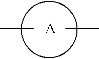

is phenylene, pyridinediyl, indolinediyl, isoindolynediyl, 3-oxo-2,3-dihydro-1H-indolediyl or 3,4-dihydro-2(1H)-isoquinolinediyl; and

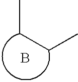

is bivalent residue derived from arene or heteroarene selected from

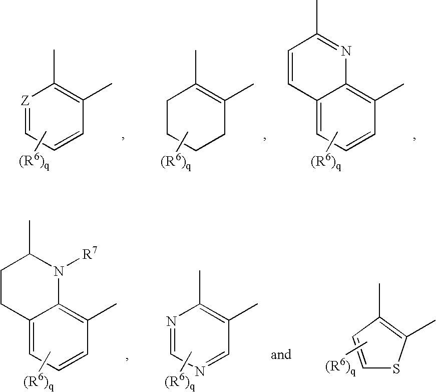

wherein

Z is N or C(R¹⁰)

R⁶ is hydrogen, halogen, lower alkyl, lower alkoxy, halo(lower)alkyl, lower alkanoyl, lower alkylthio or —NR⁸R⁹, wherein R⁸ and R⁹ are each independently lower alkyl, or

R⁸, R⁹ and nitrogen atom to which they are attached form an optionally substituted, saturated or partially saturated N-containing heterocyclic group selected from

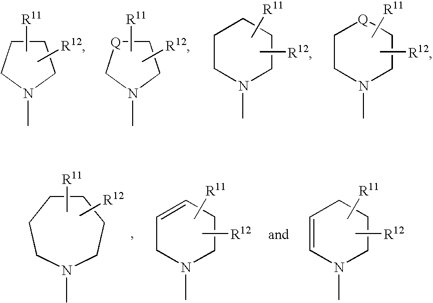

wherein R¹¹, R¹² and Q are as defined above;

R⁷ is as defined above; and

q is 1 or 2,

or a salt thereof.

Another preferred embodiment of the amide compounds of the present invention can be represented by the following general formula (I′)

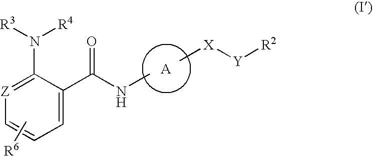

wherein

R² is aryl or heteroaryl, each of which is optionally substituted by cyano, optionally protected amino, lower alkyl or heteroaryl substituted by one or more lower alkyl(s);

R³ and R⁴ are each independently lower alkyl, or R³, R⁴ and nitrogen atom to which they are attached form an optionally substituted, saturated or partially saturated N-containing heterocyclic group;

R⁶ is hydrogen, halogen, lower alkyl, lower alkoxy, halo(lower)alkyl, lower alkanoyl or —NR⁸R⁹ (wherein R⁸ and R⁹ are each independently lower alkyl, or R⁸, R⁹ and nitrogen atom to which they are attached form an optionally substituted, saturated or partially saturated N-containing heterocyclic group);

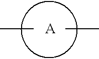

is bivalent residue derived from arene or heteroarene;

X is direct bond or bivalent residue derived from piperazine,

Y is -(A¹)ₙ-(A²)ₘ-

wherein A¹ is —O—, —NH—, —N(R⁵)—, —CO—, —CH(OH)—, —NH— CO—, —CH₂—NH—CO— or —CH₂—CO—NH—, wherein R⁵ is amino protective group,

A² is lower alkylene, and

n and m are independently 0 or 1;

Z is N or C(R¹⁰) (wherein R¹⁰ is the same as R⁶ defined above), or a salt thereof.

The preferred embodiments of the amide compound of the present invention represented by the general formula (I′) are as follows.

(1) The compound of the general formula (I′), wherein

R² is phenyl, pyridinyl, pyrimidinyl, pyrazolyl, thiazolyl, pyrrolyl, triazolyl or tetrazolyl, each of which is optionally substituted by cyano, optionally protected amino, lower alkyl or pyrrolyl substituted by one or more lower alkyl(s),

R³ and R⁴ are each independently lower alkyl, or R³, R⁴ and nitrogen atom to which they are attached form a saturated or partially saturated N-containing heterocyclic group selected from

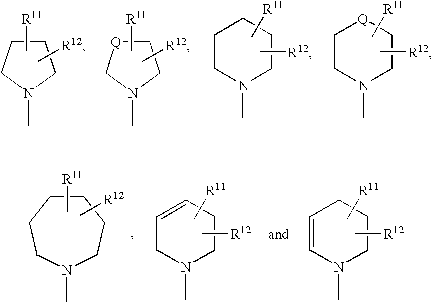

wherein R¹¹ and R¹² are each independently hydrogen or lower alkyl, and Q is —N(R¹³)—, —O—, —S—, —SO— or —SO₂— wherein R¹³ is hydrogen or lower alkyl;

R⁶ is hydrogen, halogen, lower alkyl, lower alkoxy, halo(lower)alkyl, lower alkanoyl or —NR⁸R⁹ (wherein R⁸ and R⁹ are each independently lower alkyl, or R¹¹, R¹² and nitrogen atom to which they are attached form a saturated or partially saturated N-containing heterocyclic group selected from

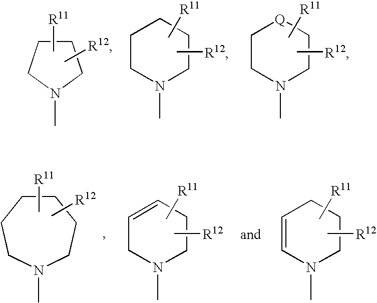

wherein R¹¹, R¹² and Q are as defined above); and

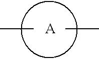

is phenylene, pyridinediyl, indolinediyl or isoindolinediyl, or a salt thereof.

(2) The compound of the general formula (I′), wherein

R² is phenyl, pyridinyl, pyrimidinyl, pyrazolyl, thiazolyl, pyrrolyl, triazolyl or tetrazolyl, each of which is optionally substituted by cyano, optionally protected amino, lower alkyl or pyrrolyl substituted by one or more lower alkyl(s);

R³ and R⁴ are each independently lower alkyl;

R⁶ is hydrogen, halogen, lower alkyl, lower alkoxy, lower alkanoyl or halo(lower)alkyl; and

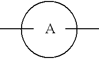

is phenylene, or a salt thereof.

(3) The compound of the general formula (I′), wherein

R² is phenyl, pyridinyl, pyrimidinyl, pyrazolyl, thiazolyl, pyrrolyl, triazolyl or tetrazolyl, each of which is optionally substituted by cyano, optionally protected amino, lower alkyl or pyrrolyl substituted by one or more lower alkyl(s);

R³ and R⁴ are each independently lower alkyl;

R⁶ is hydrogen, halogen, lower alkyl, lower alkoxy, lower alkanoyl or halo(lower)alkyl; and

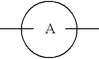

is indolinediyl or isoindolinediyl, or a salt thereof.

(4) The compound of the general formula (I′), wherein

R² is phenyl, pyridinyl, pyrimidinyl, pyrazolyl, thiazolyl, pyrrolyl, triazolyl or tetrazolyl, each of which is optionally substituted by cyano, optionally protected amino, lower alkyl or pyrrolyl substituted by one or more lower alkyl(s);

R³, R⁴ and nitrogen atom to which they are attached form a saturated N-containing heterocyclic group of the formula

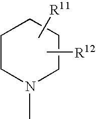

wherein R¹¹ and R¹² are each independently hydrogen or lower alkyl;

R⁶ is hydrogen, halogen, lower alkyl, lower alkoxy, lower alkanoyl or halo(lower)alkyl; and

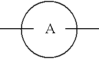

is phenylene, or a salt thereof.

(5) The compound of the general formula (I′), wherein

R² is phenyl, pyridinyl, pyrimidinyl, pyrazolyl, thiazolyl, pyrrolyl, triazolyl or tetrazolyl, each of which is optionally substituted by cyano, optionally protected amino, lower alkyl or pyrrolyl substituted by one or more lower alkyl(s);

R³, R⁴ and nitrogen atom to which they are attached form a saturated N-containing heterocyclic group of the formula

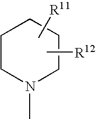

wherein R¹¹ and R¹² are each independently hydrogen or lower alkyl;

R⁶ is hydrogen, halogen, lower alkyl, lower alkoxy, lower alkanoyl or halo(lower)alkyl; and

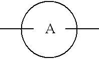

is indolinediyl or isoindolinediyl, or a salt thereof.

Another preferred embodiment of the amide compounds of the present invention can be represented by the following general formula (I″)

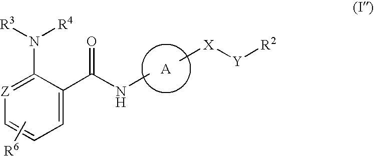

wherein

R² is aryl or heteroaryl, each of which is optionally substituted by cyano, amino, lower alkyl or heteroaryl substituted by one or more lower alkyl(s);

R³ and R⁴ are each independently lower alkyl, or R³ , R⁴ and nitrogen atom to which they are attached form an optionally substituted, saturated or partially saturated N-containing heterocyclic group;

R⁶ is hydrogen, halogen, lower alkyl, lower alkoxy, halo(lower)alkyl or —NR⁸R⁹ (wherein R⁸ and R⁹ are each independently lower alkyl, or R⁸, R⁹ and nitrogen atom to which they are attached form an optionally substituted, saturated or partially saturated N-containing heterocyclic group);

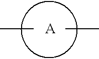

is bivalent residue derived from arene or heteroarene;

X is direct bond or bivalent residue derived from piperazine,

Y is -(A¹)ₙ-(A²)ₘ-

wherein A¹ is —O—, —NH—, —N(R⁵)—, —CO— or —NH—CO—,

wherein R⁵ is amino protective group,

A² is lower alkylene, and

n and m are independently 0 or 1; and

Z is N or C(R¹⁰) (wherein R¹⁰ is the same as R⁶ defined above), or a salt thereof.

The preferred embodiments of the amide compound of the present invention represented by the general formula (I″) are as follows.

(1) The compound of the general formula (I″), wherein

R² is phenyl, pyridinyl, pyrimidinyl or thiazolyl, each of which is optionally substituted with cyano, amino, lower alkyl or pyrrolyl substituted with one or more lower alkyl;

R³ and R⁴ are each independently lower alkyl, or R³, R⁴ and nitrogen atom to which they are attached form a saturated or partially saturated N-containing heterocyclic group selected from

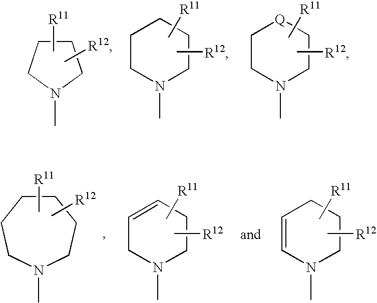

wherein R¹¹ and R¹² are each independently hydrogen or lower alkyl, and Q is —N(R¹³)—, —O—, —S—, —SO— or —SO₂— wherein R¹³ is hydrogen or lower alkyl;

R⁶ is hydrogen, halogen, lower alkyl, lower alkoxy, halo(lower)alkyl or —NR⁸R⁹ (wherein R⁸ and R⁹ are each independently lower alkyl, or R⁸, R⁹ and nitrogen atom to which they are attached form a saturated or partially saturated N-containing heterocyclic group selected from

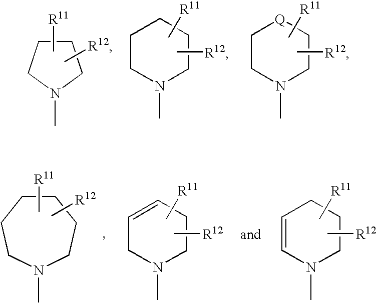

wherein R¹¹, R¹² and Q are as defined above); and

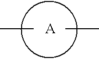

is phenylene, pyridinediyl or indolinediyl, or a salt thereof.

(2) The compound of the general formula (I″), wherein

R² is phenyl, pyridinyl, pyrimidinyl or thiazolyl, each of which is optionally substituted with cyano, amino, lower alkyl or pyrrolyl substituted with one or more lower alkyl;

R³ and R⁴ are each independently lower alkyl;

R⁶ is hydrogen, halogen, lower alkyl, lower alkoxy or halo(lower)alkyl; and

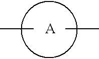

is phenylene, or a salt thereof.

(3) The compound of the general formula (I″), wherein

R² is phenyl, pyridinyl, pyrimidinyl or thiazolyl, each of which is optionally substituted with cyano, amino, lower alkyl or pyrrolyl substituted with one or more lower alkyl;

R³ and R⁴ are each independently lower alkyl;

R⁶ is hydrogen, halogen, lower alkyl, lower alkoxy or halo(lower)alkyl; and

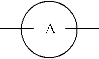

is indolinediyl, or a salt thereof.

(4) The compound of the general formula (I″), wherein

R² is phenyl, pyridinyl, pyrimidinyl or thiazolyl, each of which is optionally substituted with cyano, amino, lower alkyl or pyrrolyl substituted with one or more lower alkyl;

R³, R⁴ and nitrogen atom to which they are attached form a saturated N-containing heterocyclic group of the formula

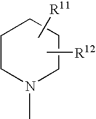

wherein R¹¹ and R¹² are each independently hydrogen or lower alkyl;

R⁶ is hydrogen, halogen, lower alkyl, lower alkoxy or halo(lower)alkyl; and

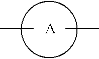

is phenylene, or a salt thereof.

(5) The compound of the general formula (I″), wherein

R² is phenyl, pyridinyl, pyrimidinyl or thiazolyl, each of which is optionally substituted with cyano, amino, lower alkyl or pyrrolyl substituted with one or more lower alkyl;

R³, R⁴ and nitrogen atom to which they are attached form a saturated N-containing heterocyclic group of the formula

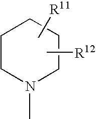

wherein R¹¹ and R¹² are each independently hydrogen or lower alkyl;

R⁶ is hydrogen, halogen, lower alkyl, lower alkoxy or halo(lower)alkyl; and

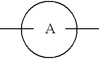

is indolinediyl, or a salt thereof.

The above-mentioned amide compounds represented by the general formulas (I′) and (I″) are also encompassed in the scope of the compound represented by the general formula (I). Hereinafter “compound (I)” also encompasses “compound (I′)” and “compound (I″)”.

Suitable salts of the object compound (I) may be pharmaceutically acceptable salts such as conventional non-toxic salts and include, for example, a salt with a base or an acid addition salt such as a salt with an inorganic base, for example, an alkali metal salt (e.g., sodium salt, potassium salt, etc.), an alkaline earth metal salt (e.g., calcium salt, magnesium salt, etc.), an ammonium salt; a salt with an organic base, for example, an organic amine salt (e.g., triethylamine salt, pyridine salt, picoline salt, ethanolamine salt, triethanolamine salt, dicyclohexylamine salt, N,N′-dibenzylethylenediamine salt, etc.); an inorganic acid addition salt (e.g., hydrochloride, hydrobromide, sulfate, phosphate, etc.); an organic carboxylic or sulfonic acid addition salt (e.g., formate, acetate, trifluoroacetate, maleate, tartrate, citrate, fumarate, methanesulfonate, benzenesulfonate, toluenesulfonate, etc.); and a salt with a basic or acidic amino acid (e.g., arginine, aspartic acid, glutamic acid, etc.).

In the above and subsequent descriptions of the present specification, suitable examples and illustration of the various definitions which the present invention intends to include within the scope thereof are explained in detail as follows.

The term “lower” is used to intend a group having 1 to 6, preferably 1 to 4, carbon atom(s), unless otherwise provided.

Suitable “lower alkyl” includes straight or branched alkyl having 1 to 6 carbon atom(s), such as methyl, ethyl, propyl, isopropyl, butyl, isobutyl, sec-butyl, tert-butyl, pentyl, tert-pentyl and hexyl, in which more preferred one is C₁-C₄ alkyl.

Suitable “cyclo(lower)alkyl” includes cycloalkyl having 3 to 6 carbon atom(s), such as cyclopropyl, cyclobutyl, cyclopentyl and cyclohexyl, in which the preferred one is cyclohexyl.

Suitable “lower alkenyl” includes straight or branched alkenyl having 2 to 6 carbon atom(s), such as ethenyl, propenyl, isopropenyl, butenyl, isobutenyl, sec-butenyl, tert-butenyl, pentenyl, tert-pentenyl and hexenyl, in which more preferred one is C₂-C₄ alkenyl, and the particularly preferred one is isopropenyl.

Suitable “lower alkoxy” includes straight or branched alkoxy having 1 to 6 carbon atom(s), such as methoxy, ethoxy, propoxy, isopropoxy, butoxy, isobutoxy, sec-butoxy, tert-butoxy, pentyloxy, tert-pentyloxy and hexyloxy, in which more preferred one is C₁-C₄ alkoxy.

Suitable “halogen” and “halogen” moiety in the term “halo(lower)alkyl” may be fluorine, bromine, chlorine and iodine.

Suitable “halo(lower)alkyl” includes straight or branched haloalkyl having 1 to 6 carbon atom(s) such as fluoromethyl, bromomethyl, chloromethyl, difluoromethyl, dibromomethyl, dichloromethyl, trifluoromethyl, trichloromethyl and tribromomethyl, in which more preferred one is halo(C₁-C₄)alkyl, and the particularly preferred one is trifluoromethyl.

Suitable “lower alkylthio” includes alkylthio wherein alkyl moiety is straight or branched alkyl having 1 to 6 carbon atom(s) such as methylthio, ethylthio, propylthio, isopropylthio, butylthio, isobutylthio, sec-butylthio, tert-butylthio, pentylthio, tert-pentylthio and hexylthio, in which more preferred one is C₁-C₄ alkylthio, and the particularly preferred one is methylthio.

Suitable “lower alkylene” includes straight or branched alkylene having 1 to 6 carbon atom(s), such as methylene, ethylene, trimethylene, tetramethylene, propylene, ethylidene and propylidene, in which more preferred one is C₁-C₃ alkylene, and the particularly preferred ones are methylene and ethylene.

Suitable examples of “amino protective group” include acyl such as lower alkanoyl (e.g., formyl, acetyl, etc.), lower alkoxycarbonyl, mono(or di or tri)phenyl(lower)alkoxycarbonyl, and a conventional protective group such as mono(or di or tri)aryl(lower)alkyl, for example, mono(or di or tri)phenyl(lower)alkyl.

Suitable “acyl” includes “lower alkanoyl”, “lower alkoxycarbonyl”, “aryl(lower)alkoxycarbonyl”, “carbamoyl”, “N-(lower)alkylcarbamoyl”, “N,N-di(lower)alkylcarbamoyl” and “lower alkylsulfonyl”.

Suitable “lower alkanoyl” includes alkanoyl having 1 to 6 carbon atom(s) such as formyl, acetyl, propionyl, butyryl, isobutyryl, valeryl, isovaleryl, pivaloyl and hexanoyl, in which more preferred one is C₁-C₄ alkanoyl.

Suitable “N-(lower)alkylcarbamoyl” includes N-alkylcarbamoyl wherein alkyl moiety is alkyl having 1 to 6 carbon atom(s) such as N-methylcarbamoyl, N-ethylcarbamoyl, N-isopentylcarbamoyl and N-hexylcarbamoyl.

Suitable “N,N-di(lower)alkylcarbamoyl” includes N,N-dialkylcarbamoyl wherein two alkyl moieties may be same or different, such as N,N-dimethylcarbamoyl, N,N-diethylcarbamoyl, N-methyl-N-ethylcarbamoyl, N-pentyl-N-hexylcarbamoyl, etc.

Suitable “lower alkylsulfonyl” includes alkylsulfonyl wherein alkyl moiety is alkyl having 1 to 6 carbon atom(s) such as methylsulfonyl, ethylsulfonyl, propylsulfonyl, isopropylsulfonyl, butylsulfonyl, isobutylsulfonyl, sec-butylsulfonyl, tert-butylsulfonyl, pentylsulfonyl, tert-pentylsulfonyl and hexylsulfonyl, in which the preferred one is C₁-C₄ alkylsulfonyl.

Suitable “lower alkoxycarbonyl” includes alkoxycarbonyl wherein alkoxy moiety has 1 to 6 carbon atom(s) such as methoxycarbonyl, ethoxycarbonyl, propoxycarbonyl, isopropoxycarbonyl, butoxycarbonyl, isobutoxycarbonyl, sec-butoxycarbonyl, tert-butoxycarbonyl, pentyloxycarbonyl, tert-pentyloxycarbonyl and hexyloxycarbonyl, in which more preferred one is alkoxycarbonyl wherein alkoxy moiety has 1 to 4 carbon atom(s).

Suitable “aryl(lower)alkoxycarbonyl” includes “mono(or di or tri)phenyl(lower)alkoxycarbonyl”, etc. The “mono(or di or tri)phenyl(lower)alkoxycarbonyl” includes mono(or di or tri)phenylalkoxycarbonyl wherein alkoxy moiety has 1 to 6 carbon atom(s) such as benzyloxycarbonyl and phenethyloxycarbonyl. Suitable “mono(or di or tri)phenyl(lower)alkyl” includes mono(or di or tri)phenyl(C₁-C₆)alkyl such as benzyl, benzhydryl and trityl.

Suitable “saturated or partially saturated N-containing heterocyclic group” includes a saturated or partially saturated 4 to 8-membered (more preferably 5 to 7-membered) heteromonocyclic group containing 1 or 2 nitrogen atom(s) and optionally containing oxygen atom or sulfur atom, such as pyrrolidinyl, piperidinyl, piperazinyl, morpholinyl, thiomorpholinyl, hexahydroazepinyl and tetrahydropyridinyl.

“Saturated or partially saturated N-containing heterocyclic group” is optionally substituted by suitable substituent(s) such as lower alkyl and oxo.

Suitable “aryl” includes C₆-C₁₂ aryl. “Aryl” includes fused carbocyclic group wherein benzene ring is fused with a saturated or unsaturated carbon ring.

Suitable examples of “aryl” include phenyl, naphthyl, indenyl and indanyl, in which more preferred one is phenyl.

Suitable “heteroaryl” includes 5 to 10-membered aromatic heteromonocyclic or fused heterocyclic group containing 1 to 4 heteroatom(s) selected from sulfur atom, oxygen atom and nitrogen atom. “Heteroaryl” includes fused heterocyclic group wherein benzene ring is fused with a saturated or unsaturated heterocyclic ring.

Suitable examples of “heteroaryl” include pyridinyl, pyrimidinyl, pyrazinyl, pyridazinyl, pyrrolyl, imidazolyl, pyrazolyl, triazolyl, tetrazolyl, thiazolyl, isothiazolyl, thiadiazolyl, oxazolyl, isoxazolyl, furanyl, thienyl, indolyl, isoindolyl, indolizinyl, indazolyl, benzimidazolyl, benzotriazolyl, quinolyl, isoquinolyl, phthalazinyl, quinoxalinyl, quinazolinyl, cinnolinyl, benzofuranyl, benzothienyl, benzoxazolyl, benzothiazolyl, benzimidazolyl, indolinyl, isoindolinyl, tetrahydroquinolinyl and tetrahydroisoquinolinyl.

Suitable “bivalent residue derived from arene” includes C₆-C₁₂ arylene. “Bivalent residue derived from arene” include bivalent fused carbocyclic group wherein benzene ring is fused with a saturated or unsaturated carbon ring.

Suitable examples of “bivalent residue derived from arene” include phenylene, naphthylene, indenediyl and indandiyl, in which more preferred one is phenylene.

Suitable “bivalent residue derived from heteroarene” includes bivalent 5 to 10-membered aromatic heteromonocyclic or fused heterocyclic group containing 1 to 4 heteroatom(s) selected from sulfur atom, oxygen atom and nitrogen atom. “Bivalent residue derived from heteroarene” includes bivalent fused heterocyclic group wherein benzene ring is fused with a saturated or unsaturated heterocyclic ring.

Suitable examples of “bivalent residue derived from heteroarene” include pyridinediyl, pyrimidinediyl, pyrazinediyl, pyridazinediyl, pyrrolediyl, imidazolediyl, pyrazolediyl, triazolediyl, tetrazolediyl, thiazolediyl, isothiazolediyl, thiadiazolediyl, oxazolediyl, isoxazolediyl, furandiyl, thiophenediyl, indolediyl, isoindolediyl, indolizinediyl, indazolediyl, benzimidazolediyl, benzotriazolediyl, quinolinediyl, isoquinolinediyl, phthalazinediyl, quinoxalinediyl, quinazolinediyl, cinnolinediyl, benzofurandiyl, benzothiophenediyl, benzoxazolediyl, benzothiazolediyl, benzimidazolediyl, indolinediyl, isoindolinediyl, tetrahydroquinolinediyl and tetrahydroisoquinolinediyl.

Suitable examples of “carboxy protective group” include lower alkyl (e.g., methyl, ethyl, tert-butyl, etc.), mono(or di or tri)phenyl(lower)alkyl optionally substituted by nitro (e.g., benzyl, 4-nitrobenzyl, benzhydryl, trityl, etc.) and lower alkylcarbonyloxy(lower)alkyl (e.g., pivaloyloxymethyl).

Preferable examples of “optionally substituted, saturated or partially saturated N-containing heterocyclic group” include groups of the following formulas:

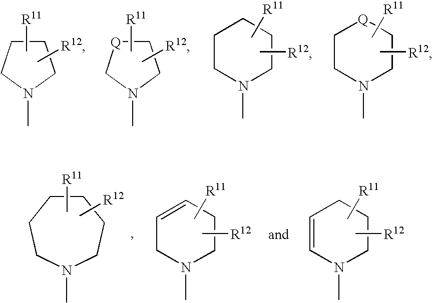

wherein R¹¹ and R¹² are each independently hydrogen or lower alkyl, and Q is —N(R¹³)—, —O—, —S—, —SO— or —SO₂— wherein R¹³ is hydrogen or lower alkyl.

Preferable example of “aryl” at R² is phenyl.

Preferable examples of “heteroaryl” at R² include 5 or 6-membered aromatic heteromonocyclic group containing 1 to 4 nitrogen atom(s) such as pyridinyl, pyrimidinyl, pyrazinyl, pyridazinyl, pyrrolyl, imidazolyl, pyrazolyl, triazolyl, tetrazolyl and thiazolyl, and more preferably pyridinyl, pyrrolyl, pyrazolyl, triazolyl, tetrazolyl and thiazolyl, particularly preferably, pyridinyl.

Preferable examples of “heteroaryl substituted by one or more lower alkyl(s)” include pyrrolyl substituted by one or more lower alkyl(s), and more preferably 2,5-dimethyl-1H-pyrrol-1-yl.

Preferable example of “bivalent residue derived from arene” at

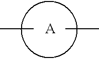

is phenylene.

Preferable examples of “bivalent residue derived from heteroarene” at

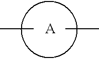

include bivalent 5 or 6-membered aromatic heteromonocyclic group containing 1 to 4 nitrogen atom(s) such as pyridinediyl, pyrimidinediyl, pyrazinediyl, pyridazinediyl, pyrrolediyl, imidazolediyl, pyrazolediyl, triazolediyl and tetrazolediyl; and bivalent 8 to 10-membered fused heterocyclic group containing 1 to 4 nitrogen atom(s) wherein benzene ring is fused with a saturated or unsaturated heterocyclic ring such as indolinediyl, isoindolinediyl, tetrahydroquinolinediyl and tetrahydroisoquinolinediyl.

More preferably, “bivalent residue derived from heteroarene” at

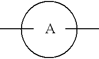

is pyridinediyl, indolinediyl or isoindolinediyl.

Particularly preferable examples of “bivalent residue derived from arene or heteroarene” at

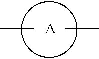

include

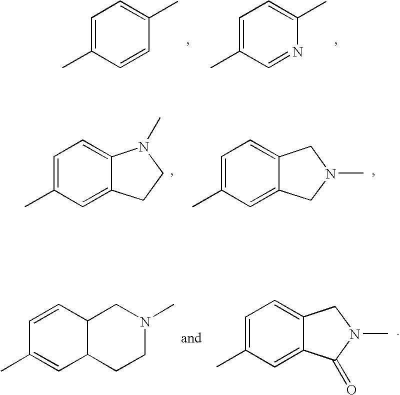

Preferable example of “bivalent residue derived from piperazine” at X is 1,4-piperazinediyl.

Preferable examples of a group represented by Y include —NH—CO—CH₂—, —N(R⁵)—(CH₂)₂—, —O—CH₂—, —CH₂—, —CO—CH₂—, —CH(OH)—, —O—(CH₂)₂—, —(CH₂)₂—, —CO—(CH₂)₂—, —CH(OH)—(CH₂)₂—, —(CH₂)₃—, —CH₂—CO—NH—, —CH₂—NH—CO—, —NH(CH₂)₂—, —CONH—, —(CH₂)₂—NH—CO—, —CONHCH₂—, —CONH(CH₂)₂—, —NHCOCH(CH₃)—, —CONHCH(CH₃)— and

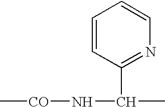

and more preferably, —NH—CO—CH₂—, —N(R⁵)—(CH₂)₂—, —O—CH₂—, —CH₂—, —CO—CH₂—, —CH(OH)—, —O(CH₂)₂—, —NH(CH₂)₂—, —CONH—, —CONHCH₂—, —CONH(CH₂)₂—, —NHCOCH(CH₃)— and —CONHCH(CH₃)—.

The object compound (I) of the present invention can be prepared by the following processes.

Process (1)

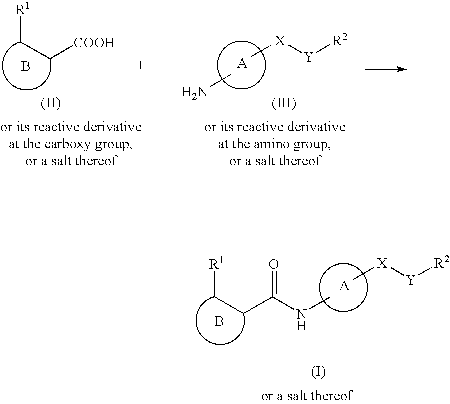

Process (2)

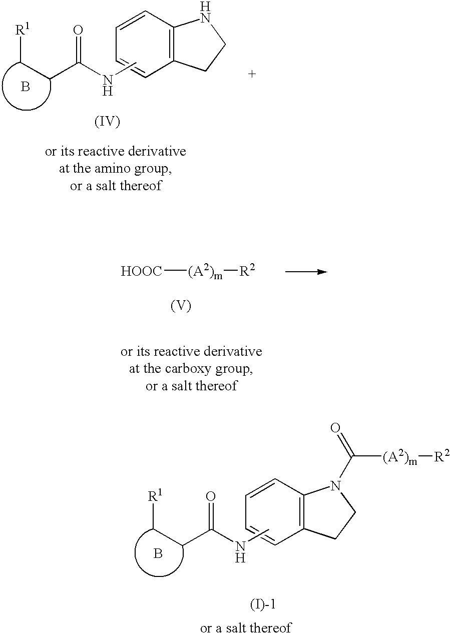

Process (3)

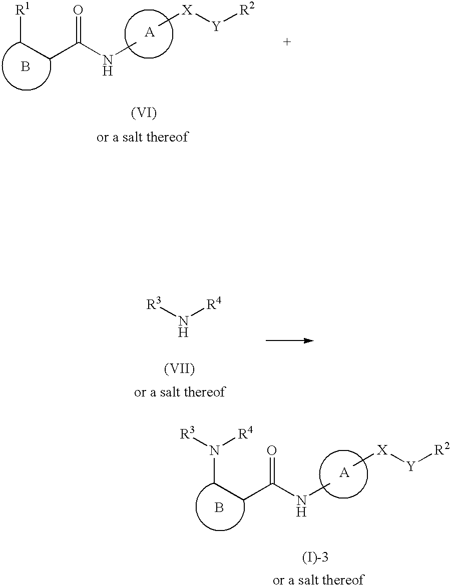

Process (4)

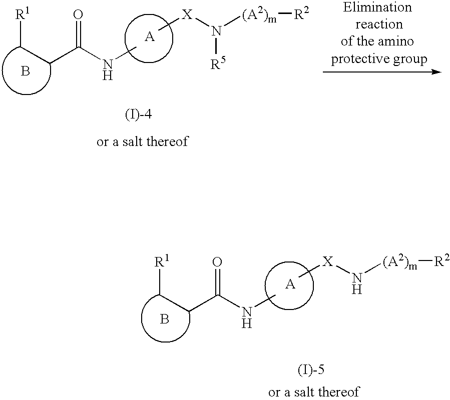

Process (5)

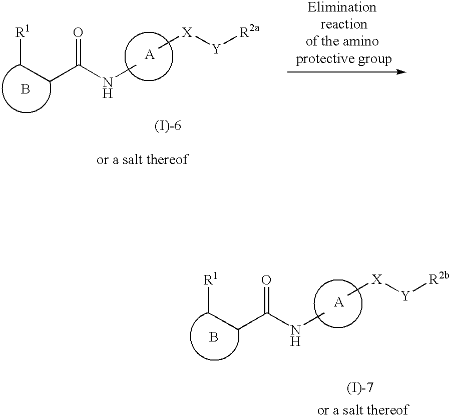

Process (6)

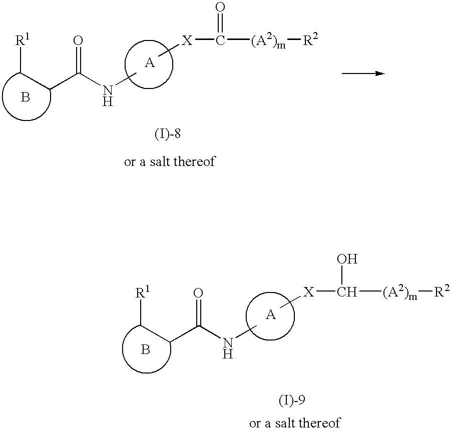

Process (7)

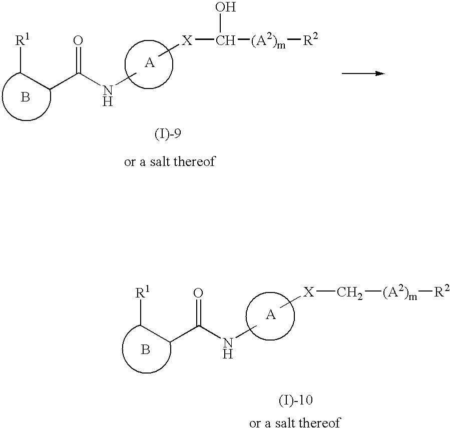

Process (8)

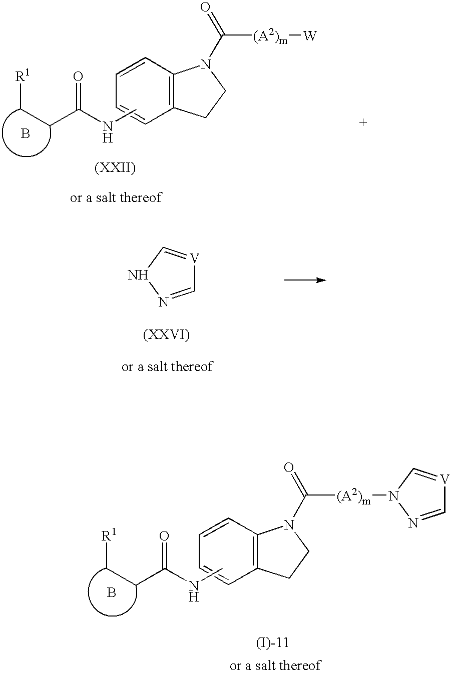

Process (9)

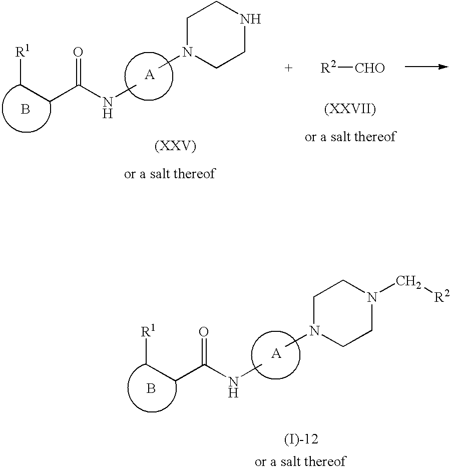

Process (10)

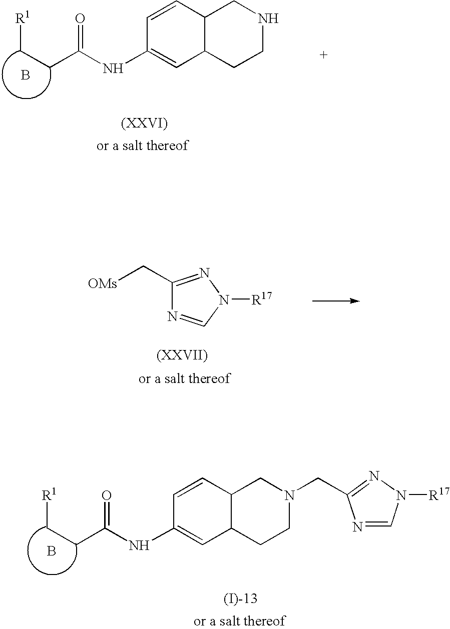

Process (11)

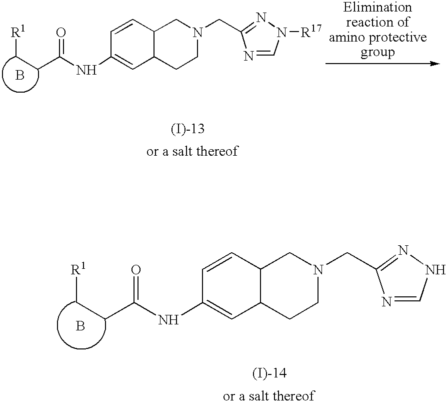

wherein R¹, R², R³, R⁴, R⁵,

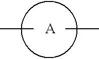

X, Y, Z, A² and m are as defined above,

X¹ is leaving group such as halogen (e.g., chlorine, bromine or fluorine) and trifluoromethanesulfonyloxy,

W is halogen (e.g., chlorine, bromine or fluorine),

V is CH or nitrogen atom,

R^{2a} is aryl or heteroaryl, each of which is substituted by protected amino,

R^{2b} is aryl or heteroaryl, each of which is substituted by amino, and

R¹⁷ is amino protective group.

The starting compounds can be prepared by the following processes or by the method of Preparation mentioned below or by a process known in the art for preparing their structurally analogous compounds.

Process (A)

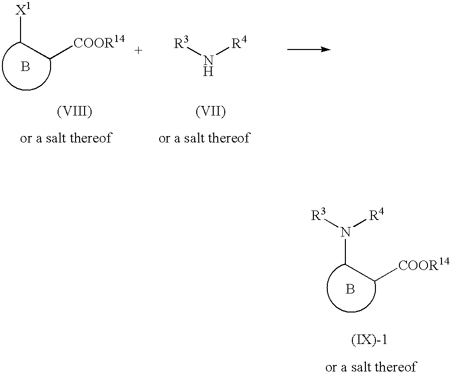

Process (B)

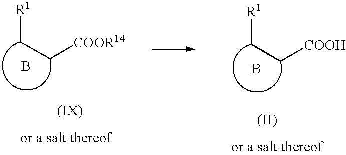

Process (C)

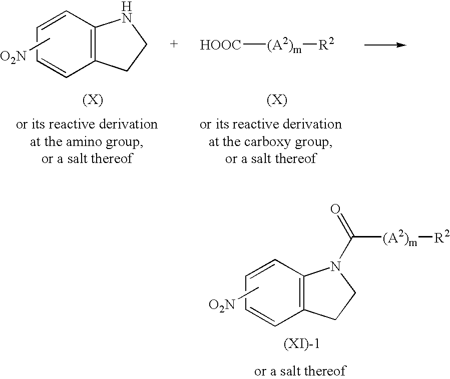

Process (D)

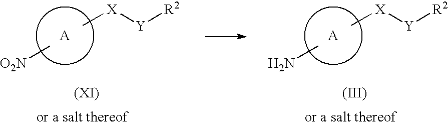

Process (E)

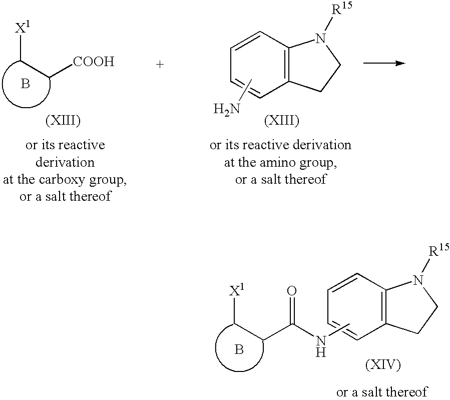

Process (F)

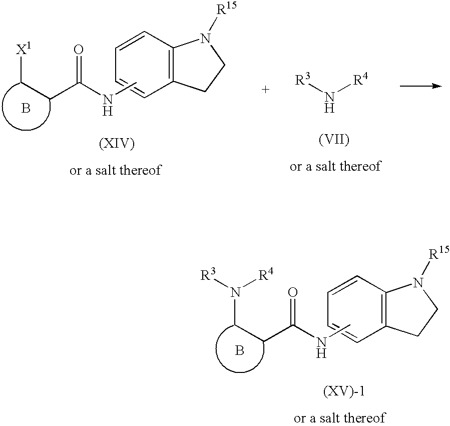

Process (G)

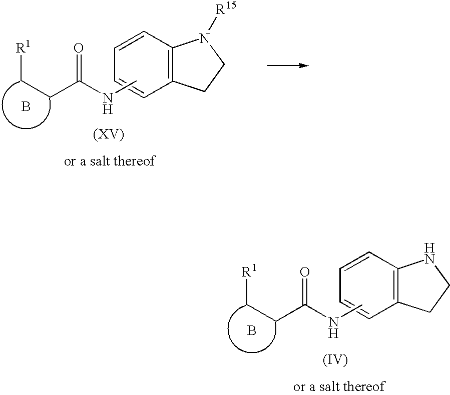

Process (H)

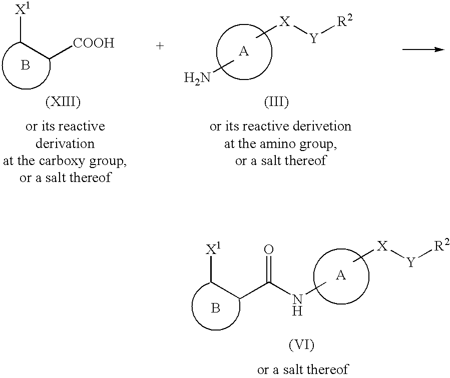

Process (I)

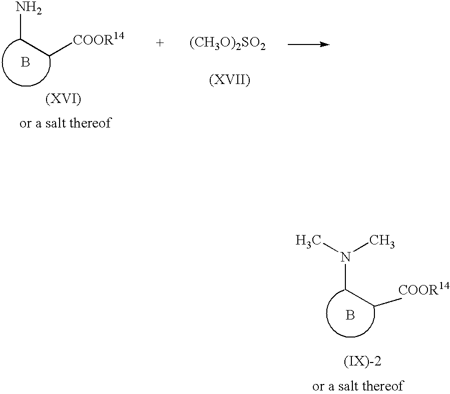

Process (J)

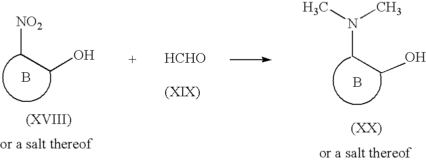

Process (K)

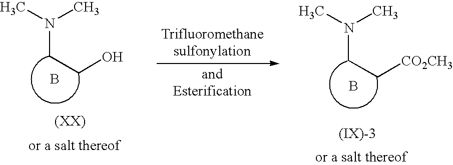

Process (L)

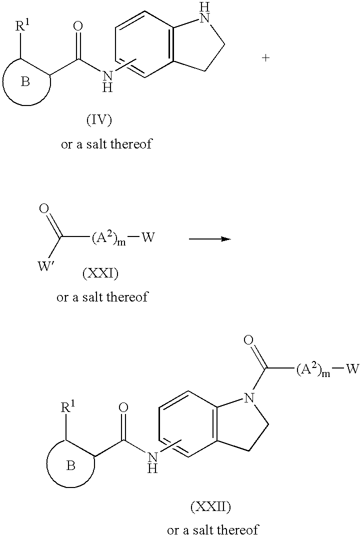

Process (M)

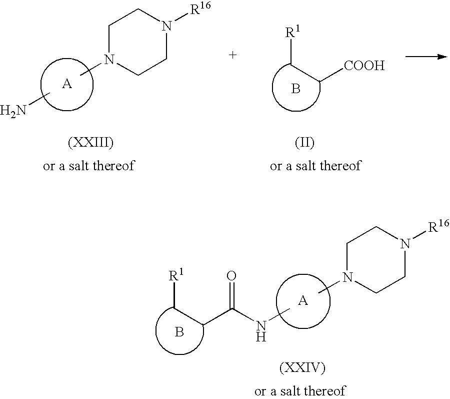

Process (N)

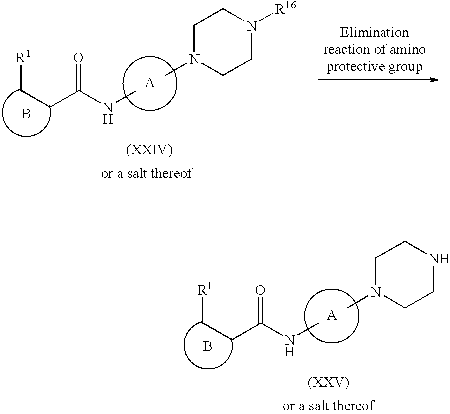

wherein R¹, R², R³, R⁴ ,

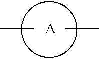

X, Y, Z, A², m and X¹ are as defined above,

W and W′ are each halogen such as fluorine, chlorine, bromine, etc.,

R¹⁴ is carboxy protective group, and

R¹⁵ and R¹⁶ are each amino protective group.

The processes for preparing the object and starting compounds are explained in detail in the following.

Process (1)

The compound (I) or a salt thereof can be prepared by reacting the compound (II) or its reactive derivative at the carboxy group, or a salt thereof with the compound (III) or its reactive derivative at the amino group, or a salt thereof.

Suitable reactive derivative of the compound (III) includes Schiff's base type imino or its tautomeric enamine type isomer formed by the reaction of the compound (III) with a carbonyl compound such as aldehyde, ketone or the like; a silyl derivative formed by the reaction of the compound (III) with a silyl compound such as N,O-bis(trimethylsilyl)acetamide, N-trimethylsilylacetamide or the like; a derivative formed by the reaction of the compound (III) with phosphorus trichloride or phosgene.

Suitable reactive derivative of the compound (II) includes an acid halide, an acid anhydride and an activated ester. The suitable example may be an acid chloride; an acid azide; a mixed acid anhydride with an acid such as substituted phosphoric acid (e.g., dialkylphosphoric acid, phenylphosphoric acid, diphenylphosphoric acid, dibenzylphosphoric acid, halogenated phosphoric acid, etc.), dialkylphosphorous acid, sulfurous acid, thiosulfuric acid, alkanesulfonic acid (e.g., methanesulfonic acid, ethanesulfonic acid, etc.), sulfuric acid, alkylcarbonic acid, aliphatic carboxylic acid (e.g., pivalic acid, pentanoic acid, isopentanoic acid, 2-ethylbutyric acid, trichloroacetic acid, etc.); aromatic carboxylic acid (e.g., benzoic acid, etc.); a symmetrical acid anhydride; an activated amide with imidazole, 4-substituted imidazole, dimethylpyrazole, triazole or tetrazole; an activated ester (e.g., cyanomethyl ester, methoxymethyl ester, dimethyliminomethyl[(CH₃)₂N⁺═CH—]ester, vinyl ester, propargyl ester, p-nitrophenyl ester, 2,4-dinitrophenyl ester, trichlorophenyl ester, pentachlorophenyl ester, mesylphenyl ester, phenylazophenyl ester, phenyl thioester, p-nitrophenyl thioester, p-cresyl thioester, carboxymethyl thioester, pyranyl ester, pyridinyl ester, piperidyl ester, 8-quinolyl thioester, etc.); or an ester with an N-hydroxy compound (e.g., N,N-dimethylhydroxylamine, 1-hydroxy-2-(1H)-pyridone, N-hydroxysuccinimide, N-hydroxybenzotriazole, N-hydroxyphthalimide, 1-hydroxy-6-chloro-1H-benzotriazole, etc.). These reactive derivatives can optionally be selected from them according to the kind of the compound (II) to be used.

The reaction is usually carried out in a conventional solvent such as water, acetone, dioxane, acetonitrile, chloroform, methylene chloride, ethylene dichloride, tetrahydrofuran, ethyl acetate, N,N-dimethylformamide, pyridine or any other organic solvents which do not adversely affect the reaction, or a mixture thereof.

When the compound (II) is used in free acid form or its salt form in the reaction, the reaction is preferably carried out in the presence of a conventional condensing agent such as N,N′-dicyclohexylcarbodiimide; N-cyclohexyl-N′-morpholinoethylcarbodiimide; N-cyclohexyl-N′-(4-diethylaminocyclohexyl)carbodiimide; N,N′-diisopropylcarbodiimide; N-ethyl-N′-(3-dimethylaminopropyl)carbodiimide; N,N-carbonyl-bis-(2-methylimidazole); pentamethyleneketene-N-cyclohexylimine; diphenylketene-N-cyclohexylimine; ethoxyacetylene; 1-alkoxy-1-chloroethylene; trialkyl phosphite; isopropyl polyphosphate; phosphorus oxychloride(phosphoryl chloride); phosphorus trichloride; thionyl chloride; oxalyl chloride; triphenylphosphine; 2-ethyl-7-hydroxybenzisoxazolium salt; 2-ethyl-5-(m-sulfophenyl)isoxazolium hydroxide intramolecular salt; 1-(p-chlorobenzenesulfonyloxy)-6-chloro-1H-benzotriazole; so-called Vilsmeier reagent prepared by the reaction of N,N-dimethylformamide with thionyl chloride, phosgene, phosphorus oxychloride, etc.; or the like.

The reaction may also be carried out in the presence of an organic or inorganic base such as an alkali metal bicarbonate, tri(lower)alkylamine, pyridine, N-(lower)alkylmorpholine, N,N-di(lower)alkylbenzylamine, or the like.

The reaction temperature is not critical, and the reaction is usually carried out under cooling to heating.

Process (2)

The compound (I)-1 or a salt thereof can be prepared by reacting the compound (IV) or its reactive derivative at the amino group, or a salt thereof with the compound (V) or its reactive derivative at the carboxy group, or a salt thereof.

This reaction can be carried out in the same manner as in the aforementioned Process (1), and therefore the reagents to be used and the reaction conditions (e.g., solvent, reaction temperature, etc.) can be referred to those of Process (1).

Process (3)

The compound (I)-3 or a salt thereof can be prepared by reacting the compound (VI) or a salt thereof with the compound (VII) or a salt thereof.

The reaction is usually carried out in a conventional solvent such as tetrahydrofuran, dioxane, toluene, methylene chloride, ethylene dichloride, chloroform, N,N-dimethylformamide, N,N-dimethylacetamide or any other organic solvents which do not adversely affect the reaction, or a mixture thereof.

The reaction temperature is not critical, and the reaction is usually carried out under cooling to warming.

Process (4)

The compound (I)-5 or a salt thereof can be prepared by subjecting the compound (I)-4 or a salt thereof to elimination reaction of the amino protective group.

Suitable method of this elimination reaction includes conventional one such as hydrolysis, reduction and the like.

(i) For Hydrolysis:

The hydrolysis is preferably carried out in the presence of a base or an acid including Lewis acid.

Suitable base includes an inorganic base and an organic base such as an alkali metal [e.g., sodium, potassium, etc.], an alkaline earth metal [e.g., magnesium, calcium, etc.], the hydroxide or carbonate or hydrogencarbonate thereof, trialkylamine [e.g., trimethylamine, triethylamine, etc.], picoline, 1,5-diazabicyclo[4.3.0]non-5-ene, or the like.

Suitable acid includes an organic acid [e.g., formic acid, acetic acid, propionic acid, trichloroacetic acid, trifluoroacetic acid, etc.], and an inorganic acid [e.g., hydrochloric acid, hydrobromic acid, sulfuric acid, hydrogen chloride, hydrogen bromide, etc.].

The elimination using Lewis acid such as trihaloacetic acid [e.g., trichloroacetic acid, trifluoroacetic acid, etc.], or the like is preferably carried out in the presence of cation trapping agents [e.g., anisole, phenol, etc.]. This reaction is usually carried out without solvent.

The reaction may be carried out in a conventional solvent such as water, alcohol (e.g., methanol, ethanol, isopropyl alcohol, etc.), tetrahydrofuran, dioxane, toluene, methylene chloride, ethylene dichloride, chloroform, N,N-dimethylformamide, N,N-dimethylacetamide or any other organic solvents which do not adversely affect the reaction, or a mixture thereof.

The reaction temperature is not critical and the reaction is usually carried out under cooling to warming.

(ii) For Reduction:

Reduction is carried out in a conventional manner, including chemical reduction and catalytic reduction.

Suitable reducing reagent to be used in chemical reduction are hydrides (e.g., hydrogen iodide, hydrogen sulfide, lithium aluminum hydride, sodium borohydride, sodium cyanoborohydride, etc.), or a combination of a metal (e.g., tin, zinc, iron, etc.) or metallic compound (e.g., chromium chloride, chromium acetate, etc.) and an organic acid or inorganic acid (e.g., formic acid, acetic acid, propionic acid, trifluoroacetic acid, p-toluenesulfonic acid, hydrochloric acid, hydrobromic acid, etc.).

Suitable catalysts to be used in catalytic reduction are conventional ones such as platinum catalysts (e.g., platinum plate, spongy platinum, platinum black, colloidal platinum, platinum oxide, platinum wire, etc.), palladium catalysts (e.g., spongy palladium, palladium black, palladium oxide, palladium on carbon, palladium hydroxide on carbon, colloidal palladium, palladium on barium sulfate, palladium on barium carbonate, etc.), nickel catalysts (e.g., reduced nickel, nickel oxide, Raney nickel, etc.), cobalt catalysts (e.g., reduced cobalt, Raney cobalt, etc.), iron catalysts (e.g., reduced iron, Raney iron, Ullman iron, etc.), and the like.

The reduction is usually carried out in a conventional solvent such as water, alcohol (e.g., methanol, ethanol, isopropyl alcohol, etc.), tetrahydrofuran, dioxane, toluene, methylene chloride, ethylene dichloride, chloroform, N,N-dimethylformamide, N,N-dimethylacetamide or any other organic solvents which do not adversely affect the reaction, or a mixture thereof.

Additionally, in case that the above-mentioned acids to be used in chemical reduction are in a liquid state, they can also be used as a solvent.

The reaction temperature of this reduction is not critical and the reaction is usually carried out under cooling to warming.

Process (5)

The compound (I)-7 or a salt thereof can be prepared by subjecting the compound (I)-6 or a salt thereof to elimination reaction of the amino protective group.

This reaction can be carried out in the same manner as in the aforementioned Process (4), and therefore the reagents to be used and the reaction conditions (e.g., solvent, reaction temperature, etc.) can be referred to those of Process (4).

Process (6)

The compound (I)-9 can be prepared by subjecting the compound (I)-8 to reduction using a suitable reducing agent.

Suitable reducing agents to be used in the reduction are hydrides (e.g., sodium borohydride, sodium cyanoborohydride, lithium aluminum hydride, etc.).

The reduction is usually carried out in a conventional solvent such as water, alcohol (e.g., methanol, ethanol, isopropyl alcohol, etc.), tetrahydrofuran, dioxane, toluene, methylene chloride, ethylene dichloride, chloroform, N,N-dimethylformamide, N,N-dimethylacetamide or any other organic solvents which do not adversely affect the reaction, or a mixture thereof.

The reaction temperature is not critical, and the reaction is usually carried out under cooling to warming.

Process (7)

The compound (I)-10 can be prepared by subjecting the compound (I)-9 to catalytic hydrogenation in the presence of an acid.

Suitable catalysts to be used in the catalytic hydrogenation are conventional ones such as platinum catalysts (e.g., platinum plate, spongy platinum, platinum black, colloidal platinum, platinum oxide, platinum wire, etc.), palladium catalysts (e.g., spongy palladium, palladium black, palladium oxide, palladium on carbon, palladium hydroxide on carbon, colloidal palladium, palladium on barium sulfate, palladium on barium carbonate, etc.), and the like.

Suitable acid to be used in the catalytic hydrogenation includes hydrochloric acid, hydrogen chloride, and the like.

The hydrogenation is usually carried out in a conventional solvent such as water, alcohol (e.g., methanol, ethanol, isopropyl alcohol, etc.), tetrahydrofuran, dioxane, toluene, methylene chloride, ethylene dichloride, chloroform, N,N-dimethylformamide, N,N-dimethylacetamide or any other organic solvents which do not adversely affect the reaction, or a mixture thereof.

The reaction temperature is not critical, and the reaction is usually carried out under cooling to warming.

Process (8)

The compound (I)-11 or a salt thereof can be prepared by reacting the compound (XXII) or a salt thereof with the compound (XXVI) or a salt thereof.

This reaction is generally carried out in the presence of an organic or inorganic base such as potassium tert-butoxide, sodium bicarbonate, sodium hydride, triethylamine, etc., and in a solvent such as N,N-dimethylformamide, chloroform, diethyl ether, dioxane, tetrahydrofuran, acetonitrile, etc., or any other organic solvents which do not adversely affect the reaction, or a mixture thereof.

The reaction temperature is not critical, and the reaction is usually carried out under cooling to warming.

Process (9)

The compound (I)-12 or a salt thereof can be prepared by reacting the compound (XXV) or a salt thereof with the compound (XXVII) or a salt thereof.

The reaction is usually carried out in the presence of a reducing agent such as sodium triacetoxyborohydride, sodium cyanoborohydride, etc., and in a conventional solvent such as chloroform, ethylene chloride, acetonitrile, diethyl ether, tetrahydrofuran, methanol, etc., or any other organic solvents which do not adversely affect the reaction, or a mixture thereof.

The reaction temperature is not critical, and the reaction is usually carried out under cooling to warming.

Process (10)

The compound (I)-13 or a salt thereof can be prepared by reacting the compound (XXVI) with the compound (XXVII) in the presence of an organic base such as triethylamine, pyridine, etc., and in a conventional solvent such as tetrahydrofuran, chloroform, diethyl ether, N,N-dimethylformamide, etc., or any other organic solvents which do not adversely affect the reaction, or a mixture thereof.

The reaction temperature is not critical, and the reaction is usually carried out under cooling to warming.

Process (11)

The compound (I)-14 or a salt thereof can be prepared by subjecting the compound (I)-13 or a salt thereof to elimination reaction of the amino protective group.

This reaction can be carried out in the same manner as the elimination reaction of the amino protective group in the aforementioned Process (4), and therefore the reagents to be used and the reaction conditions (e.g., solvent, reaction temperature, etc.) can be referred to those of Process (4).

Process (A)

The compound (IX)-1 or a salt thereof can be prepared by reacting the compound (VIII) or a salt thereof with the compound (VII) or a salt thereof.

This reaction can be carried out in the same manner as in the aforementioned Process (3), and therefore the reagents to be used and the reaction conditions (e.g., solvent, reaction temperature, etc.) can be referred to those of Process (3).

Process (B)

The compound (II) or a salt thereof can be prepared by subjecting the compound (IX) or a salt thereof to elimination reaction of the carboxy protective group.

Suitable method of this elimination reaction includes conventional one such as hydrolysis, reduction and the like.

This reaction can be carried out in the same manner as the elimination reaction of the amino protective group in the aforementioned Process (4), and therefore the reagents to be used and the reaction conditions (e.g., solvent, reaction temperature, etc.) can be referred to those of Process (4).

Process (C)

The compound (XI)-1 or a salt thereof can be prepared by reacting the compound (X) or its reactive derivative at the amino group, or a salt thereof with the compound (V) or its reactive derivative at the carboxy group, or a salt thereof.

This reaction can be carried out in the same manner as in the aforementioned Process (1), and therefore the reagents to be used and the reaction conditions (e.g., solvent, reaction temperature, etc.) can be referred to those of Process (1).

Process (D)

The compound (III) can be prepared by subjecting the compound (XI) to reduction.

Suitable method of the reduction is catalytic hydrogenation.

Suitable catalysts to be used in the catalytic hydrogenation are conventional ones such as platinum catalysts (e.g., platinum plate, spongy platinum, platinum black, colloidal platinum, platinum oxide, platinum wire, etc.), palladium catalysts (e.g., spongy palladium, palladium black, palladium oxide, palladium on carbon, palladium hydroxide on carbon, colloidal palladium, palladium on barium sulfate, palladium on barium carbonate, etc.), and the like.

The hydrogenation is usually carried out in a conventional solvent such as water, alcohol (e.g., methanol, ethanol, isopropyl alcohol, etc.), tetrahydrofuran, dioxane, toluene, methylene chloride, ethylene dichloride, chloroform, N,N-dimethylformamide, N,N-dimethylacetamide or any other organic solvents which do not adversely affect the reaction, or a mixture thereof.

The reaction temperature is not critical, and the reaction is usually carried out under cooling to warming.

Process (E)

The compound (XIV) or a salt thereof can be prepared by reacting the compound (XII) or its reactive derivative at the carboxy group, or a salt thereof with the compound (XIII) or its reactive derivative at the amino group, or a salt thereof.

This reaction can be carried out in the same manner as in the aforementioned Process (1), and therefore the reagents to be used and the reaction conditions (e.g., solvent, reaction temperature, etc.) can be referred to those of Process (1).

Process (F)

The compound (XV)-1 or a salt thereof can be prepared by reacting the compound (XIV) or a salt thereof with the compound (VII) or a salt thereof.

This reaction can be carried out in the same manner as in the aforementioned Process (3), and therefore the reagents to be used and the reaction conditions (e.g., solvent, reaction temperature, etc.) can be referred to those of Process (3).

Process (G)

The compound (IV) or a salt thereof can be prepared by subjecting the compound (XV) or a salt thereof to elimination reaction of the amino protective group.

This reaction can be carried out in the same manner as in the aforementioned Process (4), and therefore the reagents to be used and the reaction conditions (e.g., solvent, reaction temperature, etc.) can be referred to those of Process (4).

Process (H)

The compound (VI) or a salt thereof can be prepared by reacting the compound (XII) or its reactive derivative at the carboxy group, or a salt thereof with the compound (III) or its reactive derivative at the amino group, or a salt thereof.

This reaction can be carried out in the same manner as in the aforementioned Process (1), and therefore the reagents to be used and the reaction conditions (e.g., solvent, reaction temperature, etc.) can be referred to those of Process (1).

Process (I)

The compound (IX)-2 or the salt thereof can be prepared by reacting the compound (XVI) or a salt thereof with the compound (XVII) or a salt thereof.

This reaction is usually carried out in accordance with a conventional method.

This methylation is preferably carried out without a solvent, or in an any solvent which do not adversely affect the reaction, or a mixture thereof.

The reaction temperature is not critical, and the reaction is usually carried out under warming to heating.

Process (J)

The compound (XX) or the salt thereof can be prepared by reacting the compound (XVIII) or a salt thereof with the compound (XIX).

This reaction is usually carried out in accordance with a conventional method.

This reductive methylation is usually carried out in the presence of catalysts, and the suitable catalysts to be used in this reaction are conventional ones such as platinum catalysts (e.g., platinum plate, spongy platinum, platinum black, colloidal platinum, platinum oxide, platinum wire, etc.), palladium catalysts (e.g., spongy palladium, palladium black, palladium oxide, palladium on carbon, palladium hydroxide on carbon, colloidal palladium, palladium on barium sulfate, palladium on barium carbonate, etc.), and the like.

This reaction is preferably in a conventional solvent such as water, alcohol (e.g., methanol, ethanol, isopropyl alcohol, etc.), tetrahydrofuran, dioxane, toluene, methylene chloride, ethylene dichloride, chloroform, N,N-dimethylformamide, N,N-dimethylacetamide or any other organic solvents which do not adversely affect the reaction, or a mixture thereof.

The reaction temperature is not critical, and the reaction is usually carried out under cooling to warming.

Process (K)

The compound (IX)-3 can be synthesized by functional transformation of hydroxyl group to carboxyl group that comprises successive trifluoromethanesulfonylation and esterification, which is obvious to the person skilled in the organic chemistry, exemplified by the methods disclosed in e.g. Preparation 72 and Preparation 73 mentioned later or the similar manner thereby.

Process (L)

The compound (XXII) or the salt thereof can be prepared by reacting the compound (IV) or a salt thereof with the compound (XXI).

This reaction can be carried out in the same manner as in the aforementioned Process (10), and therefore the reagents to be used and the reaction conditions (e.g., solvent, reaction temperature, etc.) can be referred to those of Process (10).

Process (M)

The compound (XXIV) or a salt thereof can be prepared by reacting the compound (XXIII) or its reactive derivative at the amino group, or a salt thereof with the compound (II) or its reactive derivative at the carboxy group, or a salt thereof.

This reaction can be carried out in the same manner as in the aforementioned Process (1), and therefore the reagents to be used and the reaction conditions (e.g., solvent, reaction temperature, etc.) can be referred to those of Process (1).

Process (N)

The compound (XXV) or a salt thereof can be prepared by subjecting the compound (XXIV) or a salt thereof to elimination reaction of the amino protective group of the nitrogen atom on the pipirazine ring.

This reaction can be carried out in the same manner as in the aforementioned Process (4), and therefore the reagents to be used and the reaction conditions (e.g., solvent, reaction temperature, etc.) can be referred to those of Process (4).

Suitable salts of the starting compounds and their reactive derivatives in Processes (1) to (11) and (A) to (N) can be referred to the ones as exemplified for the compound (I).

The compounds obtained by the above processes can be isolated and purified by a conventional method such as pulverization, recrystallization, column chromatography, reprecipitation, or the like.

It is to be noted that the compound (I) and the other compounds may include one or more stereoisomer(s) such as optical isomer(s) and geometrical isomer(s) due to asymmetric carbon atom(s) and double bond(s), and all of such isomers and mixtures thereof are included within the scope of this invention.

The object compounds (I) and pharmaceutically acceptable salts thereof include solvates [e.g., enclosure compounds (e.g., hydrate, etc.)].

The object compounds (I) and pharmaceutically acceptable salts thereof possess a strong inhibitory activity on the secretion of Apo B.

Accordingly, the object compounds (I) and pharmaceutically acceptable salts thereof are useful as an Apo B secretion inhibitor.

The object compounds (I) and pharmaceutically acceptable salts thereof are useful as a medicament for the prophylaxis or treatment of diseases or conditions resulting from elevated circulating levels of Apo B such as hyperlipemia, hyperlipidemia, hyperlipoproteinemia, hypoalphalipoproteinemia, hypercholesterolemia, hypertriglyceridemia, atherosclerosis, pancreatitis, non-insulin dependent diabetes mellitus (NIDDM), obesity, coronary heart diseases, myocardial infarction, stroke, restenosis and Syndrome X.

The present invention therefore provides a method for inhibiting or decreasing Apo B secretion in a mammal, in particular in human, which comprises administering an Apo B secretion inhibiting or decreasing amount of a compound of formula (I) or a pharmaceutically acceptable salt thereof to the mammal.

The present invention also provides a method for preventing or treating diseases or conditions resulting from elevated circulating levels of Apo B in a mammal, in particular in human, which comprises administering an effective amount of a compound of formula (I) or a pharmaceutically acceptable salt thereof to the mammal.

The object compounds (I) and pharmaceutical acceptable salts thereof are also useful in reducing intestinal fat absorption and reducing food intake for the prophylaxis or treatment of obesity. Furthermore, the object compounds (I) and pharmaceutical acceptable salts thereof possess an inhibitory activity on the lipid transfer of microsomal triglyceride transfer protein (MTP).

In order to illustrate the usefulness of the object compound (I), the pharmacological test result of the compound (I) is shown in the following.

Test Compounds:

2-(dimethylamino)-4-methyl-N-(4-{[2-(2-pyridinyl)ethyl]amino}phenyl)benzamide (Example 42)

2-(4-methyl-1-piperidinyl)-N-(4-{[2-(2-pyridinyl)ethyl]amino}phenyl)nicotinamide (Example 54)

2-(dimethylamino)-4-methyl-N-(4-{[2-(1H-pyrazol-1-yl)ethyl]amino}phenyl)benzamide (Example 183)

6-methyl-2-(4-methyl-1-piperidinyl)-N-{4-[(1H-pyrazol-1-ylacetyl)amino]phenyl}nicotinamide (Example 193)

6-methyl-2-(4-methyl-1-piperidinyl)-N-(4-{[2-(2-pyridinyl)propanoyl]amino}phenyl)nicotinamide (Example 415)

N-(4-{[2-(6-amino-2-pyridinyl)ethyl]amino}phenyl)-4-chloro-2-(dimethylamino)benzamide (Example 435)

2-(dimethylamino)-4-ethyl-N-(4-{[2-(1H-pyrazol-1-yl)ethyl]amino}phenyl)benzamide (Example 473)

Test 1: Measurement of Inhibition of Apo B Secretion

HepG2 cells were seeded in Eagles medium containing 10% fetal calf serum (FCS) at a density of 30000 cells/well in 96-well plates and allowed to grow for 3 days before treatment. At this time, the medium was replaced with fresh medium containing 0.1% dimethyl sulfoxide (DMSO) and the indicated concentrations of a test compound. After 15-hour incubation, the amount of Apo B and Apo AI accumulated in the media was determined by ELISA.

The assay was carried out at ambient temperature. A flat bottomed micro ELISA plate (manufactured by Nunc) was coated with an anti Apo B monoclonal antibody solution (5 mg/ml in 0.05% carbonate buffer, pH 9.6) by adding the antibody solution at a volume of 100 μl per well. After 1-hour incubation on a plate mixer, the unbound materials were removed by washing the well 3 times with a washing buffer (phosphate buffered saline, pH 7.2 containing 0.1% bovine serum albumin and 0.05% Tween-20). Then 20 μl of a solution of the test compound (dissolved in the culture medium) and 100 μl of a solution of peroxidase coupled anti Apo B antibody were added. After 1-hour incubation on a plate mixer, washing was performed 3 times to remove the unbound materials. A freshly prepared substrate solution (2.5 mg/ml ortho-phenylene diamine and 0.018% H₂O₂ in 0.11 M Na₂HPO₄-0.044 M sodium citrate buffer, pH 5.4) at a volume of 200 μl was then added to each well. After 20-minute incubation, the enzyme reaction was terminated by adding 50 μl of 0.5 M sulfuric acid. Absorbance of each well was determined at 490 nm using a microplate reader. Apo B concentration was calculated from a standard curve generated from purified Apo B standard that was run in parallel in the same plate. Inhibition of Apo B secretion by the test compound was calculated taking 0.1% DMSO treated cells as controls.

Measurement of Apo AI was performed similar to that of Apo B, except for diluting the sample 11-fold with a dilution buffer (phosphate buffered saline, pH 7.2 containing 0.5% bovine serum albumin and 0.05% Tween-20).

Apo B secretion inhibitors are identified as compounds that decrease Apo B secretion without affecting the secretion of Apo AI.

Test Results:

TABLE 1

| | Inhibition of Apo B |
|---|---|
| Test compound | secretion at 10⁻⁸ M |
| (Example No.) | (%) |
| 42 | 85.8 |
| 54 | 86.3 |
| 183 | 81.2 |
| 193 | 71.5 |
| 415 | 76.2 |
| 435 | 85.9 |
| 473 | 75.7 |

Test 2: Lipid Lowering Effect on ddY-Mice

Male ddY-mice were housed in temperature- and humidity-controlled rooms and fed with laboratory chow. The animals were randomized according to their body weight and food was deprived about 16 hours before experiment. Baseline blood sample was collected from the retro orbital venous plexus then the animals were orally dosed with drugs in olive oil (10 ml/kg). For control group, 10 ml/kg of olive oil was loaded orally. Blood samples were drawn at 2 hours after drug administration for the measurement of triglyceride (TG) elevation. Plasma TG was determined by conventional enzyme method (The triglyceride E-test Wako).

Lipid lowering effects were shown in percent of the TG increase in drug treated group, relative to the TG increase in control group.

Lipid lowering effect(%)=(TG increase in drug treated group/TG increase in control group)×100

TABLE 2

| Test compound | Dose | Lipid lowering |
|---|---|---|
| (Example No.) | (mg/kg) | effect (%) |
| 42 | 0.32 | 33 |
| 54 | 0.32 | 28 |
| 183 | 0.32 | 27 |
| 435 | 0.32 | 52 |

For therapeutic administration, the object compound (I) of the present invention and pharmaceutically acceptable salts thereof are used in the form of a conventional pharmaceutical preparation in admixture with a conventional pharmaceutically acceptable carrier such as an organic or inorganic solid or liquid excipient which is suitable for oral, parenteral or external administration. The pharmaceutical preparation may be compounded in a solid form such as granule, capsule, tablet, dragee, suppository or ointment, or in a liquid form such as solution, suspension or emulsion for injection, intravenous drip, ingestion, eye drop, endermism, inhalation, etc. If needed, there may be included in the above preparation auxiliary substance such as stabilizing agent, wetting or emulsifying agent, buffer or any other commonly used additives.

The effective ingredient may usually be administered in a unit dose of 0.01 mg/kg to 100 mg/kg, preferably 0.1 mg/kg to 10 mg/kg, 1 to 4 times a day. However, the above dosage may be increased or decreased according to age, body weight and conditions of the patient or administering method.

Suitable mammal to which the object compounds (I) and pharmaceutical acceptable salts thereof or above preparations are applied, includes a human being, a companion animal such as a dog and a cat, livestock such as a cow and a pig, and the like.

The object compounds (I) and pharmaceutical acceptable salts thereof may, if desired, be administered with one or more therapeutic agents and formulated for administration by any convenient route in a conventional manner. Appropriate doses will be readily appreciated by those skilled in the art. For example, the object compounds (I) and pharmaceutical acceptable salts thereof may be administered in combination with an HMG CoA reductase inhibitor. The object compounds (I) and pharmaceutical acceptable salts thereof may be also administered in combination with a known anti-obesity agent, for example, β₃-adrenergic receptor agonist, a cholecystokinin-A agonist, a monoamine reuptake inhibitor, a sympathomimetic agent, a serotoninergic agent, a dopamine agonist, a melanocyte-stimulating hormone receptor agonist or mimetic, a melanocyte-stimulating hormone receptor analog, a cannabinoid receptor antagonist, a melanin concentrating hormone antagonist, leptin, a leptin analog, a leptin receptor agonist, a galanin antagonist, a lipase inhibitor, a bombesin agonist, a Neuropeptide-Y antagonist, a thyromimetic agent, dehydroepiandrosterone or an analog thereof, a glucocorticoid receptor agonist or antagonist, an orexin receptor antagonist, a urocortin binding protein antagonist, a glucagon-like peptide-1 receptor agonist, a ciliary neurotrophic factor, a human agouti-related protein antagonist, and the like, for the prophylaxis or treatment of obesity.

The following Preparations and Examples are given for the purpose of illustrating the present invention in detail.

Preparation 1

To a suspension of 5-nitroindoline (3.28 g), 2-pyridylacetic acid hydrochloride (3.82 g), 1-[3-(dimethylamino)propyl]-3-ethylcarbodiimide hydrochloride (4.22 g) and 1-hydroxybenzotriazole hydrate (3.37 g) in dichloromethane (100 ml) was added dropwise triethylamine (4.45 g) at ambient temperature and the resultant solution was stirred at ambient temperature for 18 hours. The mixture was poured into water and the separated organic layer was washed with water and brine, dried over magnesium sulfate and evaporated in vacuo. The residue was purified by column chromatography on silica gel eluting with ethyl acetate to give 5-nitro-1-(2-pyridinylacetyl)indoline (3.58 g) as a yellow solid.

¹H-NMR(DMSO-d₆): δ 3.26(2H, t, J=8.5 Hz), 4.10(2H, s), 4.33(2H, t, J=8.5 Hz), 7.25-7.35(1H, m), 7.38(1H, d, J=7.8 Hz), 7.75-7.9(1H, m), 8.1-8.2(3H, m), 8.50-8.55(1H, m) APCI-MS(m/z): 284(M+H)⁺

Preparation 2

To a solution of 5-nitro-1-(2-pyridinylacetyl)indoline (3.54 g) in methanol (50 ml) and tetrahydrofuran (50 ml) was added 10% palladium on carbon (50% wet, 3.5 g) and the mixture was hydrogenated under hydrogen at atmospheric pressure for 5 hours. After removing the palladium on carbon by filtration, the filtrate was evaporated in vacuo. The residue was purified by column chromatography on silica gel eluting with ethyl acetate: methanol (10:1 v/v) to give 1-(2-pyridinylacetyl)-5-indolinamine (2.16 g) as pale brown crystals.

¹H-NMR(DMSO-d₆): δ 3.01(2H, t, J=8.4 Hz), 3.92(2H, s), 4.11(2H, t, J=8.4 Hz), 4.84(2H, br s), 6.32(1H, d, J=8.4 Hz), 6.45(1H, s), 7.1-7.2(1H, m), 7.33(1H, d, J=7.8 Hz), 7.7-7.85(2H, m), 8.48(1H, d, J=4.0 Hz) APCI-MS(m/z): 254(M+H)⁺

### EXAMPLE 1

1-[3-(Dimethylamino)propyl]-3-ethylcarbodiimide (0.19 g) was added to a solution of 1-(2-pyridinylacetyl)-5-indolinamine (0.25 g), 2-(1-pyrrolidinyl)benzoic acid (0.23 g), 1-hydroxybenzotriazole hydrate (0.16 g) and 4-dimethylaminopyridine (6 mg) in N,N-dimethylformamide (5 ml) under ice-cooling and the mixture was stirred at ambient temperature for 18 hours. The reaction mixture was poured into a mixture of ethyl acetate and water. The separated organic layer was washed with water, dried over magnesium sulfate and evaporated in vacuo. The residue was triturated with ethyl acetate to give N-[1-(2-pyridinylacetyl)-2,3-dihydro-1H-indol-5-yl]-2-(1-pyrrolidinyl)benzamide (0.27 g).

¹H-NMR(DMSO-d₆): δ 1.75-1.95(4H, m), 3.08-3.29(4H, m), 3.16(2H, t, J=8.4 Hz), 4.00(2H, s), 4.21(2H, t, J=8.4 Hz), 6.65-6.82(2H, m), 7.21-7.47(5H, m), 7.69(1H, s), 7.76(1H, dt, J=1.8 Hz, 7.6 Hz), 7.96(1H, d, J=8.7 Hz), 8.50(1H, dd, J=0.9 Hz, 4.2 Hz), 10.27(1H, s) (−)ESI-MS: 425(M−H)⁻

### EXAMPLE 2

### 2-(1-Piperidinyl)-N-[1-(2-pyridinylacetyl)-2,3-dihydro-1H-indol-5-yl]benzamide

The title compound was obtained in a similar manner as in Example 1 from 1-(2-pyridinylacetyl)-5-indolinamine and 2-(1-piperidinyl)benzoic acid.

¹H-NMR(DMSO-d₆): δ 1.45-1.76(6H, m), 2.87-3.01(4H, m), 3.19(2H, t, J=8.4 Hz), 4.01(2H, s), 4.23(2H, t, J=8.4 Hz), 7.16-7.57(6H, m), 7.72-7.90(3H, m), 8.02(1H, d, J=8.6 Hz), 8.48-8.55(1H, m), 11.68(1H, s) (+)APCI-MS: 441(M+H)⁺

### EXAMPLE 3

### 2-(3,6-Dihydro-1(2H)-pyridinyl)-N-[1-(2-pyridinylacetyl)-2,3-dihydro-1H-indol-5-yl]benzamide

The title compound was obtained in a similar manner as in Example 1 from 1-(2-pyridinylacetyl)-5-indolinamine and 2-(3,6-dihydro-1(2H)-pyridinyl)benzoic acid.

¹H-NMR(DMSO-d₆): δ 2.21-2.37(2H, m), 3.07-3.27(4H, m), 3.42-3.54(2H, m), 4.00(2H, s), 4.22(2H, t, J=8.4 Hz), 5.77-5.97(2H, m), 7.18-7.44(5H, m), 7.46-7.60(1H, m), 7.67-7.82(2H, m), 7.89(1H, dd, J=1.4 Hz, 7.6 Hz), 7.98(1H, d, J=8.6 Hz), 8.47-8.55(1H, m), 11.95(1H, s) (+)ESI-MS: 439(M+H)⁺, 461(M+Na)⁺

### EXAMPLE 4

### 2-(4-Methyl-1-piperidinyl)-N-[1-(2-pyridinylacetyl)-2,3-dihydro-1H-indol-5-yl]benzamide

The title compound was obtained in a similar manner as in Example 1 from 1-(2-pyridinylacetyl)-5-indolinamine and 2-(4-methyl-1-piperidinyl)benzoic acid.

¹H-NMR(DMSO-d₆): δ 0.93(3H, d, J=6.0 Hz), 1.21-1.62(3H, m), 1.62-1.80(2H, m), 2.67-2.88(2H, m), 3.05-3.27(4H, m), 4.01(2H, s), 4.23(2H, t, J=8.4 Hz), 7.15-7.57(6H, m), 7.70-7.90(3H, m), 8.02(1H, d, J=8.6 Hz), 8.47-8.57(1H, m), 11.63(1H, s) (+)ESI-MS: 455(M+H)⁺, 477(M+Na)⁺

Preparation 3

A mixture of methyl 4-methyl-2-(trifluoromethanesulfonyloxy)benzoate (5.0 g) and pyrrolidine (4.2 ml) in acetonitrile (15.0 ml) was stirred under reflux for 20 hours. The solvent was removed by concentration. The residue was purified by column chromatography on silica gel using a mixture of hexane and ethyl acetate (9:1 v/v) as an eluant. The eluted fractions containing the desired product were collected and evaporated in vacuo to give methyl 4-methyl-2-(1-pyrrolidinyl)benzoate (2.07 g).

¹H-NMR(DMSO-d₆): δ 1.83-1.90(4H, m), 2.26(3H, s), 3.09-3.16(4H, m), 3.76(3H, s), 6.50(1H, dd, J=0.8 Hz, 7.9 Hz), 6.61(1H, d, J=0.8 Hz), 7.33(1H, d, J=7.9 Hz) (+)APCI-MS: 220(M+H)⁺

Preparation 4

A mixture of methyl 4-methyl-2-(1-pyrrolidinyl)benzoate (2.0 g) and sodium hydroxide (1.1 g) in a mixture of methanol (30 ml) and water (7.3 ml) was stirred under reflux for 24 hours. The solvent was removed by concentration. To the residue was added a mixture of ethyl acetate, tetrahydrofuran and water and the mixture was adjusted to pH 5.5 with 6N-hydrochloric acid. The separated organic layer was dried over magnesium sulfate and evaporated in vacuo. The residue was triturated with diisopropyl ether to give 4-methyl-2-(1-pyrrolidinyl)benzoic acid (1.48 g).

¹H-NMR(DMSO-d₆): δ 1.81-1.99(4H, m), 2.29(3H, s), 3.08-3.26(4H, m), 6.66(1H, d, J=7.8 Hz), 6.82(1H, s), 7.50(1H, d, J=7.8 Hz), 13.66(1H, s) (−)ESI-MS: 204 (M−H)⁻

### EXAMPLE 5

### 4-Methyl-N-[1-(2-pyridinylacetyl)-2,3-dihydro-1H-indol-5-yl]-2-(1-pyrrolidinyl)benzamide

The title compound was obtained in a similar manner as in Example 1 from 1-(2-pyridinylacetyl)-5-indolinamine and 4-methyl-2-(1-pyrrolidinyl)benzoic acid.

¹H-NMR(DMSO-d₆): δ 1.72-1.94(4H, m), 2.28(3H, s), 3.06-3.29(6H, m), 4.00(2H, s), 4.21(2H, t, J=8.3 Hz), 6.55(1H, d, J=7.7 Hz), 6.60(1H, s), 7.19(1H, d, J=7.7 Hz), 7.23-7.46(3H, m), 7.69(1H, s), 7.71-7.82(1H, m), 7.96(1H, d, J=8.7 Hz), 8.46-8.55(1H, m), 10.23(1H, s) (−)ESI-MS: 439(M−H)⁻

Preparation 5

### Benzyl 4-methyl-2-(1-piperidinyl)benzoate

The title compound was obtained in a similar manner as in Preparation 3 from benzyl 4-methyl-2-(trifluoromethanesulfonyloxy)benzoate and piperidine.

¹H-NMR(DMSO-d₆): δ 1.38-1.60(6H, m), 2.29(3H, s), 2.82-2.93(4H, m), 5.28(2H, s), 6.78(1H, d, J=8.0 Hz), 6.87(1H, s), 7.29-7.55(6H, m)

Preparation 6

To a mixture of benzyl 4-methyl-2-(1-piperidinyl)benzoate (5.6 g) in methanol (60 ml) was added 10% palladium on carbon (2.0 g, 50% wet). The reaction mixture was stirred at ambient temperature for 5 hours under hydrogen atmosphere. The catalyst was filtered off and the solvent was removed by concentration. The residue was triturated with a mixture of hexane and diisopropyl ether to give 4-methyl-2-(1-piperidinyl)benzoic acid (3.52 g).

¹H-NMR(DMSO-d₆): δ 1.54-1.83(6H, m), 2.38(3H, s), 2.96-3.10(4H, m), 7.25(1H, d, J=8.0 Hz), 7.56(1H, s), 7.92(1H, d, J=8.0 Hz), 18.13(1H, s) (−)ESI-MS: 218(M−H)⁻

### EXAMPLE 6

### 4-Methyl-2-(1-piperidinyl)-N-[1-(2-pyridinylacetyl)-2,3-dihydro-1H-indol-5-yl]benzamide

The title compound was obtained in a similar manner as in Example 1 from 1-(2-pyridinylacetyl)-5-indolinamine and 4-methyl-2-(1-piperidinyl)benzoic acid.

¹H-NMR(DMSO-d₆): δ 1.45-1.77(6H, m), 2.35(3H, s), 2.86-3.00(4H, m), 3.18(2H, t, J=8.4 Hz), 4.01(2H, s), 4.23(2H, t, J=8.4 Hz), 7.05(1H, d, J=8.0 Hz), 7.17(1H, s), 7.23-7.32(1H, m), 7.32-7.46(2H, m), 7.71-7.87(3H, m), 8.02(1H, d, J=8.7 Hz), 8.47-8.54(1H, m), 11.90(1H, s) (+)APCI-MS: 455(M+H)⁺

Preparation 7

### Benzyl 4-methyl-2-(4-methyl-1-piperidinyl)benzoate

The title compound was obtained in a similar manner as in Preparation 3 from benzyl 4-methyl-2-(trifluoromethanesulfonyloxy)benzoate and 4-methylpiperidine.

¹H-NMR(DMSO-d₆): δ 0.87(3H, d, J=6.2 Hz), 1.04-1.27(2H, m), 1.27-1.48(1H, m), 1.48-1.62(2H, m), 2.29(3H, s), 2.54-2.71(2H, m), 3.08-3.22(2H, m), 5.27(2H, s), 6.78(1H, d, J=8.0 Hz), 6.87(1H, s), 7.30-7.56(6H, m)

Preparation 8

### 4-Methyl-2-(4-methyl-1-piperidinyl)benzoic acid

The title compound was obtained in a similar manner as in Preparation 6 from benzyl 4-methyl-2-(4-methyl-1-piperidinyl)benzoate.

¹H-NMR(DMSO-d₆): δ 1.00(3H, d, J=6.4 Hz), 1.20-1.45(2H, m), 1.54-1.77(1H, m), 1.77-1.73(2H, m), 2.38(3H, s), 2.94-3.17(4H, m), 7.24(1H, d, J=8.0 Hz), 7.57(1H, s), 7.92(1H, d, J=8.0 Hz) (+)ESI-MS: 234(M+H)⁺

### EXAMPLE 7

### 4-Methyl-2-(4-methyl-1-piperidinyl)-N-[1-(2-pyridinylacetyl)-2,3-dihydro-1H-indol-5-yl]benzamide

The title compound was obtained in a similar manner as in Example 1 from 1-(2-pyridinylacetyl)-5-indolinamine and 4-methyl-2-(4-methyl-1-piperidinyl)benzoic acid.

¹H-NMR(DMSO-d₆): δ 0.95(3H, d, J=6.0 Hz), 1.18-1.65(3H, m), 1.65-1.80(2H, m), 2.34(3H, s), 2.69-2.86(2H, m), 3.04-3.25(4H, m), 4.01(2H, s), 4.23(2H, t, J=8.4 Hz), 7.04(1H, d, J=8.0 Hz), 7.16(1H, s), 7.24-7.33(1H, m), 7.33-7.43(2H, m), 7.71-7.84(3H, m), 8.02(1H, d, J=8.6 Hz), 8.47-8.54(1H, m), 11.85(1H, s) (+)ESI-MS: 469(M+H)⁺, 491(M+Na)⁺

Preparation 9

### Benzyl 2-(4,4-dimethyl-1-piperidinyl)-4-methylbenzoate

The title compound was obtained in a similar manner as in Preparation 3 from benzyl 4-methyl-2-(trifluoromethanesulfonyloxy)benzoate and 4,4-dimethylpiperidine.

¹H-NMR(DMSO-d₆): δ 0.89(6H, s), 1.32(4H, t, J=5.5 Hz), 2.29(3H, s), 2.88(4H, t, J=5.5 Hz), 5.27(2H, s), 6.78(1H, d, J=7.9 Hz), 6.91(1H, s), 7.30-7.54(6H, m)

Preparation 10

### 2-(4,4-Dimethyl-1-piperidinyl)-4-methylbenzoic acid

The title compound was obtained in a similar manner as in Preparation 6 from benzyl 2-(4,4-dimethyl-1-piperidinyl)-4-methylbenzoate.

¹H-NMR(DMSO-d₆): δ 1.07(6H, s), 7.56(4H, t, J=5.6 Hz), 2.39(3H, s), 3.03(4H, t, J=5.6 Hz), 7.24(1H, d, J=7.9 Hz), 7.71(1H, s), 7.92(1H, d, J=7.9 Hz) (−)ESI-MS: 246(M−H)⁻

### EXAMPLE 8

### 2-(4,4-Dimethyl-1-piperidinyl)-4-methyl-N-[1-(2-pyridinylacetyl)-2,3-dihydro-1H-indol-5-yl]benzamide

The title compound was obtained in a similar manner as in Example 1 from 1-(2-pyridinylacetyl)-5-indolinamine and 2-(4,4-dimethyl-1-piperidinyl)-4-methylbenzoic acid.

¹H-NMR(DMSO-d₆): δ 0.98(6H, s), 1.45-1.59(4H, m), 2.35(3H, s), 2.87-3.00(4H, m), 3.17(2H, t, J=8.4 Hz), 4.01(2H, s), 4.23(2H, t, J=8.4 Hz), 7.04(1H, d, J=8.0 Hz), 7.21-7.33(2H, m), 7.33-7.45(2H, m), 7.71-7.85(3H, m), 8.02(1H, d, J=8.6 Hz), 8.48-8.54(1H, m), 11.92(1H, s) (+)ESI-MS: 483(M+H)⁺, 505(M+Na)⁺

Preparation 11

### Benzyl 4-methyl-2-(4-morpholinyl)benzoate

The title compound was obtained in a similar manner as in Preparation 3 from benzyl 4-methyl-2-(trifluoromethanesulfonyloxy)benzoate and morpholine.

¹H-NMR(DMSO-d₆): δ 2.31(3H, s), 2.83-2.96(4H, m), 3.52-3.64(4H, m), 5.28(2H, s), 6.85(1H, d, J=8.0 Hz), 6.90(1H, s), 7.30-7.50(5H, m), 7.58(1H, d, J=8.0 Hz)

Preparation 12

### 4-Methyl-2-(4-morpholinyl)benzoic acid

The title compound was obtained in a similar manner as in Preparation 6 from benzyl 4-methyl-2-(4-morpholinyl)benzoate.

¹H-NMR(DMSO-d₆): δ 2.38(3H, s), 2.98-3.10(4H, m), 3.73-3.86(4H, m), 7.20(1H, d, J=8.0 Hz), 7.50(1H, s), 7.88(1H, d, J=8.0 Hz), 16.41(1H, s) (−)ESI-MS: 220(M−H)⁻

### EXAMPLE 9

### 4-Methyl-2-(4-morpholinyl)-N-[1-(2-pyridinylacetyl)-2,3-dihydro-1H-indol-5-yl]benzamide

The title compound was obtained in a similar manner as in Example 1 from 1-(2-pyridinylacetyl)-5-indolinamine and 4-methyl-2-(4-morpholinyl)benzoic acid.

¹H-NMR(DMSO-d₆): δ 2.35(3H, s), 2.89-3.04(4H, m), 3.18(2H, t, J=8.3 Hz), 3.65-3.80(4H, m), 4.01(2H, s), 4.22(2H, t, J=8.3 Hz), 7.03(1H, d, J=8.1 Hz), 7.12(1H, s), 7.23-7.33(1H, m), 7.37(1H, d, J=7.7 Hz), 7.43-7.53(1H, m), 7.65-7.84(3H, m), 8.02(1H, d, J=8.7 Hz), 8.47-8.54(1H, m), 11.20(1H, s) (+)APCI-MS: 457(M+H)⁺

Preparation 13

### Benzyl 4-methyl-2-(4-methyl-1-piperazinyl)benzoate

The title compound was obtained in a similar manner as in Preparation 3 from benzyl 4-methyl-2-(trifluoromethanesulfonyloxy)benzoate and 1-methylpiperazine.

¹H-NMR(DMSO-d₆): δ 2.15(3H, s), 2.25-2.39(4H, m), 2.30(3H, s), 2.86-2.97(4H, m), 5.27(2H, s), 6.81(1H, d, J=8.0 Hz), 6.88(1H, s), 7.31-7.50(5H, m), 7.53(1H, d, J=8.0 Hz)

Preparation 14

### 4-Methyl-2-(4-methyl-1-piperazinyl)benzoic acid

The title compound was obtained in a similar manner as in Preparation 6 from benzyl 4-methyl-2-(4-methyl-1-piperazinyl)benzoate.

¹H-NMR(DMSO-d₆): δ 2.37(3H, s), 2.46(3H, s), 2.70-2.94(4H, m), 3.06-3.22(4H, m), 7.16(1H, d, J=7.9 Hz), 7.39(1H, s), 7.86(1H, d, J=7.9 Hz), 14.51-17.40(1H, br) (−)ESI-MS: 233(M−H)⁻

### EXAMPLE 10

### 4-Methyl-2-(4-methyl-1-piperazinyl)-N-[1-(2-pyridinylacetyl)-2,3-dihydro-1H-indol-5-yl]benzamide

The title compound was obtained in a similar manner as in Example 1 from 1-(2-pyridinylacetyl)-5-indolinamine and 4-methyl-2-(4-methyl-1-piperazinyl)benzoic acid.

¹H-NMR(DMSO-d₆): δ 2.20(3H, s), 2.35(3H, s), 2.40-2.57(4H, m), 2.90-3.04(4H, m), 3.18(2H, t, J=8.3 Hz), 4.01(2H, s), 4.23(2H, t, J=8.3 Hz), 7.03(1H, d, J=8.0 Hz), 7.14(1H, s), 7.28(1H, dd, J=5.1 Hz, 6.8 Hz), 7.33-7.48(2H, m), 7.70-7.85(3H, m), 8.02(1H, d, J=8.7 Hz), 8.47-8.55(1H, m), 11.44(1H, s) (+)APCI-MS: 470(M+H)⁺

Preparation 15

### Benzyl 4-methyl-2-(4-thiomorpholinyl)benzoate

The title compound was obtained in a similar manner as in Preparation 3 from benzyl 4-methyl-2-(trifluoromethanesulfonyloxy)benzoate and thiomorpholine.

¹H-NMR(DMSO-d₆): δ 2.31(3H, s), 2.55-2.67(4H, m), 3.11-3.22(4H, m), 5.29(2H, s), 6.87(1H, d, J=8.0 Hz), 6.95(1H, s), 7.31-7.52(5H, m), 7.56(1H, d, J=8.0 Hz)

Preparation 16

### 4-Methyl-2-(4-thiomorpholinyl)benzoic acid

The title compound was obtained in a similar manner as in Preparation 6 from benzyl 4-methyl-2-(4-thiomorpholinyl)benzoate.

¹H-NMR(DMSO-d₆): δ 2.38(3H, s), 2.79-2.92(4H, m), 3.18-3.32(4H, m), 7.21(1H, d, J=8.0 Hz), 7.50(1H, s), 7.89(1H, d, J=8.0 Hz), 16.43(1H, s) (−)ESI-MS: 236(M−H)⁻

### EXAMPLE 11

### 4-Methyl-N-[1-(2-pyridinylacetyl)-2,3-dihydro-1H-indol-5-yl]-2-(4-thiomorpholinyl)benzamide

The title compound was obtained in a similar manner as in Example 1 from 1-(2-pyridinylacetyl)-5-indolinamine and 4-methyl-2-(4-thiomorpholinyl)benzoic acid.

¹H-NMR(DMSO-d₆): δ 2.35(3H, s), 2.68-2.83(4H, m), 3.10-3.30(6H, m), 4.01(2H, s), 4.23(2H, t, J=8.4 Hz), 7.03(1H, d, J=7.9 Hz), 7.12(1H, s), 7.23-7.50(3H, m), 7.68(1H, d, J=7.9 Hz), 7.71-7.84(2H, m), 8.02(1H, d, J=8.6 Hz), 8.47-8.55(1H, m), 11.14(1H, s) (+)ESI-MS: 473(M+H)⁺, 495(M+Na)⁺

Preparation 17

OXONE® (potassium peroxymonosulfate) (2.9 g) was added to a mixture of 4-methyl-2-(4-thiomorpholinyl)benzoic acid (0.5 g) and tetra-n-butylammonium hydrogensulfate (0.14 g) in a mixture of ethyl acetate (7.5 ml) and water (17.5 ml) and the mixture was stirred at 30° C. for 5 hours. The mixture was extracted with ethyl acetate. The extract layer was washed with water, dried over magnesium sulfate and evaporated in vacuo. The residue was triturated with diisopropyl ether to give 2-(1,1-dioxido-4-thiomorpholinyl)-4-methylbenzoic acid (0.18 g).

¹H-NMR(DMSO-d₆): δ 2.33(3H, s), 3.21-3.37(4H, m), 3.37-3.53(4H, m), 6.99(1H, d, J=7.9 Hz), 7.18(1H, s), 7.71(1H, d, J=7.9 Hz), 13.33(1H, s) (−)ESI-MS: 268(M−H)⁻

### EXAMPLE 12

### 2-(1,1-Dioxido-4-thiomorpholinyl)-4-methyl-N-[1-(2-pyridinylacetyl)-2,3-dihydro-1H-indol-5-yl]benzamide

The title compound was obtained in a similar manner as in Example 1 from 1-(2-pyridinylacetyl)-5-indolinamine and 2-(1,1-dioxido-4-thiomorpholinyl)-4-methylbenzoic acid.

¹H-NMR(DMSO-d₆): δ 2.34(3H, s), 3.08-3.26(6H, m), 3.36-3.50(4H, m), 4.01(2H, s), 4.23(2H, t, J=8.4 Hz), 6.99(1H, d, J=7.9 Hz), 7.09(1H, s), 7.23-7.33(1H, m), 7.33-7.52(3H, m), 7.70-7.85(2H, m), 8.01(1H, d, J=8.7 Hz), 8.46-8.56(1H, m), 10.36(1H, s) (+)ESI-MS: 505(M+H)⁺, 527(M+Na)⁺

Preparation 18

### Benzyl 2-(hexahydro-1H-azepin-1-yl)-4-methylbenzoate

The title compound was obtained in a similar manner as in Preparation 3 from benzyl 4-methyl-2-(trifluoromethanesulfonyloxy)benzoate and hexamethyleneimine.

1H-NMR(DMSO-d₆): δ 1.41-1.55(4H, m), 1.55-1.74(4H, m), 2.26(3H, s), 3.12-3.27(4H, m), 5.26(2H, s), 6.55(1H, d, J=7.5 Hz), 6.77(1H, s), 7.30-7.50(6H, m)

Preparation 19

### 2-(Hexahydro-1H-azepin-1-yl)-4-methylbenzoic acid

The title compound was obtained in a similar manner as in Preparation 6 from benzyl 2-(hexahydro-1H-azepin-1-yl)-4-methylbenzoate.

¹H-NMR(DMSO-d₆): δ 1.61-1.91(8H, m), 2.37(3H, s), 3.13-3.27(4H, m), 7.20(1H, d, J=8.0 Hz), 7.48(1H, s), 7.87(1H, d, J=8.0 Hz), 18.19(1H, s) (−)ESI-MS: 232(M−H)⁻

### EXAMPLE 13

2-(Hexahydro-1H-azepin-1-yl)-4-methyl-N-[1-(2-pyridinylacetyl)-2,3-dihydro-1H-indol-5-yl]benzamide

The title compound was obtained in a similar manner as in Example 1 from 1-(2-pyridinylacetyl)-5-indolinamine and 2-(hexahydro-1H-azepin-1-yl)-4-methylbenzoic acid.

¹H-NMR(DMSO-d₆): δ 1.52-1.65(4H, m), 1.65-1.84(4H, m), 2.31(3H, s), 3.08-3.29(6H, m), 4.01(2H, s), 4.22(2H, t, J=8.3 Hz), 6.84(1H, d, J=7.6 Hz), 7.01(1H, s), 7.24-7.43(3H, m), 7.51(1H, d, J=7.8 Hz), 7.70-7.83(2H, m), 7.99(1H, d, J=8.7 Hz), 8.47-8.54(1H, m), 11.23(1H, s) (+)ESI-MS: 469(M+H)⁺, 491(M+Na)⁺

Preparation 20

A mixture of 2-fluoro-4-(trifluoromethyl)benzonitrile (5.0 g) and piperidine (7.8 ml) in acetonitrile (25.0 ml) was stirred under reflux for 18 hours. The solvent was removed by concentration. To the residue was added a mixture of ethyl acetate and water, and the mixture was adjusted to pH 2 with 6N-hydorochloric acid. The separated organic layer was washed with water, dried over magnesium sulfate and evaporated in vacuo to give 2-(1-piperidinyl)-4-(trifluoromethyl)-benzonitrile (6.7 g).

¹H-NMR(DMSO-d₆): δ 2.50-2.77(6H, m), 3.16-3.27(4H, m), 7.30-7.41(2H, m), 7.92(1H, d, J=8.5 Hz)

Preparation 21

A mixture of 2-(1-piperidinyl)-4-(trifluoromethyl)benzonitrile (6.7 g) and sodium hydroxide (2.1 g) in ethylene glycol (27 ml) was stirred at 180° C. for 6 hours. After the mixture was added to water (27 ml) at 80° C., the mixture was stirred at 80° C. for 1 hour. The reaction mixture was poured into a mixture of ethyl acetate and water, and the mixture was adjusted to pH 3 with 6N-hydrochloric acid. The separated organic layer was washed with water, dried over magnesium sulfate and evaporated in vacuo. The residue was triturated with diisopropyl ether to give 2-(1-piperidinyl)-4-(trifluoromethyl)benzoic acid (6.5 g).

1H-NMR(DMSO-d₆): δ 1.54-1.83(6H, m), 3.06-3.21(4H, m), 7.68(1H, d, J=8.1 Hz), 7.99(1H, s), 8.12(1H, d, J=8.1 Hz), 17.19(1H, s) (−)ESI-MS: 272(M−H)⁻

### EXAMPLE 14

### 2-(1-Piperidinyl)-N-[1-(2-pyridinylacetyl)-2,3-dihydro-1H-indol-5-yl]-4-(trifluoromethyl)benzamide

The title compound was obtained in a similar manner as in Example 1 from 1-(2-pyridinylacetyl)-5-indolinamine and 2-(1-piperidinyl)-4-(trifluoromethyl)benzoic acid.

¹H-NMR(DMSO-d₆): δ 1.40-1.70(6H, m), 2.94-3.07(4H, m), 3.18(2H, t, J=8.4 Hz), 4.01(2H, s), 4.23(2H, t, J=8.4 Hz), 7.28(1H, dd, J=5.0 Hz, 6.7 Hz), 7.34-7.52(4H, m), 7.71-7.87(3H, m), 8.02(1H, d, J=8.6 Hz), 8.47-8.54(1H, m), 10.93(1H, s) (−)ESI-MS: 507(M−H)⁻

Preparation 22

### 4-Chloro-2-(1-piperidinyl)benzonitrile

The title compound was obtained in a similar manner as in Preparation 20 from 4-chloro-2-fluorobenzonitrile and piperidine.

¹H-NMR(DMSO-d₆): δ 1.48-1.75(6H, m), 3.08-3.21(4H, m), 7.09(1H, dd, J=1.9 Hz, 8.2 Hz), 7.15(1H, d, J=1.9 Hz), 7.70(1H, d, J=8.2 Hz)

Preparation 23

### 4-Chloro-2-(1-piperidinyl)benzoic acid

The title compound was obtained in a similar manner as in Preparation 21 from 4-chloro-2-(1-piperidinyl)benzonitrile.

¹H-NMR(DMSO-d₆): δ 1.51-1.82(6H, m), 2.98-3.17(4H, m), 7.44(1H, dd, J=2.0 Hz, 8.3 Hz), 7.80(1H, d, J=2.0 Hz), 7.97(1H, d, J=8.3 Hz), 17.23(1H, s) (−)ESI-MS: 238(M−H)⁻

### EXAMPLE 15

### 4-Chloro-2-(1-piperidinyl)-N-[1-(2-pyridinylacetyl)-2,3-dihydro-1H-indol-5-yl]benzamide

The title compound was obtained in a similar manner as in Example 1 from 1-(2-pyridinylacetyl)-5-indolinamine and 4-chloro-2-(1-piperidinyl)benzoic acid.

¹H-NMR(DMSO-d₆): δ 1.04-1.75(6H, m), 2.86-3.03(4H, m), 3.18(2H, t, J=8.4 Hz), 4.01(2H, s), 4.23(2H, t, J=8.4 Hz), 7.17-7.46(5H, m), 7.69-7.84(3H, m), 8.01(1H, d, J=8.6 Hz), 8.46-8.54(1H, m), 11.16(1H, s) (−)ESI-MS: 473(M−H)⁻

Preparation 24

### 4-Methoxy-2-(1-piperidinyl)benzonitrile

The title compound was obtained in a similar manner as in Preparation 20 from 2-fluoro-4-methoxybenzonitrile and piperidine.

¹H-NMR(DMSO-d₆): δ 1.47-1.75(6H, m), 3.03-3.16(4H, m), 3.81(3H, s), 6.57(1H, d, J=2.3 Hz), 6.62(1H, dd, J=2.3 Hz, 8.5 Hz), 7.59(1H, d, J=8.5 Hz)

Preparation 25

### 4-Methoxy-2-(1-piperidinyl)benzoic acid

The title compound was obtained in a similar manner as in Preparation 21 from 4-methoxy-2-(1-piperidinyl)benzonitrile. ¹H-NMR(DMSO-d₆): δ 1.56-1.81(6H, m), 2.97-3.09(4H, m), 3.85(3H, s), 6.99(1H, dd, J=2.5 Hz, 8.7 Hz), 7.25(1H, d, J=2.5Hz), 7.97(1H, d, J=8.7 Hz), 17.71(1H, s) (−)ESI-MS: 234(M−H)⁻

### EXAMPLE 16

### 4-Methoxy-2-(1-piperidinyl)-N-[1-(2-pyridinylacetyl)-2,3-dihydro-1H-indol-5-yl]benzamide

The title compound was obtained in a similar manner as in Example 1 from 1-(2-pyridinylacetyl)-5-indolinamine and 4-methoxy-2-(1-piperidinyl)benzoic acid.

¹H-NMR(DMSO-d₆): δ 1.47-1.80(6H, m), 2.85-3.00(4H, m), 3.18(2H, t, J=8.3 Hz), 3.82(3H, s), 4.01(2H, s), 4.22(2H, t, J=8.3 Hz), 6.77-6.88(2H, m), 7.28(1H, dd, J=5.2 Hz, 7.1 Hz), 7.34-7.46(2H, m), 7.72-7.85(2H, m), 7.89(1H, d, J=8.3 Hz), 8.02(1H, d, J=8.6 Hz), 8.47-8.56(1H, m), 11.82(1H, s) (+)ESI-MS: 471(M+H)⁺, 493(M+Na)⁺

Preparation 26

### Benzyl 5-methyl-2-(1-pyrrolidinyl)benzoate

The title compound was obtained in a similar manner as in Preparation 3 from benzyl 5-methyl-2-(trifluoromethanesulfonyloxy)benzoate and pyrrolidine.

1H-NMR(DMSO-d₆): δ 1.73-1.90(4H, m), 2.19(3H, s), 2.99-3.13(4H, m), 5.27(2H, s), 6.71(1H, d, J=8.5 Hz), 7.13(1H, dd, J=2.0 Hz, 8.5 Hz), 7.27(1H, d, J=2.0 Hz), 7.33-7.50(5H, m)

Preparation 27

### 5-Methyl-2-(1-pyrrolidinyl)benzoic acid

The title compound was obtained in a similar manner as in Preparation 6 from benzyl 5-methyl-2-(1-pyrrolidinyl)benzoate.

¹H-NMR(DMSO-d₆): δ 1.86-2.01(4H, m), 2.26(3H, s), 3.10-3.25(4H, m), 7.06(1H, d, J=8.4 Hz), 7.25(1H, dd, J=1.8 Hz, 8.4 Hz), 7.50(1H, d, J=1.8 Hz), 14.75(1H, s) (+)ESI-MS: 206(M+H)⁺, 228(M+Na)⁺

### EXAMPLE 17

### 5-Methyl-N-[1-(2-pyridinylacetyl)-2,3-dihydro-1H-indol-5-yl]-2-(1-pyrrolidinyl)benzamide

The title compound was obtained in a similar manner as in Example 1 from 1-(2-pyridinylacetyl)-5-indolinamine and 5-methyl-2-(1-pyrrolidinyl)benzoic acid.

¹H-NMR(DMSO-d₆): δ 1.75-1.94(4H, m), 2.23(3H, s), 3.06-3.25(6H, m), 4.00(2H, s), 4.21(2H, t, J=8.4 Hz), 6.71(1H, d, J=8.2 Hz), 7.05-7.17(2H, m), 7.23-7.46(3H, m), 7.69(1H, s), 7.74(1H, dt, J=1.8 Hz, 7.7Hz), 7.97(1H, d, J=8.7 Hz), 8.47-8.54(1H, m), 10.36(1H, s) (+)ESI-MS: 441(M+H)⁺, 463(M+Na)⁺

Preparation 28

### Benzyl 5-methyl-2-(1-piperidinyl)benzoate

The title compound was obtained in a similar manner as in Preparation 3 from benzyl 5-methyl-2-(trifluoromethanesulfonyloxy)benzoate and piperidine.

¹H-NMR(DMSO-d₆): δ 1.36-1.59(6H, m), 2.24(3H, s), 2.76-2.88(4H, m), 5.29(2H, s), 6.99(1H, d, J=8.3 Hz), 7.19-7.51(7H, m)

Preparation 29

### 5-Methyl-2-(1-piperidinyl)benzoic acid

The title compound was obtained in a similar manner as in Preparation 6 from benzyl 5-methyl-2-(1-piperidinyl)benzoate.

¹H-NMR(DMSO-d₆): δ 1.52-1.87(6H, m), 2.35(3H, s), 2.90-3.14(4H, m), 7.47(1H, d, J=8.2 Hz), 7.62(1H, d, J=8.2 Hz), 7.85(1H, s), 17.20(1H, s) (+)ESI-MS: 220(M+H)⁺, 242(M+Na)⁺

### EXAMPLE 18

### 5-Methyl-2-(1-piperidinyl)-N-[1-(2-pyridinylacetyl)-2,3-dihydro-1H-indol-5-yl]benzamide

The title compound was obtained in a similar manner as in Example 1 from 1-(2-pyridinylacetyl)-5-indolinamine and 5-methyl-2-(1-piperidinyl)benzoic acid

¹H-NMR(DMSO-d₆): δ 1.46-1.86(6H, m), 2.31(3H, s), 2.82-2.97(4H, m), 3.18(2H, t, J=8.3 Hz), 4.01(2H, s), 4.23(2H, t, J=8.3 Hz), 7.21-7.46(5H, m), 7.71-7.84(3H, m), 8.02(1H, d, J=8.6 Hz), 8.47-8.54(1H, m), 12.06(1H, s) (+)ESI-MS: 455(M+H)⁺, 477(M+Na)⁺

Preparation 30

### 2-(1-Piperidinyl)-3-(trifluoromethyl)benzonitrile

The title compound was obtained in a similar manner as in Preparation 20 from 2-fluoro-3-(trifluoromethyl)benzonitrile and piperidine.

¹H-NMR(DMSO-d₆): δ 1.46-1.71(6H, m), 2.98-3.21(4H, m), 7.56(1H, t, J=7.7 Hz), 8.02(1H, dd, J=1.4 Hz, 7.7 Hz), 8.09(1H, dd, J=1.4 Hz, 7.7 Hz) (+)ESI-MS: 255(M+H)⁺, 277(M+Na)⁺

Preparation 31

### 2-(1-Piperidinyl)-3-(trifluoromethyl)benzoic acid

The title compound was obtained in a similar manner as in Preparation 21 from 2-(1-piperidinyl)-3-(trifluoromethyl)benzonitrile.

¹H-NMR(DMSO-d₆): δ 1.35-1.70(6H, m), 2.87-3.13(4H, m), 7.40(1H, dd, J=7.5 Hz, 8.0 Hz), 7.71-7.86(2H, m), 13.45(1H, s)

### EXAMPLE 19

### 2-(1-Piperidinyl)-N-[1-(2-pyridinylacetyl)-2,3-dihydro-1H-indol-5-yl]-3-(trifluoromethyl)benzamide

The title compound was obtained in a similar manner as in Example 1 from 1-(2-pyridinylacetyl)-5-indolinamine and 2-(1-piperidinyl)-3-(trifluoromethyl)benzoic acid.

¹H-NMR(DMSO-d₆): δ 1.25-1.63(6H, m), 2.89-3.05(4H, m), 3.18(2H, t, J=8.3 Hz), 4.01(2H, s), 4.23(2H, t, J=8.3 Hz), 7.23-7.33(1H, m), 7.33-7.49(3H, m), 7.61-7.83(4H, m), 8.00(1H, d, J=8.7 Hz), 8.47-8.53(1H, m), 10.45(1H, s) (−)ESI-MS: 507(M−H)⁻

Preparation 32

To a solution of 6-methyl-2-pyridinamine (25.0 g) and 2,5-hexanedione (29.0 g) in toluene (150 ml) was added p-toluenesulfonic acid hydrate (4.4 g) at ambient temperature and the mixture was refluxed for 18 hours. The mixture was evaporated in vacuo and the residue was purified by column chromatography on silica gel eluting with n-hexane: ethyl acetate (4:1 v/v) to give 2-(2,5-dimethyl-1H-pyrrol-1-yl)-6-methylpyridine (35.8 g) as a yellow oil.

¹H-NMR(DMSO-d₆): δ 2.04(6H, s), 2.51(3H, s), 5.78(2H, s), 7.18(1H, d, J=7.8 Hz), 7.29(1H, d, J=7.6 Hz), 7.86(1H, dd, J=7.8 Hz, 7.6 Hz) APCI-MS(m/z): 187(M+H)⁺

Preparation 33

To a solution of diisopropylamine (11.1 g) in tetrahydrofuran (80 ml) was added dropwise n-butyllithium (1.59 M solution in hexane, 69.1 ml) at -60° C. under a nitrogen atmosphere and the mixture was stirred at −60° C. for 30 minutes. To the mixture was added dropwise a solution of 2-(2,5-dimethyl-1H-pyrrol-1-yl)-6-methylpyridine (18.63 g) in tetrahydrofuran (200 ml) at −60° C. over 50 minutes and the reaction mixture was stirred for 30 minutes. Powdered Dry Ice was added carefully and the mixture was gradually warmed to ambient temperature. The mixture was quenched by addition of a saturated aqueous solution of ammonium chloride and poured into a mixture of ethyl acetate and water. The mixture was adjusted to pH 2 with 6N hydrochloric acid. The separated organic layer was washed with water and brine, dried over magnesium sulfate and evaporated in vacuo. The residue was purified by column chromatography on silica gel to give [6-(2,5-dimethyl-1H-pyrrol-1-yl)-2-pyridinyl]acetic acid (9.69 g) as pale brown crystals.

1H-NMR(DMSO-d₆): δ 2.04(6H, s), 3.79(2H, s), 5.79(2H, s), 7.28(2H, d, J=7.9 Hz), 7.38(2H, d, J=7.9 Hz), 7.93(1H, dd, J=7.9 Hz, 7.9 Hz), 12.30(1H, br) ESI-MS(m/z): 253(M+Na)⁺, 231(M+H)⁺

Preparation 34

To a solution of 5-nitroindoline (4.925 g), [6-(2,5-dimethyl-1H-pyrrol-1-yl)-2-pyridinyl]acetic acid (8.29 g) and PyBOP (benzotriazol-1-yl-oxy-tris-pyrrolidino-phosphonium hexafluorophosphate (18.7 g) in N,N-dimethylformamide (40 ml) was added dropwise diisopropylethylamine (7.76 g) at 5° C. The mixture was gradually warmed to ambient temperature and stirred for 18 hours. The reaction mixture was poured into a mixture of ethyl acetate and water and the separated organic layer was washed with water and brine, dried over magnesium sulfate and evaporated in vacuo. The residue was purified by column chromatography on silica gel eluting with ethyl acetate to give 1-{[6-(2,5-dimethyl-1H-pyrrol-1-yl)-2-pyridinyl]acetyl}-5-nitroindoline (6.67 g) as light yellow crystals.

¹H-NMR(DMSO-d₆): δ 2.02(6H, s), 3.25(2H, t, J=8.6 Hz), 4.16(2H, s), 4.30(2H, t, J=8.6 Hz), 5.77(2H, s), 7.31(1H, d, J=8.6 Hz), 7.31(1H, d, J=8.6 Hz), 7.98(1H, dd, J=8.6 Hz, 8.6 Hz), 8.00-8.15(3H, m) APCI-MS(m/z): 377(M+H)⁺

Preparation 35

### 1-{[6-(2,5-Dimethyl-1H-pyrrol-1-yl)-2-pyridinyl]acetyl}-5-indolinamine

The title compound was obtained in a similar manner as in Preparation 2 from 1-{[6-(2,5-dimethyl-1H-pyrrol-1-yl)-2-pyridinyl]acetyl}-5-nitroindoline as light yellow crystals.

¹H-NMR(DMSO-d₆): δ 2.22(6H, s), 2.99(2H, t, J=8.4 Hz), 3.98(2H, s), 4.08(2H, t, J=8.4 Hz), 4.84(2H, br s), 5.77(2H, s), 6.32(1H, dd, J=8.5 Hz, 2.2 Hz), 6.45(1H, d, J=2.2 Hz), 7.27(1H, d, J=7.7 Hz), 7.39(1H, d, J=7.3 Hz), 7.73(1H, d, J=8.5 Hz), 7.94(1H, dd, J=7.7 Hz, 7.3 Hz) ESI-MS (m/z) : 369 (M+Na)⁺, 347 (M+H)⁺

### EXAMPLE 20

### N-(1-{[6-(2,5-Dimethyl-1H-pyrrol-1-yl)-2-pyridinyl]acetyl}-2,3-dihydro-1H-indol-5-yl)-2-(1-piperidinyl)benzamide

The title compound was obtained in a similar manner as in Example 1 from 1-{[6-(2,5-dimethyl-1H-pyrrol-1-yl)-2-pyridinyl]acetyl}-5-indolinamine and 2-(1-piperidinyl)benzoic acid.

¹H-NMR(DMSO-d₆): δ 1.47-1.75(6H, m), 2.03(6H, s), 2.88-2.99(4H, m), 3.17(2H, t, J=8.4 Hz), 4.07(2H, s), 4.20(2H, t, J=8.4 Hz), 5.77(2H, s), 7.16-7.55(6H, m), 7.79-8.07(4H, m), 11.70(1H, s) (+)ESI-MS: 534(M+H)⁺, 556(M+Na)⁺

### EXAMPLE 21

A mixture of N-(1-{[6-(2,5-dimethyl-1H-pyrrol-1-yl)-2-pyridinyl]acetyl}-2,3-dihydro-1H-indol-5-yl)-2-(1-piperidinyl)benzamide (0.45 g), hydroxylamine hydrochloride (0.59 g) and triethylamine (0.24 ml) in a mixture of ethanol (18 ml) and water (9 ml) was stirred under reflux for 28 hours. The solvent was removed by concentration. To the residue was added a mixture of ethyl acetate, tetrahydrofuran and water and the reaction mixture was adjusted to pH 9 with 20% aqueous potassium carbonate solution. The separated organic layer was washed with water, dried over magnesium sulfate and evaporated in vacuo. The residue was triturated with a mixture of ethyl acetate and tetrahydrofuran to give N-{1-[(6-amino-2-pyridinyl)acetyl]-2,3-dihydro-1H-indol-5-yl}-2-(1-piperidinyl)benzamide (0.11 g).

¹H-NMR(DMSO-d₆): δ 1.46-1.82(6H, m), 2.88-3.02(4H, m), 3.17(2H, t, J=8.3 Hz), 3.71(2H, s), 4.21(2H, t, J=8.3 Hz), 5.87(2H, s), 6.31(1H, d, J=8.2 Hz), 6.44(1H, d, J=7.1 Hz), 7.16-7.57(5H, m), 7.77-7.90(2H, m), 8.03(1H, d, J=8.6 Hz), 11.68(1H, s) (−)ESI-MS: 454 (M−H)⁻

### EXAMPLE 22

### N-(1-{[6-(2,5-Dimethyl-1H-pyrrol-1-yl)-2-pyridinyl]acetyl}-2,3-dihydro-1H-indol-5-yl)-4-methyl-2-(1-piperidinyl)benzamide

The title compound was obtained in a similar manner as in Example 1 from 1-{[6-(2,5-dimethyl-1H-pyrrol-1-yl)-2-pyridinyl]acetyl}-5-indolinamine and 4-methyl-2-(1-piperidinyl)benzoic acid.

¹H-NMR(DMSO-d₆): δ 1.48-1.80(6H, m), 2.03(6H, s), 2.35(3H, s), 2.87-3.00(4H, m), 3.17(2H, t, J=8.3 Hz), 4.07(2H, s), 4.20(2H, t, J=8.3 Hz), 5.77(2H, s), 7.05(1H, d, J=8.0 Hz), 7.17(1H, s), 7.30(1H, d, J=7.8 Hz), 7.36-7.47(2H, m), 7.78-7.85(2H, m), 7.91-8.06(2H, m), 11.92(1H, s) (+)ESI-MS: 548(M+H)⁺, 570(M+Na)⁺

### EXAMPLE 23

### N-{1-[(6-Amino-2-pyridinyl)acetyl]-2,3-dihydro-1H-indol-5-yl}-4-methyl-2-(1-piperidinyl)benzamide

The title compound was obtained in a similar manner as in Example 21 from N-(1-{[6-(2,5-dimethyl-1H-pyrrol-1-yl)-2-pyridinyl]acetyl}-2,3-dihydro-1H-indol-5-yl)-4-methyl-2-(1-piperidinyl)benzamide. ¹H-NMR(DMSO-d₆): δ 1.46-1.80(6H, m), 2.35(3H, s), 2.84-3.00(4H, m), 3.16(2H, t, J=8.3 Hz), 3.71(2H, s), 4.21(2H, t, J=8.3 Hz), 5.87(2H, s), 6.31(1H, d, J=8.1 Hz), 6.44(1H, d, J=7.2 Hz), 7.05(1H, d, J=7.9 Hz), 7.17(1H, s), 7.26-7.46(2H, m), 7.75-7.87(2H, m), 8.03(1H, d, J=8.6 Hz), 11.90(1H, s) (−)ESI-MS: 468(M−H)⁻

Preparation 36

2-Nitrobenzoyl chloride (0.88 g) was added to a mixture of 1-(2-pyridinylacetyl)-5-indolinamine (1.0 g) and triethylamine (0.66 ml) in N,N-dimethylformamide (15 ml) under ice-cooling and the mixture was stirred at ambient temperature for 4 hours. The mixture was poured into a mixture of water and ethyl acetate and the mixture was adjusted to pH 9 with 20% aqueous potassium carbonate solution. The resultant precipitate was collected by filtration to give 2-nitro-N-[1-(2-pyridinylacetyl)-2,3-dihydro-1H-indol-5-yl]benzamide (1.00 g).

¹H-NMR(DMSO-d₆): δ 3.18(2H, t, J=8.3 Hz), 4.02(2H, s), 4.23(2H, t, J=8.3 Hz), 7.23-7.42(3H, s), 7.65(1H, s), 7.69-7.93(4H, m), 8.00(1H, d, J=8.7 Hz), 8.14(1H, d, J=7.8 Hz), 8.47-8.55(1H, m), 10.61(1H, s) (−)ESI-MS: 401(M−H)⁻

Preparation 37

To a mixture of 2-nitro-N-[1-(2-pyridinylacetyl)-2,3-dihydro-1H-indol-5-yl]benzamide (0.8 g) in a mixture of methanol (30 ml) and tetrahydrofuran (30 ml) was added 10% palladium on carbon (0.4 g, 50% wet). The reaction mixture was stirred at ambient temperature for 5 hours under hydrogen atmosphere. The catalyst was filtered off and the solvent was removed by concentration. The residue was triturated with a mixture of diethyl ether and ethyl acetate to give 2-amino-N-[1-(2-pyridinylacetyl)-2,3-dihydro-1H-indol-5-yl]benzamide (3.52 g).

¹H-NMR(DMSO-d₆): δ 3.16(2H, t, J=8.3 Hz), 4.01(2H, s), 4.22(2H, t, J=8.3 Hz), 6.31(2H, s), 6.53-6.63(1H, m), 6.70-6.77(1H, m), 7.14-7.32(2H, m), 7.33-7.47(2H, m), 7.60(1H, dd, J=1.1 Hz, 7.9 Hz), 7.66(1H, s), 7.77(1H, dt, J=1.8 Hz, 7.6 Hz), 7.98(1H, d, J=8.7 Hz), 8.48-8.54(1H, m), 9.93(1H, s) (−) ESI-MS: 371 (M−H)⁻

### EXAMPLE 24

### 2-(Dimethylamino)-N-[1-(2-pyridinylacetyl)-2,3-dihydro-1H-indol-5-yl]benzamide

The title compound was obtained in a similar manner as in Example 1 from 1-(2-pyridinylacetyl)-5-indolinamine and 2-(dimethylamino)benzoic acid.

¹H-NMR(DMSO-d₆): δ 2.77(6H, s), 3.17(2H, t, J=8.4 Hz), 4.01(2H, s), 4.22(2H, t, J=8.4 Hz), 7.04-7.15(1H, m), 7.18-7.50(5H, m), 7.64-7.83(3H, m), 8.00(1H, d, J=8.7 Hz), 8.47-8.54(1H, m), 11.25(1H, s) (+)APCI-MS: 401(M+H)⁺

Preparation 38

To a mixture of 2-amino-4-methylbenzoic acid (3.0 g) and 37% aqueous formaldehyde (29.7 ml) in methanol (60 ml) was added 10% palladium on carbon (2.0 g, 50% wet). The reaction mixture was stirred at ambient temperature for 16 hours under hydrogen atmosphere. The catalyst was filtered off and the solvent was removed by concentration and the residue was triturated with ethyl acetate to give 2-(dimethylamino)-4-methylbenzoic acid (1.91 g).

¹H-NMR(DMSO-d₆): δ 2.38(3H, s), 2.80(6H, s), 7.20(1H, d, J=7.9 Hz), 7.56(1H, s), 7.88(1H, d, J=7.9 Hz) (+)ESI-MS: 180(M+H)⁺, 202(M+Na)⁺

### EXAMPLE 25

### 2-(Dimethylamino)-4-methyl-N-[1-(2-pyridinylacetyl)-2,3-dihydro-1H-indol-5-yl]benzamide

The title compound was obtained in a similar manner as in Example 1 from 1-(2-pyridinylacetyl)-5-indolinamine and 2-(dimethylamino)-4-methylbenzoic acid.

¹H-NMR(DMSO-d₆): δ 2.34(3H, s), 2.76(6H, s), 3.17(2H, t, J=8.3 Hz), 4.01(2H, s), 4.22(2H, t, J=8.3 Hz), 6.95(1H, t, J=8.0 Hz), 7.10(1H, s), 7.24-7.47(3H, m), 7.64-7.82(3H, m), 8.00(1H, d, J=8.6 Hz), 8.48-8.53(1H, m), 11.50(1H, s) (+)ESI-MS: 415(M+H)⁺, 437(M+Na)⁺

Preparation 39

A mixture of 2-chloro-6-methylnicotinic acid (3.43 g), tert-butyl 5-amino-1-indolinecarboxylate (5.15 g), 1-hydroxybenzotriazole hydrate (3.21 g) and 1-[3-(dimethylamino)propyl]-3-ethylcarbodiimide (3.26 g) in N,N-dimethylformamide (30 ml) was stirred at ambient temperature overnight. The reaction mixture was poured into a mixture of ethyl acetate and water, and the organic layer was washed with brine and dried over magnesium sulfate. The solvent was evaporated in vacuo and the residue was chromatographed on silica gel eluting with ethyl acetate and n-hexane (6:4 v/v). The eluted fractions containing the desired product were collected and evaporated in vacuo to give tert-butyl 5-{[(2-chloro-6-methyl-3-pyridinyl)carbonyl]amino}-1-indolinecarboxylate (6.65 g).

¹H-NMR(DMSO-d₆): δ 1.51(9H, s), 2.51(3H, s), 3.07(2H, t, J=8.5 Hz), 3.91(2H, t, J=8.5 Hz), 7.37-7.41(2H, m), 7.52-7.69(2H, m), 7.92(1H, d, J=7.6 Hz), 10.43 (1H, s)

Preparation 40

A mixture of tert-butyl 5-{[(2-chloro-6-methyl-3-pyridinyl)carbonyl]amino}-1-indolinecarboxylate (1.55 g) and piperidine (1.6 ml) in tetrahydrofuran (10 ml) was refluxed under stirring for 4.5 hours. The reaction mixture was poured into a mixture of ethyl acetate and water, and the organic layer was washed with brine and dried over magnesium sulfate. The solvent was concentrated in vacuo and the precipitate was collected by filtration to give tert-butyl 5-({[6-methyl-2-(1-piperidinyl)-3-pyridinyl]carbonyl}amino)-1-indolinecarboxylate (1.01 g).

¹H-NMR(DMSO-d₆): δ 1.51(9H, s), 1.51-1.53(6H, m), 2.40(3H, s), 3.35(2H, t, J=8.4 Hz), 3.35(4H, m), 3.90(2H, t, J=8.4 Hz), 6.83(1H, d, J=7.7 Hz), 7.40-7.43(2H, m), 7.67(1H, s), 7.75(1H, d, J=7.6 Hz), 10.47(1H, s) (+)ESI-MS(m/z): 437(M+H)⁺, 459(M+Na)⁺

Preparation 41

A mixture of tert-butyl 5-({[6-methyl-2-(1-piperidinyl)-3-pyridinyl]carbonyl}amino)-1-indolinecarboxylate (1.0 g) and trifluoroacetic acid (1.8 ml) in dichloromethane (5 ml) was stirred at ambient temperature for 5 hours. The reaction mixture was evaporated in vacuo. The residue was dissolved in a mixture of ethyl acetate and water and the mixture was adjusted to pH 8.5 with aqueous potassium carbonate solution. The organic layer was washed with brine and dried over magnesium sulfate. The solvent was concentrated in vacuo and the precipitate was collected by filtration to give N-(2,3-dihydro-1H-indol-5-yl)-6-methyl-2-(1-piperidinyl)nicotinamide (595 mg).

¹H-NMR(DMSO-d₆): δ 1.52-1.58(6H, m), 2.39(3H, s), 2.90(2H, t, J=8.4 Hz), 3.19-3.21(4H, m), 3.35-3.42(2H, m), 5.35(1H, s), 6.46(1H, d, J=8.3 Hz), 6.83(1H, d, J=7.6 Hz), 7.20(1H, d, J=8.3 Hz), 7.417(1H, s), 7.75(1H, d, J=7.6 Hz), 10.29(1H, s)

### EXAMPLE 26

A mixture of N-(2,3-dihydro-1H-indol-5-yl)-6-methyl-2-(1-piperidinyl)nicotinamide (330 mg), 2-pyridylacetic acid dihydrochloride (179 mg), 1-hydroxybenzotriazole hydrate (158 mg), 1-[3-(dimethylamino)propyl]-3-ethylcarbodiimide (160 mg) and N,N-dimethylaminopyridine (2.4 mg) in N,N-dimethylformamide (15 ml) was stirred at ambient temperature overnight. The reaction mixture was poured into a mixture of ethyl acetate and water, and the organic layer was washed with brine and dried over magnesium sulfate. The solvent was concentrated in vacuo and the precipitate was collected by filtration to give 6-methyl-2-(1-piperidinyl)-N-[1-(2-pyridinylacetyl)-2,3-dihydro-1H-indol-5-yl]nicotinamide (305 mg).

¹H-NMR(DMSO-d₆): δ 1.53(6H, br,s), 2.39(3H, s), 3.13-3.55(8H, m), 4.01(2H, s), 4.22(2H, t, J=8.30 Hz), 6.83(1H, d, J=7.64 Hz), 7.24-7.43(3H, m), 7.73-7.81(3H, m), 7.89(1H, d, J=8.66 Hz), 8.48-8.51(1H, m), 10.52(1H, s) (+)ESI-MS(m/z): 456(M+H)⁺, 478(M+Na)⁺

Preparation 42

### tert-Butyl 5-({[6-methyl-2-(4-methyl-1-piperidinyl)-3-pyridinyl]carbonyl}amino)-1-indolinecarboxylate

The title compound was obtained in a similar manner as in Preparation 40 from tert-butyl 5-{[(2-chloro-6-methyl-3-pyridinyl)carbonyl]amino}-1-indolinecarboxylate and 4-methylpiperidine.

¹H-NMR(DMSO-d₆): δ 0.98(3H, d, J=6.2 Hz), 1.13-1.28(2H, m), 1.40(9H, s), 1.40-1.65(3H, m), 2.39(3H, s), 2.74-2.80(2H, m), 3.10(2H, t, J=8.4 Hz), 3.60-3.68(2H, m), 3.90(2H, t, J=8.4 Hz), 6.82(1H, d, J=7.6 Hz), 7.39-7.42(1H, m), 7.42-7.67(1H, m), 7.67(1H, s), 7.74(1H, d, J=7.6 Hz), 10.44(1H, s) (+)ESI-MS(m/z): 451(M+H)⁺, 473(M+Na)⁺

Preparation 43

### N-(2,3-Dihydro-1H-indol-5-yl)-6-methyl-2-(4-methyl-1-piperidinyl)nicotinamide

The title compound was obtained in a similar manner as in Preparation 41 from tert-butyl 5-({[6-methyl-2-(4-methyl-1-piperidinyl)-3-pyridinyl]carbonyl}amino)-1-indolinecarboxylate.

¹H-NMR(DMSO-d₆): δ 0.90(3H, d, J=6.1 Hz), 1.18-1.31(2H, m), 1.46-1.66(3H, m), 2.38(3H, s), 2.74-2.94(4H, m), 3.33-3.44(2H, m), 3.60-3.67(2H, m), 5.34(1H, s), 6.46(1H, d, J=8.2 Hz), 6.82(1H, d, J=7.6 Hz), 7.20(1H, dd, J=1.9 Hz, 8.2 Hz), 7.46(1H, d, J=1.9 Hz), 7.74(1H, d, J=7.6 Hz), 10.24(1H, s) (+)ESI-MS(m/z): 351(M+H)⁺, 373(M+Na)⁺

### EXAMPLE 27

### 6-Methyl-2-(4-methyl-1-piperidinyl)-N-[1-(2-pyridinylacetyl)-2,3-dihydro-1H-indol-5-yl]nicotinamide

The title compound was obtained in a similar manner as in Example 26 from N-(2,3-dihydro-1H-indol-5-yl)-6-methyl-2-(4-methyl-1-piperidinyl)nicotinamide and 2-pyridylacetic acid dihydrochloride.

¹H-NMR(DMSO-d₆): δ 0.88(3H, d, J=6.1 Hz), 1.14-1.21(2H, m), 1.52-1.70(3H, m), 2.39(3H, s), 2.70-2.80(2H, m), 3.17-3.21(2H, m), 3.61-3.68(2H, m), 4.00(2H, s), 4.12-4.22(2H, m), 6.82(1H, d, J=7.6 Hz), 7.28-7.42(3H, m), 7.72-7.77(3H, m), 7.98 (1H, d, J=8.7 Hz), 8.49-8.52(1H, m), 10.47(1H, s) (+)ESI-MS(m/z): 470(M+1)⁺, 492(M+Na)⁺

Preparation 44

A mixture of 2-chloro-nicotinic acid (1.58 g), 1-(2-pyridinylacetyl)-5-indolinamine (2.67 g), 1-hydroxybenzotriazole hydrate (1.61 g) and 1-[3-(dimethylamino)propyl]-3-ethylcarbodiimide (1.63 g) in N,N-dimethylformamide (30 ml) was stirred at ambient temperature overnight. The reaction mixture was poured into a mixture of ethyl acetate and water and stirred at ambient temperature for 20 minutes. The precipitate was collected by filtration and washed successively with water, ethyl acetate and diisopropyl ether and dried to give 2-chloro-N-[1-(2-pyridinylacetyl)-2,3-dihydro-1H-indol-5-yl]nicotinamide (2.95 g).

¹H-NMR(DMSO-d₆): δ 3.18(2H, t, J=8.32 Hz), 4.01(2H, s), 4.23(2H, t, J=8.32 Hz), 7.25-7.39(1H, m), 7.52-7.59(2H, m), 7.68-7.69(1H, m), 7.76-7.77(2H, m), 7.97-8.08(2H, m), 8.49-8.54(2H, m), 10.57(1H, s)

### EXAMPLE 28

A mixture of 2-chloro-N-[1-(2-pyridinylacetyl)-2,3-dihydro-1H-indol-5-yl]nicotinamide (432 mg) and piperidine (0.45 ml) in chloroform (20 ml) was refluxed under stirring for 12 hours. The reaction mixture was poured into a mixture of chloroform and water and the organic layer was washed with brine and dried over magnesium sulfate. The solvent was evaporated in vacuo and the residue was chromatographed on silica gel eluting with chloroform and methanol (97:3 v/v). The fractions containing the desired product were collected and concentrated in vacuo and the precipitate was collected by filtration to give 2-(1-piperidinyl)-N-[1-(2-pyridinylacetyl)-2,3-dihydro-1H-indol-5-yl]nicotinamide (335 mg).

¹H-NMR(DMSO-d6): δ 1.53(6H, s), 3.22-3.25(4H, m), 4.01(2H, s), 6.90-6.98(1H, m), 7.21-7.43(3H, m), 7.70-7.82(3H, m), 7.96-8.02(1H, m), 8.23-8.26(1H, m), 8.45-8.47(1H, m), 10.46(1H, s) (+)ESI-MS (m/z) : 442(M+H)⁺, 464(M+Na)⁺

### EXAMPLE 29

### 2-(4-Methyl-1-piperidinyl)-N-[1-(2-pyridinylacetyl)-2,3-dihydro-1H-indol-5-yl]nicotinamide

The title compound was obtained in a similar manner as in Example 28 from 2-chloro-N-[1-(2-pyridinylacetyl)-2,3-dihydro-1H-indol-5-yl]nicotinamide and 4-methylpiperidine.

¹H-NMR(DMSO-d₆): δ 0.87(3H, d, J=6.1 Hz), 1.14-1.21(2H, m), 1.21-1.64(3H, m), 2.76-2.88(2H, m), 3.17(2H, t, J=8.3 Hz), 3.66-3.73(2H, m), 4.01(2H, s), 4.22(2H, t, J=8.3 Hz), 6.90-6.96(1H, m), 7.28-7.34(3H, m), 7.72-7.82(3H, m), 7.98(1H, d, J=8.6 Hz), 8.26-8.29(1H, m), 8.49-8.51(1H, m), 10.45(1H, s) (+)ESI-MS(m/z): 456(M+H)⁺, 478(M+Na)⁺

Preparation 45

### 2-Chloro-N-(2,3-dihydro-1H-indol-5-yl)-6-methylnicotinamide

The title compound was obtained in a similar manner as in Preparation 41 from tert-butyl 5-{[(2-chloro-6-methyl-3-pyridinyl)carbonyl]amino}-1-indolinecarboxylate.

¹H-NMR(DMSO-d₆): δ 2.50(3H, s), 2.90(2H, t, J=8.3 Hz), 3.34-3.45(2H, m), 5.39(1H, s), 6.46(1H, d, J=8.3 Hz), 7.18(1H, dd, J=1.9 Hz, 8.3 Hz), 7.35-7.40(2H, m), 7.88(1H, d, J=7.6 Hz), 10.13(1H, s)

Preparation 46

### 2-Chloro-6-methyl-N-[1-(2-pyridinylacetyl)-2,3-dihydro-1H-indol-5-yl]nicotinamide

The title compound was obtained in a similar manner as in Preparation 41 from 2-chloro-N-(2,3-dihydro-1H-indol-5-yl)-6-methylnicotinamide and 2-pyridylacetic acid dihydrochloride.

¹H-NMR(DMSO-d₆): δ 2.50(3H, s), 3.20(2H, t, J=8.3 Hz), 3.96(2H, s), 4.23(2H, t, J=8.3 Hz), 7.27-7.28(1H, m), 7.36-7.41(3H, m), 7.67 (1H, s), 7.74-7.78(1H, m), 7.98-8.00(1H, m), 8.80(1H, d, J=3.4 Hz), 10.46(1H, s)

### EXAMPLE 30

### 6-Methyl-N-[1-(2-pyridinylacetyl)-2,3-dihydro-1H-indol-5-yl]-2-(4-morpholinyl)nicotinamide

The title compound was obtained in a similar manner as in Example 28 from 2-chloro-6-methyl-N-[1-(2-pyridinylacetyl)-2,3-dihydro-1H-indol-5-yl]nicotinamide and morpholine.

¹H-NMR(DMSO-d₆) : δ 2.49(3H, s), 3.13-3.34(6H, m) , 3.61-3.66(4H, m), 3.94(2H, s), 4.22(2H, t, J=8.3 Hz), 6.85(1H, d, J=7.6 Hz), 7.25-7.45(3H, m), 7.71-7.81(3H, m), 7.94-8.17(1H, m), 8.80(1H, d, J=3.9 Hz), 10.39(1H, s) (+)ESI-MS(m/z): 458(M+H)⁺, 480(M+Na)⁺

### EXAMPLE 31

A mixture of N-(2,3-dihydro-1H-indol-5-yl)-6-methyl-2-(1-piperidinyl)nicotinamide (286 mg), {6-[(tert-butoxycarbonyl)amino]-2-pyridinyl}acetic acid (225 mg), 1-hydroxybenzotriazole hydrate (137 mg, 1-[3-(dimethylamino)propyl]-3-ethylcarbodiimide (139 mg) and N,N-dimethylaminopyridine (2.4 mg) in N,N-dimethylformamide (15 ml) was stirred at ambient temperature overnight. The reaction mixture was poured into a mixture of ethyl acetate and water, and the organic layer was washed with brine and dried over magnesium sulfate. The solvent was evaporated in vacuo and the residue was chromatographed on silica gel eluting with ethyl acetate and n-hexane (7:3 v/v). The fractions containing the desired product were collected and evaporated in vacuo to give tert-butyl 6-{2-[5-({[6-methyl-2-(1-piperidinyl)-3-pyridinyl]carbonyl}amino)-2,3-dihydro-1H-indol-1-yl]-2-oxoethyl}-2-pyridinylcarbamate (470 mg).

¹H-NMR(DMSO-d₆): δ 1.46(9H, s), 1.53(6H, br.s), 2.39(3H, s), 3.14-3.33(6H, m), 6.83(1H, d, J=7.7 Hz), 6.96-7.00(1H, m), 7.37-7.42(1H, m), 7.67-7.77(4H, m), 7.98(1H, d, J=8.7 Hz), 9.67(1H, s), 10.52(1H, s)

### EXAMPLE 32

A mixture of tert-butyl 6-{2-[5-({[6-methyl-2-(1-piperidinyl)-3-pyridinyl]carbonyl}amino)-2,3-dihydro-1H-indol-1-yl]-2-oxoethyl}-2-pyridinylcarbamate (460 mg) and trifluoroacetic acid (0.6 ml) in dichloromethane (5 ml) was stirred at ambient temperature for 5 hours. The reaction mixture was evaporated in vacuo and the residue was dissolved in a mixture of ethyl acetate and water and adjusted to pH 8.5 with aqueous potassium carbonate solution. The organic layer was washed with brine and dried over magnesium sulfate. The solvent was concentrated in vacuo and the precipitate was collected by filtration to give N-{1-[(6-amino-2-pyridinyl)acetyl]-2,3-dihydro-1H-indol-5-yl}-6-methyl-2-(1-piperidinyl)nicotinamide (306 mg).

¹H-NMR(DMSO-d₆): δ 1.53(6H, br.s), 2.39(3H, s), 3.11-3.30(6H, m), 4.20(2H, t, J=8.3 Hz), 5.86(2H, s), 6.30(1H, d, J=7.9 Hz), 6.43(1H, d, J=7.0 Hz), 6.83(1H, d, J=7.6 Hz), 7.28-7.43(2H, m), 7.72-7.78(2H, m), 7.98(1H, d, J=8.7 Hz), 10.51(1H, s) (+)ESI-MS(m/z): 471(M+H)⁺

Preparation 47

A mixture of tert-butyl 5-{[(2-chloro-6-methyl-3-pyridinyl)carbonyl]amino}-1-indolinecarboxylate (3.1 g) in 2M dimethylamine-tetrahydrofuran solution (20 ml) was refluxed under stirring for 10 hours. The reaction mixture was poured into a mixture of ethyl acetate and water, and the organic layer was washed with brine and dried over magnesium sulfate. The solvent was concentrated in vacuo and the precipitate was collected by filtration to give tert-butyl 5-({[2-(dimethylamino)-6-methyl-3-pyridinyl]carbonyl}amino)-1-indolinecarboxylate (2.19 g).

¹H-NMR(DMSO-d₆): δ 1.51(9H, s), 2.36(3H, s), 2.94(6H, s), 3.05 (2H, t, J=8.4 Hz), 3.90(2H, t, J=8.4 Hz), 6.61(1H, d, J=7.5 Hz), 7.39-7.43(1H, m), 7.54-7.60(3H, m), 10.18(1H, s) (+)ESI-MS(m/z): 397(M+H)⁺, 419(M+Na)⁺

Preparation 48

### N-(2,3-Dihydro-1H-indol-5-yl)-2-(dimethylamino)-6-methylnicotinamide

The title compound was obtained in a similar manner as in Preparation 41 from tert-butyl 5-({[2-(dimethylamino)-6-methyl-3-pyridinyl]carbonyl}amino)-1-indolinecarboxylate.

¹H-NMR(DMSO-d₆): δ 2.35(3H, s), 2.89(2H, t, J=8.4 Hz), 2.94(6H, s), 3.39(2H, t, J=8.4 Hz), 5.33(1H, s), 6.43(1H, d, J=7.5 Hz), 6.60(1H, d, J=7.5 Hz), 7.18(1H, m), 7.40(1H, s), 7.53(1H, d, J=7.4 Hz), 9.90(1H, s) (+)ESI-MS(m/z): 297(M+H)⁺

### EXAMPLE 33

### tert-Butyl 6-{2-[5-({[2-(dimethylamino)-6-methyl-3-pyridinyl]carbonyl}amino)-2,3-dihydro-1H-indol-1-yl]-2-oxoethyl}-2-pyridinylcarbamate

The title compound was obtained in a similar manner as in Example 31 from N-(2,3-dihydro-1H-indol-5-yl)-2-(dimethylamino)-6-methylnicotinamide and {6-[(tert-butoxycarbonyl)amino]-2-pyridinyl}acetic acid.

¹H-NMR(DMSO-d₆): δ 1.46(9H, s), 2.36(3H, s), 2.89(6H, s), 3.17(2H, t, J=8.3 Hz), 3.86(2H, s), 4.27(2H, t, J=8.3 Hz), 6.61(1H, d, J=7.5 Hz), 6.96-7.00(1H, m), 7.35-7.40(1H, m), 7.57(1H, d, J=7.5 Hz), 7.64-7.69(2H, m), 7.94-7.98(2H, m), 9.67(1H, s), 10.23(1H, s) (+)ESI-MS(m/z): 531(M+H)⁺, 553(M+Na)⁺

### EXAMPLE 34

### N-{1-[(6-Amino-2-pyridinyl)acetyl]-2,3-dihydro-1H-indol-5-yl}-2-(dimethylamino)-6-methylnicotinamide

The title compound was obtained in a similar manner as in Example 32 from tert-butyl 6-(2-[5-({[2-(dimethylamino)-6-methyl-3-pyridinyl]carbonyl}amino)-2,3-dihydro-1H-indol-1-yl]-2-oxoethyl}-2-pyridinylcarbamate.

¹H-NMR(DMSO-d₆): δ 2.35(3H, s), 2.94(6H, s), 3.14(2H, t, J=8.4 Hz), 3.71(2H, s), 4.19(2H, t, J=8.4 Hz), 5.87(2H, s), 6.31(1H, d, J=8.2 Hz), 6.43(1H, d, J=7.2 Hz), 6.61 (1H, d, J=7.5 Hz), 7.30-7.40(2H, m), 7.57(1H, d, J=7.5 Hz), 7.66(1H, s), 7.98(1H, d, J=8.7 Hz), 10.22(1H, s) (+)ESI-MS(m/z): 431(M+H)⁺, 453(M+Na)⁺

### EXAMPLE 35

### 2-(Dimethylamino)-6-methyl-N-[1-(2-pyridinylacetyl)-2,3-dihydro-1H-indol-5-yl]nicotinamide

The title compound was obtained in a similar manner as in Example 26 from N-(2,3-dihydro-1H-indol-5-yl)-2-(dimethylamino)-6-methylnicotinamide and 2-pyridylacetic acid dihydrochloride.

¹H-NMR(DMSO-d6): δ 2.37(3H, s), 2.95(6H, s), 3.19(2H, t, J=8.4 Hz), 3.92(2H, s), 3.93(2H, t, J=8.4 Hz), 6.63(1H, d, J=7.6 Hz), 7.51-7.62(2H, m), 7.73-7.82(2H, m), 7.91(1H, d, J=8.6 Hz), 8.11-8.23(2H, m), 8.79-8.81(1H, m), 10.34(1H, s)

### EXAMPLE 36

1-[3-(Dimethylamino)propyl]-3-ethylcarbodiimide (0.19 g) was added to a solution of N-(4-aminophenyl)-2-(2-pyridinyl)acetamide (0.23 g), 4-methyl-2-(4-methyl-1-piperidinyl)benzoic acid (0.28 g), 1-hydroxybenzotriazole hydrate (0.16 g) and 4-dimethylaminopyridine (6 mg) in dichloromethane (5 ml) under ice-cooling and the mixture was stirred at ambient temperature for 18 hours. The reaction mixture was poured into a mixture of ethyl acetate and water. The separated organic layer was washed with water, dried over magnesium sulfate and evaporated in vacuo. The residue was triturated with ethyl acetate to give 4-methyl-2-(4-methyl-1-piperidinyl)-N-{4-[(2-pyridinylacetyl)amino]phenyl}benzamide (0.14 g).

¹H-NMR(DMSO-d₆): δ 0.95(3H, d, J=6.0 Hz), 1.20-1.62(3H, m), 1.67-1.82(2H, m), 2.35(3H, s), 2.69-2.87(2H, m), 3.04-3.17(2H, m), 3.84(2H, s), 7.04(1H, d, J=7.9 Hz), 7.17(1H, s), 7.23-7.32(1H, m), 7.41(1H, d, J=7.8 Hz), 7.60(2H, d, J=9.1 Hz), 7.69(2H, d, J=9.1 Hz), 7.69-7.86(2H, m), 8.48-8.54(1H, m), 10.23(1H, s), 11.87(1H, s) (+)ESI-MS: 443(M+H)⁺, 465(M+Na)⁺

### EXAMPLE 37

### 2-(Dimethylamino)-4-methyl-N-{4-[(2-pyridinylacetyl)amino]phenyl}benzamide

The title compound was obtained in a similar manner as in Example 36 from N-(4-aminophenyl)-2-(2-pyridinyl)acetamide and 2-(dimethylamino)-4-methylbenzoic acid.

¹H-NMR(DMSO-d₆): δ 2.34(3H, s), 2.76(6H, s), 3.84(2H, s), 6.95(1H, d, J=7.8 Hz), 7.10(1H, s), 7.22-7.32(1H, m), 7.40(1H, d, J=7.8 Hz), 7.53-7.83(6H, m), 8.47-8.54(1H, m), 10.22(1H, s), 11.51(1H, s) (+)ESI-MS: 389(M+H)⁺, 411(M+Na)⁺

Preparation 49

To a solution of 4-fluoronitrobenzene (12.71 g) and 2-(2-pyridinyl)ethylamine (12.22 g) in N,N-dimethylformamide (70 ml) was added triethylamine (10.12 g) at ambient temperature and the mixture was stirred at 60° C. for 16 hours. The mixture was cooled to 5° C. and poured into a mixture of ethyl acetate and water. The separated organic layer was washed with water and brine, dried over magnesium sulfate and evaporated in vacuo. The residue was triturated with diisopropyl ether, collected by filtration, washed with diisopropyl ether and dried in vacuo to give 2-[2-(4-nitroanilino)ethyl]pyridine (21.21 g) as a yellow solid.

¹H-NMR(DMSO-d₆): δ 3.02(2H, t, J=7.0 Hz), 3.55(2H, td, J=7.0 Hz, 5.6 Hz), 6.65(2H, d, J=9.3 Hz), 7.24(1H, dd, J=7.8 Hz, 4.9 Hz), 7.31(1H, d, J=7.8 Hz), 7.39(1H, t, J=5.6 Hz), 7.65-7.8(1H, m), 7.98(1H, d, J=9.3 Hz), 8.52(1H, d, J=4.0 Hz) APCI-MS(m/z): 244(M⁺+1)

Preparation 50

To a solution of 2-[2-(4-nitroanilino)ethyl]pyridine (17.87 g) in tetrahydrofuran (150 ml) were added di-tert-butyl dicarbonate (19.25 g) and triethylamine (8.92 g) at ambient temperature and the mixture was refluxed for 16 hours. The mixture was evaporated in vacuo and the residue was purified by column chromatography on silica gel eluting with hexane: ethyl acetate (2:1 v/v) to give tert-butyl 4-nitrophenyl[2-(2-pyridinyl)ethyl]carbamate (18.21 g) as a yellow solid.

¹H-NMR(DMSO-d₆): δ 1.37(9H, s), 2.95(2H, t, J=8.0 Hz), 4.09(2H, t, J=8.0 Hz), 7.2-7.3(2H, m), 7.52(2H, d, J=9.1 Hz), 7.65-7.75(1H, m), 8.17(2H, d, J=9.1 Hz), 8.23(1H, d, J=4.8 Hz) APCI-MS(m/z): 344(M⁺+1)

Preparation 51

To a suspension of tert-butyl 4-nitrophenyl[2-(2-pyridinyl)ethyl]carbamate (20.03 g) in ethanol (400 ml) were added iron(III) chloride (anhydrous) (189 mg) and active-charcoal (20 g) and the mixture was heated to 80° C. To the mixture was added dropwise hydrazine hydrate (11.67 g) and the mixture was stirred at 80° C. for 4 hours. The active-charcoal was filtered off by celite and washed with ethanol. The filtrate was evaporated in vacuo and the residue was purified by column chromatography on silica gel eluting with ethyl acetate to give tert-butyl 4-aminophenyl[2-(2-pyridinyl)ethyl]carbamate (15.03 g) as a light brown solid.

¹H-NMR(DMSO-d₆): δ 1.29(9H, s), 2.86(2H, t, J=7.0 Hz), 3.78(2H, t, J=7.0 Hz), 5.04(2H, br s), 6.52 (2H, d, J=8.5 Hz), 6.80(2H, d, J=8.5 Hz), 7.15-7.3(2H, m), 7.65-7.75(1H, m), 8.45(1H, d, J=4.2 Hz) APCI-MS(m/z): 314(M+H)⁺

### EXAMPLE 38

1-[3-(Dimethylamino)propyl]-3-ethylcarbodiimide (0.19 g) was added to a solution of tert-butyl 4-aminophenyl[2-(2-pyridinyl)ethyl]carbamate (0.31 g), 4-methyl-2-(1-pyrrolidinyl)benzoic acid (0.25 g), 1-hydroxybenzotriazole hydrate (0.16 g) and 4-dimethylaminopyridine (6 mg) in dichloromethane (5 ml) under ice-cooling and the mixture was stirred at ambient temperature for 18 hours. To the reaction mixture was added a solution of 10% hydrogen chloride in methanol (9 ml) and the mixture was stirred at ambient temperature for 20 hours. The reaction mixture was poured into a mixture of ethyl acetate and water, and the mixture was adjusted to pH 9 with 20% aqueous potassium carbonate solution. The separated organic layer was washed with water, dried over magnesium sulfate and evaporated in vacuo. The residue was purified by column chromatography on silica gel using a mixture of ethyl acetate and diisopropyl ether (1:1 v/v) as an eluant. The eluted fractions containing the desired product were collected and evaporated in vacuo to give 4-methyl-N-(4-{[2-(2-pyridinyl)ethyl]amino}phenyl)-2-(1-pyrrolidinyl)benzamide (0.18 g).

¹H-NMR(DMSO-d₆): δ 1.77-1.93(4H, m), 2.27(3H, s), 2.98(2H, t, J=7.2 Hz), 3.14-3.28(4H, m), 3.28-3.43(2H, m), 5.51(1H, t, J=5.7 Hz), 6.50-6.64(4H, m), 7.13-7.27(2H, m), 7.31(1H, d, J=7.8 Hz), 7.41(2H, d, J=8.7 Hz), 7.71(1H, dt, J=1.7 Hz, 7.6 Hz), 8.49-8.55(1H, m), 9.91(1H, s) (+)ESI-MS: 401(M+H)⁺, 423(M+Na)⁺

### EXAMPLE 39

### 4-Methyl-2-(1-piperidinyl)-N-(4-{[2-(2-pyridinyl)ethyl]amino}phenyl)benzamide

The title compound was obtained in a similar manner as in Example 38 from tert-butyl 4-aminophenyl[2-(2-pyridinyl)ethyl]carbamate and 4-methyl-2-(1-piperidinyl)benzoic acid.

¹H-NMR(DMSO-d₆): δ 1.47-1.80(6H, m), 2.34(3H, s), 2.85-3.07(6H, m), 3.31-3.44(2H, m), 5.59(1H, t, J=5.7 Hz), 6.61(2H, d, J=8.8 Hz), 7.04(1H, d, J=8.0 Hz), 7.14-7.28(2H, m), 7.33(1H, d, J=7.8 Hz), 7.49(2H, d, J=8.8 Hz), 7.71(1H, dt, J=1.8 Hz, 7.6 Hz), 7.84(1H, d, J=8.0 Hz), 8.49-8.56(1H, m), 11.77(1H, s) (+)ESI-MS: 415(M+H)⁺, 437(M+Na)⁺

### EXAMPLE 40

### 2-(Hexahydro-1H-azepin-1-yl)-4-methyl-N-(4-{[2-(2-pyridinyl)ethyl]amino}phenyl)benzamide

The title compound was obtained in a similar manner as in Example 38 from tert-butyl 4-aminophenyl[2-(2-pyridinyl)ethyl]carbamate and 2-(hexahydro-1H-azepin-1-yl)-4-methylbenzoic acid.

¹H-NMR(DMSO-d₆): δ 1.52-1.67(4H, m), 1.67-1.85(4H, m), 2.31(3H, s), 2.98(2H, t, J=7.2 Hz), 3.12-3.27(4H, m), 3.29-3.44(2H, m), 5.56(1H, t, J=5.7 Hz), 6.59(2H, d, J=8.8 Hz), 6.86(1H, d, J=7.7 Hz), 7.03(1H, s), 7.17-7.28(1H, m), 7.32(1H, d, J=7.7 Hz), 7.42(2H, d, J=8.8 Hz), 7.58(1H, d, J=7.7 Hz), 7.65-7.77(1H, m), 8.48-8.56(1H, m), 11.19(1H, s) (+)ESI-MS: 429(M+H)⁺, 451(M+Na)⁺

### EXAMPLE 41

### 4-Methyl-2-(4-methyl-1-piperidinyl)-N-(4-{[2-(2-pyridinyl)ethyl]amino}phenyl)benzamide

The title compound was obtained in a similar manner as in Example 38 from tert-butyl 4-aminophenyl[2-(2-pyridinyl)ethyl]carbamate and 4-methyl-2-(4-methyl-1-piperidinyl)benzoic acid.

¹H-NMR(DMSO-d₆): δ 0.97(3H, d, J=6.4 Hz), 1.29-1.41(2H, m), 1.47-1.59(1H, m), 1.71-1.79(2H, m), 2.34(3H, s), 2.73-2.82(2H, m), 2.99(2H, t, J=7.3 Hz), 3.06-3.12(2H, m), 3.32-3.42(2H, m), 5.58(1H, t, J=5.7 Hz), 6.61(2H, d, J=8.8 Hz), 7.03(1H, d, J=7.9 Hz), 7.16(1H, s), 7.20-7.26(1H, m), 7.33(1H, d, J=7.9 Hz), 7.48(2H, d, J=8.8 Hz), 7.68-7.74(1H, m), 7.83(1H, d, J=7.9 Hz), 8.50-8.55(1H, m), 11.70(1H, s) (+)ESI-MS: 429(M+H)⁺, 451(M+Na)⁺

### EXAMPLE 42

### 2-(Dimethylamino)-4-methyl-N-(4-{[2-(2-pyridinyl)ethyl]amino}phenyl)benzamide

The title compound was obtained in a similar manner as in Example 38 from tert-butyl 4-aminophenyl[2-(2-pyridinyl)ethyl]carbamate and 2-(dimethylamino)-4-methylbenzoic acid.

¹H-NMR(DMSO-d₆): δ 2.33(3H, s), 2.75(6H, s), 2.99(2H, t, J=7.2 Hz), 3.30-3.44(2H, m), 5.56(1H, t, J=5.7 Hz), 6.59(2H, d, J=8.8 Hz), 6.94(1H, d, J=8.0 Hz), 7.08(1H, s), 7.18-7.27(1H, m), 7.32(1H, d, J=7.8 Hz), 7.43(2H, d, J=8.8 Hz), 7.64-7.77(2H, m), 8.49-8.55(1H, m), 11.18(1H, s) (+)ESI-MS: 375(M+H)⁺, 397(M+Na)⁺

Preparation 52

A mixture of 2-chloro-6-methylnicotinic acid (3.43 g), tert-butyl 4-aminophenyl[2-(2-pyridinyl)ethyl]carbamate (5.15 g), 1-hydroxybenzotriazole hydrate (3.21 g) and 1-[3-(dimethylamino)propyl]-3-ethylcarbodiimide (3.26 g) in N,N-dimethylformamide (30 ml) was stirred at ambient temperature overnight. The reaction mixture was poured into a mixture of ethyl acetate and water, and the organic layer was washed with brine and dried over magnesium sulfate. The solvent was evaporated in vacuo and the residue was chromatographed on silica gel eluting with ethyl acetate and n-hexane (5:5 v/v). The fractions containing the desired product were collected and evaporated in vacuo to give tert-butyl 4-{[(2-chloro-6-methyl-3-pyridinyl)carbonyl]amino}phenyl[2-(2-pyridinyl)ethyl]carbamate (8.43 g).

¹H-NMR(DMSO-d₆): δ 1.18(9H, s), 2.35(3H, s), 2.27(2H, t, J=7.3 Hz), 3.79(2H, t, J=7.3 Hz), 7.03-7.11(4H, m), 7.26(1H, d, J=7.8 Hz), 7.50-7.58(3H, m), 7.81(1H, d, J=7.6 Hz), 8.31-8.33(1H, m), 10.47(1H, s)

### EXAMPLE 43

A mixture of tert-butyl 4-{[(2-chloro-6-methyl-3-pyridinyl)carbonyl]amino}phenyl[2-(2-pyridinyl)ethyl]carbamate (700 mg) and piperidine (0.5 ml) in tetrahydrofuran (10 ml) was refluxed under stirring for 5 hours. The reaction mixture was poured into a mixture of ethyl acetate and water, and the organic layer was washed with brine and dried over magnesium sulfate. The solvent was evaporated in vacuo and the residue was chromatographed on silica gel eluting with ethyl acetate and n-hexane (5:5 v/v). The fractions containing the desired product were collected and evaporated in vacuo to give tert-butyl 4-({[6-methyl-2-(1-piperidinyl)-3-pyridinyl]carbonyl}amino)phenyl[2-(2-pyridinyl)ethyl]carbamate (520 mg).

¹H-NMR(DMSO-d₆): δ 1.32(9H, s), 1.55-1.57(6H, m), 2.40(3H, s), 2.91(2H, t, J=7.4 Hz), 3.22-3.33(4H, m), 3.91(2H, t, J=7.4 Hz), 6.84(1H, d, J=7.6 Hz), 7.16-7.25(4H, m), 7.64-7.71(3H, m), 7.77(1H, d, J=7.6 Hz), 8.45-8.46(1H, m), 10.62(1H, s)

### EXAMPLE 44

A mixture of tert-butyl 4-({[6-methyl-2-(1-piperidinyl)-3-pyridinyl]carbonyl}amino)phenyl[2-(2-pyridinyl)ethyl]carbamate (520 mg) and trifluoroacetic acid (1.0 ml) in dichloromethane (5 ml) was stirred at ambient temperature for 5 hours. The reaction mixture was evaporated in vacuo. The residue was dissolved in a mixture of ethyl acetate and water, and the mixture was adjusted to pH 8.5 with aqueous potassium carbonate solution. The organic layer was washed with brine and dried over magnesium sulfate. The solvent was concentrated in vacuo and the precipitate was collected by filtration to give 6-methyl-2-(1-piperidinyl)-N-(4-{[2-(2-pyridinyl)ethyl]amino}phenyl)nicotinamide (398 mg).

¹H-NMR(DMSO-d₆): δ 1.52-1.58(6H, m), 2.39(3H, s), 2.99(2H, t, J=7.4 Hz), 3.18-3.21(4H, m), 3.34-3.39(2H, m), 5.55-5.58(1H, m), 6.59(2H, d, J=8.8 Hz), 6.84 (1H, d, J=7.6 Hz), 7.21-7.24(1H, m), 7.32(1H, d, J=7.8 Hz), 7.45(2H, d, J=8.8 Hz), 7.69-7.73(1H, m), 7.77(1H, d, J=7.6 Hz), 8.51-8.52(1H, m), 10.33 (1H, s) (+)ESI-MS(m/z) : 416(M+H)⁺, 438(M+Na)⁺

### EXAMPLE 45

### tert-Butyl 4-({[6-methyl-2-(4-methyl-1-piperidinyl)-3-pyridinyl]carbonyl}amino)phenyl[2-(2-pyridinyl)ethyl]carbamate

The title compound was obtained in a similar manner as in Example 43 from tert-butyl 4-{[(2-chloro-6-methyl-3-pyridinyl)carbonyl]amino}phenyl[2-(2-pyridinyl)ethyl]carbamate and 4-methylpiperidine.

¹H-NMR(DMSO-d6): δ 0.89(3H, d, J=6.1 Hz), 1.14-1.46(2H, m), 1.47(9H, s), 1.50-1.52(1H, m), 1.60-1.66(2H, m), 2.40(3H, s), 2.76-2.95(4H, m), 3.64-3.70(2H, m), 3.88-3.97(2H, m), 6.82(1H, d, J=7.7 Hz), 7.15-7.26(4H, m), 7.65-7.78(4H, m), 8.44-8.47(1H, m), 10.57(1H, s)

### EXAMPLE 46

### 6-Methyl-2-(4-methyl-1-piperidinyl)-N-(4-{[2-(2-pyridinyl)ethyl]amino}phenyl)nicotinamide

The title compound was obtained in a similar manner as in Example 44 from tert-butyl 4-({[6-methyl-2-(4-methyl-1-piperidinyl)-3-pyridinyl]carbonyl}amino}phenyl[2-(2-pyridinyl)ethyl]carbamate.

¹H-NMR(DMSO-d₆): δ 0.90(6H, d, J=6.5 Hz), 1.17-1.26(2H, m), 1.49-1.51(1H, m), 1.62-1.65(2H, m), 2.39(3H, s), 2.99(2H, t, J=7.4 Hz), 3.34-3.39(2H, m), 3.61-3.65(2H, m), 5.56-5.59(1H, m), 6.58(2H, d, J=8.9 Hz), 6.82(1H, d, J=7.6 Hz), 7.21-7.24(1H, m), 7.32(1H, d, J=7.8 Hz), 7.45(2H, d, J=8.9 Hz), 7.69-7.76(2H, m), 8.51-8.52(1H, m), 10.26(1H, s) (+)ESI-MS(m/z): 430(M+H)⁺, 452(M+Na)⁺

### EXAMPLE 47

### tert-Butyl 4-({[6-methyl-2-(4-thiomorpholinyl)-3-pyridinyl]carbonyl}amino)phenyl[2-(2-pyridinyl)ethyl]carbamate

The title compound was obtained in a similar manner as in Example 43 from tert-butyl 4-{[(2-chloro-6-methyl-3-pyridinyl)carbonyl]amino}phenyl[2-(2-pyridinyl)ethyl]carbamate and thiomorpholine.

¹H-NMR(DMSO-d₆): δ 1.32(9H, s), 2.41(3H, s), 2.63-2.68(4H, m), 2.91(2H, t, J=7.4 Hz), 3.52-3.57(4H, m), 3.91(2H, t, J=7.4 Hz), 6.85(1H, d, J=7.7 Hz), 7.15-7.26(4H, m), 7.65-7.75(4H, m), 8.44-8.47(1H, m), 10.42(1H, s)

### EXAMPLE 48

### 6-Methyl-N-(4-{[2-(2-pyridinyl)ethyl]amino}phenyl)-2-(4-thiomorpholinyl)nicotinamide

The title compound was obtained in a similar manner as in Example 44 from tert-butyl 4-({[6-methyl-2-(4-thiomorpholinyl)-3-pyridinyl]carbonyl}amino)phenyl[2-(2-pyridinyl)ethyl]carbamate.

¹H-NMR(DMSO-d₆): δ 2.39(3H, s), 2.63-2.68(4H, m), 2.98(2H, t, J=7.4 Hz), 3.33-3.40(2H, m), 3.50-3.55(4H, m), 5.60(1H, s), 6.59(2H, d, J=8.8 Hz), 6.86 (1H, d, J=7.6 Hz), 7.19-7.26(1H, m), 7.32(1H, d, J=7.6 Hz), 7.44(2H, d, J=8.8 Hz), 7.67-7.75(2H, m), 8.50-8.53(1H, m), 10.05(1H, s) (+)ESI-MS(m/z): 434(M+H)⁺, 456(M+Na)⁺

### EXAMPLE 49

### tert-Butyl 4-({[6-methyl-2-(4-morpholinyl)-3-pyridinyl]carbonyl}amino)phenyl[2-(2-pyridinyl)ethyl]carbamate

The title compound was obtained in a similar manner as in Example 43 from tert-butyl 4-{[(2-chloro-6-methyl-3-pyridinyl)carbonyl]amino}phenyl[2-(2-pyridinyl)ethyl]carbamate and morpholine.

¹H-NMR(DMSO-d₆): δ 1.29(9H, s), 2.48(3H, s), 2.91(2H, t, J=7.4 Hz), 3.23-3.28(4H, m), 3.63-3.67(4H, m), 3.96(2H, t, J=7.4 Hz), 6.86(1H, d, J=7.7Hz), 7.15-7.26(4H, m), 7.65-7.77(4H, m), 8.45-8.47(1H, m), 10.49(1H, s)

### EXAMPLE 50

### 6-Methyl-N-(4-{[2-(2-pyridinyl)ethyl]amino}phenyl)-2-(4-morpholinyl)nicotinamide

The title compound was obtained in a similar manner as in Example 44 from tert-butyl 4-{[(2-chloro-6-methyl-3-pyridinyl)carbonyl]amino}phenyl[2-(2-pyridinyl)ethyl]carbamate.

¹H-NMR(DMSO-d₆): δ 2.40(3H, s), 2.98(2H, t, J=7.4 Hz), 3.21-3.26(4H, m), 3.33-3.40(4H, m), 3.66-3.68(2H, m), 5.58(1H, br.s), 6.58(2H, d, J=8.9 Hz), 6.85(1H, d, J=7.7 Hz), 7.19-7.26(1H, m), 7.32(1H, d, J=7.7 Hz), 7.45(2H, d, J=8.9 Hz), 7.67-7.75(2H, m), 8.50-8.53(1H, m), 10.11(1H, s) (+)ESI-MS(m/z): 418(M+H)⁺, 440(M+Na)⁺

Preparation 53

### tert-Butyl 4-{[(2-chloro-3-pyridinyl)carbonyl]amino}-phenyl[2-(2-pyridinyl)ethyl]carbamate

The title compound was obtained in a similar manner as in Preparation 52 from 2-chloronicotinic acid and tert-butyl 4-aminophenyl[2-(2-pyridinyl)ethyl]carbamate.

¹H-NMR(DMSO-d₆): δ 1.29(9H, s), 2.90(2H, t, J=7.4 Hz), 3.92(2H, t, J=7.4 Hz), 7.20-7.26(4H, m), 7.56-7.59(1H, m), 7.66-7.70(3H, m), 8.08-8.10(1H, m), 8.54-8.55(1H, m), 10.69(1H, s)

### EXAMPLE 51

### tert-Butyl 4-({[2-(1-piperidinyl)-3-pyridinyl]carbonyl}amino)phenyl[2-(2-pyridinyl)ethyl]carbamate

The title compound was obtained in a similar manner as in Example 43 from tert-butyl 4-{[(2-chloro-6-methyl-3-pyridinyl)carbonyl]amino}phenyl[2-(2-pyridinyl)ethyl]carbamate and piperidine.

¹H-NMR(DMSO-d₆): δ 1.32(9H, s), 1.55(6H, s), 2.91(2H, t, J=7.4 Hz), 3.26(4H, s), 3.91(2H, t, J=7.4 Hz), 6.95(1H, dd, J=4.7 Hz, 7.4 Hz), 7.16-7.27(4H, m), 7.66-7.72(3H, m), 7.81-7.85(1H, m), 8.28-8.31(1H, m), 8.46(1H, d, J=4.1 Hz), 10.57(1H, s)

### EXAMPLE 52

### 2-(1-Piperidinyl)-N-(4-{[2-(2-pyridinyl)ethyl]amino}phenyl)nicotinamide

The title compound was obtained in a similar manner as in Example 44 from tert-butyl 4-({[2-(1-piperidinyl)-3-pyridinyl]carbonyl}amino)phenyl[2-(2-pyridinyl)ethyl]carbamate.

¹H-NMR(DMSO-d₆): δ 1.52-1.58(6H, m), 2.39(3H, s), 2.99(2H, t, J=7.4 Hz), 3.18-3.21(4H, m), 3.34-3.39(2H, m), 5.55-5.58(1H, m), 6.59(2H, d, J=8.8 Hz), 6.84(1H, d, J=7.6 Hz), 7.21-7.24(1H, m), 7.32(1H, d, J=7.8 Hz), 7.45(2H, d, J=8.8 Hz), 7.69-7.73(1H, m), 7.77(1H, d, J=7.6 Hz), 8.51-8.52(1H, m), 10.33(1H, s) (+)ESI-MS(m/z): 402(M+H)⁺, 424(M+Na)⁺

### EXAMPLE 53

### tert-Butyl 4-({[2-(4-methyl-1-piperidinyl)-3-pyridinyl]carbonyl}amino)phenyl[2-(2-pyridinyl)ethyl]carbamate

The title compound was obtained in a similar manner as in Example 43 from tert-butyl 4-{[(2-chloro-6-methyl-3-pyridinyl)carbonyl]amino}phenyl[2-(2-pyridinyl)ethyl]carbamate and 4-methylpiperidine.

¹H-NMR(DMSO-d₆): δ 0.89(3H, d, J=6.1 Hz), 1.21(9H, s), 1.14-1.18(2H, m), 1.21-1.32(3H, m), 2.78-2.95(4H, m), 3.69-3.75(2H, m), 3.92(2H, t, J=7.4Hz), 6.93-6.97(1H, m), 7.16-7.26(4H, m), 7.65-7.70(3H, m), 7.71-7.84(1H, m), 8.27-8.31(1H, m), 8.45-8.47(1H, m), 10.54(1H, s)

### EXAMPLE 54

### 2-(4-Methyl-1-piperidinyl)-N-(4-{[2-(2-pyridinyl)ethyl]amino}phenyl)nicotinamide

The title compound was obtained in a similar manner as in Example 44 from tert-butyl 4-({[2-(4-methyl-1-piperidinyl)-3-pyridinyl]carbonyl}amino)phenyl[2-(2-pyridinyl)ethyl]carbamate.

¹H-NMR(DMSO-d₆): δ 0.87(3H, d, J=6.2 Hz), 1.05-1.30(2H, m), 1.35-1.66(3H, m), 2.76-2.87(2H, m), 2.99(2H, t, J=7.3 Hz), 3.33-3.41(2H, m), 3.66-3.72(2H, m), 5.63(1H, br.s), 6.59(2H, d J=8.8 Hz), 6.90-6.96(1H, m), 7.23-7.26(1H, m), 7.33(1H, d, J=7.7 Hz), 7.44(2H, d, J=8.8 Hz), 7.68-7.83(2H, m), 8.25-8.28(1H, m), 8.50-8.53(1H, m), 10.21(1H, s) (+)ESI-MS(m/z): 416(M+H)⁺, 438(M+Na)⁺

### EXAMPLE 55

A mixture of tert-butyl 4-{[(2-chloro-6-methyl-3-pyridinyl)carbonyl]amino}phenyl[2-(2-pyridinyl)ethyl]carbamate (700 mg) in 2M dimethylamine-tetrahydrofuran solution (10 ml) was stirred at 65-70° C. for 10 hours. The reaction mixture was poured into a mixture of ethyl acetate and water, and the organic layer was washed with brine and dried over magnesium sulfate. The solvent was concentrated in vacuo and the precipitate was collected by filtration to give tert-butyl 4-({[2-(dimethylamino)-6-methyl-3-pyridinyl]carbonyl}amino)-phenyl[2-(2-pyridinyl)ethyl]carbamate (460 mg).

¹H-NMR(DMSO-d₆): δ 1.33(9H, s), 2.37(3H, s), 2.90(2H, t, J=7.4 Hz), 2.96(6H, s), 3.91(2H, t, J=7.4 Hz), 6.62(1H, d, J=7.6 Hz), 7.15-7.25(4H, m), 7.60(1H, d, J=7.6 Hz), 7.66-7.69(3H, m), 8.46-8.47(1H, m), 10.35(1H, s)

### EXAMPLE 56

### 2-(Dimethylamino)-6-methyl-N-(4-{[2-(2-pyridinyl)ethyl]amino}phenyl)nicotinamide

The title compound was obtained in a similar manner as in Example 44 from tert-butyl 4-({[2-(dimethylamino)-6-methyl-3-pyridinyl]carbonyl}amino)phenyl[2-(2-pyridinyl)ethyl]carbamate.

¹H-NMR(DMSO-d₆): δ 2.35(3H, s), 2.94(6H, s), 3.00(2H, t, J=7.40 Hz), 3.35-3.39(2H, m), 6.57-6.61(3H, m), 7.24-7.34(1H, m), 7.35(1H, d, J=7.8 Hz), 7.41(2H, d, J=8.8 Hz), 7.55 (1H, d, J=7.5 Hz), 7.72-7.76(1H, m), 8.52-8.54(1H, m), 9.94(1H, s) (+)ESI-MS(m/z): 376(M+H)⁺, 398(M+Na)⁺

### EXAMPLE 57

tert-Butyl 4-({[2-(dimethylamino)-3-pyridinyl]carbonyl}amino)phenyl[2-(2-pyridinyl)ethyl]carbamate

The title compound was obtained in a similar manner as in Example 55 from tert-butyl 4-{[(2-chloro-3-pyridinyl)carbonyl]amino}phenyl[2-(2-pyridinyl)ethyl]carbamate and dimethylamine.

¹H-NMR(DMSO-d₆): δ 1.33(9H, s), 2.90(2H, t, J=7.4 Hz), 2.97(6H, s), 3.91(2H, t, J=7.4 Hz), 6.72-6.78(1H, m), 7.15-7.26(4H, m), 7.65-7.74(4H, m), 8.19-8.22(1H, m), 8.45-8.48(1H, m), 10.42(1H, s)

### EXAMPLE 58

### 2-(Dimethylamino)-N-(4-{[2-(2-pyridinyl)ethyl]amino}-phenyl)nicotinamide

The title compound was obtained in a similar manner as in Example 44 from tert-butyl 4-({[2-(dimethylamino)-3-pyridinyl]carbonyl}amino)phenyl[2-(2-pyridinyl)ethyl]carbamate.

¹H-NMR(DMSO-d₆): δ 2.98(2H, t, J=7.4 Hz), 2.96(6H, s), 3.34-3.40(2H, m), 6.57(2H, d, J=8.8 Hz), 6.70-6.76(1H, m), 7.23-7.33(2H, m), 7.41(2H, d, J=8.8 Hz), 7.60-7.71(2H, m), 8.16-8.18(1H, m), 8.52(1H, d, J=4.0 Hz), 9.99(1H, s) (+)ESI-MS(m/z): 362(M+H)⁺, 384(M+Na)⁺

Preparation 54

### 2-Chloro-6-methyl-N-(4-{[2-(2-pyridinyl)ethyl]amino}-phenyl)nicotinamide

The title compound was obtained in a similar manner as in Example 44 from tert-butyl 4-{[(2-chloro-6-methyl-3-pyridinyl)carbonyl]amino}phenyl[2-(2-pyridinyl)ethyl]carbamate.

¹H-NMR(DMSO-d₆): δ 2.49(3H, s), 2.98(2H, t, J=7.4 Hz), 3.33-3.42(2H, m), 5.62(1H, t, J=5.7 Hz), 6.58(2H, d, J=8.9 Hz), 7.20-7.43(5H, m), 7.67-7.71(1H, m), 7.89(1H, d, J=7.7 Hz), 8.50-8.53(1H, m), 10.14(1H, s) (+)ESI-MS(m/z): 367(M+H)⁺, 389(M+Na)⁺

Preparation 55

A mixture of 2-chloro-6-methylnicotinic acid (2.06 g), 4-[2-(2-pyridinyl)ethoxy]phenylamine (2.70 g), 1-hydroxybenzotriazole hydrate (1.93 g) and 1-[3-(dimethylamino)propyl]-3-ethylcarbodiimide (1.96 g) in N,N-dimethylformamide (30 ml) was stirred at ambient temperature overnight. The reaction mixture was poured into a mixture of ethyl acetate and water, and the organic layer was washed with brine and dried over magnesium sulfate. The solvent was evaporated in vacuo and the residue was chromatographed on silica gel eluting with ethyl acetate and n-hexane (7:3-9:1 v/v). The fractions containing the desired product were collected and evaporated in vacuo to give 2-chloro-6-methyl-N-{4-[2-(2-pyridinyl)ethoxy]phenyl}nicotinamide (2.95 g). ¹H-NMR(DMSO-d₆): δ 2.49(3H, s), 3.19(2H, t, J=6.6 Hz), 4.34(2H, t, J=6.6 Hz), 6.92-6.94(2H, m), 7.24-7.25(1H, m), 7.37-7.42(2H, m), 7.58-7.60(2H, m), 7.72-7.74(1H, m), 7.93(1H, d, J=7.7 Hz), 8.52-8.53(1H, m), 10.41(1H, s) (+)ESI-MS(m/z): 368(M+H)⁺, 390(M+Na)⁺

### EXAMPLE 59

A mixture of 2-chloro-6-methyl-N-{4-[2-(2-pyridinyl)ethoxy]phenyl}nicotinamide (440 mg) and piperidine (0.5 ml) in tetrahydrofuran (10 ml) was refluxed under stirring for 5 hours. The reaction mixture was poured into a mixture of ethyl acetate and water, and the organic layer was washed with brine and dried over magnesium sulfate. The solvent was evaporated in vacuo and the residue was chromatographed on silica gel eluting with ethyl acetate and n-hexane (7:3 v/v). The fractions containing the desired product were collected and concentrated in vacuo and the precipitate was collected by filtration to give 6-methyl-2-(1-piperidinyl)-N-{4-[2-(2-pyridinyl)ethoxy]phenyl}nicotinamide (425 mg).

¹H-NMR(DMSO-d₆): δ 1.53(6H, br.s), 2.39(3H, s), 3.18(2H, t, J=6.6 Hz), 4.33(2H, t, J=6.6 Hz), 6.82(1H, d, J=7.6 Hz), 6.92(2H, d, J=9.0 Hz), 7.21-7.28(1H, m), 7.37(1H, d, J=7.8 Hz), 7.62(2H, d, J=9.0 Hz), 7.69-7.77(2H, m), 8.50-8.53(1H, m), 10.44(1H, s) (+)ESI-MS(m/z): 417(M+H)⁺, 439(M+Na)⁺

### EXAMPLE 60

### 6-Methyl-2-(4-methyl-1-piperidinyl)-N-{4-[2-(2-pyridinyl)ethoxy]phenyl}nicotinamide

The title compound was obtained in a similar manner as in Example 44 from 2-chloro-6-methyl-N-{4-[2-(2-pyridinyl)ethoxy]phenyl}nicotinamide and 4-methylpiperidine.

¹H-NMR(DMSO-d₆): δ 0.88(3H, d, J=6.2 Hz), 1.14-1.25(2H, m), 1.28-1.61(3H, m), 2.39 (3H, s), 2.52-2.86(2H, m), 3.18 (2H, t, J=6.6 Hz), 3.62-3.68(2H, m), 4.33(2H, t, J=6.6 Hz), 6.81(1H, d, J=7.6 Hz), 6.92(2H, d, J=9.0 Hz), 7.23-7.28(1H, m), 7.37(1H, d, J=7.7Hz), 7.62(2H, d, J=9.0 Hz), 7.69-7.77(2H, m), 8.50-8.53(1H, m), 10.40(1H, s) (+)ESI-MS(m/z): 431(M+H)⁺, 453(M+Na)⁺

### EXAMPLE 61

A mixture of 2-chloro-6-methyl-N-{4-[2-(2-pyridinyl)ethoxy]phenyl}nicotinamide (736 mg) in 2M dimethylamine-tetrahydrofuran solution (10 ml) was stirred at 65-70° C. for 10 hours. The reaction mixture was poured into a mixture of ethyl acetate and water, and the organic layer was washed with brine and dried over magnesium sulfate. The solvent was evaporated in vacuo and the residue was chromatographed on silica gel eluting with ethyl acetate and n-hexane (7:3 v/v). The fractions containing the desired product were collected and concentrated in vacuo and the precipitate was collected by filtration to give 2-(dimethylamino)-6-methyl-N-{4-[2-(2-pyridinyl)ethoxy]phenyl}-nicotinamide (205 mg).

¹H-NMR(DMSO-d₆): δ 2.35(3H, s), 3.14(6H, s), 3.29(2H, t, J=6.7 Hz), 4.33(2H, t, J=6.7 Hz), 6.61(1H, d, J=7.5 Hz), 6.90(2H, d, J=9.0 Hz), 7.21-7.28(1H, dm), 7.36(1H, d, J=7.7 Hz), 7.54-7.60(3H, m), 7.69-7.77(1H, m), 8.50-8.52(1H, m), 10.14(1H, s) (+)ESI-MS (m/z) : 377 (M+H)⁺, 399 (M+Na)⁺

Preparation 56

### 2-Chloro-N-{4-[2-(2-pyridinyl)ethoxy]phenyl}nicotinamide

The title compound was obtained in a similar manner as in Preparation 55 from 2-chloronicotinic acid and 4-[2-(2-pyridinyl)ethoxy]phenylamine.

¹H-NMR(DMSO-d₆): δ 3.19(2H, t, J=6.6 Hz), 4.34(2H, t, J=6.6 Hz), 6.94(2H, d, J=9.0 Hz), 7.25-7.28(1H, m), 7.39(1H, d, J=7.8 Hz), 7.54-7.61(3H, m), 7.74-7.76(1H, m), 8.04-8.07(1H, m), 8.51-8.53(2H, m), 10.49(1H, s) (+)ESI-MS(m/z): 354(M+H)⁺, 376(M+Na)⁺

### EXAMPLE 62

### 2-(1-Piperidinyl)-N-{4-[2-(2-pyridinyl)ethoxy]phenyl}-nicotinamide

The title compound was obtained in a similar manner as in Example 59 from N-{4-[2-(2-pyridinyl)ethoxy]phenyl}nicotinamide and piperidine.

¹H-NMR(DMSO-d₆): δ 1.53(6H, br.s), 3.15-3.24(6H, m), 4.34(2H, t, J=6.6 Hz), 6.90-6.97(3H, m), 7.37(1H, d, J=7.7Hz), 7.63(2H, d, J=9.0 Hz), 7.69-7.83(2H, m), 8.16-8.29(1H, m), 8.51-8.53(1H, m), 10.40(1H, s) (+)ESI-MS(m/z) : 403(M+H)⁺, 425(M+Na)⁺

### EXAMPLE 63

### 2-(4-Methyl-1-piperidinyl)-N-{4-[2-(2-pyridinyl)ethoxy]-phenyl}nicotinamide

The title compound was obtained in a similar manner as in Example 59 from N-{4-[2-(2-pyridinyl)ethoxy]phenyl}nicotinamide and 4-methylpiperidine.

¹H-NMR(DMSO-d₆): δ 0.88(3H, d, J=6.1 Hz), 1.02-1.27(2H, m), 1.30-1.64(3H, m), 2.76-2.88(2H, m), 3.19(2H, t, J=6.6 Hz), 3.68-3.74(2H, m), 4.34(2H, t, J=6.6 Hz), 6.90-6.95(3H, m), 7.24-7.25(1H, m), 7.37(1H, d, J=7.7 Hz), 7.62-7.83(4H, m), 8.26-8.29(1H, m), 8.51-8.53(1H, m), 10.39(1H, s) (+) ESI-MS (m/z): 417 (M+H)⁺, 439 (M+Na)⁺

Preparation 57

2-Chloro-5-nitropyridine (4.76 g) was added portionwise to a solution of 2-hydroxyethylpyridine (4.43 g) and potassium tert-butoxide (4.04 g) in tetrahydrofuran (60 ml). The mixture was stirred at a temperature between 5 and 20° C. under ice-cooling and the resultant mixture was stirred at ambient temperature for 3 hours. The reaction mixture was poured into a mixture of ethyl acetate and water. The organic layer was washed with 5% aqueous potassium carbonate solution and brine and dried over magnesium sulfate. The solvent was evaporated in vacuo and the residue was chromatographed on silica gel eluting with ethyl acetate: n-hexane (5:5 v/v). The fractions containing the desired product were collected and concentrated in vacuo and the precipitate was collected by filtration to give 5-nitro-2-[2-(2-pyridinyl)ethoxy]pyridine (2.42 g).

¹H-NMR(DMSO-d₆): δ 3.24(2H, t, J=6.68 Hz), 4.80(2H, t, J=6.68 Hz), 6.98(1H, d, J=9.16 Hz), 7.24-7.28(1H, m), 7.35(1H, d, J=7.78 Hz), 7.69-7.77(1H, m), 8.42-8.52(2H, m), 9.09(1H, d, J=2.86 Hz)

Preparation 58

A mixture of 5-nitro-2-[2-(2-pyridinyl)ethoxy]pyridine (736 mg), iron powder (900 mg) and ammonium chloride (101 mg) in ethanol (40 ml) and water (8 ml) was refluxed under stirring for 2.5 hours. After removal of the insoluble materials by filtration, the solvent was evaporated in vacuo and the residue was dissolved in ethyl acetate and water. The organic layer was washed with brine and dried over magnesium sulfate. The solvent was evaporated in vacuo to give 6-[2-(2-pyridinyl)ethoxy]-3-pyridinamine (664 mg).

Preparation 59

### 2-Chloro-6-methyl-N-{6-[2-(2-pyridinyl)ethoxy]-3-pyridinyl}nicotinamide

The title compound was obtained in a similar manner as in Preparation 55 from 2-chloro-6-methylnicotinic acid and 6-[2-(2-pyridinyl)ethoxy]-3-pyridinamine.

¹H-NMR(DMSO-d₆): δ 2.50(3H, s), 3.19(2H, t, J=6.8 Hz), 4.34(2H, t, J=6.8Hz), 6.80(1H, d, J=8.9 Hz), 7.23-7.43(3H, m), 7.68-7.73(1H, m), 7.95-8.01(2H, m), 8.45-8.53(2H, m), 10.61(1H, s)

### EXAMPLE 64

### 6-Methyl-2-(4-methyl-1-piperidinyl)-N-{6-[2-(2-pyridinyl)ethoxy]-3-pyridinyl}nicotinamide

The title compound was obtained in a similar manner as in Example 59 from 2-chloro-6-methyl-N-{6-[2-(2-pyridinyl)ethoxy]-3-pyridinyl}nicotinamide and 4-methylpiperidine.

¹H-NMR(DMSO-d₆): δ 0.89(3H, d, J=6.2 Hz), 1.06-1.30(2H, m), 1.32-1.72(3H, m), 2.39(3H, s), 2.72-2.90(2H, m), 3.18(2H, t, J=6.7 Hz), 3.65-3.70(2H, m), 4.60(2H, t, J=6.7 Hz), 6.76-6.81(2H, m), 7.32-7.36(2H, m), 7.71-7.75(2H, m), 7.97-8.03(1H, m), 8.47-8.51(2H, m), 10.46(1H, s) (+)ESI-MS(m/z): 432(M+H)⁺, 454(M+Na)⁺

### EXAMPLE 65

### 2-(Dimethylamino)-6-methyl-N-{6-[2-(2-pyridinyl)ethoxy]-3-pyridinyl}nicotinamide

The title compound was obtained in a similar manner as in Example 61 from 2-chloro-6-methyl-N-{6-[2-(2-pyridinyl)ethoxy]-3-pyridinyl}nicotinamide and dimethylamine.

¹H-NMR(DMSO-d₆): δ 2.36(3H, s), 2.95(6H, s), 3.18(2H, t, J=6.7 Hz), 4.61(2H, t, J=6.7 Hz), 6.62(1H, d, J=7.5 Hz), 6.77(1H, d, J=8.9 Hz), 7.20-7.26(1H, m), 7.34(1H, d, J=7.8 Hz), 7.68-7.76(1H, m), 7.95-8.00(1H, m), 8.46-8.52(2H, m), 10.30(1H, s) (+)ESI-MS(m/z): 378(M+H)⁺, 400(M+Na)⁺

Preparation 60

A mixture of 2-chloro-6-methylnicotinic acid (772 mg), 3-{[4-(4-aminophenyl)-1-piperazinyl]methyl}benzonitrile (1.38 g), 1-hydroxybenzotriazole hydrate (723 mg) and 1-[3-(dimethylamino)propyl]-3-ethylcarbodiimide (733 mg) in N,N-dimethylformamide (15 ml) was stirred at ambient temperature overnight. The reaction mixture was poured into a mixture of ethyl acetate and water, and the organic layer was washed with brine and dried over magnesium sulfate. The solvent was concentrated in vacuo and the precipitate was collected by filtration to give 2-chloro-N-{4-[4-(3-cyanobenzyl)-1-piperazinyl]phenyl}-6-methylnicotinamide (1.69 g).

¹H-NMR(DMSO-d₆): δ 2.51(3H, s), 2.51-2.54(4H, m), 3.09-3.11(4H, m), 3.60(2H, s), 6.92(2H, d, J=9.0 Hz), 7.38(1H, d, J=7.8 Hz), 7.60-7.68(3H, m), 7.72-7.76(3H, m), 7.91(1H, d, J=7.7 Hz), 10.31(1H, s) (+)ESI-MS(m/z): 446(M+H)⁺, 468(M+Na)⁺

### EXAMPLE 66

A mixture of 2-chloro-N-{4-[4-(3-cyanobenzyl)-1-piperazinyl]phenyl}-6-methylnicotinamide (400 mg) and 4-methylpiperidine (0.5 ml) in tetrahydrofuran (5 ml) was refluxed under stirring for 12 hours. The reaction mixture was poured into a mixture of ethyl acetate and water, and the organic layer was washed with brine and dried over magnesium sulfate. The solvent was concentrated in vacuo and the precipitate was collected by filtration to give N-{4-[4-(3-cyanobenzyl)-1-piperazinyl]phenyl}-6-methyl-2-(4-methyl-1-piperidinyl)nicotinamide (380 mg).

¹H-NMR(DMSO-d₆): δ 0.90(3H, d, J=6.2 Hz), 1.17-1.24(2H, m), 1.27-1.69(3H, m), 2.39(3H, s), 2.50-2.52(4H, m), 2.75-2.86(2H, m), 3.09-3.10(4H, m), 3.59(2H, s), 3.59-3.67(2H, m), 6.82(1H, d, J=7.7 Hz), 6.92(2H, d, J=9.0 Hz), 7.53-7.60(3H, m), 7.68-7.77(4H, m), 10.39(1H, s) (+)ESI-MS(m/z) : 509(M+H)⁺, 531(M+Na)⁺

### EXAMPLE 67

A mixture of 2-chloro-N-{4-[4-(3-cyanobenzyl)-1-piperazinyl]phenyl}-6-methylnicotinamide (400 mg) in 2M dimethylamine-tetrahydrofuran solution (10 ml) was stirred at 65-70° C. for 10 hours. The reaction mixture was poured into a mixture of ethyl acetate and water, and the organic layer was washed with brine and dried over magnesium sulfate. The solvent was evaporated in vacuo and the residue was chromatographed on silica gel eluting with ethyl acetate and n-hexane (7:3 v/v). The fractions containing the desired product were collected and concentrated in vacuo and the precipitate was collected by filtration to give N-{4-[4-(3-cyanobenzyl)-1-piperazinyl]phenyl}-2-(dimethylamino)-6-methylnicotinamide (90 mg).

¹H-NMR(DMSO-d₆): δ 2.35(3H, s), 2.49-2.54(4H, m), 2.94(6H, s), 3.07-3.09(4H, m), 3.59(2H, s), 6.60(1H, d, J=7.5 Hz), 6.89(2H, d, J=9.0 Hz), 7.51-7.60(4H, m), 7.68-7.77(3H, m), 10.07(1H, s) (+)ESI-MS(m/z): 455(M+H)⁺, 477(M+Na)⁺

Preparation 61

### 2-Chloro-N-{6-[4-(3-cyanobenzyl)-1-piperazinyl]-3-pyridinyl}-6-methylnicotinamide

The title compound was obtained in a similar manner as in Preparation 60 from 3-{[4-(5-amino-2-pyridinyl)-1-piperazinyl]methyl}benzonitrile and 2-chloro-6-methylnicotinic acid.

¹H-NMR(DMSO-d₆): δ 2.45(3H, s), 2.48-2.51(4H, m), 3.43-3.48(4H, m), 3.59(2H, s), 6.85(1H, d, J=9.1 Hz), 7.40(1H, d, J=7.8 Hz), 7.53-7.60(1H, m), 7.69-7.90(5H, m), 8.38(1H, d, J=2.6 Hz), 10.40(1H, s) (+)ESI-MS(m/z): 447 (M+H)⁺, 469 (M+Na)⁺

### EXAMPLE 68

N-{6-[4-(3-Cyanobenzyl)-1-piperazinyl]-3-pyridinyl}-6-methyl-2-(4-methyl-1-piperidinyl)nicotinamide

The title compound was obtained in a similar manner as in Example 66 from 2-chloro-N-{6-[4-(3-cyanobenzyl)-1-piperazinyl]-3-pyridinyl}-6-methylnicotinamide and 4-methylpiperidine.

¹H-NMR(DMSO-d₆): δ 0.89(3H, d, J=6.2 Hz), 1.14-1.21(2H, m), 1.26-1.66(3H, m), 2.39(3H, s), 2.45-2.51(4H, m), 2.75-2.86(2H, m), 3.43-3.58(4H, m), 3.58-3.69(4H, m), 6.79-6.87(2H, m), 7.53-7.60(1H, m), 7.69-7.78(4H, m), 7.87-7.93(1H, m), 8.42(1H, d, J=2.6 Hz), 10.36(1H, s) (+)ESI-MS(m/z): 510(M+H)⁺, 532(M+Na)⁺

### EXAMPLE 69

### N-{6-[4-(3-Cyanobenzyl)-1-piperazinyl]-3-pyridinyl}-2-(dimethylamino)-6-methylnicotinamide

The title compound was obtained in a similar manner as in Example 67 from 2-chloro-N-{6-[4-(3-cyanobenzyl)-1-piperazinyl]-3-pyridinyl}-6-methylnicotinamide and dimethylamine.

¹H-NMR(DMSO-d₆): δ 2.35(3H, s), 2.45-2.51(4H, m), 3.34(6H, s), 3.42-3.46(4H, m), 3.58(2H, s), 6.61(1H, d, J=7.6 Hz), 6.83(2H, d, J=9.1 Hz), 7.53-7.60(2H, m), 7.68-7.88(4H, m), 8.39-8.40(1H, m), 10.12(1H, s) (+)ESI-MS(m/z): 456(M+H)⁺, 478(M+Na)⁺

### EXAMPLE 70

1-[3-(Dimethylamino)propyl]-3-ethylcarbodiimide (10.0 g) was added to a solution of N-(4-aminophenyl)-N-[2-(2-pyridinyl)ethyl]formamide (13.0 g), 2-(dimethylamino)-4-methylbenzoic acid (11.6 g), 1-hydroxybenzotriazole (8.7 g) and 4-dimethylaminopyridine (0.33 g) in N,N-dimethylformamide (130 ml) under ice-cooling and the mixture was stirred at ambient temperature for 18 hours.

The reaction mixture was poured into a mixture of ethyl acetate and water. The separated organic layer was washed with water, dried over magnesium sulfate and evaporated in vacuo. The residue was purified by column chromatography on silica gel using ethyl acetate as an eluent. The eluted fractions containing the desired product were collected and evaporated in vacuo to give 2-(dimethylamino)-N-(4-{formyl[2-(2-pyridinyl)ethyl]amino}phenyl)-4-methylbenzamide (20.18 g).

¹H-NMR(DMSO-d₆): δ 2.35(3H, s), 2.77(6H, s), 2.91(2H, t, J=7.5 Hz), 4.11(2H, t, J=7.5 Hz), 6.95(1H, d, J=7.9 Hz), 7.10(1H, s), 7.17-7.33(4H, m), 7.62-7.82(4H, m), 8.34(1H, s), 8.45-8.52(1H, m), 11.55 (1H, s)

### EXAMPLE 71

conc. Hydrochloric acid (24.8 g) was added to a solution of 2-(dimethylamino)-N-(4-{formyl[2-(2-pyridinyl)ethyl]amino}phenyl)-4-methylbenzamide (20.0 g) in methanol (100 ml) under ice-cooling and the mixture was stirred at ambient temperature for 30 hours. The reaction mixture was evaporated in vacuo and to the residue was added a mixture of ethyl acetate and water. The mixture was adjusted to pH 9 with 20% aqueous potassium carbonate solution. The separated organic layer was washed with water, dried over magnesium sulfate and evaporated in vacuo. The residue was crystallized from a mixture of ethanol and heptane to give 2-(dimethylamino)-4-methyl-N-(4-{[2-(2-pyridinyl)ethyl]amino}phenyl)benzamide (9.33 g).

¹H-NMR(DMSO-d₆): δ 2.33(3H, s), 2.75(6H, s), 2.99(2H, t, J=7.2 Hz), 3.30-3.44(2H, m), 5.56(1H, t, J=5.7 Hz), 6.59(2H, d, J=8.8 Hz), 6.94(1H, d, J=8.0 Hz), 7.08(1H, s), 7.18-7.27(1H, m), 7.32(1H, d, J=7.8 Hz), 7.43(2H, d, J=8.8 Hz), 7.64-7.77(2H, m), 8.49-8.55(1H, m), 11.18(1H, s) (+)ESI-MS(m/z): 375(M+H)⁺, 397(M+Na)⁺

Preparation 62

A mixture of methyl 4-chloro-2-aminobenzoate (5.4 g) and dimethyl sulfate (7.5 ml) was stirred for 70 hours at 100° C. To the mixture was added a saturated aqueous sodium hydrogencarbonate solution and the mixture was extracted with ethyl acetate. The extract layer was washed with water, dried over magnesium sulfate and evaporated in vacuo. The residue was purified by column chromatography on silica gel using a mixture of hexane and ethyl acetate (19:1 v/v) as an eluent. The eluted fractions containing the desired product were collected and evaporated in vacuo to give methyl 4-chloro-2-(dimethylamino)benzoate (5.31 g).

¹H-NMR(DMSO-d₆): δ 2.79(6H, s), 3.81(3H, s), 6.82(1H, dd, J=1.9 Hz, 8.3 Hz), 6.95(1H, d, J=1.9 Hz), 7.51(1H, d, J=8.3 Hz) (+)ESI-MS(m/z): 214(M+H)⁺, 236(M+Na)⁺

Preparation 63

A mixture of methyl 4-chloro-2-{[(trifluoromethyl)sulfonyl]oxy}benzoate (5.0 g) and 2 mol/l tetrahydrofuran solution of dimethylamine (19.6 ml) was heated at 70° C. in sealed tube for 60 hours. To the reaction mixture was added a mixture of ethyl acetate and water. The separated organic layer was washed with water, dried over magnesium sulfate and evaporated in vacuo. The residue was purified by column chromatography on silica gel using a mixture of hexane and ethyl acetate (9:1 v/v) as an eluent. The eluted fractions containing the desired product were collected and evaporated in vacuo to give methyl 4-chloro-2-(dimethylamino)benzoate (2.24 g).

¹H-NMR(DMSO-d₆): δ 2.79(6H, s), 3.81(3H, s), 6.82(1H, dd, J=1.9 Hz, 8.3 Hz), 6.95(1H, d, J=1.9 Hz), 7.51(1H, d, J=8.3 Hz) (+)ESI-MS(m/z): 214(M+H)⁺, 236(M+Na)⁺

Preparation 64

A mixture of methyl 4-chloro-2-(dimethylamino)benzoate (5.3 g) and sodium hydroxide (2.0 g) in a mixture of methanol (53 ml) and water (10 ml) was stirred under reflux for 20 hours. To the reaction mixture was added conc. hydrochloric acid (4.1 ml) and the mixture was evaporated in vacuo. The residue was purified by column chromatography on silica gel using a mixture of chloroform and methanol (19:1 v/v) as an eluent. The eluted fractions containing the desired product were collected and evaporated in vacuo to give 4-chloro-2-(dimethylamino)benzoic acid (4.27 g).

¹H-NMR(DMSO-d₆): δ 2.82(6H, s), 7.18(1H, dd, J=2.0 Hz, 8.4 Hz), 7.49(1H, d, J=2.0 Hz), 7.79(1H, d, J=8.4 Hz), 15.48(1H, s) (−)ESI-MS(m/z): 397(M−H)⁻

### EXAMPLE 72

The following compound was obtained in substantially the same manner as in Example 70.

### 4-Chloro-2-(dimethylamino)-N-(4-{formyl[2-(2-pyridinyl)ethyl]amino}phenyl)benzamide

¹H-NMR(DMSO-d₆): δ 2.82(6H, s), 2.91(2H, t, J=7.5 Hz), 4.11(2H, t, J=7.5 Hz), 7.02(1H, dd, J=2.0 Hz, 8.2 Hz), 7.10(1H, d, J=2.0 Hz), 7.19-7.32(4H, m), 7.52(1H, d, J=8.2 Hz), 7.66-7.73(1H, m), 7.76(2H, d, J=8.8 Hz), 8.34(1H, s), 8.47-8.50(1H, m), 10.80(1H, s)

### EXAMPLE 73

The following compound was obtained in substantially the same manner as in Example 71.

### 4-Chloro-2-(dimethylamino)-N-(4-{[2-(2-pyridinyl)ethyl]amino}phenyl)benzamide

¹H-NMR(DMSO-d₆): δ 2.79(6H, s), 2.98(2H, t, J=7.2 Hz), 3.29-3.44(2H, m), 5.57(1H, t, J=5.8 Hz), 6.58(2H, d, J=8.7 Hz), 7.01(1H, dd, J=1.9 Hz, 8.1 Hz), 7.08(1H, d, J=1.9 Hz), 7.18-7.27(1H, m), 7.32(1H, d, J=7.7 Hz), 7.41(2H, d, J=8.7 Hz), 7.52(1H, d, J=8.1 Hz), 7.66-7.77(1H, m), 8.49-8.54(1H, m), 10.40(1H, s) (+)ESI-MS(m/z): 395(M+H)⁺, 417(M+Na)⁺

### EXAMPLE 74

1-[3-(Dimethylamino)propyl]-3-ethylcarbodiimide (0.19 g) was added to a solution of tert-butyl 4-aminophenyl[2-(2-pyridinyl)ethyl]carbamate (0.31 g), 4-chloro-2-(dimethylamino)benzoic acid (0.24 g), 1-hydroxybenzotriazole (0.16 g) and 4-dimethylaminopyridine (6 mg) in tetrahydrofuran (4 ml) and the mixture was stirred at ambient temperature for 18 hours. To the reaction mixture was added a solution of 4N hydrogen chloride in 1,4-dioxane (7.5 ml) and the mixture was stirred at ambient temperature for 30 hours. The reaction mixture was poured into a mixture of ethyl acetate and water, and the mixture was adjusted to pH 9 with potassium carbonate. The separated organic layer was washed with water, dried over magnesium sulfate and evaporated in vacuo. The residue was crystallized from ethyl acetate to give 4-chloro-2-(dimethylamino)-N-(4-{[2-(2-pyridinyl)ethyl]amino}phenyl)benzamide (0.33 g).

¹H-NMR(DMSO-d₆): δ 2.79(6H, s), 2.98(2H, t, J=7.2 Hz), 3.29-3.44(2H, m), 5.57(1H, t, J=5.8 Hz), 6.58(2H, d, J=8.7 Hz), 7.01(1H, dd, J=1.9 Hz, 8.1 Hz), 7.08(1H, d, J=1.9 Hz), 7.18-7.27(1H, m), 7.32(1H, d, J=7.7 Hz), 7.41(2H, d, J=8.7 Hz), 7.52(1H, d, J=8.1 Hz), 7.66-7.77(1H, m), 8.49-8.54(1H, m), 10.40(1H, s) (+)ESI-MS(m/z): 395(M+H)⁺, 417(M+Na)⁺

Preparation 65

The following compound was obtained in substantially the same manner as in Preparation 62.

### Methyl 2-(dimethylamino)-4-fluorobenzoate

¹H-NMR(DMSO-d₆): δ 2.79(6H, s), 3.80(3H, s), 6.54-6.65(1H, m), 6.73(1H, dd, J=2.4 Hz, 12.7 Hz), 7.57(1H, dd, J=7.2 Hz, 8.5 Hz)

Preparation 66

The following compound was obtained in substantially the same manner as in Preparation 64.

### 2-(Dimethylamino)-4-fluorobenzoic acid

¹H-NMR(DMSO-d₆): δ 2.89(6H, s), 6.99-7.11(1H, m), 7.41(1H, dd, J=2.5 Hz, 11.2 Hz), 7.91(1H, dd, J=6.8 Hz, 8.7 Hz), 10.43-13.22(1H, br-s) (−)ESI-MS(m/z): 182(M−H)⁻

### EXAMPLE 75

The following compound was obtained in substantially the same manner as in Example 74.

### 2-(Dimethylamino)-4-fluoro-N-(4-{[2-(2-pyridinyl)ethyl]amino}phenyl)benzamide

¹H-NMR(DMSO-d₆): δ 2.79(6H, s), 2.99(2H, t, J=7.2 Hz), 3.31-3.43(2H, m), 5.58(1H, s), 6.59(2H, d, J=8.8 Hz), 6.73-6.84(1H, m), 6.89(1H, dd, J=2.4 Hz, 12.1 Hz), 7.18-7.27(1H, m), 7.32(1H, d, J=7.7 Hz), 7.42(2H, d, J=8.8 Hz), 7.57(1H, dd, J=7.2 Hz, 8.4 Hz), 7.67-7.76(1H, m), 8.50-8.56(1H, m), 10.38(1H, s) (+) ESI-MS (m/z): 379 (M+H)⁺, 401 (M+Na)⁺

Preparation 67

A mixture of 2-fluoro-4-(trifluoromethyl)benzonitrile (5.0 g) and 2 mol/l tetrahydrofuran solution of dimethylamine (39.7 ml) was heated at 80° C. in sealed tube for 15 hours. To the reaction mixture was added a mixture of ethyl acetate and water. The separated organic layer was washed with water, dried over magnesium sulfate and evaporated in vacuo to give 2-(dimethylamino)-4-(trifluoromethyl)benzonitrile (5.55 g).

¹H-NMR(DMSO-d₆): δ 3.09(6H, s), 7.15(1H, d, J=8.0 Hz), 7.21(1H, s), 7.82(1H, d, J=8.0 Hz)

Preparation 68

A mixture of 2-(dimethylamino)-4-(trifluoromethyl)benzonitrile (5.0 g) and sodium hydroxide (2.1 g) in ethylene glycol (22 ml) was stirred at 180° C. for 6 hours. The reaction mixture was added to water (22 ml) at 80° C. and the mixture was stirred at the same temperature for an hour. To the mixture was added a saturated aqueous sodium chloride solution and adjusted to pH 4 with 6N hydrochloric acid. The mixture was extracted with a mixture of ethyl acetate and tetrahydrofuran. The organic layer was dried over magnesium sulfate and evaporated in vacuo. The residue was triturated with diisopropyl ether to give 2-(dimethylamino)-4-(trifluoromethyl)benzoic acid (4.51 g).

¹H-NMR(DMSO-d₆): δ 2.88(6H, s), 7.35(1H, dd, J=0.9 Hz, 8.0 Hz), 7.56(1H, d, J=0.9 Hz), 7.87(1H, d, J=8.0 Hz), 15.03(1H, s) (−)ESI-MS(m/z): 232(M−H)⁻

### EXAMPLE 76

The following compound was obtained in substantially the same manner as in Example 74.

### 2-(Dimethylamino)-N-(4-{[2-(2-pyridinyl)ethyl]amino}phenyl)-4-(trifluoromethyl)benzamide

¹H-NMR(DMSO-d₆): δ 2.86(6H, s), 2.99(2H, t, J=7.2 Hz), 3.29-3.45(2H, m), 5.59(1H, t, J=5.7 Hz), 6.59(2H, d, J=8.8 Hz), 7.17-7.28(3H, m), 7.32(1H, d, J=7.8 Hz), 7.43(2H, d, J=8.8 Hz), 7.62(1H, d, J=8.1 Hz), 7.66-7.77(1H, m), 8.48-8.56(1H, m), 10.28(1H, s) (+)ESI-MS(m/z): 429(M+H)⁺, 451(M+Na)⁺

Preparation 69

The following compound was obtained in substantially the same manner as in Preparation 63.

### Benzyl 2-(dimethylamino)-4-methoxybenzoate

¹H-NMR(DMSO-d₆): δ 2.74(6H, s), 3.78(3H, s), 5.26(2H, s), 6.39-6.46(2H, m), 7.32-7.49(5H, m), 7.57-7.64(1H, m)

Preparation 70

To a mixture of benzyl 2-(dimethylamino)-4-methoxybenzoate (19.2 g) in methanol (200 ml) was added 10% palladium on carbon (6.0 g, 50% wet). The reaction mixture was stirred at ambient temperature for 3 hours under hydrogen atmosphere.

The catalyst was filtered off and the solvent was removed by concentration. The residue was triturated with diisopropyl ether to give 2-(dimethylamino)-4-methoxybenzoic acid (11.46 g).

¹H-NMR(DMSO-d₆): δ 2.78(6H, s), 3.84(3H, s), 6.91(1H, dd, J=2.4 Hz, 8.8 Hz), 7.20(1H, d, J=2.4 Hz), 7.90(1H, d, J=8.8 Hz), 17.20(1H, s) (−)ESI-MS(m/z): 194(M−H)⁻

### EXAMPLE 77

The following compound was obtained in substantially the same manner as in Example 74.

### 2-(Dimethylamino)-4-methoxy-N-(4-{[2-(2-pyridinyl)ethyl]amino}phenyl)benzamide

¹H-NMR(DMSO-d₆): δ 2.75(6H, s), 2.99(2H, t, J=7.2 Hz), 3.30-3.45(2H, m), 3.81(3H, s), 5.57(1H, t, J=5.7 Hz), 6.59(2H, d, J=8.8 Hz), 6.67-6.78(2H, m), 7.18-7.27(1H, m), 7.32(1H, d, J=7.8 Hz), 7.43(2H, d, J=8.8 Hz), 7.66-7.79(2H, m), 8.50-8.55(1H, m), 11.08(1H, s) (+)ESI-MS(m/z): 391(M+H)⁺

Preparation 71

To a mixture of 4-acetyl-2-nitrophenol (23.0 g) and 37% aqueous formaldehyde (190 ml) in methanol (460 ml) was added 10% palladium on carbon (11.5 g, 50% wet). The reaction mixture was stirred at ambient temperature for 16 hours under hydrogen atmosphere. The catalyst was filtered off and the solvent was removed by concentration. To the residue was added ethyl acetate and the mixture was washed with brine, dried over magnesium sulfate and evaporated in vacuo. The residue was triturated with diisopropyl ether to give 1-[3-(dimethylamino)-4-hydroxyphenyl]ethanone (15.37 g).

¹H-NMR(DMSO-d₆):δ 2.47(3H, s), 2.70(6H, s), 6.84(1H, d, J=8.2 Hz), 7.40(1H, d, J=2.0 Hz), 7.50(1H, dd, J=2.0 Hz, 8.2 Hz), 10.10(1H, s) (+)ESI-MS(m/z): 180(M+H)⁺, 202(M+Na)⁺

Preparation 72

Trifluoromethanesulfonic anhydride (25.6 ml) was added dropwise to a mixture of 1-[3-(dimethylamino)-4-hydroxyphenyl]ethanone (22.7 g) and triethylamine (21.2 ml) in dichloromethane (227 ml) under ice-cooling and the mixture was stirred at the same temperature for 1.5 hours. The reaction mixture was poured into a saturated aqueous sodium hydrogencarbonate solution. The separated organic layer was washed with water, dried over magnesium sulfate and evaporated in vacuo to give 4-acetyl-2-(dimethylamino)phenyl trifluoromethanesulfonate (49.27 g) as a crude oil.

¹H-NMR(DMSO-d₆):δ 2.62(3H, s), 2.78(6H, s), 7.47-7.54(1H, m), 7.66-7.73(2H, m)

Preparation 73

A mixture of 4-acetyl-2-(dimethylamino)phenyl trifluoromethanesulfonate (39.4 g), palladium (II) acetate (1.4 g), 1,3-bis(diphenylphosphino)propane (2.6 g) and triethylamine (52.9 ml) in a mixture of dimethyl sulfoxide (200 ml) and methanol (100 ml) was purged with carbon monoxide for 30 minutes at ambient temperature and the mixture was stirred under a carbon monoxide balloon at 70° C. for 5 hours. The reaction mixture was diluted with water and extracted with ethyl acetate. The organic layer was washed with water, dried over magnesium sulfate and evaporated in vacuo. The residue was purified by column chromatography on silica gel using a mixture of hexane and ethyl acetate (4:1 v/v) as an eluent. The eluted fractions containing the desired product were collected and evaporated in vacuo to give methyl 4-acetyl-2-(dimethylamino)benzoate (14.36 g).

¹H-NMR(DMSO-d₆):δ 2.59(3H, s), 2.82(6H, s), 3.84(3H, s), 7.37(1H, dd, J=1.5 Hz, 7.9 Hz), 7.42(1H, d, J=1.5 Hz), 7.59(1H, d, J=7.9 Hz)

Preparation 74

Sodium borohydride (0.56 g) was added to a mixture of methyl 4-acetyl-2-(dimethylamino)benzoate (6.5 g) in methanol (65 ml) under ice-cooling and the mixture was stirred for 30 minutes at the same temperature. The solvent was removed by concentration. To the residue was added ethyl acetate and the mixture was washed with water, dried over magnesium sulfate and evaporated in vacuo to give methyl 2-(dimethylamino)-4-(1-hydroxyethyl)benzoate (6.5 g).

¹H-NMR(DMSO-d₆):δ 1.31(3H, d, J=6.5 Hz), 2.76(6H, s), 3.78(3H, s), 4.61-4.76(1H, m), 5.19(1H, d, J=4.3 Hz), 4.79(1H, dd, J=1.2 Hz, 7.9 Hz), 6.96(1H, d, J=1.2 Hz), 7.46(1H, d, J=7.9 Hz)

Preparation 75

To a mixture of methyl 2-(dimethylamino)-4-(1-hydroxyethyl)benzoate (6.4 g) and 4N hydrogen chloride in 1,4-dioxane (21.5 ml) in methanol (64 ml) was added 10% palladium on carbon (2.0 g, 50% wet). The reaction mixture was stirred at 35° C. for 16 hours under hydrogen atmosphere. The catalyst was filtered off and the solvent was removed by concentration. To the residue was added ethyl acetate and adjusted to pH 9 with potassium carbonate. The separated organic layer was washed with water, dried over magnesium sulfate and evaporated in vacuo to give methyl 2-(dimethylamino)-4-ethylbenzoate (5.47 g).

¹H-NMR(DMSO-d₆):δ 1.17(3H, t, J=7.6 Hz), 2.58(2H, q, J=7.6 Hz), 2.75(6H, s), 3.78(3H, s), 6.68(1H, d, J=7.9 Hz), 6.79(1H, s), 7.45(1H, d, J=7.9 Hz)

Preparation 76

The following compound was obtained in substantially the same manner as in Preparation 64.

2-(Dimethylamino)-4-ethylbenzoic acid

¹H-NMR(DMSO-d₆):δ 1.21(3H, t, J=7.6 Hz), 2.68(2H, q, J=7.6 Hz), 2.81(6H, s), 7.22(1H, d, J=7.9 Hz), 7.57(1H, s), 7.89(1H, d, J=7.9 Hz), 17.79(1H, s)

(+)ESI-MS(m/z): 194(M+H)⁺, 216(M+Na)⁺

### EXAMPLE 78

The following compound was obtained in substantially the same manner as in Example 74.

2-(Dimethylamino)-4-ethyl-N-(4-{[2-(2-pyridinyl)ethyl]amino}phenyl)benzamide

¹H-NMR(DMSO-d₆):δ 1.20(3H, t, J=7.5 Hz), 2.63(2H, q, J=7.5 Hz), 2.76(6H, s), 2.99(2H, t, J=7.2 Hz), 3.30-3.43(2H, m), 5.57(1H, t, J=5.7 Hz), 6.60(2H, d, J=8.7 Hz), 6.97(1H, d, J=7.9 Hz), 7.08(1H, s), 7.22(1H, dd, J=5.4 Hz, 7.2 Hz), 7.32(1H, d, J=7.9 Hz), 7.44(2H, d, J=8.7 Hz), 7.64-7.76(2H, m), 8.49-8.56(1H, m), 11.13(1H, s)

(+)ESI-MS(m/z): 389(M+H)⁺, 411(M+Na)⁺

Preparation 77

Methyl 4-acetyl-2-(dimethylamino)benzoate (5.0 g) was added to a mixture of methyltriphenylphosphonium bromide (12.1 g) and potassium tert-butoxide (3.55 g) in tetrahydrofuran (120 ml) at ambient temperature and the mixture was stirred for 3 hours at 57° C. The reaction mixture was poured into a mixture of ethyl acetate and water and adjusted to pH 2 with 6N hydrochloric acid. The separated organic layer was washed with water, dried over magnesium sulfate and evaporated in vacuo. The residue was purified by column chromatography on silica gel using a mixture of hexane and ethyl acetate (9:1 v/v) as an eluent. The eluted fractions containing the desired product were collected and evaporated in vacuo to give methyl 2-(dimethylamino)-4-isopropenylbenzoate (4.83 g).

¹H-NMR(DMSO-d₆):δ 2.11(3H, s), 2.78(6H, s), 3.80(3H, s), 5.14-5.18(1H, m), 5.45-5.48(1H, m), 6.95(1H, dd, J=1.7 Hz, 8.0 Hz), 6.99(1H, d, J=1.7 Hz), 7.50(1H, d, J=8.0 Hz)

Preparation 78

To a mixture of methyl 2-(dimethylamino)-4-isopropenylbenzoate (4.8 g) in methanol (50 ml) was added 10% palladium on carbon (1.0 g, 50% wet). The reaction mixture was stirred at ambient temperature for 6 hours under hydrogen atmosphere. The catalyst was filtered off and the solvent was removed by concentration to give methyl 2-(dimethylamino)-4-isopropylbenzoate (4.56 g).

¹H-NMR(DMSO-d₆):δ 1.19(6H, d, J=6.8 Hz), 2.73 -2.97(1H, m), 2.76(6H, s), 3.78(3H, s), 6.71(1H, dd, J=1.4 Hz, 7.9 Hz), 6.80(1H, d, J=1.4 Hz), 7.45(1H, d, J=7.9 Hz)

Preparation 79

The following compound was obtained in substantially the same manner as in Preparation 64.

2-(Dimethylamino)-4-isopropylbenzoic acid

¹H-NMR(DMSO-d₆):δ 1.23(6H, d, J=7.0 Hz), 2.82(6H, s), 2.88-3.06(1H, m), 7.27(1H, d, J=8.0 Hz), 7.61(1H, s), 7.92(1H, d, J=8.0 Hz), 17.82(1H, s)

(−)ESI-MS(m/z): 206(M−H)⁻

### EXAMPLE 79

The following compound was obtained in substantially the same manner as in Example 74.

2-(Dimethylamino)-4-isopropyl-N-(4-([2-(2-pyridinyl)ethyl]amino}phenyl)benzamide

¹H-NMR(DMSO-d₆):δ 1.22(6H, d, J=6.7 Hz), 2.76(6H, s), 2.82-3.00(1H, m), 2.99(2H, t, J=7.3 Hz), 3.30-3.44(2H, m), 5.57(1H, t, J=5.8 Hz), 6.59(2H, d, J=8.8 Hz), 6.99(1H, dd, J=1.3 Hz, 8.0 Hz), 7.09(1H, d, J=1.3 Hz), 7.18-7.27(1H, m), 7.32(1H, d, J=7.8 Hz), 7.44(2H, d, J=8.8 Hz), 7.63-7.77(2H, m), 8.50-8.55(1H, m), 11.06(1H, s)

### EXAMPLE 80

4N Hydrogen chloride in ethyl acetate (0.85 ml) was added to a mixture of 2-(dimethylamino)-4-isopropyl-N-(4-{[2-(2-pyridinyl)ethyl]amino}phenyl)benzamide (0.34 g) in ethyl acetate (20 ml) and the mixture was stirred at ambient temperature for an hour. The isolated precipitate was collected by filtration to give 2-(dimethylamino)-4-isopropyl-N-(4-([2-(2-pyridinyl)ethyl]amino}phenyl)benzamide trihydrochloride (0.35 g).

¹H-NMR(DMSO-d₆):δ 1.27(6H, d, J=6.9 Hz), 2.97-3.14(1H, m), 3.25(6H, s), 3.54(2H, t, J=6.6 Hz), 3.74(2H, t, J=6.6 Hz), 7.30(2H, d, J=8.7 Hz), 7.51(1H, d, J=8.0 Hz), 7.78(2H, d, J=8.7 Hz), 7.88-7.98(2H, m), 8.04(1H, d, J=8.0 Hz), 8.14(1H, d, J=8.0 Hz), 8.47-8.57(1H, m), 8.78-8.86(1H, m), 11.19(1H, s) (+)ESI-MS(m/z): 403(M+H)⁺, 425(M+Na)⁺

Preparation 80

The following compound was obtained in substantially the same manner as in Preparation 71.

4-tert-Butyl-2-(dimethylamino)phenol

¹H-NMR(DMSO-d₆):δ 1.23(9H, s), 2.66(6H, s), 6.65(1H, d, J=8.1 Hz), 6.77(1H, dd, J=2.2 Hz, 8.1 Hz), 6.85(1H, d, J=2.2 Hz), 8.74(1H, s)

Preparation 81

The following compound was obtained in substantially the same manner as in Preparation 72.

4-tert-Butyl-2-(dimethylamino)phenyl trifluoromethanesulfonate

¹H-NMR(DMSO-d₆):δ 1.29(9H, s), 2.73(6H, s), 7.07-7.16(1H, m), 7.17-7.26(2H, m)

Preparation 82

The following compound was obtained in substantially the same manner as in Preparation 73.

Methyl 4-tert-butyl-2-(dimethylamino)benzoate

¹H-NMR(DMSO-d₆):δ 1.27(9H, s), 2.77(6H, s), 3.78(3H, s), 6.65(1H, d, J=8.1 Hz), 6.85(1H, s), 7.46(1H, d, J=8.1 Hz)

Preparation 83

The following compound was obtained in substantially the same manner as in Preparation 64.

4-tert-Butyl-2-(dimethylamino)benzoic acid

¹H-NMR(DMSO-d₆):δ 1.32(9H, s), 2.84(6H, s), 7.43(1H, dd, J=1.8 Hz, 8.3 Hz), 7.73(1H, d, J=1.8 Hz), 7.93(1H, d, J=8.3 Hz), 17.99(1H, s)

(−)ESI-MS(m/z): 220(M−H)⁻

### EXAMPLE 81

The following compound was obtained in substantially the same manner as in Example 74.

4-tert-Butyl-2-(dimethylamino)-N-(4-{[2-(2-pyridinyl)ethyl]amino}phenyl)benzamide

¹H-NMR(DMSO-d₆):δ 1.30(9H, s), 2.77(6H, s), 2.99(2H, t, J=7.4 Hz), 3.37(2H, t, J=7.4 Hz), 5.67(1H, s), 6.59(2H, d, J=8.8 Hz), 7.14(1H, dd, J=1.8 Hz, 8.1 Hz), 7.18-7.27(2H, m), 7.32(1H, d, J=7.7 Hz), 7.44(2H, d, J=8.8 Hz), 7.64-7.77(2H, m), 8.50-8.55(1H, m), 11.11 (1H, s)

### EXAMPLE 82

The following compound was obtained in substantially the same manner as in Example 80.

4-tert-Butyl-2-(dimethylamino)-N-(4-{[2-(2-pyridinyl)ethyl]amino}phenyl)benzamide trihydrochloride

¹H-NMR(DMSO-d₆):δ 1.36(9H, s), 3.27(6H, s), 3.53(2H, t, J=6.5 Hz), 3.73(2H, t, J=6.5 Hz), 7.25(2H, d, J=8.7 Hz), 7.63(1H, d, J=8.3 Hz), 7.76(2H, d, J=8.7 Hz), 7.87-7.97(1H, m), 7.99-8.08(2H, m), 8.15(1H, d, J=8.3 Hz), 8.45-8.56(1H, m), 8.78-8.85(1H, m), 11.18(1H, s) (+)ESI-MS(m/z): 417(M+H)⁺, 439(M+Na)⁺

### EXAMPLE 83

The following compound was obtained in substantially the same manner as in Example 74.

2-(Diethylamino)-4-methyl-N-(4-{[2-(2-pyridinyl)ethyl]amino}phenyl)benzamide

¹H-NMR(DMSO-d₆):δ 0.97(6H, t, J=7.1 Hz), 2.36(3H, s), 2.94-3.17(6H, m), 3.30-3.45(2H, m), 2.28(1H, t, J=5.7 Hz), 6.62(2H, d, J=8.8 Hz), 7.13(1H, d, J=8.0 Hz), 7.18-7.30(2H, m), 7.32(1H, d, J=7.8 Hz), 7.45(2H, d, J=8.8 Hz), 7.66-7.76(1H, m), 8.01(1H, d, J=8.0 Hz), 8.49-8.55(1H, m), 12.80(1H, s) (+)ESI-MS(m/z): 403(M+H)⁺, 425(M+Na)⁺

Preparation 84

A mixture of benzyl 4-methoxy-2-{[(trifluoromethyl)sulfonyl]oxy}benzoate (34.0 g) and 4-methylpiperidine (30.9 ml) in acetonitrile (100 ml) was stirred under reflux for 30 hours. The solvent was removed by concentration. The residue was purified by column chromatography on silica gel using a mixture of hexane and ethyl acetate (9:1 v/v) as an eluent. The eluted fractions containing the desired product were collected and evaporated in vacuo to give benzyl 4-methoxy-2-(4-methyl-1-piperidinyl)benzoate (22.88 g).

¹H-NMR(DMSO-d₆):δ 0.87(3H, d, J=6.1 Hz), 1.06-1.29(2H, m), 1.29-1.48(1H, m), 1.48-1.63(2H, m), 2.53-2.71(2H, m), 3.12-3.25(2H, m), 3.78(3H, s), 5.26(2H, s), 6.48-6.57(2H, m), 7.29-7.49(5H, m), 7.68(1H, d, J=8.3 Hz)

Preparation 85

The following compound was obtained in substantially the same manner as in Preparation 70.

4-Methoxy-2-(4-methyl-1-piperidinyl)benzoic acid

¹H-NMR(DMSO-d₆):δ 1.99(3H, d, J=6.4 Hz), 1.20-1.43(2H, m), 1.55-1.78(1H, m), 1.78-1.93(2H, m), 2.93-3.17(4H, m), 3.85(3H, s), 6.99(1H, dd, J=2.5 Hz, 8.8 Hz), 7.26(1H, d, J=2.5 Hz), 7.98(1H, d, J=8.8 Hz), 17.63(1H, s) (−)ESI-MS(m/z): 248(M−H)⁻

### EXAMPLE 84

The following compound was obtained in substantially the same manner as in Example 74.

4-Methoxy-2-(4-methyl-1-piperidinyl)-N-(4-{[2-(2-pyridinyl)ethyl]amino}phenyl)benzamide

¹H-NMR(DMSO-d₆):δ 0.97(3H, d, J=6.0 Hz), 1.23-1.65(3H, m), 1.68-1.83(2H, m), 2.70-2.86(2H, m), 2.99(2H, t, J=7.1 Hz), 3.04-3.16(2H, m), 3.30-3.43(2H, m), 3.81(3H, s), 5.58(1H, t, J=5.7 Hz), 6.61(2H, d, J=8.8 Hz), 6.77-6.87(2H, m), 7.18-7.27(1H, m), 7.33(1H, d, J=7.7 Hz), 7.47(2H, d, J=8.8 Hz), 7.66-7.76(1H, m), 7.90(1H, d, J=8.4 Hz), 8.49-8.55(1H, m), 11.58(1H, s) (+)ESI-MS(m/z): 445(M+H)⁺

Preparation 86

The following compound was obtained in substantially the same manner as in Preparation 62.

Methyl 2-(dimethylamino)-3-methylbenzoate

¹H-NMR(DMSO-d₆):δ 2.27(3H, s), 2.69(6H, s), 3.84(3H, s), 7.02(1H, t, J=7.5 Hz), 7.23-7.36(2H, m) (+)ESI-MS(m/z): 194(M+H)⁺, 216(M+Na)⁺

Preparation 87

The following compound was obtained in substantially the same manner as in Preparation 64.

2-(Dimethylamino)-3-methylbenzoic acid

¹H-NMR(DMSO-d₆):δ 2.48(3H, s), 2.90(6H, s), 7.31(1H, t, J=7.6 Hz), 7.45(1H, dd, J=1.5 Hz, 7.6 Hz), 7.89(1H, dd, J=1.5 Hz, 7.6 Hz), 18.19(1H, s) (−)ESI-MS(m/z): 178(M−H)⁻

### EXAMPLE 85

The following compound was obtained in substantially the same manner as in Example 74.

2-(Dimethylamino)-3-methyl-N-(4-{[2-(2-pyridinyl)ethyl]amino}phenyl)benzamide

¹H-NMR(DMSO-d₆):δ 2.31(3H, s), 2.75(6H, s), 2.99(2H, t, J=7.2 Hz), 3.29-3.45(2H, m), 5.55(1H, t, J=5.7 Hz), 6.58(2H, d, J=8.8 Hz), 7.06(1H, t, J=7.5 Hz), 7.18-7.37(4H, m), 7.45(2H, d, J=8.8 Hz), 7.71(1H, dt, J=1.8 Hz, 7.6 Hz), 8.47-8.55(1H, m), 10.45(1H, s) (+)ESI-MS(m/z): 375(M+H)⁺, 397(M+Na)⁺

### EXAMPLE 86

The following compound was obtained in substantially the same manner as in Example 74.

2-(Dimethylamino)-5-methyl-N-(4-{[2-(2-pyridinyl)ethyl]amino}phenyl)benzamide

¹H-NMR(DMSO-d₆):δ 2.30(3H, s), 2.72(6H, s), 2.99(2H, t, J=7.2 Hz), 3.31-3.44(2H, m), 5.58(1H, t, J=5.7 Hz), 6.60(2H, d, J=8.8 Hz), 7.15-7.36(4H, m), 7.45(2H, d, J=8.8 Hz), 7.62(1H, d, J=1.8 Hz), 7.71(1H, dt, J=1.7 Hz, 7.6 Hz), 8.50-8.56(1H, m), 11.41(1H, s) (+)ESI-MS(m/z): 375(M+H)⁺, 397(M+Na)⁺

Preparation 88

The following compound was obtained in substantially the same manner as in Preparation 62.

Methyl 5-chloro-2-(dimethylamino)benzoate

¹H-NMR(DMSO-d₆):δ 2.77(6H, s), 3.82(3H, s), 6.98(1H, d, J=8.9 Hz), 7.39(1H, dd, J=2.6 Hz, 8.9 Hz), 7.49(1H, d, J=2.6 Hz)

Preparation 89

The following compound was obtained in substantially the same manner as in Preparation 64.

5-Chloro-2-(dimethylamino)benzoic acid

¹H-NMR(DMSO-d₆):δ 2.82(6H, s), 7.49-7.66(2H, m), 7.76(1H, s), 15.37-17.48(1H, br) (−)ESI-MS(m/z): 198(M−H)⁻

### EXAMPLE 87

The following compound was obtained in substantially the same manner as in Example 74.

5-Chloro-2-(dimethylamino)-N-(4-{[2-(2-pyridinyl)ethyl]amino}phenyl)benzamide

¹H-NMR(DMSO-d₆):δ 2.77(6H, s), 2.99(2H, t, J=7.2 Hz), 3.31-3.44(2H, m), 5.61(1H, t, J=5.7 Hz), 6.60(2H, d, J=8.8 Hz), 7.16(1H, d, J=8.7 Hz), 7.18-7.27(1H, m), 7.32(1H, d, J=7.8 Hz), 7.39-7.48(3H, m), 7.56(1H, d, J=2.7 Hz), 7.66-7.77(1H, m), 8.50-8.56(1H, m), 10.73(1H, s)

### EXAMPLE 88

The following compound was obtained in substantially the same manner as in Example 80.

5-Chloro-2-(dimethylamino)-N-(4-{[2-(2-pyridinyl)ethyl]amino}phenyl)benzamide trihydrochloride

¹H-NMR(DMSO-d₆):δ 2.96(6H, s), 3.51(2H, t, J=6.9 Hz), 3.74(2H, t, J=6.9 Hz), 7.28(2H, d, J=8.7 Hz), 7.52(1H, d, J=8.8 Hz), 7.63(1H, dd, J=2.3 Hz, 8.8 Hz), 7.73(2H, d, J=8.8 Hz), 7.80(1H, d, J=2.3 Hz), 7.86-7.96(1H, m), 8.02(1H, d, J=8.0 Hz), 8.45-8.55(1H, m), 8.78-8.85(1H, m), 11.07(1H, s)

### EXAMPLE 89

1-[3-(Dimethylamino)propyl]-3-ethylcarbodiimide (0.19 g) was added to a solution of 4-(2-pyridinylmethyl)aniline (0.18 g), 2-(dimethylamino)-4-methylbenzoic acid (0.22 g), 1-hydroxybenzotriazole (0.16 g) and 4-dimethylaminopyridine (6 mg) in tetrahydrofuran (5 ml) and the mixture was stirred at ambient temperature for 18 hours. The reaction mixture was poured into a mixture of ethyl acetate and water. The separated organic layer was washed with water, dried over magnesium sulfate and evaporated in vacuo. The residue was purified by column chromatography on silica gel using a mixture of ethyl acetate and diisopropyl ether (2:3 v/v) as an eluent. The eluted fractions containing the desired product were collected and evaporated in vacuo to give 2-(dimethylamino)-4-methyl-N-[4-(2-pyridinylmethyl)phenyl]benzamide (0.14 g).

¹H-NMR(DMSO-d₆):δ 2.34(3H, s), 2.75(6H, s), 4.05(2H, s), 6.94(1H, d, J=8.2 Hz), 7.09(1H, s), 7.16-7.30(4H, m), 7.59-7.75(4H, m), 8.46-8.52(1H, m), 11.46(1H, s) (+)ESI-MS(m/z): 346(M+H)⁺, 368(M+Na)⁺

### EXAMPLE 90

The following compound was obtained in substantially the same manner as in Example 89.

4-Methyl-2-(4-methyl-1-piperidinyl)-N-{4-[2-(2-pyridinyl)ethoxy]phenyl}benzamide

¹H-NMR(DMSO-d₆):δ 0.95(3H, d, J=6.1 Hz), 1.20-1.62(3H, m), 1.66-1.82(2H, m), 2.34(3H, s), 2.68-2.88(2H, m), 3.02-3.23(4H, m), 4.34(2H, t, J=6.6 Hz), 6.94(2H, d, J=8.9 Hz), 7.04(1H, d, J=8.0 Hz), 7.16(1H, s), 7.20-7.29(1H, m), 7.37(1H, d, J=7.8 Hz), 7.60-7.85(4H, m), 8.48-8.55(1H, m), 11.79(1H, s) (+)ESI-MS(m/z): 430(M+H)⁺, 452(M+Na)⁺

Preparation 90

A mixture of benzyl 2-chloro-6-methylnicotinate (8.15 g) and 4-methylpiperidine (12.4 g) in tetrahydrofuran (50 ml) was stirred at 75-80° C. for 2.5 hours.

The reaction mixture was poured into a mixture of ethyl acetate and water, and the organic layer was washed with brine and dried over magnesium sulfate. The solvent was evaporated in vacuo and the residue was chromatographed on silica gel eluting with ethyl acetate: n-hexane (2:8 v/v). The eluted fractions containing the desired product were collected and evaporated in vacuo to give benzyl 6-methyl-2-(4-methyl-1-piperidinyl)nicotinate (9.49 g).

¹H-NMR(DMSO-d₆):δ 0.86 (3H, d, J=6.0 Hz), 0.96-1.21 (2H, m), 1.42-1.57 (3H, m), 2.34 (3H, s), 2.72-2.83 (2H, m), 4.02-4.05 (2H, m), 5.28 (2H, s), 6.63 (1H, d, J=7.7 Hz), 7.31-7.48 (5H, m), 7.83 (1H, d, J=7.7 Hz)

Preparation 91

A mixture of benzyl 6-methyl-2-(4-methyl-1-piperidinyl)nicotinate (9.45 g) in methanol (80 ml) was hydrogenated over 10% palladium on carbon (4.5 g) under atmospheric pressure of hydrogen at ambient temperature for 5 hours.

After removal of the catalyst, the solvent was evaporated in vacuo and the residue was dissolved in a ethyl acetate and dried over magnesium sulfate. The solvent was evaporated in vacuo to give 6-methyl-2-(4-methyl-1-piperidinyl)nicotinic acid (6.57 g).

¹H-NMR(DMSO-d₆):δ 0.93 (3H, d, J=6.1 Hz), 1.16-1.28 (2H, m), 1.50-1.70 (3H, m), 2.37 (3H, s), 2.52-2.92 (2H, m), 3.54-3.68 (2H, m), 6.77 (1H, d, J=7.7 Hz), 7.87(1H, d, J=7.7 Hz)

### EXAMPLE 91

A mixture of 6-methyl-2-(4-methyl-1-piperidinyl)nicotinic acid (2.46 g), N-(4-aminophenyl)-N-[2-(2-pyridinyl)ethyl]formamide (2.41 g), 1-hydroxybenzotriazole hydrate (1.61 g) and 1-[3-(dimethylamino)propyl]-3-ethylcarbodiimide (1.63 g) in N,N-dimethylformamide (25 ml) was stirred at ambient temperature for 15 hours.

The reaction mixture was poured into a mixture of ethyl acetate and water, and the organic layer was washed with brine and dried over magnesium sulfate. The solvent was evaporated in vacuo and the residue was chromatographed on silica gel eluting with ethyl acetate. The eluted fractions containing the desired product were collected and concentrated in vacuo and the precipitate was collected by filtration to give N-(4-{formyl[2-(2-pyridinyl)ethyl]amino}phenyl)-6-methyl-2-(4-methyl-1-piperidinyl)nicotinamide (3.49 g).

¹H-NMR(DMSO-d₆):δ 0.89 (3H, d, J=6.2 Hz), 1.16-1.22 (2H, m), 1.60-1.66 (3H, m), 2.40 (3H, s), 2.74-2.95 (4H, m), 3.65-3.71 (2H, m), 4.10 (2H, t, J=7.2 Hz), 6.82 (1H, d, J=7.6 Hz), 7.17-7.32 (4H, m), 7.63-7.70 (4H, m), 8.35 (1H, s), 8.45-8.48 (1H, m), 10.60 (1H, s) (+)ESI-MS(m/z): 541(M+H)⁺, 563(M+Na)⁺

### EXAMPLE 92

A mixture of N-(4-{formyl[2-(2-pyridinyl)ethyl]amino}phenyl)-6-methyl-2-(4-methyl-1-piperidinyl)nicotinamide (3.45 g) and concentrated hydrochloric acid (1.93 ml) in methanol (40 ml) was stirred at ambient temperature for 15 hours. The reaction mixture was evaporated in vacuo and the residue was dissolved in a mixture of water and ethyl acetate and adjusted to pH 8.0 with aqueous potassium carbonate solution.

The organic layer was washed with brine and dried over magnesium sulfate. The solvent was concentrated in vacuo and the precipitate was collected by filtration to give 6-methyl-2-(4-methyl-1-piperidinyl)-N-(4-{[2-(2-pyridinyl)ethyl]amino}phenyl)nicotinamide (2.78 g).

¹H-NMR(DMSO-d₆):δ 0.91 (3H, d, J=6.1 Hz), 1.14-1.31 (2H, m), 1.48-1.67 (2H, m), 2.39 (3H, s), 2.75-2.86 (2H, m), 2.99 (2H, t, J=7.3 Hz), 3.32-3.42 (2H, m), 3.60-3.66 (2H, m), 5.57 (1H, t, J=5.7 Hz), 6.60 (2H, d, J=8.8 Hz), 6.82 (1H, d, J=7.7 Hz), 7.19-7.25 (1H, m), 7.32 (1H, d, J=7.9 Hz), 7.45 (2H, d, J=8.8 Hz), 7.66-7.79 (2H, m), 8.50-8.53 (1H, m), 10.29 (1H, s)

### EXAMPLE 93

The following compound was obtained in substantially the same manner as in Example 91.

2-(Dimethylamino)-N-(4-{formyl[2-(2-pyridinyl)ethyl]amino}phenyl)benzamide

¹H-NMR(DMSO-d₆):δ 2.79 (6H, s), 2.29 (2H, t, J=7.9 Hz), 4.12 (2H, t, J=7.9 Hz), 7.05-7.13 (1H, m), 7.20-7.30 (5H, m), 7.44 (1H, d, J=7.0 Hz), 7.64-7.72 (2H, m), 7.78 (2H, d, J=8.8 Hz), 8.35 (1H, s), 8.48-8.49 (1H, m), 11.30 (1H,s)

### EXAMPLE 94

The following compound was obtained in substantially the same manner as in Example 92.

2-(Dimethylamino)-N-(4-{[2-(2-pyridinyl)ethyl]amino}phenyl)benzamide

¹H-NMR(DMSO-d₆):δ 2.77 (6H, s), 2.99 (2H, t, J=7.4 Hz), 3.33-3.43 (2H, m), 5.56 (1H, t, J=5.7 Hz), 6.60 (2H, d, J=8.8 Hz), 7.05-7.39 (5H, m), 7.44 (2H, d, J=8.8 Hz), 7.67-7.75 (2H, m), 8.51-8.53 (1H, m), 10.95 (1H, s) (+)ESI-MS(m/z): 361(M+H)⁺, 383(M+Na)⁺

### EXAMPLE 95

The following compound was obtained in substantially the same manner as in Example 91.

N-(4-{2-[6-(Acetylamino)-2-pyridinyl]ethyl)phenyl)-6-methyl-2-(4-methyl-1-piperidinyl)nicotinamide

¹H-NMR(DMSO-d₆):δ 0.98 (3H, d, J=6.1 Hz), 1.09-1.28 (2H, m), 1.43-1.65 (3H, m), 2.09 (3H, s), 2.39 (3H, s), 2.75-2.87 (2H, m), 2.94 (4H, s), 3.62-3.68 (2H, m), 6.82 (1H, d, J=7.6 Hz), 6.94 (1H, d, J=7.3 Hz), 7.18 (2H, d, J=8.4 Hz), 7.60-7.68 (3H, m), 7.76 (1H, d, J=7.6 Hz), 7.90 (1H, d, J=8.2 Hz), 10.43 (1H, s), 10.50 (1H, s)

### EXAMPLE 96

A mixture of N-(4-{2-[6-(acetylamino)-2-pyridinyl]ethyl}phenyl)-6-methyl-2-(4-methyl-1-piperidinyl)nicotinamide (610 mg) and 6N hydrochloric acid (1.5 ml) in methanol (10 ml) was refluxed under stirring for 8 hours. The reaction mixture was evaporated in vacuo and the residue was dissolved in a mixture of water and ethyl acetate and adjusted to pH 8.0 with aqueous potassium carbonate solution.

The organic layer was washed with brine and dried over magnesium sulfate. The solvent was concentrated in vacuo and the precipitate was collected by filtration to give N-{4-[2-(6-amino-2-pyridinyl)ethyl]phenyl}-6-methyl-2-(4-methyl-1-piperidinyl)nicotinamide (450 mg).

¹H-NMR(DMSO-d₆):δ 0.90 (3H, d, J=6.2 Hz), 1.06-1.29 (2H, m), 1.48-1.65 (3H, m), 2.39 (3H, s), 2.72-2.91 (6H, m), 3.62-3.69 (2H, m), 5.81 (2H, s), 6.24-6.36 (2H, m), 6.82 (1H, d, J=7.7 Hz), 7.18 (2H, d, J=8.4 Hz), 7.27 (1H, d, J=7.7 Hz), 7.62 (2H, d, J=8.4 Hz), 7.75 (1H, d, J=7.5 Hz), 10.49 (1H, s) (+)ESI-MS(m/z): 430(M+H)⁺, 452(M+Na)⁺

Preparation 92

A mixture of 2-chloro-6-methylnicotinic acid (17.2 g), N-(4-aminophenyl)-N-[2-(2-pyridinyl)ethyl]formamide (24.9 g), 1-hydroxybenzotriazole hydrate (16.1 g) and 1-[3-(dimethylamino)propyl]-3-ethylcarbodiimide (16.3 g) in N,N-dimethylformamide (100 ml) was stirred at ambient temperature for 15 hours.

The reaction mixture was poured into a mixture of ethyl acetate and water, and the organic layer was washed with brine and dried over magnesium sulfate. The solvent was evaporated in vacuo and the residue was chromatographed on silica gel eluting with ethyl acetate:methanol (95:5 v/v). The eluted fractions containing the desired product were collected and evaporated in vacuo to give 2-chloro-N-(4-{formyl[2-(2-pyridinyl)ethyl]amino}phenyl)-6-methylnicotinamide (26.8 g).

¹H-NMR(DMSO-d₆):δ 2.51 (3H, s), 2.89 (2H, t, J=7.2 Hz), 4.12 (2H, t, J=7.2 Hz), 7.18-7.34 (4H, m), 7.42 (1H, d, J=7.7 Hz), 7.64-7.76 (3H, m), 7.96 (1H, d, J=7.7 Hz), 8.14 (1H, s), 8.35-8.47 (1H, m), 10.67 (1H, s)

### EXAMPLE 97

A mixture of 2-chloro-N-(4-{formyl[2-(2-pyridinyl)ethyl]amino}phenyl)-6-methylnicotinamide (11.2 g) and 4-methylpiperidine(13.4 ml) in tetrahydrofuran (50 ml) was refluxed under stirring for 9 hours. The reaction mixture was poured into a mixture of ethyl acetate and water, and the organic layer was washed with brine and dried over magnesium sulfate. The solvent was evaporated in vacuo and the residue was chromatographed on silica gel eluting with ethyl acetate: methanol (95:5 v/v). The eluted fractions containing the desired product were collected and the solvent was concentrated in vacuo and the precipitate was collected by filtration to give N-(4-{formyl[2-(2-pyridinyl) ethyl]amino}phenyl)-6-methyl-2-(4-methyl-1-piperidinyl)nicotinamide (7.21 g).

¹H-NMR(DMSO-d₆):δ 0.89 (3H, d, J=6.2 Hz), 1.16-1.22 (2H, m), 1.60-1.66 (3H, m), 2.40 (3H, s), 2.74-2.95 (4H, m), 3.65-3.71 (2H, m), 4.10 (2H, t, J=7.2 Hz), 6.82 (1H, d, J=7.6 Hz), 7.17-7.32 (4H, m), 7.63-7.70 (4H, m), 8.35 (1H, s), 8.45-8.48 (1H, m), 10.60 (1H, s) (+)ESI-MS(m/z): 541(M+H)⁺, 563(M+Na)⁺

### EXAMPLE 98

A mixture of 4-methyl-2-(4-methyl-1-piperidinyl)benzoic acid (350 mg), N-2-[2-(2-pyridinyl)ethyl]-2,5-pyridinediamine (337 mg), 1-hydroxybenzotriazole hydrate (241 mg) and 1-[3-(dimethylamino)propyl]-3-ethylcarbodiimide (245 mg) in N,N-dimethylformamide (15 ml) was stirred at ambient temperature for 15 hours. The reaction mixture was poured into a mixture of ethyl acetate and water, and the organic layer was washed with brine and dried over magnesium sulfate. The solvent was evaporated in vacuo and the residue was chromatographed on silica gel eluting with ethyl acetate:methanol (97:3 v/v). The eluted fractions containing the desired product were collected and the solvent was evaporated in vacuo. The residue was recrystallized from a mixture of acetone and diisopropyl ether to give 4-methyl-2-(4-methyl-1-piperidinyl)-N-(6-{[2-(2-pyridinyl)ethyl]amino}-3-pyridinyl)benzamide (85 mg).

¹H-NMR(DMSO-d₆):δ 0.95 (3H, d, J=6.2 Hz), 1.29-1.51 (3H, m), 1.73-7.79 (2H, m), 2.34 (3H, s), 2.72-2.83 (2H, m), 2.96-3.13 (4H, m), 3.53-3.63 (2H, m), 6.47-6.56 (2H, m), 7.03 (1H, d, J=8.1 Hz), 7.16-7.31 (3H, m), 7.66-7.83 (3H, m), 8.30 (1H, d, J=2.5 Hz), 8.49-8.52 (1H, m), 11.64 (1H, s)

### EXAMPLE 99

The following compound was obtained in substantially the same manner as in Example 98.

2-(Dimethylamino)-4-methyl-N-(6-{[2-(2-pyridinyl)ethyl]amino}-3-pyridinyl)benzamide

¹H-NMR(DMSO-d₆):δ 2.34 (3H, s), 2.76 (6H, s), 2.99 (2H, t, J=7.4 Hz), 3.54-3.64 (2H, m), 6.47-6.51 (2H, m), 6.94 (1H, d, J=8.1 Hz), 7.08 (1H, s), 7.24-7.30 (2H, m), 7.64 (3H, m), 8.29 (1H, d, J=2.5 Hz), 8.50-8.53 (1H, m), 11.20 (1H, s)

### EXAMPLE 100

The following compound was obtained in substantially the same manner as in Example 43.

tert-Butyl 4-({[6-methyl-2-(1-pyrrolidinyl)-3-pyridinyl]carbonyl}amino)phenyl[2-(2-pyridinyl)ethyl]carbamate

¹H-NMR(DMSO-d₆):δ 1.33 (9H, s), 1.80-1.86 (4H, m), 2.34 (3H, s), 2.90 (2H, t, J=7.4 Hz), 3.36-3.41 (4H, m), 3.90 (2H, t, J=7.4 Hz), 6.53 (1H, d, J=7.5 Hz), 7.13-7.26 (4H, m), 7.53 (1H, d, J=7.5 Hz), 7.64-7.70 (3H, m), 7.43-8.45 (1H, m), 10.31 (1H, s)

### EXAMPLE 101

The following compound was obtained in substantially the same manner as in Example 44.

6-Methyl-N-(4-{[2-(2-pyridinyl)ethyl]amino)phenyl)-2-(1-pyrrolidinyl)nicotinamide

¹H-NMR(DMSO-d₆):δ 1.79-1.85 (4H, m), 2.32 (3H, s), 2.98 (2H, t, J=7.4 Hz), 3.31-3.38 (6H, m), 5.53 (1H, m), 6.53 (1H, d, J=7.5 Hz), 6.56 (2H, d, J=8.8 Hz), 7.19-7.47 (5H, m), 7.47-7.71 (1H, m), 8.50-8.53 (1H, m), 9.87 (1H, s) (+)ESI-MS(m/z): 402(M+H)⁺, 424(M+Na)⁺

### EXAMPLE 102

The following compound was obtained in substantially the same manner as in Example 43. The product was used in the next step without purification.

tert-Butyl 4-({[2-(diethylamino)-6-methyl-3-pyridinyl]carbonyl}amino)phenyl[2-(2-pyridinyl)ethyl]carbamate

### EXAMPLE 103

The following compound was obtained in substantially the same manner as in Example 44.

2-(Diethylamino)-6-methyl-N-(4-{[2-(2-pyridinyl)ethyl]amino}phenyl)nicotinamide

¹H-NMR(DMSO-d₆):δ 1.05 (6H, t, J=6.9 Hz), 2.36 (3H, s), 2.98 (2H, t, J=7.4 Hz), 3.30-3.40 (6H, m), 5.56 (1H, t, J=5.7 Hz), 6.57 (2H, d, J=8.9 Hz), 6.70 (1H, d, J=7.6 Hz), 7.1-7.25 (1H, m), 7.32 (1H, d, J=7.7 Hz), 7.40 (2H, d, J=8.9 Hz), 7.63-7.75 (2H, m), 8.50-8.52 (1H, m), 10.43 (1H, s) (+)ESI-MS(m/z): 404(M+H)⁺, 426(M+Na)⁺

### EXAMPLE 104

The following compound was obtained in substantially the same manner as in Example 43. The product was used in the next step without purification.

tert-Butyl 4-({[2-(diethylamino)-3-pyridinyl]carbonyl}amino)phenyl[2-(2-pyridinyl)ethyl]carbamate

### EXAMPLE 105

The following compound was obtained in substantially the same manner as in Example 44.

2-(Diethylamino)-N-(4-{[2-(2-pyridinyl)ethyl]amino}phenyl)nicotinamide

¹H-NMR(DMSO-d₆):δ 1.06 (6H, t, J=6.9 Hz), 2.99 (2H, t, J=7.4 Hz), 3.34-3.44 (6H, m), 5.58 (1H, t, J=5.7 Hz), 6.59 (2H, d, J=8.8 Hz), 6.79 (1H, dd, J=4.9 Hz, 7.4 Hz), 7.23-7.25 (1H, m), 7.32 (1H, d, J=7.7 Hz), 7.42 (2H, d, J=8.8 Hz), 7.66-7.71 (2H, m), 8.20-8.24 (1H, m), 8.50-8.52 (1H, m), 10.34 (1H, s) (+)ESI-MS(m/z): 390(M+H)⁺, 412(M+Na)⁺

### EXAMPLE 106

The following compound was obtained in substantially the same manner as in Example 97.

2-[Ethyl(methyl)amino]-N-(4-{formyl[2-(2-pyridinyl)ethyl]amino}phenyl)-6-methylnicotinamide

¹H-NMR(DMSO-d₆):δ 1.08 (3H, t, J=7.0 Hz), 2.37 (3H, s), 2.87 (3H, s), 2.91 (2H, t, J=7.3 Hz), 3.46 (2H, q, J=7.0 Hz), 4.10 (2H, t, J=7.3 Hz), 6.63 (1H, d, J=7.6 Hz), 7.17-7.30 (3H, m), 7.59 (1H, d, J=7.6 Hz), 7.64-7.77 (3H, m), 8.34 (1H, s), 7.46-8.48 (1H, m), 10.47 (1H, s)

### EXAMPLE 107

The following compound was obtained in substantially the same manner as in Example 92.

2-[Ethyl(methyl)amino]-6-methyl-N-(4-{[2-(2-pyridinyl)ethyl]amino}phenyl)nicotinamide

¹H-NMR(DMSO-d₆):δ 1.05 (3H, t, J=6.9 Hz), 2.35 (3H, s), 2.86 (3H, s), 2.98 (2H, t, J=7.4 Hz), 3.33-3.48 (4H, m), 5.56 (1H, br.s), 6.55-6.63 (3H, m), 7.19-7.25 (1H, m), 7.31 (1H, d, J=7.7 Hz), 7.40 (2H, d, J=8.8 Hz), 7.55 (1H, d, J=7.5 Hz), 7.66-7.75 (1H, m), 8.50-8.53 (1H, m), 10.01 (1H, s) (+)ESI-MS(m/z): 390(M+H)⁺, 412(M+Na)⁺

Preparation 93

The following compound was obtained in substantially the same manner as in Preparation 92.

2,6-Dichloro-N-(4-{formyl[2-(2-pyridinyl)ethyl]amino}phenyl)nicotinamide

¹H-NMR(DMSO-d₆):δ 2.92 (2H, t, J=7.3 Hz), 4.13 (2H, t, J=7.3 Hz), 7.21-7.37 (3H, m), 7.65-7.80 (5H, m), 8.21 (1H, d, J=8.0 Hz), 8.23 (1H, s), 8.48-8.51 (1H, m), 10.78 (1H, s)

### EXAMPLE 108

The following compound was obtained in substantially the same manner as in Example 97.

N-(4-{Formyl[2-(2-pyridinyl)ethyl]amino}phenyl)-2,6-bis(4-methyl-1-piperidinyl)nicotinamide

¹H-NMR(DMSO-d₆):δ 0.90-0.95 (6H, m), 10.2-1.34 (4H, m), 1.51-1.71 (6H, m), 2.76-2.95 (4H, m), 3.30-3.47 (4H, m), 4.10 (2H, t, J=7.2 Hz), 4.32-4.39 (4H, m), 6.48 (1H, d, J=8.7 Hz), 7.16-7.30 (4H, m), 7.63-7.86 (4H, m), 8.12 (1H, s), 8.45 (1H, m), 10.85 (1H, s)

### EXAMPLE 109

The following compound was obtained in substantially the same manner as in Example 92.

2,6-Bis(4-methyl-1-piperidinyl)-N-(4-{[2-(2-pyridinyl)ethyl]amino}phenyl)nicotinamide

¹H-NMR(DMSO-d₆):δ 0.90-0.94 (6H, m), 1.02-1.71 (10H, m), 2.74-2.89 (4H, m), 2.98 (2H, t, J=7.3 Hz), 3.33-3.42 (6H, m), 4.30-4.37 (2H, m), 5.52 (1H, s), 6.48 (1H, J=8.6 Hz), 6.58 (2H, d, J=8.8 Hz), 7.15-7.25 (1H, m), 7.32 (1H, d, J=7.7 Hz), 7.42 (2H, d, J=8.8 Hz), 7.67-7.75 (1H, m), 7.84 (1H, d, J=8.6 Hz), 8.50-8.53 (1H, m), 10.53 (1H, s) (+)ESI-MS(m/z): 513(M+H)⁺, 535(M+Na)⁺

Preparation 94

The following compound was obtained in substantially the same manner as in Preparation 92.

2-Chloro-6-methyl-N-(6-{[2-(2-pyridinyl)ethyl]amino}-3-pyridinyl)nicotinamide

¹H-NMR(DMSO-d₆):δ 2.51 (3H, s), 3.00 (2H, t, J=7.4 Hz), 3.54-3.64 (2H, m), 6.48-6.58 (2H, m), 7.21-7.30 (2H, m), 7.39 (1H, d, J=7.7 Hz), 7.66-7.73 (2H, m), 7.93 (1H, d, J=7.6 Hz), 8.27 (1H, d, J=2.5 Hz), 8.50-8.53 (1H, m), 10.27 (1H, s)

### EXAMPLE 110

The following compound was obtained in substantially the same manner as in Example 97.

6-Methyl-2-(4-methyl-1-piperidinyl)-N-(6-{[2-(2-pyridinyl)ethyl]amino}-3-pyridinyl)nicotinamide

¹H-NMR(DMSO-d₆):δ 0.90 (3H, d, J=6.2 Hz), 1.14-1.29 (2H, m), 1.44-1.67 (3H, m), 2.39 (3H, s), 2.75-2.87 (2H, m), 2.99 (2H, t, J=7.4 Hz), 3.53-3.69 (4H, m), 6.46-6.51 (2H, m), 6.80 (1H, d, J=7.6 Hz), 7.18-7.25 (1H, m), 7.28 (1H, d, J=7.8 Hz), 7.66-7.75 (3H, m), 8.27 (1H, d, J=2.5 Hz), 8.49-8.51 (1H, m), 10.24 (1H, s) (+)ESI-MS(m/z): 431(M+H)⁺, 453(M+Na)⁺

### EXAMPLE 111

The following compound was obtained in substantially the same manner as in Example 97.

6-Methyl-2-(1-piperidinyl)-N-(6-{[2-(2-pyridinyl)ethyl]amino}-3-pyridinyl)nicotinamide

¹H-NMR(DMSO-d₆):δ 1.55 (6H, s), 2.39 (3H, s), 2.98 (2H, t, J=7.4 Hz), 3.22 (4H, m), 3.52-3.62 (2H, m), 6.46-6.50 (2H, m), 6.82 (1H, d, J=7.6 Hz), 7.21-7.25 (1H, m), 7.28 (1H, d, J=7.7 Hz), 7.66-7.07 (3H, m), 8.28 (1H, d, J=2.5 Hz), 8.49-8.51 (1H, m), 10.30 (1H, s) (+)ESI-MS(m/z): 417(M+H)⁺, 439(M+Na)⁺

Preparation 95

A solution of 2-chloro-6-methylnicotinoyl chloride (1.9 g) in tetrahydrofuran (5 ml) was added to a mixture of 1-(4-aminophenyl)-3-(2-pyridinyl)propan-1-one (2.26 g) and triethylamine (4.04 g) in tetrahydrofuran (50 ml) at ambient temperature. The mixture was stirred at ambient temperature for 5 hours. The resultant mixture was poured into a mixture of ethyl acetate and water, and the organic layer was washed with a 5% potassium carbonate solution and brine and dried over magnesium sulfate. The solvent was evaporated in vacuo and the residue was recrystallized from a mixture of ethyl acetate and n-hexane to give 2-chloro-6-methyl-N-{4-[3-(2-pyridinyl)propanoyl]phenyl}nicotinamide (3.11 g).

¹H-NMR(DMSO-d₆):δ 3.11 (2H, t, J=7.2 Hz), 3.47 (2H, t, J=7.2 Hz), 7.16-7.22 (1H, m), 7.34 (1H, d, J=7.8 Hz), 7.43 (1H, d, J=7.7 Hz), 7.65-7.74 (1H, m), 7.83 (2H, d, J=8.7 Hz), 7.98-8.05 (3H, m), 8.44-8.47 (1H, s), 10.90 (1H, s)

### EXAMPLE 112

A mixture of 2-chloro-6-methyl-N-{4-[3-(2-pyridinyl)propanoyl]phenyl}nicotinamide (1.52 g) and 4-methylpiperidine (1.9 ml) in tetrahydrofuran (10 ml) was refluxed under stirring for 7 hours. The reaction mixture was poured into a mixture of ethyl acetate and water, and the organic layer was washed with brine and dried over magnesium sulfate. The solvent was concentrated in vacuo and the precipitate was collected by filtration to give 6-methyl-2-(4-methyl-1-piperidinyl)-N-{4-[3-(2-pyridinyl)propanoyl]phenyl}nicotinamide (1.346 g).

¹H-NMR(DMSO-d₆):δ 0.88 (3H, d, J=6.1 Hz), 1.14-1.26 (2H, m), 1.47-1.64 (3H, m), 2.40 (3H, s), 2.76-2.87 (2H, m), 3.11 (2H, t, J=7.2 Hz), 3.62 (2H, t, J=7.2 Hz), 4.01-4.05 (2H, m), 6.83 (1H, J=7.7 Hz), 7.15-7.22 (1H, m), 7.34 (1H, d, J=7.7 Hz), 7.65-7.79 (2H, m), 7.85 (2H, d, J=8.0 Hz), 8.02 (2H, d, J=8.0 Hz), 8.45-8.47 (1H, m), 10.81 (1H, s) (+)ESI-MS(m/z): 443(M+H)⁺, 465(M+Na)⁺

### EXAMPLE 113

Sodium borohydrate (182 mg) was added to a solution of 6-methyl-2-(4-methyl-1-piperidinyl)-N-{4-[3-(2-pyridinyl)propanoyl]phenyl}nicotinamide (1.06 g) in methanol (30 ml) at ambient temperature under stirring. The mixture was stirred at ambient temperature for 4 hours. The resultant solution was evaporated in vacuo and the residue was dissolved in a mixture of ethyl acetate and water. The organic layer was washed with a 5% aqueous potassium carbonate solution and brine and dried over magnesium sulfate. The solvent was evaporated in vacuo and the residue was recrystallized from a mixture of ethyl acetate and diisopropyl ether to give N-{4-[1-hydroxy-3-(2-pyridinyl)propyl]phenyl}-6-methyl-2-(4-methyl-1-piperidinyl)nicotinamide (738 mg).

¹H-NMR(DMSO-d₆):δ 0.89 (3H, d, J=6.1 Hz), 1.02-1.29 (2H, m), 1.48-1.66 (3H, m), 1.93-2.04 (2H, m), 2.63-2.87 (4H, m), 3.62-3.69 (2H, m), 4.50-4.58 (1H, m), 5.27 (1H, d, J=4.4 Hz), 6.83 (1H, d, J=7.6 Hz), 7.14-7.24 (2H, m), 7.31 (2H, d, J=7.6 Hz), 7.63-7.71 (3H, m), 7.76 (1H, d, J=7.6 Hz), 8.46 (1H, d, J=4.5 Hz), 10.53 (1H, s) (−)ESI-MS(m/z): 443(M−H)⁻

### EXAMPLE 114

A solution of N-{4-[1-hydroxy-3-(2-pyridinyl)propyl]phenyl}-6-methyl-2-(4-methyl-1-piperidinyl)nicotinamide (610 mg) in methanol (30 ml) and 4N hydrogen chloride in 1,4-dioxane (1.5 ml) was hydrogenated over 10% palladium on carbon (300 mg) under an atmospheric pressure of hydrogen at ambient temperature under stirring for 10 hours. After removal of the catalyst, the solvent was evaporated in vacuo. The residue was dissolved in a mixture of water and ethyl acetate and adjusted to pH 8.0 with a 5% aqueous potassium carbonate solution. The organic layer was washed with brine and dried over magnesium sulfate. The solvent was evaporated in vacuo and the residue was chromatographed on silica gel eluting with ethyl acetate: n-hexane (6:4 v/v). The eluted fractions containing the desired product were collected and the solvent was evaporated. The residue was crystallized from a mixture of diisopropyl ether and n-hexane to give 6-methyl-2-(4-methyl-1-piperidinyl)-N-{4-[3-(2-pyridinyl)propyl]phenyl}nicotinamide (190 mg).

¹H-NMR(DMSO-d₆):δ 0.98 (3H, d, J=6.1 Hz), 1.05-1.29 (2H, m), 1.48-1.92 (3H, m), 1.92-2.04 (2H, m), 2.39 (3H, s ), 2.51-2.87 (6H, m), 3.62-3.69 (2H, m), 6.82 (1H, d, J=7.7 Hz), 7.16-7.30 (4H, m), 7.62-7.85 (4H, m), 8.48 (1H, d, J=4.4 Hz), 10.51 (1H, s) (+)ESI-MS(m/z): 429(M+H)⁺, 451(M+Na)⁺

Preparation 96

A mixture of 2-(1H-pyrazol-1-yl)ethanol (6.76 g) and potassium tert-butoxide (6.75 g) in tetrahydrofuran (100 ml) was stirred at ambient temperature for an hour. A solution of 1-fluoro-4-nitrobenzene (7.1 g) in tetrahydrofuran (5 ml) was added to the above mixture and refluxed under stirring for 2.5 hours. The reaction mixture was poured into a mixture of ethyl acetate and water, and the organic layer was washed with brine and dried over magnesium sulfate. The solvent was concentrated in vacuo and the precipitate was collected by filtration to give 1-[2-(4-nitrophenoxy)ethyl]-1H-pyrazole (10.75 g).

¹H-NMR(DMSO-d₆):δ 4.47-4.60 (4H, m), 6.27 (1H, m), 7.08-7.16 (2H, m), 7.49 (1H, d, J=1.7 Hz), 7.81 (1H, d, J=2.0 Hz), 8.16-8.23 (2H, m)

Preparation 97

A mixture of 1-[2-(4-nitrophenoxy)ethyl]-1H-pyrazole (1.63 g) in methanol (25 ml) and tetrahydrofuran (25 ml) was hydrogenated over 10% palladium on carbon (0.8 g) under atmospheric pressure of hydrogen at ambient temperature for 6 hours. After removal of the catalyst by filtration, the solvent was evaporated in vacuo to give 4-[2-(1H-pyrazol-1-yl)ethoxy]phenylamine (1.4 g).

¹H-NMR(DMSO-d₆):δ 4.15-4.19 (2H, m), 4.39-4.64 (2H, m), 4.64 (2H, s), 6.23 (1H, s),6.45-6.51 (2H, m), 6.59-6.68 (2H, m), 7.45 (1H, s), 7.74 (1H, s)

Preparation 98

A mixture of 2-(1H-pyrazol-1-yl)ethanol (5.41 g) and potassium tert-butoxide (5.41 g) in tetrahydrofuran (50 ml) was stirred at ambient temperature for an hour. 2-Chloro-5-nitropyridine (6.38 g) was added to the above mixture and the resultant mixture was stirred at ambient temperature for 6.5 hours. The reaction mixture was poured into a mixture of ethyl acetate and water, and the organic layer was washed with brine and dried over magnesium sulfate. The solvent was concentrated in vacuo and the residue was chromatographed on silica gel eluting with ethyl acetate and n-hexane (6:4 v/v). The fraction was concentrated in vacuo and the precipitate was collected by filtration to give 5-nitro-2-[2-(1H-pyrazol-1-yl)ethoxy]pyridine (6.48 g).

¹H-NMR(DMSO-d₆):δ 4.55 (2H, t, J=4.9 Hz),4.76 (2H, t, J=4.9 Hz), 6.23-6.25 (1H, m),7.10 (1H, d, J=9.2 Hz), 7.45 (1H, d, J=1.7 Hz), 7.78 (1H, d, J=2.3 Hz), 8.46 (1H, dd, J=2.8 Hz, 9.2 Hz), 9.07 (1H, d, J=2.8 Hz)

Preparation 99

The following compound was obtained in substantially the same manner as in Preparation 97.

6-[2-(1H-Pyrazol-1-yl)ethoxy]-3-pyridinamine

¹H-NMR(DMSO-d₆):δ 4.43 (4H, m), 4.79 (2H, s), 6.22-6.24 (1H, m), 6.50 (1H, d, J=8.7 Hz), 7.99 (1H, dd, J=2.8 Hz, 8.7 Hz), 7.43 (1H, d, J=3.4 Hz), 7.49 (1H, d, J=2.8 Hz), 7.71 (1H, d, J=2.0 Hz)

Preparation 100

A mixture of 1-(2-chloroethoxy)-4-nitrobenzene (2.82 g) and 1,2,4-triazole sodium salt (1.78 g) in N,N-dimethylformamide (30 ml) was stirred at 75-80° C. for 5 hours. The reaction mixture was poured into a mixture of ethyl acetate and water, and the organic layer was washed with brine and dried over magnesium sulfate. The solvent was concentrated in vacuo and the precipitate was collected by filtration to give 1-[2-(4-nitrophenoxy)ethyl]-1H-1,2,4-triazole (2.27 g).

¹H-NMR(DMSO-d₆):δ 4.92 (2H, t, J=4.8 Hz), 4.65 (2H, t, J=4.8 Hz), 7.08-7.20 (2H, m), 8.00 (1H, s), 8.15-8.23 (2H, m), 8.60 (1H, s) (+)ESI-MS(m/z): 235(M+H)⁺, 257(M+Na)⁺

Preparation 101

The following compound was obtained in substantially the same manner as in Preparation 97.

4-[2-(1H-1,2,4-Triazol-1-yl)ethoxy]phenylamine

¹H-NMR(DMSO-d₆):δ 4.18 (2H, t, J=5.1 Hz), 4.50 (2H, t, J=5.1 Hz), 4.65 (2H, s), 6.43-6.53 (2H, m), 6.57-6.71 (2H, m), 7.99 (1H, s), 8.54 (1H,s)

Preparation 102

A mixture of N-(2-chloroethyl)-4-nitroaniline hydrochloride (12.0 g), 1,2,4-triazole sodium salt (6.45 g) and potassium carbonate (8.38 g) in N,N-dimethylformamide (30 ml) was stirred at 75-80° C. for 6 hours.

The reaction mixture was poured into a mixture of ethyl acetate and water, and the organic layer was washed with brine and dried over magnesium sulfate. The solvent was evaporated in vacuo and the residue was chromatographed on silica gel eluting with ethyl acetate:methanol (94:6 v/v). The eluted fractions were concentrated in vacuo and the precipitate was collected by filtration to give N-(4-nitrophenyl)-N-[2-(1H-1,2,4-triazol-1-yl)ethyl]amine (4.7 g).

¹H-NMR(DMSO-d₆):δ 3.60-3.69 (2H, m), 4.37 (2H, t, J=5.8 Hz), 6.61-6.69 (2H, m), 7.35 (1H, t, J=6.0 Hz), 7.95-8.02 (3H, m), 8.45 (1H, s)

Preparation 103

The following compound was obtained in substantially the same manner as in Preparation 97.

N-[2-(1H-1,2,4-Triazol-1-yl)ethyl]-1,4-benzenediamine

¹H-NMR(DMSO-d₆):δ 3.30-3.39 (2H, m), 4.29 (2H, t, J=6.0 Hz), 4.32 (2H, s), 4.85 (1H, t, J=6.3 Hz), 6.35-6.47 (4H, m), 7.98 (1H, s), 8.45 (1H, s)

Preparation 104

A mixture of (3-bromopropyl)benzene (10.0 g) and 1,2,4-triazole sodium salt (6.4 g) in N,N-dimethylformamide (50 ml) was stirred at 75-80° C. for 8.5 hours. The reaction mixture was poured into a mixture of ethyl acetate and water, and the organic layer was washed with brine and dried over magnesium sulfate. The solvent was evaporated in vacuo to give 1-(3-phenylpropyl)-1H-1,2,4-triazole (8.56 g).

¹H-NMR(DMSO-d₆):δ 2.17-2.28 (2H, m), 2.63 (2H, t, J=7.2 Hz), 4.12 (2H, t, J=7.0 Hz), 7.14-7.35 (5H, m), 7.99 (1H, s), 8.14 (1H, s)

Preparation 105

To a solution of fuming nitric acid (d=1.52) (40 ml) was portionwise added a 1-(3-phenylpropyl)-1H-1,2,4-triazole (8.5 g) at a temperature from −30° C. to −5° C. with stirring and the mixture was stirred at the same temperature for 20 minutes. The reaction mixture was poured into ice-water. The mixture was adjusted to pH 8.0 with an aqueous potassium carbonate solution and extracted with ethyl acetate. The organic layer was washed with brine and dried over magnesium sulfate. The solvent was evaporated in vacuo and the residue was chromatographed on silica gel eluting with ethyl acetate. The eluted fractions were evaporated in vacuo to give 1-[3-(4-nitrophenyl)propyl]-1H-1,2,4-triazole (4.67 g).

¹H-NMR(DMSO-d₆):δ 2.08-2.23 (2H, m), 2.68-2.76 (2H, m), 4.19 (2H, t, J=6.9 Hz), 7.51 (2H, d, J=8.6 Hz), 8.00 (1H, s), 8.18 (2H, d, J=9.6 Hz), 8.55 (1H, s)

Preparation 106

The following compound was obtained in substantially the same manner as in Preparation 97.

4-[3-(1H-1,2,4-Triazol-1-yl)propyl]phenylamine

¹H-NMR(DMSO-d₆):δ 1.90-2.03 (2H, m), 2.27-2.37 (2H, m), 4.18-4.23 (2H, m), 4.86 (2H, s), 6.45-6.63 (2H, m), 6.82-6.93 (2H, m), 7.98 (1H, s), 8.52 (1H, s)

Preparation 107

The following compound was obtained in substantially the same manner as in Preparation 92.

2-Chloro-6-methyl-N-{4-[2-oxo-2-(2-pyridinylamino)ethyl]phenyl}nicotinamide

¹H-NMR(DMSO-d₆):δ 2.52 (3H, s), 3.71 (2H, s), 7.02-7.12 (1H, m), 7.32-7.42 (3H, m), 7.63-7.76 (3H, m), 7.94 (1H, d, J=7.7 Hz), 8.06 (1H, d, J=8.3 Hz), 8.30-8.33 (1H, m), 10.54 (1H, s), 10.67 (1H, s)

### EXAMPLE 115

The following compound was obtained in substantially the same manner as in Example 113.

6-Methyl-2-(4-methyl-1-piperidinyl)-N-{4-[2-oxo-2-(2-pyridinylamino)ethyl]phenyl}nicotinamide

¹H-NMR(DMSO-d₆):δ 0.88 (3H, d, J=6.2 Hz), 1.11-1.27 (2H, m), 1.42-1.65 (3H, m), 2.39 (3H, s), 2.75-2.87 (2H, m), 3.69 (2H, s), 3.62-3.69 (2H, m), 6.82 (1H, d, J=7.7 Hz), 7.06-7.12 (1H, m), 7.31 (2H, d, J=8.4 Hz), 7.66 (2H, d, J=8.4 Hz), 7.69-7.76 (2H, m), 8.06 (1H, d, J=8.4 Hz), 8.30-8.33 (1H, m), 10.53 (1H, s), 10.67 (1H, s) (+)ESI-MS(m/z): 444(M+H)⁺, 466(M+Na)⁺

### EXAMPLE 116

A mixture of 2-(dimethylamino)-4-methylbenzoic acid (215 mg), 2-(4-aminophenyl)-N-(2-pyridinyl)acetamide (284 mg), 1-hydroxybenzotriazole (170 mg) and 1-[3-(dimethylamino)propyl]-3-ethylcarbodiimide (196 mg) in N,N-dimethylformamide (10 ml) was stirred at ambient temperature for 15 hours.

The reaction mixture was poured into a mixture of ethyl acetate and water, and the organic layer was washed with brine and dried over magnesium sulfate. The solvent was evaporated in vacuo and the residue was chromatographed on silica gel eluting with ethyl acetate: n-hexane (7:3 v/v). The eluted fractions containing the desired product were collected and the solvent was evaporated in vacuo. The residue was crystallized from a mixture of ethyl acetate and diisopropyl ether to give 2-(dimethylamino)-4-methyl-N-{4-[2-oxo-2-(2-pyridinylamino)ethyl]phenyl}benzamide (205 mg).

¹H-NMR(DMSO-d₆):δ 2.34 (3H, s), 2.76 (6H, s), 3.69 (2H, s), 6.95 (1H, d, J=7.8 Hz), 7.06-7.12 (2H, m), 7.32 (2H, d, J=8.3 Hz), 7.65-7.80 (4H, m), 8.02 (1H, d, J=8.3 Hz), 8.32 (1H, d, J=3.9 Hz), 10.68 (1H, s), 11.49 (1H, s) (+)ESI-MS(m/z): 389(M+H)⁺, 411(M+Na)⁺

### EXAMPLE 117

The following compound was obtained in substantially the same manner as in Example 116.

4-Methyl-2-(4-methyl-1-piperidinyl)-N-{4-[2-oxo-2-(2-pyridinylamino)ethyl]phenyl}benzamide

¹H-NMR(DMSO-d₆):δ 0.96 (3H, d, J=5.9 Hz), 1.14-1.49 (3H, m), 1.72-1.78 (2H, m), 2.35 (3H, s), 2.73-2.84 (2H, m), 3.08-3.13 (2H, m), 3.70 (2H, s), 7.03-7.12 (2H, m), 7.18 (1H, s), 7.34 (2H, d, J=8.4 Hz), 7.70 (2H, d, J=8.4 Hz), 7.72-7.84 (2H, m), 8.07 (1H, d, J=8.4 Hz), 8.32 (1H, d, J=3.8 Hz), 10.70 (1H, s), 11.94 (1H, s) (+)ESI-MS(m/z): 443(M+H)⁺, 465(M+Na)⁺

### EXAMPLE 118

The following compound was obtained in substantially the same manner as in Example 116.

N-[4-({[6-Methyl-2-(4-methyl-1-piperidinyl)-3-pyridinyl]carbonyl}amino)benzyl]-2-pyridinecarboxamide

¹H-NMR(DMSO-d₆):δ 0.88 (3H, d, J=6.2 Hz), 1.14-1.27 (2H, m), 1.42-1.64 (3H, m), 2.39 (3H, s), 2.74-2.86 (2H, m), 3.61-3.67 (2H, m), 4.46 (2H, d, J=6.4 Hz), 6.82 (1H, d, J=7.6 Hz), 7.30 (2H, d, J=8.4 Hz), 7.58-7.77 (4H, m), 7.96-8.08 (2H, m), 8.66 (1H, d, J=4.8 Hz), 9.32 (1H, t, J=6.4 Hz), 10.53 (1H, s) (+)ESI-MS(m/z): 444(M+H)⁺, 466(M+Na)⁺

### EXAMPLE 119

The following compound was obtained in substantially the same manner as in Example 116.

N-(4-{[4-Methyl-2-(4-methyl-1-piperidinyl)benzoyl]amino}benzyl)-2-pyridinecarboxamide

¹H-NMR(DMSO-d₆):δ 0.94 (3H, d, J=6.0 Hz), 1.17-1.50 (3H, m), 1.71-1.77 (2H, m), 2.28 (3H, s), 2.65-2.83 (2H, m), 3.07-3.13 (2H, m), 4.48 (2H, d, J=6.3 Hz), 7.05 (1H, d, J=7.9 Hz), 7.17 (1H, s), 7.33 (2H, d, J=8.4 Hz), 7.59-7.71 (3H, m), 7.82 (1H, d, J=7.9 Hz), 7.97-8.09 (2H, m), 8.66 (1H, d, J=4.7 Hz), 9.33 (1H, t, J=6.3 Hz), 11.93 (1H, s) (+)ESI-MS(m/z): 443(M+H)⁺, 465(M+Na)⁺

### EXAMPLE 120

A mixture of 6-methyl-2-(4-methyl-1-piperidinyl)nicotinic acid (350 mg), 4-[2-(1H-pyrazol-1-yl)ethoxy]phenylamine (320 mg), 1-hydroxybenzotriazole hydrate (242 mg) and 1-[3-(dimethylamino)propyl]-3-ethylcarbodiimide (245 mg) in N,N-dimethylformamide (20 ml) was stirred at ambient temperature for 15 hours.

The reaction mixture was poured into a mixture of ethyl acetate and water, and the organic layer was washed with brine and dried over magnesium sulfate. The solvent was evaporated in vacuo and the residue was chromatographed on silica gel eluting with ethyl acetate: n-hexane (6:4 v/v). The eluted fractions containing the desired product were collected and concentrated in vacuo and the precipitate was collected by filtration to give 6-methyl-2-(4-methyl-1-piperidinyl)-N-{4-[2-(1H-pyrazol-1-yl)ethoxy]phenyl}nicotinamide (532 mg).

¹H-NMR(DMSO-d₆):δ 0.89 (3H, d, J=6.1 Hz), 1.09-1.20 (2H, m), 1.42-1.64 (3H, m), 2.38 (3H, s), 2.73-2.85 (2H, m), 3.62-3.68 (2H, m), 4.30 (2H, t, J=5.2 Hz),4.48 (2H, t, J=5.2 Hz), 6.25 (1H, m), 6.81 (1H, d, J=7.6 Hz), 6.90 (2H, d, J=9.0 Hz), 7.46 (1H, d, J=1.7 Hz), 7.62 (2H, d, J=9.0 Hz), 7.73 (1H, d, J=7.6 Hz), 7.78 (1H, d, J=2.4 Hz), 10.40 (1H, s) (+)ESI-MS(m/z): 420(M+H)⁺, 442(M+Na)⁺

### EXAMPLE 121

The following compound was obtained in substantially the same manner as in Example 120.

4-Methyl-2-(4-methyl-1-piperidinyl)-N-{4-[2-(1H-pyrazol-1-yl)ethoxy]phenyl}benzamide

¹H-NMR(DMSO-d₆):δ 0.94 (3H, d, J=6.1 Hz), 1.21-1.50 (3H, m), 1.70-1.76 (2H, m), 2.71-2.82 (2H, m), 3.06-3.12 (2H, m), 4.34 (2H, t, J=5.2 Hz), 4.49 (2H, t, J=5.2 Hz), 6.25 (1H, m), 6.93 (2H, d, J=9.0 Hz), 7.03 (1H, d, J=8.0 Hz), 7.16 (1H, s), 7.46 (1H, d, J=1.3 Hz), 7.65 (2H, d, J=9.0 Hz), 7.78 (1H, s), 7.81 (1H, d, J=8.0 Hz), 11.80 (1H, s) (+)ESI-MS(m/z): 419(M+H)⁺, 441(M+Na)⁺

### EXAMPLE 122

The following compound was obtained in substantially the same manner as in Example 120.

2-(Dimethylamino)-4-methyl-N-(4-[2-(1H-pyrazol-1-yl)ethoxy]phenyl}benzamide

¹H-NMR(DMSO-d₆):δ 2.33 (3H, s), 2.76 (6H, s), 4.31 (2H, t, J=5.3 Hz), 4.49 (2H, t, J=5.3 Hz), 6.24-6.26 (1H, m), 6.88-6.96 (3H, m), 7.07 (1H, s), 7.47 (1H, d, J=1.6 Hz), 7.64-7.67 (2H, m), 7.78 (1H, d, J=2.2 Hz), 11.35 (1H, s) (+)ESI-MS(m/z): 365(M+H)⁺, 387(M+Na)⁺

### EXAMPLE 123

A mixture of 6-methyl-2-(4-methyl-1-piperidinyl)nicotinic acid (235 mg), 4-[2-(1H-1,2,4-triazol-1-yl)ethoxy]aniline (215 mg), 1-hydroxybenzotriazole (142 mg) and 1-[3-(dimethylamino)propyl]-3-ethylcarbodiimide (163 mg) in N,N-dimethylformamide (20 ml) was stirred at ambient temperature for 15 hours.

The reaction mixture was poured into a mixture of ethyl acetate and water, and the organic layer was washed with brine and dried over magnesium sulfate. The solvent was evaporated in vacuo and the residue was chromatographed on silica gel eluting with ethyl acetate:methanol (94:6 v/v). The eluted fractions containing the desired product were collected and concentrated in vacuo and the precipitate was collected by filtration to give 6-methyl-2-(4-methyl-1-piperidinyl)-N-{4-[2-(1H-1,2,4-triazol-1-yl)ethoxy]phenyl)nicotinamide (336 mg).

¹H-NMR(DMSO-d₆):δ 0.89 (3H, d, J=6.2 Hz), 1.09-1.26 (3H, m), 1.45-1.64 (2H,m), 2.39 (3H, s), 2.60-2.89 (2H, m), 3.34-3.62 (2H, m), 4.32 (2H, t, J=5.0 Hz), 4.58 (2H, t, J=5.0 Hz), 6.81 (1H, d, J=7.6 Hz), 6.40 (2H, d, J=9.0 Hz), 7.62 (2H, d, J=9.0 Hz), 7.73 (2H, J=7.6 Hz), 7.95 (1H, s), 8.58 (1H, s), 10.40 (1H, s)

### EXAMPLE 124

The following compound was obtained in substantially the same manner as in Example 123.

4-Chloro-2-(dimethylamino)-N-{4-[2-(1H-1,2,4-triazol-1-yl)ethoxy]phenyl}benzamide

¹H-NMR(DMSO-d₆):δ 2.79 (6H, s), 4.33 (2H, t, J=4.9 Hz), 4.57 (2H, t, J=4.9 Hz), 6.90 (2H, d, J=8.7 Hz), 7.00 (1H, d, J=8.2 Hz), 7.08 (1H, s), 7.51 (1H, d, J=8.2 Hz), 7.60 (2H, d, J=8.8 Hz), 8.00 (1H, s), 8.58 (1H, s), 10.59 (1H, s) (+)ESI-MS(m/z): 386(M+H)⁺, 408(M+Na)⁺

### EXAMPLE 125

The following compound was obtained in substantially the same manner as in Example 123.

6-Methyl-2-(4-methyl-1-piperidinyl)-N-(4-{[2-(1H-1,2,4-triazol-1-yl)ethyl]amino}phenyl)nicotinamide

¹H-NMR(DMSO-d₆):δ 0.90 (3H, d, J=6.1 Hz), 1.14-1.30 (2H, m), 1.46-1.67 (3H, m), 2.39 (3H, s), 2.73-2.89 (2H, m), 3.41-3.50 (2H, m), 3.60-3.66 (2H, m), 4.33 (2H, t, J=6.1 Hz), 5.65 (1H, t, J=6.0 Hz), 6.57 (2H, d, J=8.8 Hz), 6.82 (1H, d, J=7.6 Hz), 7.45 (2H, d, J=8.8 Hz), 7.65 (1H, d, J=7.6 Hz), 7.99 (1H, s), 8.48 (1H, s), 10.28 (1H, s) (+)ESI-MS(m/z): 420(M+H)⁺, 442(M+Na)⁺

### EXAMPLE 126

The following compound was obtained in substantially the same manner as in Example 123.

4-Methyl-2-(4-methyl-1-piperidinyl)-N-(4-{[2-(1H-1,2,4-triazol-1-yl)ethyl]amino}phenyl)benzamide

¹H-NMR(DMSO-d₆):δ 0.96 (3H, d, J=6.0 Hz), 1.26-1.51 (3H, m), 1.72-1.78 (2H, m), 2.34 (3H, m), 2.72-2.89 (2H, m), 3.06-3.12 (2H, m), 4.34 (2H, t, J=6.1 Hz), 5.66 (1H, t, J=6.0 Hz), 6.60 (2H, d, J=8.8 Hz), 7.03 (1H, d, J=8.0 Hz), 7.16 (1H, s), 7.49 (2H, d, J=8.8 Hz), 7.83 (1H, d, J=8.0 Hz), 7.99 (1H, s), 8.49 (1H, s), 11.73 (1H, s) (+)ESI-MS(m/z): 419(M+H)⁺, 441(M+Na)⁺

### EXAMPLE 127

The following compound was obtained in substantially the same manner as in Example 123.

2-(Dimethylamino)-4-methyl-N-(4-{[2-(1H-1,2,4-triazol-1-yl)ethyl]amino}phenyl)benzamide

¹H-NMR(DMSO-d₆):δ 2.33 (3H, s), 2.75 (6H, s), 3.41-3.50 (2H, m), 4.33 (2H, t J=6.1 Hz), 5.64 (1H, t, J=6.1 Hz), 6.57 (2H, d, J=8.8 Hz), 7.08 (1H, s), 7.44 (2H, d, J=8.8 Hz), 7.66 (1H, d, J=8.0 Hz), 7.99 (1H, s), 8.48 (1H, s), 11.19 (1H, s) (+)ESI-MS(m/z): 365(M+H)⁺, 387(M+Na)⁺

### EXAMPLE 128

The following compound was obtained in substantially the same manner as in Example 123.

6-Methyl-2-(4-methyl-1-piperidinyl)-N-{4-[3-(1H-1,2,4-triazol-1-yl)propyl[phenyl}nicotinamide

¹H-NMR(DMSO-d₆):δ 0.88 (3H, d, J=6.1 Hz), 1.02-1.28 (2H, m), 1.48-1.65 (3H, m), 2.39 (3H, s), 2.49-2.56 (2H, m), 3.57-3.69 (2H, m), 4.01-4.05 (2H, m), 4.19 (2H, t, J=7.0 Hz), 6.82 (2H, d, J=7.6 Hz), 7.18 (2H, d, J=8.4 Hz), 7.64 (2H, d, J=8.4 Hz), 7.75 (2H, d, J=7.6 Hz), 7.98 (1H, s), 8.54 (1H, s), 10.51 (1H, s) (+)ESI-MS(m/z): 419(M+H)⁺, 441(M+Na)⁺

### EXAMPLE 129

The following compound was obtained in substantially the same manner as in Example 120.

6-Methyl-2-(4-methyl-1-piperidinyl)-N-{6-[2-(1H-pyrazol-1-yl)ethoxy]-3-pyridinyl}nicotinamide

¹H-NMR(DMSO-d₆):δ 0.88 (3H, d, J=6.2 Hz), 1.02-1.08 (2H, m), 1.40-1.65 (3H, m), 2.39 (3H, s), 2.75-2.87 (2H, m), 3.65-3.71 (2H, m), 4.46-4.62 (4H, m), 6.23-6.25 (1H, m), 6.78-6.84 (2H, m), 7.45 (1H, d, J=1.4 Hz), 7.74 (1H, d, J=7.7 Hz), 7.76 (1H, d, J=2.4 Hz), 8.03 (1H, dd, J=2.6 Hz, 8.9 Hz), 8.49 (1H, d, J=2.6 Hz), 10.49 (1H, s) (+)ESI-MS(m/z): 421(M+H)⁺, 443(M+Na)⁺

### EXAMPLE 130

The following compound was obtained in substantially the same manner as in Example 120.

4-Methyl-2-(4-methyl-1-piperidinyl)-N-{6-[2-(1H-pyrazol-1-yl)ethoxy]-3-pyridinyl}benzamide

¹H-NMR(DMSO-d₆):δ 0.94 (3H, d, J=6.2 Hz), 1.02-1.53 (3H, m), 1.70-1.76 (2H, m), 2.35 (3H, s), 2.72-2.83 (2H, m), 3.14-3.34 (2H, m), 4.46-4.52 (2H, m), 4.56-4.62 (2H, m), 6.23-6.25 (1H, m), 6.83 (1H, d, J=8.8 Hz), 7.04 (1H, d, J=8.0 Hz), 7.16 (1H, s), 7.45 (1H, d, J=1.9 Hz), 7.75-7.79 (2H, m), 8.08 (1H, dd, J=2.6 Hz, 8.84 Hz), 8.49 (2H, d, J=2.5 Hz), 11.79 (1H, s) (+)ESI-MS(m/z): 420(M+H)⁺, 442(M+Na)⁺

### EXAMPLE 131

The following compound was obtained in substantially the same manner as in Example 120.

6-Methyl-2-(4-methyl-1-piperidinyl)-N-{4-[2-(1H-pyrrol-1-yl)ethoxy]phenyl}nicotinamide

¹H-NMR(DMSO-d₆):δ 0.88 (3H, d, J=6.2 Hz), 1.14-1.27 (2H, m), 1.45-1.64 (3H, m), 2.39 (3H, s), 2.74-2.85 (2H, m), 3.62-3.68 (2H, m), 4.15-4.28 (4H, m), 5.98-6.00 (2H, m), 6.79-6.95 (5H, m), 7.62 (2H, d, J=9.0 Hz), 7.73 (1H, d, J=7.6 Hz), 10.41 (1H, s) (+)ESI-MS(m/z): 419(M+H)⁺, 441(M+Na)⁺

### EXAMPLE 132

To a solution of 4-methyl-2-(4-methyl-1-piperidinyl)benzoic acid (117 mg), tert-butyl 4-aminophenyl(2-{2-[(tert-butoxycarbonyl)amino]-1,3-thiazol-4-yl)ethyl)carbamate (218 mg) and 1-hydroxybenzotriazole (99 mg) in N,N-dimethylformamide (10 ml) was added 1-[3-(dimethylamino)propyl]-3-ethylcarbodiimide hydrochloride (WSC.HCl) (124 mg), followed by triethylamine (66 mg) at ambient temperature and the mixture was stirred at ambient temperature for 3 days. The reaction mixture was poured into a mixture of ethyl acetate and water. The organic layer was washed with water and brine, dried over magnesium sulfate and concentrated in vacuo. The residue was purified by column chromatography on silica gel eluting with hexane: ethyl acetate (3:1 v/v) to give tert-butyl 2-{2-[(tert-butoxycarbonyl)amino]-1,3-thiazol-4-yl)ethyl(4-{[4-methyl-2-(4-methyl-1-piperidinyl)benzoyl]amino}phenyl)carbamate (138 mg) as a yellow tar.

¹H-NMR(DMSO-d₆):δ 1.06(3H, d, J=5.9 Hz), 1.32-1.72(3H, m), 1.49(18H, s), 1.86(2H, d, J=9.2 Hz), 2.39(3H, s), 2.84(2H, t, J=11.9 Hz), 2.97(2H, t, J=7.8 Hz), 3.18(2H, d, J=11.9 Hz), 3.92(2H, t, J=7.8 Hz), 6.79(1H, s), 7.08-7.18(4H, m), 7.73(2H, d, J=8.6 Hz), 8.18(1H, d, J=8.6 Hz), 12.63(1H, s) (+)ESI-MS(m/z): 650(M+H)⁺

### EXAMPLE 133

To a solution of tert-butyl 2-{2-[(tert-butoxycarbonyl)amino]-1,3-thiazol-4-yl)ethyl(4-{[4-methyl-2-(4-methyl-1-piperidinyl)benzoyl]amino}phenyl)carbamate (135 mg) in dichloromethane (5 ml) was added trifluoroacetic acid (474 mg). The reaction mixture was stirred at ambient temperature for 12 hours, quenched with a 10% aqueous potassium carbonate solution, and extracted with dichloromethane. The organic layer was washed with brine, dried over magnesium sulfate, filtered and concentrated in vacuo. The residue was triturated with diisopropyl ether to give N-(4-{[2-(2-amino-1,3-thiazol-4-yl)ethyl]amino}phenyl)-4-methyl-2-(4-methyl-1-piperidinyl)benzamide (68 mg) as a pale brown solid.

¹H-NMR(DMSO-d₆):δ 0.97(3H, d, J=6.3 Hz), 1.28-1.43(2H, m), 1.44-1.62(1H, m), 1.75(2H, d, J=10.9 Hz), 2.33(3H, s), 2.66(2H, t, J=7.3 Hz), 2.78(2H, t, J=10.8 Hz), 3.09(2H, d, J=11.6 Hz), 3.24(2H, t, J=7.3 Hz), 5.57(1H, brs), 6.23(1H, s), 6.59(2H, d, J=8.6 Hz), 6.88(2H, brs), 7.03(1H, d, J=7.6 Hz), 7.16(1H, s), 7.47(2H, d, J=8.9 Hz), 7.82(1H, d, J=7.9 Hz), 11.70(1H, s) (+)ESI-MS(m/z): 450(M+H)⁺

### EXAMPLE 134

The following compound was obtained in substantially the same manner as in Example 132.

tert-Butyl 2-{2-[(tert-butoxycarbonyl)amino]-1,3-thiazol-4-yl}ethyl(4-{[2-(dimethylamino)-4-methylbenzoyl]amino}phenyl)carbamate

¹H-NMR(CDCl₃):δ 1.49(18H, s), 2.40(3H, s), 2.82(6H, s), 2.96(2H, t, J=7.6 Hz), 3.92(2H, t, J=7.6 Hz), 6.79(1H, brs), 7.07-7.16(4H, m), 7.63(2H, d, J=8.9 Hz), 8.16(1H, d, J=8.9 Hz), 12.28(1H, brs) (+)ESI-MS(m/z): 596(M+H)⁺, 618(M+Na)⁺

### EXAMPLE 135

The following compound was obtained in substantially the same manner as in Example 133.

N-(4-{[2-(2-Amino-1,3-thiazol-4-yl)ethyl]amino}phenyl)-2-(dimethylamino)-4-methylbenzamide

¹H-NMR(DMSO-d₆):δ 2.33(3H, s), 2.66(2H, t, J=7.3 Hz), 2.75(6H, s), 3.19-3.30(2H, m), 5.49(1H, brs), 6.22(1H, s), 6.56(2H, d, J=8.9 Hz), 6.87(2H, brs), 6.94(1H, d, J=7.9 Hz), 7.08(1H, s), 7.42(2H, d, J=8.9 Hz), 7.66(1H, d, J=7.9 Hz), 11.17(1H, s) (+)ESI-MS(m/z): 396(M+H)⁺, 418(M+Na)⁺

### EXAMPLE 136

To a solution of 4-methyl-2-(4-methyl-1-piperidinyl)benzoic acid (323 mg), tert-butyl 6-{2-[(4-aminophenyl)amino]ethyl}-2-pyridinylcarbamate (454 mg) and 1-hydroxybenzotriazole (276 mg) in N,N-dimethylformamide (10 ml) was added 1-[3-(dimethylamino)propyl]-3-ethylcarbodiimide hydrochloride (WSC.HCl) (345 mg), followed by triethylamine (182 mg) at ambient temperature and the mixture was stirred at ambient temperature for 11 hours. The reaction mixture was poured into a mixture of ethyl acetate and water. The organic layer was washed with water and brine, dried over magnesium sulfate and concentrated in vacuo. The residue was purified by column chromatography on silica gel eluting with hexane: ethyl acetate (2:1 v/v) to give tert-butyl 6-{2-[(4-{[4-methyl-2-(4-methyl-1-piperidinyl)benzoyl]amino}phenyl)amino]-ethyl}-2-pyridinylcarbamate (226 mg) as a pale yellow foam.

¹H-NMR(CDCl₃):δ 1.03(3H, d, J=6.3 Hz), 1.44-1.54(3H, m), 1.53(9H, s), 1.84(2H, d, J=12.5 Hz), 2.38(3H, s), 2.81(2H, t, J=11.5 Hz), 2.96(2H, t, J=6.6 Hz), 3.18(2H, d, J=11.9 Hz), 3.49(2H, t, J=6.6 Hz), 6.65(2H, d, J=8.6 Hz), 6.83(1H, d, J=7.3 Hz), 7.06(2H, brs), 7.21(1H, brs), 7.55-7.61(3H, m), 7.76(1H, d, J=8.2 Hz), 8.17(1H, d, J=8.6 Hz), 12.25(1H, s) (+)ESI-MS(m/z): 544(M+H)⁺, 566(M+Na)⁺

### EXAMPLE 137

To a solution of tert-butyl 6-{2-[(4-{[4-methyl-2-(4-methyl-1-piperidinyl)benzoyl]amino}phenyl)amino]ethyl}-2-pyridinylcarbamate (220 mg) in dichloromethane (10 ml) was added trifluoroacetic acid (692 mg). The reaction mixture was stirred at ambient temperature for 16 hours, quenched with a 10% aqueous potassium carbonate solution, and extracted with dichloromethane. The organic layer was washed with brine, dried over magnesium sulfate and concentrated in vacuo. The residue was recrystallized from ethyl acetate and hexane to give N-(4-{[2-(6-amino-2-pyridinyl)ethyl]amino}phenyl)-4-methyl-2-(4-methyl-1-piperidinyl)benzamide (120 mg) as a pale yellow solid.

¹H-NMR(CDCl₃):δ 1.03(3H, d, J=6.3 Hz), 1.40-1.60(3H, m), 2.38(3H, s), 2.81(2H, t, J=11.5 Hz), 2.91(2H, t, J=6.6 Hz), 3.17(2H, d, J=11.9 Hz), 3.47(2H, t, J=6.6 Hz), 4.45(2H, brs), 6.36(1H, d, J=8.3 Hz), 6.53(1H, d, J=7.3 Hz), 6.65(2H, d, J=8.9 Hz), 7.04-7.08(2H, m), 7.36 (1H, t, J=7.3 Hz), 7.57(2H, d, J=8.9 Hz), 8.17(1H, d, J=8.6 Hz), 12.24(1H, s) (+)ESI-MS(m/z): 444(M+H)⁺

### EXAMPLE 138

The following compound was obtained in substantially the same manner as in Example 132.

tert-Butyl 6-{2-[(4-{[2-(dimethylamino)-4-methylbenzoyl]amino}phenyl)amino]ethyl}-2-pyridinylcarbamate

¹H-NMR(CDCl₃):δ 1.53(9H, s), 2.39(3H, s), 2.80(6H, s), 2.96(2H, t, J=6.6 Hz), 3.49(2H, t, J=6.6 Hz), 6.64(2H, d, J=8.9 Hz), 6.83(1H, d, J=7.3 Hz), 7.04-7.08(2H, m), 7.21(1H, brs), 7.49(2H, d, J=8.6 Hz), 7.58(1H, t, J=8.6 Hz), 7.77(1H, d, J=8.3 Hz), 8.14(1H, d, J=8.6 Hz), 11.86(1H, s), (+)ESI-MS(m/z): 512(M+Na)⁺

### EXAMPLE 139

The following compound was obtained in substantially the same manner as in Example 133.

N-(4-{[2-(6-Amino-2-pyridinyl)ethyl]amino}phenyl)-2-(dimethylamino)-4-methylbenzamide

¹H-NMR(CDCl₃):δ 2.39(3H, s), 2.80(6H, s), 2.90(2H, t, J=6.6 Hz), 3.47(2H, t, J=6.6 Hz), 4.46(2H, brs), 6.36(1H, d, J=7.9 Hz), 6.53(1H, d, J=7.3 Hz), 6.64(2H, d, J=8.9 Hz), 7.04-7.07(2H, m), 7.36(1H, t, J=7.3 Hz), 7.48(2H, d, J=8.9 Hz), 8.14(1H, d, J=8.6 Hz), 11.84(1H, s), (+)ESI-MS(m/z): 390(M+H)⁺

Preparation 108

To a solution of tert-butyl 4-aminophenyl(2-{6-[(tert-butoxycarbonyl)amino]-2-pyridinyl}ethyl)carbamate (578 mg), 2-chloro-6-methylnicotinic acid (769 mg) and 1-hydroxybenzotriazole (719 mg) in N,N-dimethylformamide (30 ml) was added 1-[3-(dimethylamino)propyl]-3-ethylcarbodiimide hydrochloride (WSC.HCl) (901 mg), followed by 4-(dimethylamino)pyridine (49 mg) at ambient temperature. The reaction mixture was stirred at the same temperature for 21 hours and concentrated in vacuo. The residue was dissolved in ethyl acetate and water, and extracted with ethyl acetate. The organic layer was washed with water and brine, dried over magnesium sulfate, filtered and concentrated in vacuo. The residue was purified by column chromatography on silica gel eluting with hexane: ethyl acetate (2:1→3:2 v/v) to give tert-butyl 2-{6-[(tert-butoxycarbonyl)amino]-2-pyridinyl}ethyl(4-{[(2-chloro-6-methyl-3-pyridinyl)carbonyl]amino}phenyl)carbamate (2.246 g) as a yellow foam.

¹H-NMR(CDCl₃):δ 1.42(18H, s), 2.60(3H, s), 3.04(2H, t, J=7.7 Hz), 3.95(2H, t, J=7.7 Hz), 7.05-7.26(5H, m), 7.57-7.61(3H, m), 8.10(1H, d, J=7.6 Hz), 8.34(1H, s), (+)ESI-MS(m/z): 583(M+H)⁺

### EXAMPLE 140

To a solution of tert-butyl 2-{6-[(tert-butoxycarbonyl)amino]-2-pyridinyl)ethyl(4-{[(2-chloro-6-methyl-3-pyridinyl)carbonyl]amino}phenyl)carbamate (681 mg) in tetrahydrofuran (30 ml) was added 4-methylpyperidine (1.16 g) at ambient temperature. The reaction mixture was refluxed for 24 hours, cooled to ambient temperature, and extracted with ethyl acetate. The organic layer was washed with water and brine, dried over magnesium sulfate, filtered and concentrated in vacuo. The residue was purified by column chromatography on silica gel eluting with hexane: ethyl acetate (2:1→3:2 v/v) to give tert-butyl 2-{6-[(tert-butoxycarbonyl)amino]-2-pyridinyl}ethyl[4-({[6-methyl-2-(4-methyl-1-piperidinyl)-3-pyridinyl]carbonyl}amino)phenyl]carbamate (640 mg) as a yellow foam.

¹H-NMR(CDCl₃):δ 1.04(3H, d, J=6.3 Hz), 1.32-1.47(2H, m), 1.40(9H, s), 1.49(9H, s), 1.50-1.72(1H, m), 1.85(2H, d, J=10.8 Hz), 2.52(3H, s), 2.88-3.05(4H, m), 3.34(2H, d, J=12.5 Hz), 3.95(2H, t, J=7.6 Hz), 6.81(1H, d, J=7.2 Hz), 7.03(1H, d, J=7.9 Hz), 7.10-7.17(3H, m), 7.54(1H, t, J=7.7 Hz), 7.67-7.73(3H, m), 8.37(1H, d, J=7.9 Hz), 11.87(1H, s) (+)ESI-MS(m/z): 645(M+H)⁺

### EXAMPLE 141

To a solution of tert-butyl 2-{6-[(tert-butoxycarbonyl)amino]-2-pyridinyl}ethyl[4-({[6-methyl-2-(4-methyl-1-piperidinyl)-3-pyridinyl]carbonyl}amino)phenyl]-carbamate (629 mg) in dichloromethane (10 ml) was added trifluoroacetic acid (1.5 ml). The reaction mixture was stirred at ambient temperature for 20 hours, quenched with a 10% aqueous potassium carbonate solution, and extracted with dichloromethane. The organic layer was washed with brine, dried over magnesium sulfate, filtered and concentrated in vacuo. The residue was recrystallized from ethyl acetate-diisopropyl ether to give N-(4-{[2-(6-amino-2-pyridinyl)ethyl]amino}phenyl)-6-methyl-2-(4-methyl-1-piperidinyl)nicotinamide (294 mg) as a pale brown solid.

¹H-NMR(DMSO-d₆):δ 0.90(3H, d, J=6.6 Hz), 1.14-1.27(2H, m), 1.32-1.59(1H, m), 1.63(2H, d, J=12.5 Hz), 2.38(3H, s), 2.70-2.84(4H, m), 3.26(2H, t, J=6.2 Hz), 3.63(2H, d, J=12.8 Hz), 5.56(1H, t, J=5.1 Hz), 5.84(2H, s), 6.27(1H, d, J=8.2 Hz), 6.39(1H, d, J=7.2 Hz), 6.57(2H, d, J=9.4 Hz), 6.82(1H, d, J=7.9 Hz), 7.27(1H, t, J=7.7 Hz), 7.42(2H, d, J=8.6 Hz), 7.75(1H, d, J=7.5 Hz), 10.25(1H, s) (+)ESI-MS(m/z): 445(M+H)⁺

### EXAMPLE 142

To a solution of tert-butyl 2-{6-[(tert-butoxycarbonyl)amino]-2-pyridinyl}ethyl(4-{[(2-chloro-6-methyl-3-pyridinyl)carbonyl]amino}phenyl)carbamate (681 mg) in tetrahydrofuran (30 ml) was added 2.0 mol/l dimethylamine in tetrahydrofuran (6.6 ml) at ambient temperature. The reaction mixture was heated at 60° C. for 20 hours, cooled to ambient temperature, and extracted with ethyl acetate. The organic layer was washed with water and brine, dried over magnesium sulfate, filtered and concentrated in vacuo. The residue was purified by column chromatography on silica gel eluting with hexane: ethyl acetate (2:1→3:2 v/v) to give tert-butyl 2-{6-[(tert-butoxycarbonyl)amino]-2-pyridinyl}ethyl[4-({[2-(dimethylamino)-6-methyl-3-pyridinyl]carbonyl)amino)phenyl]-carbamate (529 mg) as a yellow foam.

¹H-NMR(CDCl₃):δ 1.40(9H, s), 1.50(9H, s), 2.52 (3H, s), 2.90(6H, s), 2.91(2H, t, J=7.4 Hz), 3.95(2H, t, J=7.4 Hz), 6.81(1H, d, J=7.6 Hz), 6.97(1H, d, J=7.9 Hz), 7.12-7.16(3H, m), 7.54(1H, t, J=7.9 Hz), 7.62(2H, d, J=8.6 Hz), 7.72(1H, d, J=8.2 Hz), 8.27(1H, d, J=7.9 Hz), 10.90(1H, s) (+)ESI-MS(m/z): 645(M+H)⁺

### EXAMPLE 143

The following compound was obtained in substantially the same manner as in Example 141.

N-(4-{[2-(6-Amino-2-pyridinyl)ethyl]amino}phenyl)-2-(dimethylamino)-6-methylnicotinamide

¹H-NMR(DMSO-d₆):δ 2.34(3H, s), 2.72(2H, t, J=7.2 Hz), 2.94(6H, s), 3.26(2H, t, J=7.2 Hz), 5.54(1H, s), 5.84(2H, s), 6.27(1H, d, J=8.2 Hz), 6.40(1H, d, J=7.2 Hz), 6.55(2H, d, J=8.9 Hz), 6.59(1H, d, J=7.6 Hz), 7.27(1H, t, J=7.7 Hz), 7.39(2H, d, J=8.9 Hz), 7.54(1H, d, J=7.2 Hz), 9.91(1H, s) (+)ESI-MS(m/z): 391(M+H)⁺

Preparation 109

The following compound was obtained in substantially the same manner as in Preparation 108.

tert-Butyl 6-[2-(4-{[(2-chloro-6-methyl-3-pyridinyl)carbonyl]amino}phenoxy)ethyl]-2-pyridinylcarbamate

¹H-NMR(CDCl₃):δ 1.51(9H, s), 2.59(3H, s), 3.13(2H, t, J=6.7 Hz), 4.31(2H, t, J=6.7 Hz), 6.90(2H, d, J=9.2 Hz), 7.21(1H, d, J=7.2 Hz), 7.22(1H, s), 7.50-7.61(3H, m), 7.77(1H, d, J=8.2 Hz), 8.12(1H, d, J=7.9 Hz), 8.19(1H, s) (+)ESI-MS(m/z): 483(M+H)⁺

### EXAMPLE 144

The following compound was obtained in substantially the same manner as in Example 140.

tert-Butyl 6-{2-[4-({[6-methyl-2-(4-methyl-1-piperidinyl)-3-pyridinyl]carbonyl}amino)phenoxy]ethyl}-2-pyridinylcarbamate

¹H-NMR(CDCl₃):δ 1.02(3H, d, J=6.6 Hz), 1.36-1.47(2H, m), 1.51(9H, s), 1.52-1.65(1H, m), 1.83(2H, d, J=10.5 Hz), 2.51(3H, s), 2.99(2H, td, J=12.2,2.3 Hz), 3.12(1H, t, J=6.7 Hz), 3.34(2H, d, J=12.8 Hz), 4.31(2H, t, J=6.9 Hz), 6.91(2H, d, J=8.9 Hz), 7.01(1H, d, J=7.2 Hz), 7.18(1H, s), 7.59(1H, t, J=2.9 Hz), 7.63(2H, d, J=8.9 Hz), 7.76(1H, d, J=7.9 Hz), 8.35(1H, d, J=7.9 Hz), 11.63(1H, s) (+)ESI-MS(m/z): 546(M+H)⁺

### EXAMPLE 145

The following compound was obtained in substantially the same manner as in Example 141.

N-{4-[2-(6-Amino-2-pyridinyl)ethoxy]phenyl}-6-methyl-2-(4-methyl-1-piperidinyl)nicotinamide

¹H-NMR(DMSO-d₆):δ 0.88(3H, d, J=6.3 Hz), 1.11-1.26(2H, m), 1.46-1.51(1H, m), 1.62(2H, d, J=12.5 Hz), 2.38(3H, s), 2.80(2H, t, J=10.7 Hz), 2.92(2H, t, J=6.7 Hz), 3.65(2H, d, J=12.8 Hz), 4.24(2H, t, J=6.7 Hz), 5.83(1H, s), 6.28(1H, d, J=7.6 Hz), 6.44(1H, d, J=6.9 Hz), 6.81(1H, d, J=7.6 Hz), 6.91(2H, d, J=8.9 Hz), 7.28(1H, dd, J=8.2 Hz, 7.2 Hz), 7.61(2H, d, J=9.2 Hz), 7.74(1H, d, J=7.6 Hz), 10.39(1H, s), (+)ESI-MS(m/z): 446(M+H)⁺

### EXAMPLE 146

The following compound was obtained in substantially the same manner as in Example 142.

tert-Butyl 6-{2-[4-({[2-(dimethylamino)-6-methyl-3-pyridinyl]carbonyl}amino)phenoxy]ethyl}-2-pyridinylcarbamate

¹H-NMR(CDCl₃):δ 1.51(9H, s), 2.50(3H, s), 2.88(6H, s), 3.12(2H, t, J=6.7 Hz), 4.30(2H, t, J=6.7 Hz), 6.87-6.95(4H, m), 7.20(1H, br s), 7.54(2H, d, J=9.2 Hz), 7.57(1H, d, J=7.9 Hz), 7.77(1H, d, J=7.9 Hz), 8.24(1H, d, J=7.6 Hz), 10.64(1H, s), (+)ESI-MS(m/z): 514(M+Na)⁺

### EXAMPLE 147

The following compound was obtained in substantially the same manner as in Example 141.

N-{4-[2-(6-Amino-2-pyridinyl)ethoxy]phenyl}-2-(dimethylamino)-6-methylnicotinamide

¹H-NMR(DMSO-d₆):δ 2.35(3H, s), 2.93(6H, s), 2.97(2H, t, J=6.9 Hz), 4.24(2H, t, J=6.7 Hz), 6.35(2H, br s), 6.43(1H, d, J=8.2 Hz), 6.54(1H, d, J=7.2 Hz), 6.60(1H, d, J=7.6 Hz), 6.89(2H, d, J=8.9 Hz), 7.43(1H, t, J=7.7 Hz), 7.57(1H, d, J=7.6 Hz), 7.58(2H, d, J=8.9 Hz), 10.14(1H, s) (+)ESI-MS(m/z): 392(M+H)⁺

Preparation 110

The following compound was obtained in substantially the same manner as in Preparation 108.

tert-Butyl 2-{2-[(tert-butoxycarbonyl)amino]-1,3-thiazol-4-yl}ethyl(4-{[(2-chloro-6-methyl-3-pyridinyl)carbonyl]amino}phenyl)carbamate

¹H-NMR(CDCl₃):δ 1.49(18H, s), 2.60(3H, s), 2.94(2H, t, J=7.6 Hz), 3.91(2H, t, J=7.6 Hz), 6.77(1H, s), 7.15(2H, d, J=8.6 Hz), 7.24(1H, d, J=7.9 Hz), 7.59(2H, d, J=8.9 Hz), 8.11(1H, d, J=7.9 Hz), 8.32(1H, s) (+)ESI-MS(m/z): 610(M+Na)⁺

### EXAMPLE 148

The following compound was obtained in substantially the same manner as in Example 140.

tert-Butyl 2-{2-[(tert-butoxycarbonyl)amino]-1,3-thiazol-4-yl}ethyl[4-({[6-methyl-2-(4-methyl-1-piperidinyl)-3-pyridinyl]carbonyl}amino)phenyl]carbamate

¹H-NMR(CDCl₃):δ 1.03(3H, d, J=6.6 Hz), 1.40(9H, s), 1.51(9H, s), 1.45-1.73(3H, m), 1.82-1.87(2H, m), 2.52(3H, s), 2.88(2H, t, J=7.6 Hz), 3.00(2H, t, J=11.3 Hz), 3.34(2H, d, J=12.5 Hz), 3.92(2H, t, J=7.6 Hz), 6.52(1H, s), 7.02(1H, d, J=7.9 Hz), 7.15(2H, d, J=7.9 Hz), 7.68(2H, d, J=8.6 Hz), 8.36(1H, d, J=7.9 Hz), 8.39(1H, s), 11.85(1H, s) (+)ESI-MS(m/z): 651(M+H)⁺

### EXAMPLE 149

The following compound was obtained in substantially the same manner as in Example 141.

N-(4-{[2-(2-Amino-1,3-thiazol-4-yl)ethyl]amino}phenyl)-6-methyl-2-(4-methyl-1-piperidinyl)nicotinamide

¹H-NMR(DMSO-d₆):δ 0.90(3H, d, J=6.3 Hz), 1.11-1.28(2H, m), 1.44-1.6(1H, m), 1.63(2H, d, J=12.2 Hz), 2.38(3H, s), 2.65(2H, t, J=7.2 Hz), 2.79(2H, t, J=12.2 Hz), 3.23(2H, dd, J=7.2 Hz, 5.6 Hz), 3.63(2H, d, J=12.5 Hz), 5.49(1H, t, J=5.6 Hz), 6.21(1H, s), 6.54(2H, d, J=8.6 Hz), 6.82(1H, d, J=7.6 Hz), 6.85(2H, s), 7.43(2H, d, J=8.6 Hz), 7.74(1H, d, J=7.6 Hz), 10.26(1H, s) (+)ESI-MS(m/z): 451(M+H)⁺

### EXAMPLE 150

The following compound was obtained in substantially the same manner as in Example 142.

tert-Butyl 2-{2-[(tert-butoxycarbonyl)amino]-1,3-thiazol-4-yl}ethyl[4-({[2-(dimethylamino)-6-methyl-3-pyridinyl]carbonyl}amino)phenyl]carbamate

¹H-NMR(CDCl₃):δ 1.40(9H, s), 1.50(9H, s), 2.51(3H, s), 2.89(6H, s), 2.90(2H, t, J=7.6 Hz), 3.91(2H, t, J=7.6 Hz), 6.52(1H, s), 6.96(1H, d, J=7.9 Hz), 7.12(2H, d, J=8.2 Hz), 7.60(2H, d, J=8.6 Hz), 8.25(1H, d, J=7.9 Hz), 8.66(1H, br s), 10.88(1H, s) (+)ESI-MS(m/z): 597(M+H)⁺

### EXAMPLE 151

The following compound was obtained in substantially the same manner as in Example 141.

N-(4-{[2-(2-Amino-1,3-thiazol-4-yl)ethyl]amino}phenyl)-2-(dimethylamino)-6-methylnicotinamide

¹H-NMR(DMSO-d₆):δ 2.34(3H, s), 2.65(2H, t, J=7.4 Hz), 2.93(6H, s), 3.22(2H, dd, J=7.4 Hz, 5.6 Hz), 5.46(1H, t, J=5.6 Hz), 6.20(1H, s), 6.53(2H, d, J=8.6 Hz), 6.59(1H, d, J=7.2 Hz), 6.84(2H, s), 7.39(2H, d, J=8.6 Hz), 7.53(1H, d, J=7.6 Hz), 9.90(1H, s), (+)ESI-MS(m/z): 397(M+H)⁺

Preparation 111

The following compound was obtained in substantially the same manner as in Preparation 108.

tert-Butyl 4-[2-(4-{[(2-chloro-6-methyl-3-pyridinyl)carbonyl]amino}phenoxy)ethyl]-1,3-thiazol-2-ylcarbamate

¹H-NMR(CDCl₃):δ 1.53(9H, s), 2.59(3H, s), 3.13(2H, t, J=6.5 Hz), 4.24(2H, t, J=6.8 Hz), 6.62(1H, s), 6.90(2H, d, J=9.2 Hz), 7.21(1H, d, J=7.9 Hz), 7.50(2H, d, J=8.9 Hz), 8.11(1H, d, J=7.6 Hz), 8.19(1H, s) (+)ESI-MS(m/z): 489(M+H)⁺

### EXAMPLE 152

The following compound was obtained in substantially the same manner as in Example 140.

tert-Butyl 4-{2-[4-({[6-methyl-2-(4-methyl-1-piperidinyl)-3-pyridinyl]carbonyl}amino)phenoxy]ethyl}-1,3-thiazol-2-ylcarbamate

¹H-NMR(CDCl₃):δ 1.01(3H, d, J=6.3 Hz), 1.30-1.47(2H, m), 1.53(9H, s), 1.54-1.97(1H, m), 1.83(2H, d, J=12.8 Hz), 2.51(3H, s), 2.98(2H, t, J=10.8 Hz), 3.17(2H, t, J=6.6 Hz), 3.34(2H, d, J=12.5 Hz), 4.25(2H, t, J=6.6 Hz), 6.64(1H, s), 6.91(2H, d, J=8.9 Hz), 7.01(1H, d, J=7.9 Hz), 7.64(2H, d, J=9.2 Hz), 8.35(1H, d, J=7.9 Hz), 9.55(1H, br s), 11.63(1H, s) (+)ESI-MS(m/z): 552(M+H)⁺

### EXAMPLE 153

The following compound was obtained in substantially the same manner as in Example 141.

N-{4-[2-(2-Amino-1,3-thiazol-4-yl)ethoxy]phenyl}-6-methyl-2-(4-methyl-1-piperidinyl)nicotinamide

¹H-NMR(DMSO-d₆):δ 0.89(3H, d, J=6.3 Hz), 1.11-1.25(2H, m), 1.44-1.52(1H, m), 1.62(2H, d, J=12.5 Hz), 2.38(3H, s), 2.76-2.87(4H, m), 3.65(2H, d, J=12.8 Hz), 4.17(2H, t, J=6.9 Hz), 6.26(1H, s), 6.81(1H, d, J=7.6 Hz), 6.86(2H, s), 6.92(2H, d, J=8.9 Hz), 7.62(2H, d, J=8.9 Hz), 7.74(1H, d, J=7.6 Hz), 10.39(1H, s) (+)ESI-MS(m/z): 452(M+H)⁺

### EXAMPLE 154

The following compound was obtained in substantially the same manner as in Example 142.

tert-Butyl 4-{2-[4-({[2-(dimethylamino)-6-methyl-3-pyridinyl]carbonyl}amino)phenoxy]ethyl}-1,3-thiazol-2-ylcarbamate

¹H-NMR(CDCl₃):δ 1.53(9H, s), 2.51(3H, s), 2.89(6H, s), 3.14(2H, t, J=6.7 Hz), 4.24(2H, t, J=6.7 Hz), 6.63(1H, s), 6.89(2H, d, J=9.2 Hz), 6.94(1H, d, J=7.9 Hz), 7.55(2H, d, J=9.2 Hz), 8.24(1H, d, J=7.9 Hz), 9.02(1H, br s), 10.66(1H, s) (+)ESI-MS(m/z): 498(M+H)⁺

### EXAMPLE 155

The following compound was obtained in substantially the same manner as in Example 141.

N-{4-[2-(2-Amino-1,3-thiazol-4-yl)ethoxy]phenyl}-2-(dimethylamino)-6-methylnicotinamide

¹H-NMR(DMSO-d₆):δ 2.35(3H, s), 2.84(2H, t, J=6.7 Hz), 2.93(6H, s), 4.16(2H, t, J=6.7 Hz), 6.26(1H, s), 6.60(1H, d, J=7.6 Hz), 6.86(2H, s), 6.89(2H, d, J=8.9 Hz), 7.56(1H, d, J=7.2 Hz), 7.58(2H, d, J=8.9 Hz), 10.14(1H, s) (+)ESI-MS(m/z): 398(M+H)⁺

### EXAMPLE 156

To a solution of tert-butyl 4-[2-(4-aminophenoxy)ethyl]-1,3-thiazol-2-ylcarbamate (177 mg), 4-methyl-2-(4-methyl-1-piperidinyl)benzoic acid (135 mg) and 1-hydroxybenzotriazole (88.9 mg) in N,N-dimethylformamide (3.5 ml) was added 1-[3-(dimethylamino)propyl]-3-ethylcarbodiimide hydrochloride (WSC.HCl) (111 mg), followed by N,N-dimethylaminopyridine (3.2 mg) at ambient temperature. The reaction mixture was stirred at ambient temperature for 23 hours and concentrated in vacuo. The residue was dissolved in ethyl acetate and water, and extracted with ethyl acetate. The organic layer was washed with water and brine, dried over magnesium sulfate, filtered and concentrated in vacuo. The residue was purified by column chromatography on silica gel eluting with hexane: ethyl acetate (2:1→1:1 v/v) to give tert-butyl 4-[2-(4-{[4-methyl-2-(4-methyl-1-piperidinyl)benzoyl]amino}phenoxy)ethyl]-1,3-thiazol-2-ylcarbamate (0.159 g) as a pale brown foam.

¹H-NMR(DMSO-d₆):δ 1.03(3H, d, J=6.2 Hz), 1.40-1.70(1H, m), 1.47(2H, td, J=13.2, 3.5 Hz), 1.54(9H, s), 1.84(2H, dd, J=13.0 Hz, 1.6 Hz), 2.38(3H, s), 2.82(2H, t, J=11.3 Hz), 3.10-3.21(4H, m), 4.23(2H, d, J=6.8 Hz), 6.64(1H, s), 6.89(2H, d, J=9.2 Hz), 7.06(1H, d, J=7.3 Hz), 7.08(1H, s), 7.65(2H, d, J=9.2 Hz), 8.16(1H, d, J=8.1 Hz), 12.44(1H, s) (+)ESI-MS(m/z): 551(M+H)⁺

### EXAMPLE 157

To a solution of tert-butyl 4-[2-(4-{[4-methyl-2-(4-methyl-1-piperidinyl)benzoyl]amino}phenoxy)ethyl]-1,3-thiazol-2-ylcarbamate (159 mg) in dichloromethane (1.58 ml) was added trifluoroacetic acid (0.334 ml). The mixture was stirred for 12 hours at room temperature, quenched with 10% aqueous potassium carbonate solution, and extracted with dichloromethane. The organic layer was washed with brine, dried over magnesium sulfate, filtered and concentrated in vacuo. The residue was recrystallized from hexane-ethyl acetate to give N-{4-[2-(2-amino-1,3-thiazol-4-yl)ethoxy]phenyl)-4-methyl-2-(4-methyl-1-piperidinyl)benzamide (0.059 g) as a pale brown powder.

¹H-NMR(CDCl₃):δ 1.04(3H, d, J=5.9 Hz), 1.48(2H, td, J=11.3 Hz), 1.50-1.70(1H, m), 1.85(2H, dd, J=12.7 Hz, 2.7 Hz), 2.38(3H, s), 2.82(2H, td, J=11.9 Hz, 2.2 Hz), 3.02(2H, t, J=6.8 Hz), 4.25(2H, t, J=6.8 Hz), 5.03(1H, br s), 6.28(1H, s), 6.92(2H, d, J=9.2 Hz), 7.06-7.23(2H, m), 7.66(2H, d, J=9.2 Hz), 8.17(2H, d, J=8.4 Hz), 12.41(1H, s) (+)ESI-MS(m/z): 451(M+H)⁺

### EXAMPLE 158

The following compound was obtained in substantially the same manner as in Example 156.

tert-Butyl 4-[2-(4-{[2-(dimethylamino)-4-methylbenzoyl]amino}phenoxy)ethyl]-1,3-thiazol-2-ylcarbamate

¹H-NMR(CDCl₃):δ 1.55(9H, s), 2.39(3H, s), 2.80(6H, s), 3.13(2H, t, J=6.5 Hz), 4.22(2H, t, J=6.8 Hz), 6.63(1H, s), 6.87(2H, d, J=8.9 Hz), 7.06(1H, d, J=8.4 Hz), 7.08(1H, s), 7.55(2H, d, J=8.9 Hz), 8.13(1H, d, J=7.6 Hz), 12.08(1H, s) (+)ESI-MS(m/z): 497(M+H)⁺

### EXAMPLE 159

The following compound was obtained in substantially the same manner as in Example 157.

N-{4-[2-(2-Amino-1,3-thiazol-4-yl)ethoxy]phenyl}-2-(dimethylamino)-4-methylbenzamide

¹H-NMR(CDCl₃):δ 2.39(3H, s), 2.80(6H, s), 3.02(2H, t, J=6.8 Hz), 4.24(2H, t, J=6.8 Hz), 4.96(2H, br s), 6.26(1H, s), 6.91(2H, d, J=8.9 Hz), 7.07(1H, d, J=7.3 Hz), 7.08(1H, s), 7.57(2H, d, J=9.2 Hz), 8.14(2H, d, J=8.6 Hz), 12.04(1H, s) (+)ESI-MS(m/z): 397(M+H)⁺

### EXAMPLE 160

The following compound was obtained in substantially the same manner as in Example 156.

tert-Butyl 4-{2-[(5-{[4-methyl-2-(4-methyl-1-piperidinyl)benzoyl]amino}-2-pyridinyl)oxy]ethyl}-1,3-thiazol-2-ylcarbamate

¹H-NMR(CDCl₃):δ 1.05(3H, d, J=6.2 Hz), 1.46(2H, td, J=13.0, 3.8 Hz), 1.54(9H, s), 1.55-1.72(1H, m), 1.87(2H, dd, J=13.5 Hz, 1.6 Hz), 2.39(3H, s), 2.84(2H, t, J=9.7 Hz), 3.10-3.19(4H, m), 4.57(2H, t, J=7.0 Hz), 6.62(1H, s), 6.76(1H, d, J=10.0 Hz), 7.09(1H, d, J=7.3 Hz), 7.11(1H, s), 8.18(1H, d, J=8.6 Hz), 8.27-8.31(2H, m), 12.64(1H, s) (+)ESI-MS(m/z): 552(M+H)⁺

### EXAMPLE 161

The following compound was obtained in substantially the same manner as in Example 157.

N-{6-[2-(2-Amino-1,3-thiazol-4-yl)ethoxy]-3-pyridinyl}-4-methyl-2-(4-methyl-1-piperidinyl)benzamide

¹H-NMR(CDCl₃):δ 1.06(3H, d, J=6.2 Hz), 1.44-1.72(1H, m), 1.46(2H, td, J=11.9, 3.5 Hz), 1.85(2H, dd, J=13.5 Hz, 1.7 Hz), 2.39(3H, s), 2.84(2H, td, J=11.6 Hz, 2.2 Hz), 3.04(2H, t, J=6.8 Hz), 3.17(2H, br d, J=12.4 Hz), 4.56(2H, t, J=7.0 Hz), 4.89(1dH, br s), 6.27(1H, s), 6.77(1H, d, J=8.6 Hz), 7.10(1H, d, J=7.0 Hz), 7.11(1H, s), 8.18(1H, d, J=8.6 Hz), 8.23-8.33(2H, m), 12.65(1H, s) (+)ESI-MS(m/z): 452(M+H)⁺

### EXAMPLE 162

The following compound was obtained in substantially the same manner as in Example 156.

tert-Butyl 4-{2-[(5-{[2-(dimethylamino)-4-methylbenzoyl]amino)-2-pyridinyl)oxy]ethyl)-1,3-thiazol-2-ylcarbamate

¹H-NMR(CDCl₃):δ 1.56(9H, s), 2.40(3H, s), 2.80(6H, s), 3.13(2H, t, J=6.5 Hz), 4.54(2H, t, J=6.8 Hz), 6.62(1H, s), 6.71(1H, d, J=8.9 Hz), 7.09(1H, d, J=8.4 Hz), 7.11(1H, s), 8.12-8.21(3H, m), 12.37(1H, br s) (+)ESI-MS(m/z): 498(M+H)⁺

### EXAMPLE 163

The following compound was obtained in substantially the same manner as in Example 157.

N-{6-[2-(2-Amino-1,3-thiazol-4-yl)ethoxy]-3-pyridinyl)-2-(dimethylamino)-4-methylbenzamide

¹H-NMR(CDCl₃):δ 2.40(3H, s), 2.81(6H, s), 3.03(2H, t, J=6.8 Hz), 4.56(2H, t, J=6.8 Hz), 4.92(2H, br s), 6.25(1H, s), 6.76(1H, d, J=8.9 Hz), 7.10(1H, d, J=8.6 Hz), 7.11(1H, s), 8.13-8.23(3H, m), 12.32(1H, s) (+)ESI-MS(m/z): 398(M+H)⁺

### EXAMPLE 164

To a solution of tert-butyl 6-[2-(4-aminophenoxy)ethyl]-2-pyridinylcarbamate (498 mg), 4-methyl-2-(4-methyl-1-piperidinyl)benzoic acid (423 mg) and 1-hydroxybenzotriazole (278 mg) in N,N-dimethylformamide (30 ml) was added 1-[3-(dimethylamino)propyl]-3-ethylcarbodiimide hydrochloride (WSC.HCl) (348 mg), followed by 4-(dimethylamino)pyridine (18 mg) at ambient temperature. The reaction mixture was stirred at the same temperature for 21 hours and concentrated in vacuo. The residue was dissolved in ethyl acetate and water, and extracted with ethyl acetate. The organic layer was washed with water and brine, dried over magnesium sulfate, filtered and concentrated in vacuo. The residue was purified by column chromatography on silica gel eluting with hexane: ethyl acetate (4:1 v/v) to give tert-butyl 6-[2-(4-{[4-methyl-2-(4-methyl-1-piperidinyl)benzoyl]amino}phenoxy)ethyl]-2-pyridinylcarbamate (312 mg) as a yellow foam.

¹H-NMR(DMSO-d₆):δ 0.95(3H, d, J=6.3 Hz), 1.30-1.35(2H, m), 1.45(9H, s), 1.47-1.54(1H, m), 1.73(2H, d, J=11.2 Hz), 2.34(3H, s), 2.77(2H, t, J=10.5 Hz), 3.04-3.12(4H, m), 4.30(2H, t, J=6.6 Hz), 6.94(2H, d, J=9.2 Hz), 6.98-7.04(2H, m), 7.16(1H, s), 7.62-7.66(14H, m), 7.80(1H, d, J=7.9 Hz), 9.65(1H, s), 11.79(1H, s), (+)ESI-MS(m/z): 567(M+Na)⁺

### EXAMPLE 165

To a solution of tert-butyl 6-[2-(4-{[4-methyl-2-(4-methyl-1-piperidinyl)benzoyl]amino}phenoxy)ethyl]-2-pyridinylcarbamate (302 mg) in dichloromethane (5 ml) was added trifluoroacetic acid (0.854 ml). The reaction mixture was stirred at ambient temperature for 19 hours, quenched with 10% aqueous potassium carbonate solution, and extracted with dichloromethane. The organic layer was washed with brine, dried over magnesium sulfate, filtered and concentrated in vacuo. The residue was recrystallized from ethyl acetate-diisopropyl ether to give N-{4-[2-(6-amino-2-pyridinyl)ethoxy]phenyl}-4-methyl-2-(4-methyl-1-piperidinyl)benzamide (294 mg) as a white solid.

¹H-NMR(DMSO-d₆):δ 0.95(3H, d, J=6.3 Hz), 1.25-1.39(2H, m), 1.46-1.53(1H, m), 1.73(2H, d, J=10.8 Hz), 2.34(3H, s), 2.77(2H, t, J=10.2 Hz), 2.92(2H, t, J=6.7 Hz), 3.10(2H, d, J=11.5 Hz), 4.24(2H, t, J=6.7 Hz), 5.85(2H, s), 6.29(1H, d, J=8.2 Hz), 6.45(1H, d, J=6.6 Hz), 6.94(2H, d, J=8.9 Hz), 7.04(1H, d, J=7.9 Hz), 7.16(1H, s), 7.29(1H, dd, J=8.2 Hz, 7.2 Hz), 7.65(2H, d, J=9.2 Hz), 7.80(1H, d, J=7.6 Hz), 11.80(1H, s) (+)ESI-MS(m/z): 445(M+H)⁺

### EXAMPLE 166

The following compound was obtained in substantially the same manner as in of Example 164.

tert-Butyl 6-[2-(4-{[2-(dimethylamino)-4-methylbenzoyl]amino}phenoxy)ethyl]-2-pyridinylcarbamate

¹H-NMR(DMSO-d₆):δ 1.46(9H, s), 2.33(3H, s), 2.75(6H, s), 3.06(2H, t, J=6.6 Hz), 4.30(2H, t, J=6.6 Hz), 6.90-6.94(3H, m), 6.99(1H, dd, J=5.9 Hz, 2.6 Hz), 7.07(1H, s), 7.59-7.67(5H, m), 9.65(1H, s), 11.32(1H, s) (+)ESI-MS(m/z): 513(M+Na)⁺

### EXAMPLE 167

The following compound was obtained in substantially the same manner as in Example 165.

N-{4-[2-(6-Amino-2-pyridinyl)ethoxy]phenyl}-2-(dimethylamino)-4-methylbenzamide

¹H-NMR(DMSO-d₆):δ 2.33(3H, s), 2.75(6H, s), 2.92(2H, t, J=6.7 Hz), 4.24(2H, t, J=6.7 Hz), 5.85(2H, s), 6.29(1H, d, J=8.2 Hz), 6.45(1H, d, J=7.2 Hz), 6.89-6.94(3H, m), 7.07(1H, s), 7.29(1H, t, J=7.7 Hz), 7.59-7.66(3H, m), 11.32(1H, s) (+)ESI-MS(m/z): 391(M+H)⁺

### EXAMPLE 168

To a solution of tert-butyl 6-[2-(4-aminophenoxy)ethyl]-2-pyridinylcarbamate (458 mg), 2-(dimethylamino)benzoic acid (253 mg) and 1-hydroxybenzotriazole (256 mg) in N,N-dimethylformamide (10 ml) was added 1-[3-(dimethylamino)propyl]-3-ethylcarbodiimide hydrochloride (320 mg), followed by triethylamine (0.29 ml) at ambient temperature. The reaction mixture was stirred at the same temperature for 16 hours and concentrated in vacuo. The residue was dissolved in ethyl acetate and water, and extracted with ethyl acetate. The organic layer was washed with water and brine, dried over magnesium sulfate, filtered and concentrated in vacuo. The residue was purified by column chromatography on silica gel eluting with hexane: ethyl acetate (4:1 v/v) to give tert-butyl 6-[2-(4-{[2-(dimethylamino)benzoyl]amino}phenoxy)ethyl]-2-pyridinylcarbamate (549 mg) as a pale yellow foam.

¹H-NMR(CDCl₃):δ 1.51(9H, s), 2.82(6H, s), 3.12(2H, t, J=6.7 Hz), 4.31(2H, t, J=6.7 Hz), 6.88-6.92(3H, m), 7.21-7.30(3H, m), 7.43-7.50(1H, m), 7.54-7.64(3H, m), 7.77(1H, d, J=8.2 Hz), 8.25(1H, dd, J=7.9 Hz, 1.6 Hz), 11.98(1H, s) (+)ESI-MS(m/z): 477(M+H)⁺

### EXAMPLE 169

The following compound was obtained in substantially the same manner as in Example 165.

N-{4-[-2-(6-Amino-2-pyridinyl)ethoxy]phenyl}-2-(dimethylamino)benzamide

¹H-NMR(DMSO-d₆):δ 2.76(6H, s), 2.92(2H, t, J=6.7 Hz), 4.24(2H, t, J=6.7 Hz), 5.86(1H, s), 6.30(1H, d, J=8.2 Hz), 6.45(1H, d, J=7.2 Hz), 6.91(2H, d, J=9.2 Hz), 7.07(1H, td, J=7.2 Hz, 1.0 Hz), 7.20(1H, d, J=7.6 Hz), 7.27-7.33(1H, m), 7.42(1H, td, J=7.2 Hz, 1.6 Hz), 7.60-7.68(3H, m), 11.07(1H, s) (+)ESI-MS(m/z): 377(M+H)⁺

### EXAMPLE 170

The following compound was obtained in substantially the same manner as in Example 168.

tert-Butyl 2-(6-[(tert-butoxycarbonyl)amino]-2-pyridinyl}ethyl(4-{[2-(dimethylamino)benzoyl]amino}phenyl)-carbamate

¹H-NMR(CDCl₃):δ 1.41(18H, s), 2.79(6H, s), 3.04(2H, t, J=6.9 Hz), 3.95(2H, t, J=6.9 Hz), 7.06-7.18(4H, m), 7.24-7.30(3H, m), 7.45-7.51(1H, m), 7.58-7.65(3H, m), 8.26(1H, dd, J=7.9 Hz, 1.9 Hz), 12.21(1H, s) (+)ESI-MS(m/z): 576(M+H)⁺

### EXAMPLE 171

The following compound was obtained in substantially the same manner as in Example 165.

N-(4-{[2-(6-Amino-2-pyridinyl)ethyl]amino}phenyl)-2-(dimethylamino)benzamide

¹H-NMR(DMSO-d₆):δ 2.72(2H, t, J=7.3 Hz), 2.76(6H, s), 3.27(2H, t, J=7.3 Hz), 5.55(1H, s), 5.83(2H, s), 6.27(1H, d, J=8.2 Hz), 6.40(1H, d, J=7.2 Hz), 6.58(2H, d, J=8.9 Hz), 7.06(1H, td, J=7.6 Hz, 1.0 Hz), 7.21(1H, d, J=7.2 Hz), 7.28(1H, t, J=7.7 Hz), 7.38-7.45(3H, m), 7.68(1H, dd, J=7.6 Hz, 1.6 Hz), 10.93(1H, s) (+)ESI-MS(m/z): 376(M+H)⁺

### EXAMPLE 172

The following compound was obtained in substantially the same manner as in Example 168.

tert-Butyl 2-{2-[(tert-butoxycarbonyl)amino]-1,3-thiazol-4-yl)ethyl(4-{[2-(dimethylamino)benzoyl]amino}phenyl)carbamate

¹H-NMR(CDCl₃):δ 1.42(9H, s), 1.49(9H, s), 2.83(6H, s), 2.95(2H, t, J=7.7 Hz), 3.91(2H, t, J=7.7 Hz), 6.78(1H, s), 7.14(2H, d, J=8.6 Hz), 7.24-7.32(2H, m), 7.45-7.51(1H, m), 7.63(2H, d, J=8.9 Hz), 8.25(1H, dd, J=7.6 Hz, 1.3 Hz), 12.20(1H, s) (+)ESI-MS(m/z): 582(M+H)⁺

### EXAMPLE 173

The following compound was obtained in substantially the same manner as in Example 165.

N-(4-{[2-(2-Amino-1,3-thiazol-4-yl)ethyl]amino}phenyl)-2-(dimethylamino)benzamide

¹H-NMR(DMSO-d₆):δ 2.66(2H, t, J=7.2 Hz), 2.76(6H, s), 3.23(2H, q, J=7.1 Hz), 5.48(1H, t, J=5.7 Hz), 6.21(1H, s), 6.55(2H, d, J=9.2 Hz), 6.85(2H, s), 7.07(1H, td, J=7.6 Hz, 1.0 Hz), 7.20(1H, dd, J=8.2 Hz, 0.6 Hz), 7.39(1H, d, J=1.6 Hz), 7.43(2H, d, J=8.9 Hz), 7.68(1H, dd, J=7.6 Hz, 1.6 Hz), 10.93(1H, s) (+)ESI-MS(m/z): 382(M+H)⁺

### EXAMPLE 174

The following compound was obtained in substantially the same manner as in Example 168.

tert-Butyl 4-[2-(4-{[2-(dimethylamino)benzoyl]amino}-phenoxy)ethyl]-1,3-thiazol-2-ylcarbamate

¹H-NMR(CDCl₃):δ 1.54(9H, s), 2.82(6H, s), 3.14(2H, t, J=6.5 Hz), 4.25(2H, t, J=6.8 Hz), 6.63(1H, s), 6.91(2H, d, J=8.9 Hz), 7.23-7.30(2H, m), 7.47(1H, td, J=6.8 Hz, 1.6 Hz), 7.58(2H, d, J=8.9 Hz), 8.25(1H, dd, J=7.8 Hz, 1.6 Hz), 8.84(1H, br s), 11.99(1H, s) (+)ESI-MS(m/z): 505(M+Na)⁺

### EXAMPLE 175

To a solution of tert-butyl 4-[2-(4-{[2-(dimethylamino)benzoyl]amino}phenoxy)ethyl]-1,3-thiazol-2-ylcarbamate (260 mg) in dichloromethane (2.6 ml) was added trifluoroacetic acid (0.623 ml). The mixture was stirred for 11 hours, quenched with 10% aqueous potassium carbonate solution, and extracted with dichloromethane. The organic layer was washed with brine, dried over magnesium sulfate, filtered and concentrated in vacuo. The residue was purified by column chromatography on silica gel eluting with hexane: ethyl acetate (2:1 v/v) to give N-{4-[2-(2-amino-1,3-thiazol-4-yl)ethoxy]phenyl}-2-(dimethylamino)benzamide (81 mg) as pale brown powder.

¹H-NMR(CDCl₃):δ 2.82(6H, s), 3.02(2H, t, J=6.8 Hz), 4.25(2H, t, J=7.0 Hz), 6.27(1H, s), 6.92(2H, d, J=8.9 Hz), 7.22-7.30(2H, m), 7.43-7.50(1H, m), 7.57(2H, d, J=8.9 Hz), 8.25(1H, dd, J=7.6, 1.6 Hz), 11.98(1H, s) (+)ESI-MS(m/z): 583(M+H)⁺

Preparation 112

To a solution of 2-(1H-pyrazol-1-yl)ethanol (10 g), triethylamine (18.6 ml) and 4-(dimethylamino)pyridine (1.09 g) in 1,2-dichloroethane (100 ml) was added p-toluenesulfonyl chloride (18.7 g) portionwise at ambient temperature. The reaction mixture was stirred for 14 hours, quenched with water, and extracted with 1,2-dichloroethane. The organic layer was washed with brine, dried over magnesium sulfate, filtered and concentrated in vacuo. The residue was purified by column chromatography on silica gel eluting with hexane: ethyl acetate (1:1 v/v) to give 2-(1H-pyrazol-1-yl)ethyl 4-methylbenzenesulfonate (21.242 g) as a yellow oil.

¹H-NMR(CDCl₃):δ 2.43(1H, s), 4.32-4.41(4H, m), 6.21(1H, t, J=2.0 Hz), 7.28(2H, d, J=8.2 Hz), 7.41(1H, d, J=2.3 Hz), 7.44(1H, d, J=1.3 Hz), (+)ESI-MS(m/z): 267(M+H)⁺

Preparation 113

A mixture of 2-(1H-pyrazol-1-yl)ethyl 4-methylbenzenesulfonate (21.242 g) and sodium azide (10.4 g) in N,N-dimethylformamide (210 ml) was stirred at ambient temperature for 15 hours. The solvent was removed and the residue was dissolved with ethyl acetate and water, and extracted in ethyl acetate. The organic layer was washed with water and brine, dried over magnesium sulfate, filtered and concentrated in vacuo to give 1-(2-azidoethyl)-1H-pyrazole (10.927 g) as a yellow oil. The product was used in the next step without purification.

¹H-NMR(CDCl₃):δ 3.72(2H, t, J=5.6 Hz), 4.27(2H, t, J=5.6 Hz), 6.29(1H, t, J=2.0 Hz), 7.45(1H, d, J=2.0 Hz), 7.57(1H, d, J=1.6 Hz) (+)ESI-MS(m/z): 138(M+H)⁺

Preparation 114

A solution of 1-(2-azidoethyl)-1H-pyrazole (10.927 g) in ethanol (100 ml) was hydrogenated over 10% palladium on carbon (50% wet, 2.185 g) at ambient temperature under atmospheric pressure of hydrogen for an hour. The reaction mixture was filtered with pad of celite, and filtrate was concentrated in vacuo to give 2-(1H-pyrazol-1-yl)ethylamine (8.169 g) as a yellow oil. The product was used in the next step without purification.

¹H-NMR(CDCl₃):δ 3.15(2H, t, J=5.8 Hz), 4.18(2H, t, J=5.8 Hz), 6.26(1H, t, J=2.0 Hz), 7.43(1H, d, J=2.3 Hz), 7.53(1H, d, J=1.6 Hz) (+)ESI-MS(m/z): 112(M+H)⁺

Preparation 115

A mixture of 2-(1H-pyrazol-1-yl)ethylamine (8.169 g), 1-fluoro-4-nitrobenzene (12.4 g) and triethylamine (11.2 g) in 2,6-dimethyl-2-imidazolidinone (100 ml) was heated at 60° C. for 18 hours. The reaction mixture was cooled to ambient temperature, poured into water and extracted with ethyl acetate. The organic layer was washed with water and brine, dried over magnesium sulfate, filtered and concentrated in vacuo. The residue was purified by column chromatography on silica gel eluting with hexane: ethyl acetate (4:6→1:9 v/v) to give N-(4-nitrophenyl)-N-[2-(1H-pyrazol-1-yl)ethyl]amine (7.508 g) as a yellow solid.

¹H-NMR(CDCl₃):δ 3.63-3.69(2H, m), 4.37-4.41(2H, m), 5.23(1H, s), 6.27(1H, t, J=2.1 Hz), 6.51(2H, d, J=9.2 Hz), 7.38(1H, dd, J=2.3 Hz, 0.7 Hz), 7.56(1H, dd, J=2.0 Hz, 0.7 Hz), 8.05(2H, d, J=9.2 Hz) (+)ESI-MS(m/z): 255(M+Na)⁺

Preparation 116

To a solution of N-(4-nitrophenyl)-N-[2-(1H-pyrazol-1-yl)ethyl]amine (5.012 g) and 4-(dimethylamino)pyridine (264 mg) in tetrahydrofuran (100 ml) was added di-tert-butyl dicarbonate (7.07 g) and heated at 50° C. for 1 hour. The reaction mixture was cooled to ambient temperature and concentrated in vacuo. The residue was dissolved in ethyl acetate and water, and extracted with ethyl acetate. The organic layer was washed with brine, dried over magnesium sulfate, filtered and concentrated in vacuo. The residue was purified by column chromatography on silica gel eluting with hexane: ethyl acetate (2:1→1:1 v/v) to give tert-butyl 4-nitrophenyl[2-(1H-pyrazol-1-yl)ethyl]carbamate (7.051 g) as a yellow solid.

¹H-NMR(CDCl₃):δ 1.46(9H, s), 4.11(2H, t, J=5.7 Hz), 4.41(2H, t, J=5.7 Hz), 6.21(1H, t, J=2.0 Hz), 7.03(2H, d, J=9.2 Hz), 7.32(1H, d, J=2.3 Hz), 7.45(1H, d, J=2.0 Hz), 8.08(2H, d, J=9.2 Hz) (+)ESI-MS(m/z): 355(M+Na)⁺

Preparation 117

A solution of tert-butyl 4-nitrophenyl[2-(1H-pyrazol-1-yl)ethyl]carbamate (400 mg) in methanol (5 ml) was hydrogenated over 10% palladium on carbon at ambient temperature under atmospheric pressure of hydrogen for an hour. The reaction mixture was filtered with pad of Celite, and filtrate was concentrated in vacuo to give tert-butyl 4-aminophenyl[2-(1H-pyrazol-1-yl)ethyl]carbamate (363 mg) as a yellow oil. The product was used in the next step without purification.

¹H-NMR(CDCl₃):δ 1.38(9H, br s), 3.62(2H, br s), 3.96(2H, t, J=6.2 Hz), 4.32(2H, br s), 6.23(1H, t, J=2.0 Hz), 6.57(2H, d, J=8.2 Hz), 6.72(2H, br s), 7.38(1H, br s), 7.48(1H, d, J=1.6 Hz) (+)ESI-MS(m/z): 525(M+Na)⁺

### EXAMPLE 176

To a solution of 4-methyl-2-(4-methyl-1-piperidinyl)benzoic acid (314 mg), tert-butyl 4-aminophenyl[2-(1H-pyrazol-1-yl)ethyl]carbamate (371 mg) and 1-hydroxybenzotriazole (244 mg) in N,N-dimethylformamide (10 ml) was added 1-[3-(dimethylamino)propyl]-3-ethylcarbodiimide hydrochloride (WSC.HCl) (306 mg), followed by triethylamine (162 mg) at ambient temperature and the mixture was stirred at 50° C. for 16 hours. The reaction mixture was poured into a mixture of ethyl acetate and water. The separated organic layer was washed with brine, dried over magnesium sulfate and concentrated in vacuo. The residue was purified by column chromatography on silica gel eluting with hexane: ethyl acetate (1:1 v/v) to give tert-butyl 4-{[4-methyl-2-(4-methyl-1-piperidinyl)benzoyl]amino}phenyl[2-(1H-pyrazol-1-yl)ethyl]carbamate (303 mg) as a greenish yellow oil.

¹H-NMR(CDCl₃):δ 1.06(3H, d, J=6.3 Hz), 1.31-1.65(12H, m), 1.86(2H, brd, J=11.5 Hz), 2.39(3H, s), 2.84(2H, t, J=8.6 Hz), 3.17(2H, brd, J=11.5 Hz), 4.04(2H, t, J=6.3 Hz), 4.36(2H, brs), 6.24(1H, t, J=2.0 Hz), 6.95(1H, brs), 7.09(2H, brs), 7.39(1H, s), 7.48(1H, s), 7.67(2H, d, J=8.6 Hz), 8.17(1H, d, J=8.3 Hz), 12.60(1H, s) (+)ESI-MS(m/z): 540(M+Na)⁺

### EXAMPLE 177

To a solution of tert-butyl 4-{[4-methyl-2-(4-methyl-1-piperidinyl)benzoyl]amino}phenyl[2-(1H-pyrazol-1-yl)ethyl]carbamate (297 mg) in dichloromethane (10 ml) was added trifluoroacetic acid (981 mg). The reaction mixture was stirred at ambient temperature for 14 hours, quenched with 10% aqueous potassium carbonate aqueous solution, and extracted with dichloromethane. The organic layer was washed with brine, dried over magnesium sulfate, filtered and concentrated in vacuo. The residue was recrystallized from ethyl acetate-diisopropyl ether to give 4-methyl-2-(4-methyl-1-piperidinyl)-N-(4-{[2-(1H-pyrazol-1-yl)ethyl]amino)phenyl)benzamide (177 mg) as a faintly brown powder.

¹H-NMR(CDCl₃):δ 1.03(3H, d, J=6.3 Hz), 1.40-1.60(3H, m), 2.38(3H, s), 2.81(2H, t, J=11.5 Hz), 2.91(2H, t, J=6.6 Hz), 3.17(2H, d, J=11.9 Hz), 3.47(2H, t, J=6.6 Hz), 4.45(2H, brs), 6.36(1H, d, J=8.3 Hz), 6.53(1H, d, J=7.3 Hz), 6.65(2H, d, J=8.9 Hz), 7.04-7.08(2H, m), 7.36(1H, t, J=7.3 Hz), 7.57(2H, d, J=8.9 Hz), 8.17(1H, d, J=8.6 Hz), 12.24(1H, s) (+)ESI-MS(m/z): 444(M+H)⁺

### EXAMPLE 178

The following compound was obtained in substantially the same manner as in Example 176.

tert-Butyl 4-{[2-(dimethylamino)benzoyl]amino)phenyl[2-(1H-pyrazol-1-yl)ethyl]carbamate

¹H-NMR(CDCl₃):δ 1.53(9H, s), 2.39(3H, s), 2.80(6H, s), 2.96(2H, t, J=6.6 Hz), 3.49(2H, t, J=6.6 Hz), 6.64(2H, d, J=8.9 Hz), 6.83(1H, d, J=7.3 Hz), 7.04-7.08(2H, m), 7.21(1H, brs), 7.49(2H, d, J=8.6 Hz), 7.58(1H, t, J=8.6 Hz), 7.77(1H, d, J=8.3 Hz), 8.14(1H, d, J=8.6 Hz), 11.86(1H, s) (+)ESI-MS(m/z): 512(M+Na)⁺

### EXAMPLE 179

The following compound was obtained in substantially the same manner as in Example 177.

2-(Dimethylamino)-N-(4-{[2-(1H-pyrazol-1-yl)ethyl]amino}phenyl)benzamide

¹H-NMR(CDCl₃):δ 2.39(3H, s), 2.80(6H, s), 2.90(2H, t, J=6.6 Hz), 3.47(2H, t, J=6.6 Hz), 4.46(2H, brs), 6.36(1H, d, J=7.9 Hz), 6.53(1H, d, J=7.3 Hz), 6.64(2H, d, J=8.9 Hz), 7.04-7.07(2H, m), 7.36(1H, t, J=7.3 Hz), 7.48(2H, d, J=8.9 Hz), 8.14(1H, d, J=8.6 Hz), 11.84(1H, s) (+)ESI-MS(m/z): 390(M+H)⁺

### EXAMPLE 180

The following compound was obtained in substantially the same manner as in Example 176.

tert-Butyl 4-({[6-methyl-2-(4-methyl-1-piperidinyl)-3-pyridinyl]carbonyl}amino)phenyl[2-(1H-pyrazol-1-yl)ethyl]carbamate

¹H-NMR(CDCl₃):δ 1.04(3H, d, J=6.6 Hz), 1.31-1.52(2H, m), 1.41(9H, s), 1.52-1.70(1H, m), 1.85(2H, brd, J=10.6 Hz), 2.52(3H, s), 3.00(2H, t, J=10.2 Hz), 3.33(2H, brd, J=12.5 Hz), 4.04(2H, t, J=6.3 Hz), 4.37(2H, t, J=6.3 Hz), 6.24(1H, t, J=2.0 Hz), 6.96(1H, brs), 7.02(2H, d, J=7.9 Hz), 7.39(1H, d, J=2.0 Hz), 7.48(1H, d, J=2.0 Hz), 7.64(2H, d, J=8.9 Hz), 8.35(1H, d, J=7.9 Hz), 11.85(1H, s) (+)ESI-MS(m/z): 541(M+Na)⁺

### EXAMPLE 181

The following compound was obtained in substantially the same manner as in Example 177.

6-Methyl-2-(4-methyl-1-piperidinyl)-N-(4-{[2-(1H-pyrazol-1-yl)ethyl]amino}phenyl)nicotinamide

¹H-NMR(CDCl₃):δ 1.02(3H, d, J=6.3 Hz), 1.30-1.50(2H, m), 1.50-1.68(1H, m), 1.83(2H, brd, J=12.9 Hz), 2.51(3H, s), 2.98(2H, dt, J=2.3 Hz, 12.2 Hz), 3.34(2H, brd, J=12.5 Hz), 3.60(2H, brs), 3.99(1H, brs), 4.33-4.37(2H, m), 6.25(1H, t, J=2.0 Hz), 6.62(2H, d, J=8.9 Hz), 6.99(1H, d, J=7.9 Hz), 7.36(1H, d, J=2.0 Hz), 7.50-7.62(3H, m), 8.34(1H, d, J=7.9 Hz), 11.50(1H, s) (+)ESI-MS(m/z): 419(M+H)⁺, 441(M+Na)⁺

### EXAMPLE 182

To a solution of tert-butyl 4-aminophenyl[2-(1H-pyrazol-1-yl)ethyl]carbamate (363 mg), 2-(dimethylamino)-4-methylbenzoic acid (237 mg) and 1-hydroxybenzotriazole (221 mg) in N,N-dimethylformamide (7 ml) was added 1-[3-(dimethylamino)propyl]-3-ethylcarbodiimide hydrochloride (276 mg) at ambient temperature. The reaction mixture was stirred at 50° C. for 19 hours and concentrated in vacuo. The residue was dissolved in ethyl acetate and water, and extracted with ethyl acetate. The organic layer was washed with water and brine, dried over magnesium sulfate, filtered and concentrated in vacuo. The residue was purified by column chromatography on silica gel eluting with hexane: ethyl acetate (1:1 v/v) to give tert-butyl 4-{[2-(dimethylamino)-4-methylbenzoyl]amino}phenyl[2-(1H-pyrazol-1-yl)ethyl]carbamate (348 mg) as a yellow foam.

¹H-NMR(CDCl₃):δ 1.40(9H, s), 2.39(3H, s), 2.80(6H, s), 4.03(2H, t, J=6.1 Hz), 4.35(2H, t, J=6.1 Hz), 6.24(1H, t, J=2.0 Hz), 7.06-7.09(2H, m), 7.39(1H, d, J=2.0 Hz), 7.49(1H, d, J=1.4 Hz), 7.58(2H, d, J=8.9 Hz), 8.14(1H, d, J=8.6 Hz), 12.26(1H, s) (+)ESI-MS(m/z): 486(M+Na)⁺

### EXAMPLE 183

To a solution of tert-butyl 4-{[2-(dimethylamino)-4-methylbenzoyl]amino}phenyl[2-(1H-pyrazol-1-yl)ethyl]carbamate (345 mg) in dichloromethane (10 ml) was added trifluoroacetic acid (0.86 ml). The reaction mixture was stirred at ambient temperature for 19 hours, quenched with 10% aqueous potassium carbonate solution, and extracted with dichloromethane. The organic layer was washed with brine, dried over magnesium sulfate, filtered and concentrated in vacuo. The residue was recrystallized from ethyl acetate-diisopropyl ether to give 2-(dimethylamino)-4-methyl-N-(4-{[2-(1H-pyrazol-1-yl)ethyl]amino}phenyl)benzamide (215 mg) as a white solid.

¹H-NMR(DMSO-d₆):δ 2.33(3H, s), 2.74(6H, s), 3.42(2H, br s), 4.26(2H, t, J=6.2 Hz), 5.57(1H, br s), 6.22(1H, t, J=2.0 Hz), 6.57(2H, d, J=8.9 Hz), 6.93(1H, d, J=7.9 Hz), 7.07(1H, s), 7.43(2H, d, J=8.9 Hz), 7.46(1H, d, J=1.6 Hz), 7.66(1H, d, J=7.6 Hz), 7.72(1H, d, J=2.0 Hz), 11.17(1H, s) (+)ESI-MS(m/z): 364(M+H)⁺

Preparation 118

The mixture of 2-(1H-pyrazol-1-yl)ethanamine (2.13 g), 2-chloro-5-nitropyridine (3.65 g) and triethylamine (4.01 ml) in dimethylformamide (11 ml) was heated at 50° C. for 12 hours. The reaction mixture was concentrated in vacuo. To the residue was added water and the mixture was extracted with ethyl acetate. The organic layer was washed with brine, dried over magnesium sulfate, filtered and concentrated in vacuo. The residue was recrystallized from ethyl acetate-hexane to give 5-nitro-N-[2-(1H-pyrazol-1-yl)ethyl]-2-pyridinamine (4.39 g) as pale yellow powder.

¹H-NMR(DMSO-d₆):δ 3.95(2H, q, J=5.4 Hz), 4.39(2H, t, J=5.7 Hz), 5.94(1H, br s), 6.27(1H, t, J=2.4 Hz), 6.36(1H, d, J=9.2 Hz), 7.34(1H, d, J=2.2 Hz), 7.56(1H, d, J=1.4 Hz), 8.14(1H, dd, J=9.2 Hz, 2.7 Hz), 9.02(1H, d, J=2.7 Hz) (+)ESI-MS(m/z): 234(M+H)⁺

Preparation 119

To a solution of 5-nitro-N-[2-(1H-pyrazol-1-yl)ethyl]-2-pyridinamine (4.39 g) in tetrahydrofuran (35 ml) was added di-t-butyl dicarbonate (6.16 g). The mixture was stirred at ambient temperature for 15 hours. The reaction mixture was concentrated in vacuo. The residue was dissolved in ethyl acetate and water, and extracted with ethyl acetate. The organic layer was washed with water and brine, dried over magnesium sulfate, filtered and concentrated in vacuo. The residue was recrystallized from ethyl acetate-hexane to give tert-butyl 5-nitro-2-pyridinyl[2-(1H-pyrazol-1-yl)ethyl]carbamate (6.23 g) as a pale yellow powder.

¹H-NMR(CDCl₃):δ 1.50(9H, s), 4.42-4.55(4H, m), 6.19(1H, t, J=1.9 Hz), 7.30(1H, d, J=2.4 Hz), 7.44(1H, d, J=1.4 Hz), 8.05(1H, d, J=9.5 Hz), 8.35(1H, dd, J=5.9 Hz, 2.7 Hz), 9.16(1H, d, J=3.2 Hz) (+)ESI-MS(m/z): 356(M+Na)⁺

Preparation 120

A solution of tert-butyl 5-nitro-2-pyridinyl[2-(1H-pyrazol-1-yl)ethyl]carbamate (1.0 g) in methanol (10 ml) was hydrogenated over 10% palladium on carbon (0.2 g, 50% wet) at ambient temperature under atmospheric pressure of hydrogen for an hour. The reaction mixture was filtered through a short pad of celite, and the filtrate was concentrated in vacuo to give tert-butyl 5-amino-2-pyridinyl[2-(1H-pyrazol-1-yl)ethyl]carbamate (0.9 g) as a pale yellow oil.

¹H-NMR(CDCl₃):δ 1.42(9H, s), 3.65(2H, br s), 4.21(2H, t, J=5.7 Hz), 4.38(2H, t, J=5.7 Hz), 6.19(1H, t, J=1.9 Hz), 6.93(1H, dd, J=8.6 Hz, 3.0 Hz), 7.07(1H, br d, J=6.8 Hz), 7.37(1H, dd, J=2.4 Hz, 0.8 Hz), 7.44(1H, dd, J=2.2 Hz, 0.8 Hz), 7.84(1H, dd, J=3.0 Hz, 0.5 Hz) (+)ESI-MS(m/z): 326(M+Na)⁺

### EXAMPLE 184

To a solution of tert-butyl 5-amino-2-pyridinyl[2-(1H-pyrazol-1-yl)ethyl]carbamate (343 mg), 4-methyl-2-(4-methyl-1-piperidinyl)benzoic acid (317 mg) and 1-hydroxybenzotriazole (208 mg) in N,N-dimethylformamide (3 ml) was added 1-[3-(dimethylamino)propyl]-3-ethylcarbodiimide hydrochloride (WSC.HCl) (260 mg), followed by triethylamine (0.24 ml) at ambient temperature. The reaction mixture was stirred at ambient temperature for 13 hours and concentrated in vacuo. The residue was dissolved in ethyl acetate and water, and extracted with ethyl acetate. The organic layer was washed with water and brine, dried over magnesium sulfate, filtered and concentrated in vacuo. The residue was purified by column chromatography on silica gel eluting with hexane: ethyl acetate (6:1→4:1→1:1 v/v) to give tert-butyl 5-{[4-methyl-2-(4-methyl-1-piperidinyl)benzoyl]amino}-2-pyridinyl[2-(1H-pyrazol-1-yl)ethyl]carbamate (0.274 g) as a pale yellow foam.

¹H-NMR(CDCl₃):δ 1.08(3H, d, J=6.5 Hz), 1.39-1.53(11H, m), 1.48-1.69(1H, m), 1.89(2H, br d, J=12.7 Hz), 2.40(3H, s), 2.86(2H, td, J=11.6 Hz, 2.4 Hz), 3.18(2H, br d, J=11.9 Hz), 4.34(2H, t, J=5.4 Hz), 4.44(2H, t, J=5.1 Hz), 6.20(1H, t, J=2.2 Hz), 7.09-7.13(1H, br d, J=8.4 Hz), 7.13(1H, s), 7.37(1H, dd, J=2.2 Hz, 0.5 Hz), 7.42-7.46(2H, m), 8.19(1H, d, J=7.8 Hz), 8.30(1H, dd, J=8.9 Hz, 3.0 Hz), 8.56(1H, d, J=2.7 Hz), 12.90(1H, s) (+)ESI-MS(m/z): 519(M+H)⁺

### EXAMPLE 185

To a solution of tert-butyl 5-{[4-methyl-2-(4-methyl-1-piperidinyl)benzoyl]amino)-2-pyridinyl[2-(1H-pyrazol-1-yl)ethyl]carbamate (235.7 mg) in dichloromethane (2.4 ml) was added trifluoroacetic acid (0.525 ml). The mixture was stirred for 60 hours, quenched with 10% aqueous potassium carbonate solution, and extracted with dichloromethane. The organic layer was washed with brine, dried over magnesium sulfate, filtered and concentrated in vacuo. The residue was purified by column chromatography on silica gel eluting with hexane: ethyl acetate (6:1→4:1→1:1 v/v) to give 4-methyl-2-(4-methyl-1-piperidinyl)-N-(6-{[2-(1H-pyrazol-1-yl)ethyl]amino}-3-pyridinyl)benzamide (120 mg) as a pale brown powder.

¹H-NMR(CDCl₃):δ 1.05(3H, d, J=6.2 Hz), 1.44(2H, qd, J=12.7 Hz, 3.5 Hz), 1.54-1.63(1H, m), 1.86(2H, br d, J=13.5 Hz), 2.39(3H, s), 2.83(2H, td, J=11.9 Hz, 2.2 Hz), 3.17(2H, d, J=12.2 Hz), 3.81(2H, q, J=5.9 Hz), 4.38(2H, t, J=5.1 Hz), 4.67(1H, t, J=5.9 Hz), 6.24(1H, t, J=1.9 Hz), 6.41(1H, d, J=8.9 Hz), 7.08(1H, d, J=6.8 Hz), 7.09(1H, s), 7.36(1H, d, J=2.4 Hz), 7.55(1H, d, J=1.1 Hz), 8.11-8.24(3H, m), 12.45(1H, s) (+)ESI-MS(m/z): 419(M+H)⁺

### EXAMPLE 186

To a solution of 2-(2-pyridinylacetyl)-5-isoindolinamine (895 mg), 6-methyl-2-(4-methyl-1-piperidinyl)nicotinic acid (828 mg) and benzotriazol-1-yl-oxytripyrrolidinophosphonium hexafluorophosphate (PyBOP) (2.21 g) in N,N-dimethylformamide (30 ml) was added diisopropylethylamine (913 mg) at ambient temperature and the mixture was stirred at the same temperature for 20 hours. The mixture was poured into a mixture of ethyl acetate, water and 6N hydrochloric acid, and the separated organic layer was washed with water and brine, dried over magnesium sulfate and evaporated in vacuo. The residue was purified by column chromatography on silica gel eluting with ethyl acetate to give 6-methyl-2-(4-methyl-1-piperidinyl)-N-[2-(2-pyridinylacetyl)-2,3-dihydro-1H-isoindol-5-yl]nicotinamide (815 mg) as white crystals.

¹H-NMR(DMSO-d₆):δ 0.89(3H, d, J=6.1 Hz), 1.1-1.4(3H, m), 1.6-1.8(2H, m), 2.39(3H, s), 2.75-2.95(2H, m), 3.4-3.8(6H, m), 6.8-7.5(6H, m), 7.65-7.8(2H, m), 8.51(1H, d, J=4.1 Hz), 10.46(1H, s) (+)ESI-MS(m/z): 492(M+Na)⁺

### EXAMPLE 187

The following compound was obtained in substantially the same manner as in Example 186.

4-Methyl-2-(4-methyl-1-piperidinyl)-N-[2-(2-pyridinylacetyl)-2,3-dihydro-1H-isoindol-5-yl]benzamide

¹H-NMR(DMSO-d₆):δ 0.96(3H, d, J=6.0 Hz), 1.2-1.45(3H, m), 1.7-1.9(2H, m), 2.34(3H, s), 2.7-2.9(2H, m), 3.05-3.2(2H, m), 3.4-3.8(6H, m), 7.0-7.5(6H, m), 7.65-7.85(3H, m), 8.5-8.55(1H, m), 11.90(1H, s) (+)ESI-MS(m/z): 469(M+H)⁺, 491(M+Na)⁺

Preparation 121

To a solution of 2-(phenylacetyl)-5-isoindolinamine (1.008 g), 2-chloro-6-methylnicotinic acid (754 mg) and benzotriazol-1-yl-oxytripyrrolidinophosphonium hexafluorophosphate (PyBOP) (2.70 g) in N,N-dimethylformamide (30 ml) was added diisopropylethylamine (1.03 g) at ambient temperature and the mixture was stirred at the same temperature for 20 hours. The mixture was poured into a mixture of ethyl acetate, water and 6N hydrochloric acid, and the separated organic layer was washed with water and brine, dried over magnesium sulfate and evaporated in vacuo. The residue was purified by column chromatography on silica gel eluting with hexane: ethyl acetate (1:1 v/v) to give 2-chloro-6-methyl-N-[2-(phenylacetyl)-2,3-dihydro-1H-isoindol-5-yl]nicotinamide (1.19 g) as a pale brown powder.

¹H-NMR(DMSO-d₆):δ 2.53(3H, s), 3.71(2H, s), 4.6-5.0(4H, m), 6.45-6.55(2H, m), 6.9-7.0(1H, m), 7.2-7.5(7H, m), 10.62(1H, s) (+)ESI-MS(m/z): 406(M+H)⁺

### EXAMPLE 188

To a solution of 2-chloro-6-methyl-N-[2-(phenylacetyl)-2,3-dihydro-1H-isoindol-5-yl]nicotinamide (1.18 g) in acetonitrile (15 ml) was added 4-methylpiperidine (865 mg) and the mixture was refluxed for 16 hours. The mixture was evaporated in vacuo and the residue was purified by column chromatography on silica gel eluting with hexane: ethyl acetate (2:1 v/v) to give 6-methyl-2-(4-methyl-1-piperidinyl)-N-[2-(phenylacetyl)-2,3-dihydro-1H-isoindol-5-yl]nicotinamide (440 mg) as white crystals.

¹H-NMR(DMSO-d₆):δ 0.88(3H, d, J=6.2 Hz), 1.1-1.7(5H, m), 2.39(3H, s), 2.7-2.9(2H, m), 3.55-3.7(2H, m), 4.64(2H, d, J=8.5 Hz), 4.89(2H, d, J=8.5 Hz), 6.82(1H, d, J=7.6 Hz), 7.2-7.4(6H, m), 7.5-7.6(1H, m), 7.7-7.9(3H, m), 10.56(1H, s) (−)ESI-MS(m/z): 467(M−H)⁻

Preparation 122

To a solution of N-(4-aminophenyl)-2-[6-(2,5-dimethyl-1H-pyrrol-1-yl)-2-pyridinyl]acetamide (3.50 g), 2-chloro-6-methylnicotinic acid (1.87 g) and benzotriazol-1-yl-oxytripyrrolidinophosphonium hexafluorophosphate (PyBOP) (6.82 g) in N,N-dimethylformamide (50 ml) was added diisopropylethylamine (4.24 g) at ambient temperature and the mixture was stirred at the same temperature for 24 hours. The mixture was poured into a mixture of ethyl acetate, water and 6N hydrochloric acid, and the separated organic layer was washed with water and brine, dried over magnesium sulfate and evaporated in vacuo. The residue was purified by column chromatography on silica gel eluting with ethyl acetate to give 2-chloro-N-[4-({[6-(2,5-dimethyl-1H-pyrrol-1-yl)-2-pyridinyl]acetyl}amino)phenyl]-6-methylnicotinamide (4.15 g) as a brown powder.

¹H-NMR(DMSO-d₆):δ 1.99(3H, s), 2.04(6H, s), 3.87(2H, s), 5.78(2H, s), 7.29(1H, d, J=7.6 Hz), 7.38(1H, dd, J=7.6 Hz, 6.5 Hz), 7.5-7.7(4H, m), 7.9-8.0(2H, m), 10.25(1H, s), 10.49(1H, s) (+)ESI-MS(m/z): 474(M+H)⁺, 496(M+Na)⁺

### EXAMPLE 189

To a solution of 2-chloro-N-[4-({[6-(2,5-dimethyl-1H-pyrrol-1-yl)-2-pyridinyl]acetyl}amino)phenyl]-6-methylnicotinamide (1.12 g) in acetonitrile (30 ml) was added 4-methylpiperidine (703 mg) and the mixture was refluxed for 20 hours. The mixture was evaporated in vacuo and the residue was purified by column chromatography on silica gel eluting with ethyl acetate to give N-[4-({[6-(2,5-dimethyl-1H-pyrrol-1-yl)-2-pyridinyl]acetyl}amino)phenyl]-6-methyl-2-(4-methyl-1-piperidinyl)nicotinamide (975 mg) as a brown powder.

¹H-NMR(DMSO-d₆):δ 0.88(3H, d, J=6.2 Hz), 1.15-1.75(5H, m), 2.04(6H, s), 2.7-2.95(2H, m), 3.55-3.7(2H, m), 3.87(2H, s), 5.77(2H, s), 6.82(1H, d, J=7.7 Hz), 7.29(1H, d, J=7.8 Hz), 7.44(1H, d, J=7.5 Hz), 7.55(1H, d, J=9.0 Hz), 7.64(1H, d, J=9.0 Hz), 7.73(1H, d, J=7.5 Hz), 7.95(1H, dd, J=7.8 Hz, 7.7 Hz), 10.22(1H, s), 10.48(1H, s) (+)ESI-MS(m/z): 537(M+H)⁺, 559(M+Na)⁺

### EXAMPLE 190

To a suspension of N-[4-({[6-(2,5-dimethyl-1H-pyrrol-1-yl)-2-pyridinyl]acetyl}amino)phenyl]-6-methyl-2-(4-methyl-1-piperidinyl)nicotinamide (950 mg) in a mixture of ethanol (40 ml) and water (10 ml) were added hydroxylamine hydrochloride (1.23 g) and triethylamine (358 mg) at ambient temperature. The mixture was refluxed for 6 hours and evaporated to dryness. The residue was extracted from ethyl acetate and the organic layer was washed with brine, dried over magnesium sulfate and evaporated in vacuo. The residue was purified by column chromatography on silica gel eluting with ethyl acetate to give N-(4-{[(6-amino-2-pyridinyl)acetyl]amino}phenyl)-6-methyl-2-(4-methyl-1-piperidinyl)nicotinamide (458 mg) as white crystals.

¹H-NMR(DMSO-d₆):δ 0.89(3H, d, J=6.2 Hz), 1.1-1.75(4H, m), 2.3-2.4(1H, m), 2.39(3H, s), 2.7-2.9(2H, m), 3.55(2H, s), 4.55-4.75(2H, m), 5.91(2H, brs), 6.31(1H, d, J=8.0 Hz), 6.47(1H, d, J=7.1 Hz), 6.82(1H, d, J=7.6 Hz), 7.32(1H, dd, J=8.0 Hz, 7.1 Hz), 7.55-7.7(4H, m), 7.75(1H, d, J=7.6 Hz), 10.19(1H, s), 10.48(1H, s) (+)ESI-MS(m/z): 459(M+H)⁺, 481(M+Na)⁺

Preparation 123

To a 20% solution of sodium ethoxide (108 ml) was added dropwise 2-hydrazinoethanol (80%v/v aqueous solution) (31.8 ml) at 5° C., followed by addition of a solution of 2-chloroacetonitrile (27.40 g) in ethanol (100 ml). The mixture was refluxed for 18 hours and cooled to ambient temperature and the residue was purified by column chromatography on silica gel eluting with dichloromethane:methanol (5:1 v/v) to give 2-(3-amino-1H-pyrazol-1-yl)ethanol (8.94 g) as a dark brown oil.

¹H-NMR(DMSO-d₆):δ 3.62(2H, td, J=6.0 Hz, 5.4 Hz), 3.84(2H, t, J=6.0 Hz), 4.46(2H, brs), 4.77(1H, t, J=5.4 Hz), 5.34(1H, d, J=2.2 Hz), 7.26(1H, d, J=2.2 Hz) (+)APCI-MS(m/z): 128(M+H)⁺

Preparation 124

To a solution of 2-(3-amino-1H-pyrazol-1-yl)ethanol (8.90 g) in toluene (200 ml) were added 2,5-hexanedione (9.59 g) and p-toluenesulfonic acid hydrate (1.33 g) at ambient temperature and the mixture was refluxed for 20 hours. The mixture was concentrated to ca. 50 ml and purified by column chromatography on silica gel eluting with ethyl acetate to give 2-[3-(2,5-dimethyl-1H-pyrrol-1-yl)-1H-pyrazol-1-yl]ethanol (7.77 g) as a yellow oil.

¹H-NMR(DMSO-d₆):δ 2.02(6H, s), 3.74(2H, td, J=6.1 Hz, 5.2 Hz), 4.14(2H, t, J=6.1 Hz), 4.92(1H, t, J=5.2 Hz), 5.74(2H, s), 6.24(1H, d, J=2.2 Hz), 7.79(1H, d, J=2.2 Hz) (+)ESI-MS(m/z): 206(M+H)⁺, 228(M+Na)⁺

Preparation 125

To a solution of potassium tert-butoxide (2.25 g) in tetrahydrofuran (60 ml) was added dropwise a solution of 2-[3-(2,5-dimethyl-1H-pyrrol-1-yl)-1H-pyrazol-1-yl]ethanol (4.11 g) in tetrahydrofuran (40 ml) at ambient temperature, followed by addition of 4-fluronitrobenzene (2.83 g). The mixture was refluxed for 6 hours under nitrogen and poured into a mixture of ethyl acetate and ice-water. The separated organic layer was washed with brine, dried over magnesium sulfate and evaporated in vacuo. The residue was purified by column chromatography on silica gel eluting with hexane: ethyl acetate (2:1 v/v) to give 3-(2,5-dimethyl-1H-pyrrol-1-yl)-1-[2-(4-nitrophenoxy)ethyl]-1H-pyrazole (3.34 g) as a pale brown powder.

¹H-NMR(DMSO-d₆):δ 1.96(6H, s), 4.55(4H, s), 5.73(2H, s), 6.29(1H, d, J=2.4 Hz), 7.1-7.2(2H, m), 7.92(1H, d, J=2.4 Hz), 8.15-8.25(2H, m) (+)ESI-MS(m/z): 327(M+H)⁺

Preparation 126

To a solution of 3-(2,5-dimethyl-1H-pyrrol-1-yl)-1-[2-(4-nitrophenoxy)ethyl]-1H-pyrazole (3.31 g) in tetrahydrofuran (40 ml) and methanol (40 ml) was added 5% palladium on carbon (1 g, 50% wet) and the mixture was hydrogenated for 4 hours at ambient temperature. The catalyst was removed by filtration and the filtrate was evaporated in vacuo. The residue was purified by column chromatography on silica gel eluting with hexane: ethyl acetate (1:2 v/v) to give 4-{2-[3-(2,5-dimethyl-1H-pyrrol-1-yl)-1H-pyrazol-1-yl]ethoxy}aniline (2.46 g) as a pale brown powder.

¹H-NMR(DMSO-d₆):δ 2.00(6H, s), 4.20(2H, t, J=5.6 Hz), 4.41(2H, t, J=5.6 Hz), 4.63(2H, brs), 5.74(2H, s), 6.28(1H, d, J=2.4 Hz), 6.4-6.5(2H, m), 6.55-6.65(2H, m), 7.87(1H, d, J=2.4 Hz) (+)ESI-MS(m/z): 297(M+H)⁺

### EXAMPLE 191

To a solution of 4-{2-[3-(2,5-dimethyl-1H-pyrrol-1-yl)-1H-pyrazol-1-yl]ethoxy}aniline (1.30 g), 6-methyl-2-(4-methyl-1-piperidinyl)nicotinic acid (1.03 g) and benzotriazol-1-yl-oxytripyrrolidinophosphonium hexafluorophosphate (PyBOP) (2.74 g) in N,N-dimethylformamide (50 ml) was added diisopropylethylamine (1.73 g) at ambient temperature and the mixture was stirred at the same temperature for 24 hours. The mixture was poured into a mixture of ethyl acetate, water and 6N hydrochloric acid, and the separated organic layer was washed with water and brine, dried over magnesium sulfate and evaporated in vacuo. The residue was purified by column chromatography on silica gel eluting with ethyl acetate to give N-(4-{2-[3-(2,5-dimethyl-1H-pyrrol-1-yl)-1H-pyrazol-1-yl]ethoxy}phenyl)-6-methyl-2-(4-methyl-1-piperidinyl)nicotinamide (1.40 g) as a brown powder.

¹H-NMR(DMSO-d₆):δ 0.88(3H, t, J=6.1 Hz), 1.1-1.3(2H, m), 1.4-1.75(3H, m), 1.99(6H, s), 2.39(3H, s), 2.7-2.9(2H, m), 3.55-3.7(2H, m), 4.35(2H, t, J=4.9 Hz), 4.49(2H, t, J=4.9 Hz), 5.73(2H, s), 6.28(1H, d, J=2.4 Hz), 6.81(1H, d, J=7.6 Hz), 6.88(2H, d, J=9.0 Hz), 7.60(2H, d, J=9.0 Hz), 7.73(1H, d, J=7.6 Hz), 7.90(1H, d, J=2.4 Hz), 10.41(1H, s) (+)ESI-MS(m/z): 513(M+H)⁺, 535(M+Na)⁺

### EXAMPLE 192

To a suspension of N-(4-{2-[3-(2,5-dimethyl-1H-pyrrol-1-yl)-1H-pyrazol-1-yl]ethoxy}phenyl)-6-methyl-2-(4-methyl-1-piperidinyl)nicotinamide (1.39 g) in a mixture of ethanol (40 ml) and water (10 ml) were added hydroxylamine hydrochloride (1.89 g) and triethylamine (549 mg) at ambient temperature. The mixture was refluxed for 6 hours and evaporated to dryness. The residue was extracted from ethyl acetate and the organic layer was washed with brine, dried over magnesium sulfate and evaporated in vacuo. The residue was purified by column chromatography on silica gel eluting with ethyl acetate methanol (10:1 v/v) to give N-{4-[2-(3-amino-1H-pyrazol-1-yl)ethoxy]phenyl)-6-methyl-2-(4-methyl-1-piperidinyl)nicotinamide (462 mg) as white crystals.

¹H-NMR(DMSO-d₆):δ 0.88(3H, d, J=6.0 Hz), 1.0-1.3(2H, m), 1.35-1.7(3H, m), 2.38(3H, s), 2.65-2.9(2H, m), 3.55-3.75(2H, m), 4.19(2H, s), 4.56(2H, brs), 5.38(1H, d, J=2.0 Hz), 6.81(2H, d, J=8.8 Hz), 6.89(2H, d, J=8.8 Hz), 7.36(1H, d, J=2.0 Hz), 7.61(2H, d, J=8.8 Hz), 7.73(1H, d, J=7.6 Hz), 10.39(1H, s) (+)ESI-MS(m/z): 435(M+H)⁺, 457(M+Na)⁺

Preparation 127

To a solution of 4-nitroaniline (27.62 g) and triethylamine (24.3 g) in acetonitrile (280 ml) was added dropwise chloroacetyl chloride (24.8 g) at 5° C. and the mixture was stirred at ambient temperature for 20 hours. The precipitates were collected by filtration and washed with water and diisopropyl ether, and dried in vacuo over phosphorus pentoxide to give 2-chloro-N-(4-nitrophenyl)acetamide (33.99 g) as a yellow powder.

¹H-NMR(DMSO-d₆):δ 4.35(2H, s), 7.75-7.9(2H, m), 8.2-8.3(2H, m), 10.91(1H, s) (−)APCI-MS(m/z): 213(M−H)⁻

Preparation 128

To a suspension of sodium hydride (60% oil dispersion) (1.32 g) in N,N-dimethylformamide (40 ml) was added a solution of pyrazole (2.25 g) in N,N-dimethylformamide (20 ml) at 5° C. and the mixture was stirred at ambient temperature for an hour. To this mixture was added dropwise a solution of 2-chloro-N-(4-nitrophenyl)acetamide (6.44 g) in N,N-dimethylformamide (40 ml) and stirred at 50° C. for 8 hours. The mixture was poured into a mixture of ethyl acetate and ice-water and the separated organic layer was washed with water and brine, dried over magnesium sulfate and evaporated in vacuo. The residue was purified by column chromatography on silica gel eluting with hexane: ethyl acetate (1:2 v/v) to give N-(4-nitrophenyl)-2-(1H-pyrazol-1-yl)acetamide (3.49 g) as a yellow powder.

¹H-NMR(DMSO-d₆):δ 5.11(2H, s), 6.30(1H, dd, J=2.3 Hz, 1.6 Hz), 7.48(1H, d, J=1.6 Hz), 7.79(1H, d, J=2.3 Hz), 7.85-7.95(2H, m), 8.2-8.3(2H, m), 10.94(1H, s) (+)ESI-MS(m/z): 247(M+H)⁺

Preparation 129

To a solution of N-(4-nitrophenyl)-2-(1H-pyrazol-1-yl)acetamide (3.47 g) in tetrahydrofuran (40 ml) and methanol (40 ml) was added 5% palladium on carbon (1 g, 50% wet) and the mixture was hydrogenated for 4 hours at ambient temperature. The catalyst was removed by filtration and the filtrate was evaporated in vacuo. The residue was purified by column chromatography on silica gel eluting with ethyl acetate:methanol (10:1 v/v) to give N-(4-aminophenyl)-2-(1H-pyrazol-1-yl)acetamide (2.30 g) as a pale brown powder.

¹H-NMR(DMSO-d₆):δ 4.90(2H, brs), 4.92(2H, s), 6.26(1H, dd, J=2.2 Hz, 1.7 Hz), 6.51(2H, d, J=8.7 Hz), 7.21(2H, d, J=8.7 Hz), 7.45(1H, d, J=1.7 Hz), 7.73(1H, d, J=2.2 Hz), 9.87(1H, s) (+)ESI-MS(m/z): 217(M+H)⁺, 239(M+Na)⁺

### EXAMPLE 193

To a solution of N-(4-aminophenyl)-2-(1H-pyrazol-1-yl)acetamide (648 mg), 6- methyl-2-(4-methyl-1-piperidinyl)nicotinic acid (702 mg) and benzotriazol-1-yl-oxytripyrrolidinophosphonium hexafluorophosphate (PyBOP) (1.87 g) in N,N-dimethylformamide (50 ml) was added diisopropylethylamine (775 mg) at ambient temperature and the mixture was stirred at the same temperature for 24 hours. The mixture was poured into a mixture of ethyl acetate, water and 6N hydrochloric acid, and the separated organic layer was washed with water and brine, dried over magnesium sulfate and evaporated in vacuo. The residue was purified by column chromatography on silica gel eluting with ethyl acetate to give 6-methyl-2-(4-methyl-1-piperidinyl)-N-{4-[(1H-pyrazol-1-ylacetyl)amino]phenyl}nicotinamide (1.01 g) as white crystals.

¹H-NMR(DMSO-d₆):δ 0.88(3H, d, J=6.2 Hz), 1.1-1.35(2H, m), 1.4-1.8(3H, m), 2.39(3H,-s), 2.7-2.9(2H, m), 3.6-3.75(2H, m), 5.00(2H, s), 6.28(1H, dd, J=1.7 Hz, 1.5 Hz), 6.82(2H, d, J=7.6 Hz), 7.46(1H, d, J=1.5 Hz), 7.54(2H, d, J=9.0 Hz), 7.67(2H, d, J=9.0 Hz), 7.70(1H, d, J=7.6 Hz), 7.77(1H, d, J=1.7 Hz), 10.29(1H, s), 10.50(1H, s) (+)ESI-MS(m/z): 433(M+H)⁺, 455(M+Na)⁺

### EXAMPLE 194

The following compound was obtained in substantially the same manner as in Example 193.

2-(Dimethylamino)-4-methyl-N-{4-[(1H-pyrazol-1-ylacetyl)amino]phenyl}benzamide

¹H-NMR(DMSO-d₆):δ 2.34(3H, s), 2.76(6H, s), 5.00(2H, s), 6.28(1H, dd, J=2.1 Hz, 1.5 Hz), 6.95(1H, d, J=8.0 Hz), 7.10(1H, s), 7.46(1H, d, J=1.5 Hz), 7.55(2H, d, J=9.0 Hz), 7.67(2H, d, J=9.0 Hz), 7.68(1H, d, J=8.0 Hz), 7.77(1H, d, J=2.1 Hz), 10.29(1H, s), 11.53(1H, s) (+)ESI-MS(m/z): 378(M+H)⁺, 400(M+Na)⁺

Preparation 130

To a solution of 5-nitroindoline (11.72 g), 1H-pyrazol-1-ylacetic acid (9.0 g) and benzotriazol-1-yl-oxytripyrrolidinophosphonium hexafluorophosphate (PyBOP) (44.6 g) in N,N-dimethylformamide (40 ml) was added dropwise diisopropylethylamine (18.5 g) at ambient temperature and the mixture was stirred at 30° C. for 20 hours. The mixture was poured into a mixture of ethyl acetate and water and the separated organic layer was washed with water and brine, dried over magnesium sulfate and evaporated in vacuo. The residue was purified by column chromatography on silica gel eluting with ethyl acetate to give 5-nitro-1-(1H-pyrazol-1-ylacetyl)indoline (12.99 g) as a yellow powder.

¹H-NMR(DMSO-d₆):δ 3.31(2H, t, J=8.7 Hz), 4.32(2H, t, J=8.7 Hz), 5.33(2H, s), 6.31(1H, dd, J=2.4 Hz, 1.9 Hz), 7.49(1H, d, J=1.9 Hz), 7.72(1H, d, J=2.4 Hz), 8.1-8.2(3H, m) (−)ESI-MS(m/z): 271(M−H)⁻

Preparation 131

To a solution of 5-nitro-1-(1H-pyrazol-1-ylacetyl)indoline (12.2 g) in N,N-dimethylformamide (100 ml) was added 5% palladium on carbon (3 g, 50% wet) and the mixture was hydrogenated for 4 hours at 45° C. The catalyst was removed by filtration and washed with N,N-dimethylformamide (20 ml). The filtrate containing 1-(1H-pyrazol-1-ylacetyl)-5-indolinamine was used in the next step without further purification.

### EXAMPLE 195

To a solution of 1-(1H-pyrazol-1-ylacetyl)-5-indolinamine (905 mg), 4-methyl-2-(4-methyl-1-piperidinyl)benzoic acid (871 mg) and benzotriazol-1-yl-oxytripyrrolidinophosphonium hexafluorophosphate (PyBOP) (2.33 g) in N,N-dimethylformamide (30 ml) was added dropwise diisopropylethylamine (966 mg) at ambient temperature and the mixture was stirred at the same temperature for 20 hours. The mixture was poured into a mixture of ethyl acetate and water and the separated organic layer was washed with water and brine, dried over magnesium sulfate and evaporated in vacuo. The residue was purified by column chromatography on silica gel eluting with ethyl acetate to give 4-methyl-2-(4-methyl-1-piperidinyl)-N-[1-(1H-pyrazol-1-ylacetyl)-2,3-dihydro-1H-indol-5-yl]benzamide (865 mg) as a pale brown powder.

¹H-NMR(DMSO-d₆):δ 0.95(3H, d, J=6.0 Hz), 1.3-1.6(3H, m), 1.7-1.85(2H, m), 2.34(3H, s), 2.7-2.9(2H, m), 3.05-3.2(2H, m), 3.23(2H, t, J=8.3 Hz), 4.20(2H, t, J=8.3 Hz), 5.24(2H, s), 6.30(1H, dd, J=2.2 Hz, 1.7 Hz), 7.04(2H, d, J=8.0 Hz), 7.16(1H, d, J=1.5 Hz), 7.39(1H, dd, J=8.0 Hz, 1.5 Hz), 7.47(1H, d, J=1.7 Hz), 7.72(1H, d, J=2.1 Hz), 7.79(1H, d, J=8.0 Hz), 7.82(1H, s), 7.96(1H, d, J=8.0 Hz), 11.85(1H, s) (+)ESI-MS(m/z): 458(M+H)⁺, 480(M+Na)⁺

### EXAMPLE 196

The following compound was obtained in substantially the same manner as in Example 195.

6-Methyl-2-(4-methyl-1-piperidinyl)-N-[1-(1H-pyrazol-1-ylacetyl)-2,3-dihydro-1H-indol-5-yl]nicotinamide

¹H-NMR(DMSO-d₆):δ 0.88(3H, d, J=6.2 Hz), 1.0-1.3(2H, m), 1.5-1.75(3H, m), 2.39(3H, s), 2.7-2.9(2H, m), 3.15-3.3(2H, m), 3.6-3.75(2H, m), 4.20(2H, t, J=8.3 Hz), 5.23(2H, s), 6.30(1H, dd, J=1.6 Hz, 1.5 Hz), 6.81(1H, d, J=7.7 Hz), 7.40(1H, dd, J=8.6 Hz, 1.7 Hz), 7.47(1H, d, J=1.6 Hz), 7.71(1H, d, J=1.5 Hz), 7.75(1H, d, J=1.7 Hz), 7.93(1H, d, J=8.6 Hz), 10.48(1H, s) (+)ESI-MS(m/z): 459(M+H)⁺, 481(M+Na)⁺

### EXAMPLE 197

The following compound was obtained in substantially the same manner as in Example 195.

2-(Dimethylamino)-4-methyl-N-[1-(1H-pyrazol-1-ylacetyl)-2,3-dihydro-1H-indol-5-yl]benzamide

¹H-NMR(DMSO-d₆):δ 2.34(3H, s), 2.76(6H, s), 3.22(2H, t, J=8.5 Hz), 4.20(2H, t, J=8.5 Hz), 5.24(2H, s), 6.30(1H, dd, J=2.0 Hz, 1.8 Hz), 6.95(1H, d, J=7.9 Hz), 7.10(1H, d, J=1.8 Hz), 7.42(1H, dd, J=7.9 Hz, 1.8 Hz), 7.47(1H, d, J=1.8 Hz), 7.67(1H, d, J=8.6 Hz), 7.72(1H, d, J=2.0 Hz), 7.93(1H, d, J=8.6 Hz), 11.54(1H, s) (+)ESI-MS(m/z): 404(M+H)⁺, 426(M+Na)⁺

### EXAMPLE 198

The following compound was obtained in substantially the same manner as in Example 195.

4-Chloro-2-(dimethylamino)-N-[1-(1H-pyrazol-1-ylacetyl)-2,3-dihydro-1H-indol-5-yl]benzamide

¹H-NMR(DMSO-d₆):δ 2.89(6H, s), 3.21(2H, t, J=8.3 Hz), 4.20(2H, t, J=8.3 Hz), 5.24(2H, s), 6.30(1H, dd, J=1.9 Hz, 1.5 Hz), 7.02(1H, dd, J=8.2 Hz, 1.9 Hz), 7.10(1H, d, J=1.9 Hz), 7.42(1H, dd, J=8.2 Hz, 2.0 Hz), 7.52(1H, d, J=8.3 Hz), 7.72(1H, d, J=2.0 Hz), 7.72(1H, s), 7.93(1H, d, J=8.3 Hz), 10.73(1H, s)

### EXAMPLE 199

To a solution of N-(2,3-dihydro-1H-indol-5-yl)-6-methyl-2-(4-methyl-1-piperidinyl)nicotinamide (351 mg), 1H-tetrazol-1-ylacetic acid (128 mg) and benzotriazol-1-yl-oxytripyrrolidinophosphonium hexafluorophosphate (PyBOP) (325 mg) in N,N-dimethylformamide (30 ml) was added dropwise diisopropylethylamine (259 mg) at ambient temperature and the mixture was stirred at the same temperature for 20 hours. The mixture was poured into a mixture of ethyl acetate and water and the separated organic layer was washed with water and brine, dried over magnesium sulfate and evaporated in vacuo. The residue was purified by column chromatography on silica gel eluting with ethyl acetate to give 6-methyl-2-(4-methyl-1-piperidinyl)-N-[1-(1H-tetrazol-1-ylacetyl)-2,3-dihydro-1H-indol-5-yl]nicotinamide (333 mg) as a pale brown powder.

¹H-NMR(DMSO-d₆):δ 0.88(3H, d, J=6.1 Hz), 1.0-1.3(2H, m), 1.4-1.7(3H, m), 2.39(3H, s), 2.7-2.9(2H, m), 3.26(2H, t, J=8.2 Hz), 3.6-3.8(2H, m), 4.25(2H, t, J=8.2 Hz), 5.73(2H, s), 6.81(1H, d, J=7.6 Hz), 7.42(1H, dd, J=8.6 Hz, 1.7 Hz), 7.73(1H, d, J=7.6 Hz), 7.79(1H, d, J=1.7 Hz), 7.90(1H, d, J=8.6 Hz), 9.37(1H, s), 10.50(1H, s) (+)ESI-MS(m/z): 461(M+H)⁺, 483(M+Na)⁺

### EXAMPLE 200

To a solution of 2-(1H-pyrazol-1-ylacetyl)-5-isoindolinamine (895 mg), 6-methyl-2-(4-methyl-1-piperidinyl)nicotinic acid (952 mg) and benzotriazol-1-yl-oxytripyrrolidinophosphonium hexafluorophosphate (PyBOP) (2.50 g) in N,N-dimethylformamide (40 ml) was added dropwise diisopropylethylamine (955 mg) at ambient temperature and the mixture was stirred at the same temperature for 20 hours. The mixture was poured into a mixture of ethyl acetate and water and the separated organic layer was washed with water and brine, dried over magnesium sulfate and evaporated in vacuo. The residue was purified by column chromatography on silica gel eluting with ethyl acetate to give 6-methyl-2-(4-methyl-1-piperidinyl)-N-[2-(1H-pyrazol-1-ylacetyl)-2,3-dihydro-1H-isoindol-5-yl]nicotinamide (658 mg) as white powder.

¹H-NMR(DMSO-d₆):δ 0.96(3H, d, J=5.9 Hz), 1.1-1.4(3H, m), 1.6-1.8(2H, m), 2.39(3H, s), 2.75-2.95(2H, m), 3.4-3.8(6H, m), 5.16(2H, s), 6.27(1H, dd, J=1.9 Hz, 1.3 Hz), 7.0-8.0(7H, m), 10.48(1H, s) (+)ESI-MS(m/z): 459(M+H)⁺, 481(M+Na)⁺

### EXAMPLE 201

The following compound was obtained in substantially the same manner as in Example 200.

4-Methyl-2-(4-methyl-1-piperidinyl)-N-[2-(1H-pyrazol-1-ylacetyl)-2,3-dihydro-1H-isoindol-5-yl]benzamide

¹H-NMR(DMSO-d₆):δ 0.96(3H, d, J=5.9 Hz), 1.25-1.5(3H, m), 1.7-1.85(2H, m), 2.35(3H, s), 2.7-2.9(2H, m), 3.1-3.25(2H, m), 4.67(2H, d, J=8.9 Hz), 4.92(2H, d, J=8.9 Hz), 5.17(2H, s), 6.28(1H, dd, J=1.9 Hz, 1.2 Hz), 7.05(1H, d, J=7.9 Hz), 7.18(1H, s), 7.3-7.45(2H, m), 7.45(1H, d, J=1.2 Hz), 7.54(1H, d, J=9.4 Hz), 7.70(1H, d, J=1.9 Hz), 7.79(1H, d, J=7.9 Hz), 7.92(1H, d, J=4.0 Hz), 11.92 and 11.93(total 1H, s) (+)ESI-MS(m/z): 458(M+H)⁺, 480(M+Na)⁺

### EXAMPLE 202

The following compound was obtained in substantially the same manner as in Example 200.

2-(Dimethylamino)-4-methyl-N-[2-(1H-pyrazol-1-ylacetyl)-2,3-dihydro-1H-isoindol-5-yl]benzamide

¹H-NMR(DMSO-d₆):δ 2.35(3H, s), 2.77(6H, s), 4.66(2H, d, J=8.2 Hz), 4.92(2H, d, J=7.9 Hz), 5.18(2H, s), 6.28(1H, dd, J=1.7 Hz, 1.3 Hz), 6.96(1H, d, J=7.9 Hz), 7.10(1H, s), 7.3-7.4(2H, m), 7.45(1H, d, J=1.3 Hz), 7.55-7.75(3H, m), 7.83(1H, s), 11.58(1H, s) (+)ESI-MS(m/z): 404(M+H)⁺, 426(M+Na)⁺

### EXAMPLE 203

The following compound was obtained in substantially the same manner as in Example 200.

4-Chloro-2-(dimethylamino)-N-[2-(1H-pyrazol-1-ylacetyl)-2,3-dihydro-1H-isoindol-5-yl]benzamide

¹H-NMR(DMSO-d₆):δ 2.81(6H, s), 4.66(2H, d, J=8.4 Hz), 4.91(2H, d, J=8.1 Hz), 5.17(2H, s), 6.28(1H, dd, J=2.1 Hz, 1.8 Hz), 7.02(1H, dd, J=8.2 Hz, 1.8 Hz), 7.11(1H, d, J=1.8 Hz), 7.33(1H, d, J=8.2 Hz), 7.45(1H, d, J=1.8 Hz), 7.53(1H, d, J=8.2 Hz), 7.70(1H, d, J=1.8 Hz), 7.80(1H, s), 10.80(1H, s) (+)ESI-MS(m/z): 446(M+Na)⁺

### EXAMPLE 204

The following compound was obtained in substantially the same manner as in Example 1.

2,3-Dimethyl-N-[1-(2-pyridinylacetyl)-2,3-dihydro-1H-indol-5-yl]benzamide

¹H-NMR(DMSO-d₆):δ 2.24(3H, s), 2.28(3H, s), 3.16(2H, t, J=8.3 Hz), 4.01(2H, s), 4.22(2H, t, J=8.3 Hz), 7.12-7.48(6H, m), 7.69-7.83(2H, m), 7.97(1H, d, J=8.7 Hz), 8.47-8.54(1H, m), 10.22(1H, s) (+)ESI-MS(m/z): 386(M+H)⁺, 408(M+Na)⁺

### EXAMPLE 205

The following compound was obtained in substantially the same manner as in Example 1.

2,4-Dimethyl-N-[1-(2-pyridinylacetyl)-2,3-dihydro-1H-indol-5-yl]benzamide

¹H-NMR(DMSO-d₆):δ 2.31(3H, s), 2.35(3H, s), 3.16(2H, t, J=8.4 Hz), 4.00(2H, s), 4.22(2H, t, J=8.4 Hz), 7.04-7.15(2H, m), 7.22-7.50(4H, m), 7.67-7.83(2H, m), 7.97(1H, d, J=8.7 Hz), 8.46-8.54(1H, m), 10.13(1H, s) (+)ESI-MS(m/z): 386(M+H)⁺, 408(M+Na)⁺

### EXAMPLE 206

The following compound was obtained in substantially the same manner as in Preparation 36.

N-[1-(2-Pyridinylacetyl)-2,3-dihydro-1H-indol-5-yl]-2,4-bis(trifluoromethyl)benzamide

¹H-NMR(DMSO-d₆): δ 3.18(2H, t, J=8.3 Hz), 4.02(2H, s), 4.23(2H, t, J=8.3 Hz), 7.28(1H, dd, J=5.7 Hz, 7.3 Hz), 7.32-7.43(2H, m), 7.65(1H, s), 7.71-7.82(1H, m), 7.93-8.07(2H, m), 8.17-8.26(2H, m), 8.48-8.54(1H, m), 10.63(1H, s) (+)ESI-MS(m/z): 494(M+H)⁺, 516(M+Na)⁺

### EXAMPLE 207

The following compound was obtained in substantially the same manner as in Preparation 36.

N-[1-(2-Pyridinylacetyl)-2,3-dihydro-1H-indol-5-yl]-2,5-bis(trifluoromethyl)benzamide

¹H-NMR(DMSO-d₆):δ 3.18(2H, t, J=8.3 Hz), 4.02(2H, s), 4.23(2H, t, J=8.3 Hz), 7.28(1H, dd, J=5.5 Hz, 7.1 Hz), 7.32-7.42(2H, m), 7.66(1H, s), 7.71-7.82(1H, m), 8.01(1H, d, J=8.6 Hz), 8.08-8.18(3H, m), 8.48-8.53(1H, m), 10.63(1H, s) (+)ESI-MS(m/z): 494(M+H)⁺, 516(M+Na)⁺

Preparation 132

A mixture of 2-isopropoxy-4-methylbenzoic acid (2.57 g), tert-butyl 5-amino-1-indolinecarboxylate (3.41 g), 1-hydroxybenzotriazole hydrate (2.13 g) and 1-[3-(dimethylamino)propyl]-3-ethylcarbodiimide (2.16 g) in N,N-dimethylformamide (30 ml) was stirred at ambient temperature overnight. The reaction mixture was poured into a mixture of ethyl acetate and water, and the organic layer was washed with brine and dried over magnesium sulfate. The solvent was concentrated in vacuo and the precipitate was collected by filtration to give tert-butyl 5-[(2-isopropoxy-4-methylbenzoyl)amino]-1-indolinecarboxylate (4.82 g).

¹H-NMR(DMSO-d₆): δ 1.38(3H, d, J=6.02 Hz), 1.51(9H, s) 2.36(3H,s), 3.07(2H, t, J=8.36 Hz), 3.91(2H, t, J=8.36 Hz), 4.75-4.80(1H, m), 6.89(1H, d, J=7.98 Hz), 7.04(1H, s), 7.41(1H, s), 7.63-7.69(2H, m), 7.71(1H, d, J=7.98 Hz), 10.01(1H, s)

Preparation 133

A mixture of tert-butyl 5-[(2-isopropoxy-4-methylbenzoyl)amino]-1-indolinecarboxylate (1.59 g) and trifluoroacetic acid (3.0 ml) in dichloromethane (5 ml) was stirred at ambient temperature for 5 hours. The reaction mixture was evaporated in vacuo, and the residue was dissolved in a mixture of ethyl acetate and water. The solution was adjusted to pH 8.5 with aqueous potassium carbonate solution. The organic layer was washed with brine and dried over magnesium sulfate. The solvent was concentrated in vacuo and the precipitate was collected by filtration to give N-(2,3-dihydro-1H-indol-5-yl)-2-isopropoxy-4-methylbenzamide (1.1 g).

¹H-NMR(DMSO-d₆):δ 1.39(3H, d, J=6.00 Hz), 2.35(3H, s), 2.91(2H, t, J=8.30 Hz), 3.37-3.45(2H, m), 4.75-4.87(1H, s), 5.38(1H, br.s), 6.49(1H, d, J=8.28 Hz), 6.88(1H, d, J=8.02 Hz), 7.02(1H, s), 7.20(1H, dd, J=2.05 Hz, 8.28 Hz), 7.43(1H, s), 7.77(1H, d, J=8.02 Hz), 9.84(1H, s)

### EXAMPLE 208

A mixture of N-(2,3-dihydro-1H-indol-5-yl)-2-isopropoxy-4-methylbenzamide (680 mg), (6-[(tert-butoxycarbonyl)amino]-2-pyridinyl}acetic acid (580 mg), 1-hydroxybenzotriazole hydrate (352 mg) and 1-[3-(dimethylamino)propyl]-3-ethylcarbodiimide (357 mg) and N,N-dimethylaminopyridine (24 mg) in N,N-dimethylformamide (30 ml) was stirred at ambient temperature for overnight. The reaction mixture was poured into a mixture of ethyl acetate and water, and the organic layer was washed with brine and dried over magnesium sulfate. The solvent was concentrated in vacuo, and the precipitate was collected by filtration to give tert-butyl 6-(2-{5-[(2-isopropoxy-4-methylbenzoyl)amino]-2,3-dihydro-1H-indol-1-yl}-2-oxoethyl)-2-pyridinyl carbamate (1.07 g).

¹H-NMR(DMSO-d₆):δ 1.38(3H, d, J=5.96 Hz), 1.46(9H, s), 2.36(3H, s), 3.19(2H, t, J=8.16 Hz), 3.87(2H, s), 4.28(2H, t, J=8.16 Hz), 4.78-4.83(1H, m), 6.89(1H, d, J=7.76 Hz), 6.98(1H, d, J=6.16 Hz), 7.04(1H, s), 7.37(1H, d, J=8.28 Hz), 7.66-7.72(4H, m), 7.98(1H, d, J=8.60 Hz), 9.68(1H, s), 10.06(1H, s)

### EXAMPLE 209

A mixture of tert-butyl 6-(2-{5-[(2-isopropoxy-4-methylbenzoyl)amino]-2,3-dihydro-1H-indol-1-yl}-2-oxoethyl)-2-pyridinyl carbamate (1.0 g) and trifluoroacetic acid (1.42 ml) in dichloromethane (5 ml) was stirred at ambient temperature for 5 hours. The reaction mixture was evaporated in vacuo and the residue was dissolved in a mixture of ethyl acetate and water and adjusted to pH 8.5 with aqueous potassium carbonate solution. The organic layer was washed with brine and dried over magnesium sulfate. The solvent was concentrated in vacuo and the residue was recrystallized from ethyl acetate and diisopropyl ether to give N-{1-[(6-amino-2-pyridinyl)acetyl]-2,3-dihydro-1H-indol-5-yl}-2-isopropoxy-4-methylbenzamide (757 mg).

¹H-NMR(DMSO-d₆): δ 1.38 (3H, d, J=5.98 Hz), 2.36 (3H, s), 3.15 (2H,t, J=8.38 Hz), 3.71 (2H, s), 4.20 (2H, t, J=8.38 Hz), 4.75-4.86 (1H, m), 5.88 (2H, s), 6.31 (1H, d, J=7.92 Hz), 6.44 (1H, d, J=6.98 Hz), 6.89 (1H, d, J=7.76 Hz), 7.03 (1H, s), 7.28-7.40 (2H, m), 7.68-7.74 (2H, m), 7.99 (1H, d, J=8.68 Hz), 10.06 (1 H, s) (+)ESI-MS(m/z): 445(M+1)⁺, 467(M+Na)⁺

### EXAMPLE 210

N-(1-{[2-(Formylamino)-1,3-thiazol-4-yl]acetyl}-2,3-dihydro-1H-indol-5-yl)-2-isopropoxy-4-methylbenzamide

The title compound was obtained in a similar manner as in Example 208 from (2-(formylamino)-1,3-thiazol-4-yl)acetic acid, 1-hydroxybenzotriazole hydrate and 1-[3-(dimethylamino)propyl]-3-ethylcarbodiimide.

¹H-NMR(DMSO-d₆): δ 1.39(3H, d J=6.04 Hz), 2.36(3H, s), 3.17(2H, t, J=8.36 Hz), 3.86(2H, s), 4.20(2H, t, J=8.36 Hz), 4.77-4.83(1H, m), 6.89(1H, d, J=7.88 Hz), 7.04(2H, s), 7.37-7.40(1H, m), 7.69-7.82(2H, m), 8.00(1H, d, J=8.68 Hz), 8.46(1H, s), 10.06(1H, s)

### EXAMPLE 211

A solution of N-(1-{[2-(formylamino)-1,3-thiazol-4-yl]acetyl)-2,3-dihydro-1H-indol-5-yl)-2-isopropoxy-4-methylbenzamide (460 mg) and concentrated hydrochloric acid (246 mg) in methanol (30 ml) and tetrahydrofuran (30 ml) was stirred at 50-55° C. for 2 hours. The reaction mixture was evaporated in vacuo and the residue was dissolved in a mixture of ethyl acetate and water and adjusted to pH 8.5 with aqueous potassium carbonate solution. The organic layer was washed with brine and dried over magnesium sulfate. The solvent was concentrated in vacuo and the precipitate was collected by filtration to give N-{1-[{2-amino-1,3-thiazol-4-yl)acetyl]-2,3-dihydro-1H-indol-5-yl}-2-isopropoxy-4-methylbenzamide (350 mg).

¹H-NMR(DMSO-d₆): δ 1.38(3H, d, J=6.00 Hz), 2.36(3H, s), 3.15(2H, t, J=8.36 Hz), 3.57(2H, s), 4.20(2H, t, J=8.36 Hz), 4.74-4.86(1H, m), 6.32(1H, s), 6.87-6.98(3H, m), 7.04(1H, s), 7.38(1H, d, J=8.68 Hz), 7.69-7.74(2H, m), 7.99(1H, d, J=8.68 Hz), 10.06(1H, s) (+)ESI-MS(m/z): 451(M+1)⁺, 473(M+Na)⁺

### EXAMPLE 212

A mixture of N-(2,3-dihydro-1H-indol-5-yl)-2-isopropoxy-4-methylbenzamide (291 mg), 2-pyridylacetic acid dihydrochloride (417 mg), 1-hydroxybenzotriazole hydrate (241 mg), 1-[3-(dimethylamino)propyl]-3-ethylcarbodiimide (244 mg) and N,N-dimethylaminopyridine (4 mg) in N,N-dimethylformamide (15 ml) was stirred at ambient temperature overnight. The reaction mixture was poured into a mixture of ethyl acetate and water, and the organic layer was washed with brine and dried over magnesium sulfate. The solvent was concentrated in vacuo, and the precipitate was collected by filtration to give 2-isopropoxy-4-methyl-N-[1-(2-pyridinylacetyl)-2,3-dihydro-1H-indol-5-yl]benzamide (635 mg).

¹H-NMR(DMSO-d₆): δ 1.38(3H, d, J=6.02 Hz), 2.36(3H, s), 3.17(2H, t, J=8.36 Hz), 4.05(2H, s), 4.74-4.86(1H, m), 6.88(1H, d, J=7.76 Hz), 7.04(1H, s), 7.25-7.39(3H, m), 7.70-7.81(3H, m), 7.98(1H, d, J=8.68 Hz), 8.49-8.52(1H, m), 10.06(1H, s) (+)ESI-MS(m/z): 430(M+1)⁺, 452(M+Na)⁺

### EXAMPLE 213

4-Chloro-2-isopropoxy-N-[1-(2-pyridinylacetyl)-2,3-dihydro-1H-indol-5-yl]benzamide

The title compound was obtained in a similar manner as in Example 212 from 4-chloro-2-isopropoxybenzoic acid, 2-pyridylacetic acid dihydrochloride, 1-hydroxybenzotriazole hydrate and 1-[3-(dimethylamino)propyl]-3-ethylcarbodiimide.

¹H-NMR(DMSO-d₆): δ 1.35(3H, d, J=6.00 Hz), 3.21(2H, t, J=8.36 Hz), 4.05(2H, s), 4.22(2H, t, J=8.36 Hz), 4.76-4.88(1H, m), 7.11(1H, dd, J=1.80 Hz, 8.26 Hz), .7.13-7.38(4H, m), 7.68-7.81(3H, m), 7.99(1H, d, J=8.66 Hz), 8.50(1H, d, J=4.56 Hz), 10.02(1H, s) (+)ESI-MS(m/z): 450(M+1)⁺, 472(M+Na)⁺

### EXAMPLE 214

2-Isopropoxy-N-[1-(2-pyridinylacetyl)-2,3-dihydro-1H-indol-5-yl]benzamide

The title compound was obtained in a similar manner as in Example 212 from 2-isopropoxybenzoic acid, 2-pyridylacetic acid dihydrochloride, 1-hydroxybenzotriazole hydrate and 1-[3-(dimethylamino)propyl]-3-ethylcarbodiimide.

¹H-NMR(DMSO-d₆): δ 1.36(3H, d, J=6.04 Hz), 3.17(2H, t, J=8.36 Hz), 4.06(2H, s), 4.22(2H, t, J=8.36 Hz), 4.74-4.80(1H, m), 7.07(1H, d, J=7.48 Hz), 7.19(1H, d, J=8.40 Hz), 7.27-7.29(1H, m), 7.36-7.48(3H, m), 7.71-7.77(3H, m), 7.99(1H, d, J=8.64 Hz), 8.50-8.51(1H, m), 10.09(1H, s)

### EXAMPLE 215

2-Methoxy-N-[1-(2-pyridinylacetyl)-2,3-dihydro-1H-indol-5-yl]benzamide

The title compound was obtained in a similar manner as in Example 212 from 2-methoxybenzoic acid, 2-pyridylacetic acid dihydrochloride, 1-hydroxybenzotriazole hydrate and 1-[3-(dimethylamino)propyl]-3-ethylcarbodiimide.

¹H-NMR(DMSO-d₆): δ 3.17(2H, t, J=8.36 Hz), 3.90(3H, s), 4.01(2H, s), 4.22(2H, t, J=8.36 Hz), 7.08(1H, d, J=7.60 Hz), 7.17(1H, d, J=8.28 Hz), 7.31-7.50(4H, m), 7.62-7.77(3H, m), 7.98(1H, d, J=8.60 Hz), 8.50(1H, d, J=5.00 Hz), 10.05(1H, s) (+)ESI-MS(m/z): 388(M+1)⁺, 410(M+Na)⁺

### EXAMPLE 216

2-Methoxy-4-methyl-N-[1-(2-pyridinylacetyl)-2,3-dihydro-1H-indol-5-yl]benzamide

The title compound was obtained in a similar manner as in Example 212 from 2-methoxy-4-methylbenzoic acid, 2-pyridylacetic acid dihydrochloride, 1-hydroxybenzotriazole hydrate and 1-[3-(dimethylamino)propyl]-3-ethylcarbodiimide.

¹H-NMR(DMSO-d₆): δ 2.37(3H, s), 3.16(2H, t, J=8.36 Hz), 3.92(3H, s), 3.98(2H, s), 4.22(2H, t, J=8.36 Hz), 6.88(1H, d, J=7.76 Hz), 7.01(1H, s), 7.27-7.46(3H, m), 7.61(1H, d, J=7.72 Hz), 7.72-7.77(2H, m), 7.98(1H, d, J=8.66 Hz), 8.49-8.51(1H, m), 9.96(1H, s) (+)ESI-MS(m/z): 402(M+1)⁺, 424(M+Na)⁺

### EXAMPLE 217

2-Ethoxy-4-methyl-N-[1-(2-pyridinylacetyl)-2,3-dihydro-1H-indol-5-yl]benzamide

The title compound was obtained in a similar manner as in Example 212 from 2-ethoxy-4-methylbenzoic acid, 2-pyridylacetic acid dihydrochloride, 1-hydroxybenzotriazole hydrate and 1-[3-(dimethylamino)propyl]-3-ethylcarbodiimide.

¹H-NMR(DMSO-d₆): δ 1.43(3H, t, J=6.90 Hz), 2.36(3H, s), 3.17(2H, t, J=8.36 Hz), 3.93(2H, s), 4.04-4.26(4H, m), 6.89(1H, d, J=7.92 Hz), 7.00(1H, s), 7.25-7.42(3H, m), 7.65-7.80(3H, m), 7.98(1H, d, J=8.66 Hz), 8.50(1H, d, J=4.92 Hz), 10.02(1H, s) (+)ESI-MS(m/z): 416(M+1)⁺, 438(M+Na)⁺

Preparation 134

The following compound was obtained in substantially the same manner as in Preparation 64.

4-Acetyl-2-(dimethylamino)benzoic acid

¹H-NMR(DMSO-d₆): δ 2.63(3H, s), 2.87(6H, s), 7.73(1H, dd, J=0.9 Hz, 8.0 Hz), 7.87-7.97(2H, m), 15.63-17.36(1H, br) (−)ESI-MS(m/z):206(M−H)⁻

### EXAMPLE 218

The following compound was obtained in substantially the same manner as in Example 74.

4-Acetyl-2-(dimethylamino)-N-(4-{[2-(2-pyridinyl)ethyl]amino}phenyl)benzamide

¹H-NMR(DMSO-d₆): δ 2.61(3H, s), 2.83(6H, s), 2.99(2H, t, J=7.1 Hz), 3.30-3.44(2H, m), 5.60(1H, t, J=5.7 Hz), 6.59(2H, d, J=8.8 Hz), 7.18-7.27(1H, m), 7.32(1H, d, J=7.8 Hz), 7.44(2H, d, J=8.8 Hz), 7.55-7.63(2H, m), 7.63-7.77(2H, m), 8.49-8.55(1H, m), 10.58(1H, s) (+)ESI-MS(m/z):403(M+H)⁺, 425(M+Na)⁺

### EXAMPLE 219

The following compound was obtained in substantially the same manner as in Example 70.

N-(4-{Formyl[2-(2-pyridinyl)ethyl]amino}phenyl)-4-methyl-2-(methylamino)benzamide

¹H-NMR(DMSO-d₆): δ 2.30(3H, s), 2.80(3H, d, J=5.0 Hz), 2.91(2H, t, J=7.4 Hz), 4.10(2H, t, J=7.4 Hz), 6.43-6.54(2H, m), 7.17-7.30(4H, m), 7.47(1H, q, J=5.0 Hz), 7.57-7.80(4H, m), 8.34(1H, s), 8.44-8.52(1H, m), 10.04(1H, s)

### EXAMPLE 220

Acetyl chloride (0.28 mL) was added to a mixture of N-(4-{formyl[2-(2-pyridinyl)ethyl]amino}phenyl)-4-methyl-2-(methylamino)benzamide and triethylamine (0.54 mL) in tetrahydrofuran (30 mL), and the mixture was stirred for 1.5 hours at ambient temperature. The reaction mixture was poured into a mixture of ethyl acetate and saturated sodium hydrogencarbonate aqueous solution. The separated organic layer was washed with water, dried over magnesium sulfate and evaporated in vacuo. The residue was triturated with a mixture of isopropyl ether and ether (1:1 v/v) to give 2-[acetyl(methyl)amino]-N-(4-{formyl[2-(2-pyridinyl)ethyl]amino}phenyl)-4-methylbenzamide (0.94 g).

¹H-NMR(DMSO-d₆): δ 1.73(3H, s), 2.40(3H, s), 2.90(2H, t, J=7.4 Hz), 3.08(3H, s), 4.10(2H, t, J=7.4 Hz), 7.11-7.40(6H, m), 7.56(1H, d, J=7.7 Hz), 7.62-7.77(3H, m), 8.33(1H, s), 8.44-8.50(1H, m), 10.45(1H, s) (+)ESI-MS(m/z): 431(M+H)⁺, 453(M+Na)⁺

### EXAMPLE 221

The following compound was obtained in substantially the same manner as in Example 71.

2-[Acetyl(methyl)amino]-4-methyl-N-(4-{[2-(2-pyridinyl)ethyl]amino}phenyl)benzamide

¹H-NMR(DMSO-d₆): δ 1.72(3H, s), 2.38(3H, s), 2.98(2H, t, J=7.2 Hz), 3.06(3H, s), 7.26-7.44(2H, m), 5.57(1H, t, J=5.6 Hz), 6.57(2H, d, J=8.8 Hz), 7.16-7.43(6H, m), 7.49(1H, d, J=7.8 Hz), 7.71(1H, dt, J=1.7 Hz,7.6 Hz), 8.47-8.56(1H, m), 9.93(1H, s) (+)ESI-MS(m/z): 403(M+H)⁺, 425(M+Na)⁺

### EXAMPLE 222

The following compound was obtained in substantially the same manner as in Example 71.

4-Methyl-2-(methylamino)-N-(4-{[2-(2-pyridinyl)ethyl]amino}phenyl)benzamide

¹H-NMR(DMSO-d₆): δ 2.28(3H, s), 2.78(3H, d, J=5.0 Hz), 2.98(2H, t, J=7.2 Hz), 3.29-3.43(2H, m), 5.54(1H, t, J=5.7 Hz), 6.38-6.50(2H, m), 6.56(2H, d, J=8.8 Hz), 7.18-7.28(1H, m), 7.28-7.38(1H, m), 7.37(2H, d, J=8.8 Hz), 7.46-7.60(2H, m), 7.67-7.76(1H, m), 8.49-8.55(1H, m), 9.63(1H, s) (+)ESI-MS(m/z): 361(M+H)⁺, 383(M+Na)⁺

### EXAMPLE 223

The following compound was obtained in substantially the same manner as in Example 74.

4-Chloro-2-(methylamino)-N-(4-{[2-(2-pyridinyl)ethyl]amino}phenyl)benzamide

¹H-NMR(DMSO-d₆): δ 2.78(3H, d, J=4.9 Hz), 2.99(2H, t, J=7.2 Hz), 3.30-3.44(2H, m), 5.59(1H, t, J=5.6 Hz), 6.52-6.67(4H, m), 7.17-7.27(1H, m), 7.27-7.46(3H, m), 7.54-7.77(3H, m), 8.48-8.56(1H, m), 9.81(1H, s) (+)ESI-MS(m/z): 381(M+H)⁺, 403(M+Na)⁺

### EXAMPLE 224

The following compound was obtained in substantially the same manner as in Example 74.

2-Amino-4-methyl-N-(4-{[2-(2-pyridinyl)ethyl]amino}phenyl)benzamide

¹H-NMR(DMSO-d₆): δ 2.19(3H, s), 2.98(2H, t, J=7.2 Hz), 3.28-3.44(2H, m), 5.53(1H, t, J=5.5 Hz), 6.31(2H, s), 6.38(1H, dd, J=1.1 Hz,8.2 Hz), 6.49-6.63(3H, m), 7.18-7.27(1H, m), 7.27-7.43(3H, m), 7.51(1H, d, J=8.2 Hz), 7.65-7.77(1H, m), 8.48-8.56(1H, m), 9.56(1H, s) (+)ESI-MS(m/z): 347(M+H)⁺, 369(M+Na)⁺

Preparation 135

To a mixture of 2-amino-4,5-dimethoxybenzoic acid (5.0 g) and 37% aqueous formaldehyde (38.1 ml) in methanol (100 ml) was added 10% palladium on carbon (3.0 g, 50% wet). The reaction mixture was stirred at ambient temperature for 7 hours under hydrogen atmosphere. The catalyst was filtered off and the solvent was removed by concentration. The residue was purified by column chromatography on silica gel using a mixture of chloroform and methanol (9:1 v/v) as an eluant. The eluted fractions containing the desired product were collected and evaporated in vacuo. The residue was triturated with diethyl ether to give 2-(dimethylamino)-4,5-dimethoxybenzoic acid (0.54 g).

¹H-NMR(DMSO-d₆): δ 2.81(6H, s), 3.80(3H, s), 3.88(3H, s), 7.36(1H, s), 7.46(1H, s) (−)ESI-MS(m/z): 224(M−H)⁻

### EXAMPLE 225

The following compound was obtained in substantially the same manner as in Example 74.

2-(Dimethylamino)-4,5-dimethoxy-N-(4-{[2-(2-pyridinyl)ethyl]amino}phenyl)benzamide

¹H-NMR(DMSO-d₆): δ 2.75(6H, s), 2.99(2H, t, J=7.2 Hz), 3.30-3.46(2H, m), 3.79(3H, s), 3.86(3H, s), 5.56(1H, t, J=5.7 Hz), 6.61(2H, d, J=8.7 Hz), 7.03(1H, s), 7.18-7.28(1H, m), 7.32(1H, d, J=7.7 Hz), 7.45(2H, d, J=8.7 Hz), 7.57(1H, s), 7.71(1H, dt, J=1.8 Hz,7.7 Hz), 8.49-8.56(1H, m), 12.35(1H, s) (+)ESI-MS(m/z): 421(M+H)⁺, 443(M+Na)⁺

Preparation 136

The following compound was obtained in substantially the same manner as in Preparation 73.

Methyl 1-methyl-1,2,3,4-tetrahydro-8-quinolinecarboxylate

¹H-NMR(DMSO-d₆): δ 2.76-2.90(2H, m), 2.68(2H, t, J=6.2 Hz), 2.72(3H, s), 3.19(2H, t, J=5.8 Hz), 3.77(3H, s), 6.56(1H, t, J=7.6 Hz), 7.03(1H, dd, J=1.8 Hz,7.6 Hz), 7.27(1H, dd, J=1.8 Hz,7.6 Hz)

Preparation 137

The following compound was obtained in substantially the same manner as in Preparation 64.

1-Methyl-1,2,3,4-tetrahydro-8-quinolinecarboxylic acid

¹H-NMR(DMSO-d₆): δ 1.78-1.94(2H, m), 2.71(2H, t, J=6.3 Hz), 2.76(3H, s), 3.18(2H, t, J=5.8 Hz), 6.69(1H, t, J=7.5 Hz), 7.07(1H, d, J=7.5 Hz), 7.39(1H, d, J=7.5 Hz), 13.16(1H, s) (−)ESI-MS(m/z): 190(M−H)⁻

### EXAMPLE 226

The following compound was obtained in substantially the same manner as in Example 74.

1-Methyl-N-(4-{[2-(2-pyridinyl)ethyl]amino}phenyl)-1,2,3,4-tetrahydro-8-quinolinecarboxamide

¹H-NMR(DMSO-d₆): δ 1.76-1.93(2H, m), 2.73(2H, t, J=6.1 Hz), 2.77(3H, s), 2.98(2H, t, J=7.2 Hz), 3.01-3.23(2H, m), 3.28-3.44(2H, m), 5.55(1H, t, J=5.7 Hz), 6.58(2H, d, J=8.8 Hz), 6.75(1H, t, J=7.5 Hz), 7.00-7.08(1H, m), 7.18-7.37(3H, m), 7.42(2H, d, J=8.8 Hz), 7.71(1H, dt, J=1.7 Hz, 7.6 Hz), 8.48-8.55(1H, m), 10.09(1H, s) (+)ESI-MS(m/z): 387(M+H)⁺, 409(M+Na)⁺

Preparation 138

The following compound was obtained in substantially the same manner as in Preparation 73.

Methyl 1-ethyl-1,2,3,4-tetrahydro-8-quinolinecarboxylate

¹H-NMR(DMSO-d₆): δ 1.06(3H, t, J=7.0 Hz), 1.71-1.86(2H, m), 2.69(2H, t, J=6.2 Hz), 2.98(2H, q, J=7.0 Hz), 3.07-3.16(2H, m), 3.77(3H, s), 6.64(1H, t, J=7.6 Hz), 7.05(1H, dd, J=1.7 Hz,7.6 Hz), 2.77(1H, dd, J=1.7 Hz, 7.6 Hz)

Preparation 139

The following compound was obtained in substantially the same manner as in Preparation 64.

1-Ethyl-1,2,3,4-tetrahydro-8-quinolinecarboxylic acid

¹H-NMR(DMSO-d₆): δ 1.12(3H, t, J=7.1 Hz), 1.77-1.93(2H, m), 2.77(2H, t, J=6.6 Hz), 3.00(2H, q, J=7.1 Hz), 3.15(2H, t, J=5.7 Hz), 6.88(1H, t, J=7.5 Hz), 7.13-7.20(1H, m), 7.42-7.50(1H, m), 14.11(1H, s) (−)ESI-MS(m/z): 204(M−H)⁻

### EXAMPLE 227

The following compound was obtained in substantially the same manner as in Example 74.

1-Ethyl-N-(4-{[2-(2-pyridinyl)ethyl]amino}phenyl)-1,2,3,4-tetrahydro-8-quinolinecarboxamide

¹H-NMR(DMSO-d₆): δ 1.00(3H, t, J=7.0 Hz), 1.70-1.87(2H, m), 2.75(2H, t, J=6.3 Hz), 2.92-3.09(4H, m), 3.09-3.19(2H, m), 3.36(2H, t, J=7.2 Hz), 5.55(1H, s), 6.58(2H, d, J=8.8 Hz), 6.82(1H, t, J=7.5 Hz), 7.01-7.10(1H, m), 7.17-7.36(3H, m), 7.44(2H, d, J=8.8 Hz), 7.65-7.76(1H, m), 8.48-8.55(1H, m), 10.13(1H, s) (+)ESI-MS(m/z): 401(M+H)⁺, 423(M+Na)⁺

Preparation 140

The following compound was obtained in substantially the same manner as in Preparation 73.

Methyl 5-chloro-1-methyl-1,2,3,4-tetrahydro-8-quinolinecarboxylate

¹H-NMR(DMSO-d₆): δ 1.78-1.93(2H, m), 2.72(2H, t, J=6.8 Hz), 2.73(3H, s), 3.20(2H, t, J=5.7 Hz), 3.79(3H, s), 6.71(1H, d, J=8.5 Hz), 7.30(1H, d, J=8.5 Hz)

Preparation 141

The following compound was obtained in substantially the same manner as in Preparation 64.

5-Chloro-1-methyl-1,2,3,4-tetrahydro-8-quinolinecarboxylic acid

¹H-NMR(DMSO-d₆): δ 1.78-1.94(2H, m), 2.72(2H, t, J=6.3 Hz), 2.78(3H, s), 3.20(2H, t, J=5.6 Hz), 6.75(1H, d, J=8.4 Hz), 7.37(1H, d, J=8.4 Hz), 12.87(1H, s) (−)ESI-MS(m/z): 224(M−H)⁻

### EXAMPLE 228

The following compound was obtained in substantially the same manner as in Preparation 74.

5-Chloro-1-methyl-N-(4-{[2-(2-pyridinyl)ethyl]amino}phenyl)-1,2,3,4-tetrahydro-8-quinolinecarboxamide

¹H-NMR(DMSO-d₆): δ 1.78-1.96(2H, m), 2.74(2H, t, J=6.4 Hz), 2.80(3H, s), 2.98(2H, t, J=7.0 Hz), 3.12-3.22(2H, m), 3.29-3.43(2H, m), 5.57(1H, s), 6.58(2H, d, J=8.8 Hz), 6.89(1H, d, J=8.3 Hz), 7.18-7.37(3H, m), 7.40(2H, d, J=8.8 Hz), 7.71(1H, dt, J=1.8 Hz,7.6 Hz), 8.49-8.56(1H, m), 10.00(1H, s) (+)ESI-MS(m/z): 421(M+H)⁺, 443(M+Na)⁺

### EXAMPLE 229

The following compound was obtained in substantially the same manner as in Preparation 74.

N-(4-{[2-(2-Pyridinyl)ethyl]amino}phenyl)-8-quinolinecarboxamide

¹H-NMR(DMSO-d₆): δ 3.02(2H, t, J=7.2 Hz), 3.32-3.49(2H, m), 5.66(1H, t, J=5.7 Hz), 6.66(2H, d, J=8.8 Hz), 7.19-7.29(1H, m), 7.34(1H, d, J=7.8 Hz), 7.63(2H, d, J=8.8 Hz), 7.67-7.87(3H, m), 8.24(1H, dd, J=1.3 Hz,8.1 Hz), 8.50-8.72(3H, m), 9.10-9.21(1H, m), 12.96(1H, s) (+)ESI-MS(m/z): 369(M+H)⁺, 391(M+Na)⁺

Preparation 142

The following compound was obtained in substantially the same manner as in Preparation 90.

Benzyl 6-methyl-2-(1-piperidinyl)nicotinate

¹H-NMR(DMSO-d₆): δ 1.51 (6H, s), 2.34 (3H, s), 3.24-3.27 (4H, m), 5.28 (2H, s), 6.63 (1H, d, J=7.7 Hz), 7.30-7.48 (5H, m), 7.82 (1H, d, J=7.7 Hz)

Preparation 143

The following compound was obtained in substantially the same manner as in Preparation 91.

6-Methyl-2-(1-piperidinyl)nicotinic acid

¹H-NMR(DMSO-d₆): δ 1.59 (6H, s), 2.38 (3H, s), 3.25 (4H, s), 6.78 (1H, d, J=7.7 Hz), 7.87 (1H, d, J=7.7 Hz)

Preparation 144

The following compound was obtained in substantially the same manner as in Preparation 90.

Benzyl 2-[ethyl(methyl)amino]-6-methylnicotinate

¹H-NMR(DMSO-d₆): δ 1.16 (3H, t, J=7.0 Hz), 2.38 (3H, s), 2.84 (3H, s), 3.54 (2H, q, J=7.0 Hz), 5.29 (2H, s), 6.44 (1H, d, J=7.7 Hz), 7.23-7.45 (5H, m), 7.82 (1H, d, J=7.7 Hz)

Preparation 145

The following compound was obtained in substantially the same manner as in Preparation 91.

2-[Ethyl(methyl)amino]-6-methylnicotinic acid

¹H-NMR(DMSO-d₆): δ 1.10 (3H, t, J=7.0 Hz), 2.35 (3H, s), 2.83 (3H, s), 3.45 (2H, q, J=7.0 Hz), 6.61 (1H, d, J=7.7 Hz), 7.79 (1H, d, J=7.7 Hz)

Preparation 146

The following compound was obtained in substantially the same manner as in Preparation 90.

Benzyl 2-(diethylamino)-6-methylnicotinate

¹H-NMR(DMSO-d₆): δ 1.03 (6H, t, J=7.0 Hz), 2.33 (3H, s), 3.31 (4H, q, J=7.0 Hz), 5.27 (2H, s), 6.55 (1H, d, J=7.7 Hz), 7.30-7.48 (5H, m), 7.72 (1H, d, J=7.7 Hz)

Preparation 147

The following compound was obtained in substantially the same manner as in Preparation 91.

2-(Diethylamino)-6-methylnicotinic acid

¹H-NMR(DMSO-d₆): δ 1.02 (6H, t, J=7.0 Hz), 2.42 (3H, s), 3.33 (4H, q, J=7.0 Hz), 6.88 (1H, d, J=7.7 Hz), 7.92 (1H, d, J=7.7 Hz)

Preparation 148

The following compound was obtained in substantially the same manner as in Preparation 90.

Benzyl 6-methyl-2-(methylamino)nicotinate

¹H-NMR(DMSO-d₆): δ 2.39 (3H, s), 3.06 (3H, d, J=4.8 Hz), 5.30 (2H, s), 6.35 (1H, d, J=7.9 Hz), 7.32-7.44 (5H, m), 7.88 (1H, m), 8.02 (1H, d, J=7.9 Hz)

Preparation 149

The following compound was obtained in substantially the same manner as in Preparation 91.

6-Methyl-2-(methylamino)nicotinic acid

¹H-NMR(DMSO-d₆): δ 2.34 (3H, s), 2.94 (3H, s), 6.43 (1H, d, J=7.8 Hz), 7.92 (1H, d, J=7.8 Hz), 7.92-7.95 (1H, m)

Preparation 150

The following compound was obtained in substantially the same manner as in Preparation 90.

Benzyl 2-(isopropylamino)-6-methylnicotinate

¹H-NMR(DMSO-d₆): δ 1.24 (6H, d, J=6.5 Hz), 2.39 (3H, s), 4.33-4.67 (1H, m), 5.27 (2H, s), 6.32 (1H, d, J=7.9 Hz), 7.24 -7.74 (5H, m), 7.80 (1H, d, J=6.8 Hz), 8.01 (1H, d, J=7.9 Hz)

Preparation 151

The following compound was obtained in substantially the same manner as in Preparation 91.

2-(Isopropylamino)-6-methylnicotinic acid

¹H-NMR(DMSO-d₆): δ 1.24 (6H, d, J=6.4 Hz), 2.37 (3H, s), 4.36-4.49 (1H, m), 6.31 (1H, d, J=7.7 Hz), 8.13 (1H, d, J=7.7 Hz)

Preparation 152

The following compound was obtained in substantially the same manner as in Preparation 90.

Benzyl 2-(cyclohexylamino)-6-methylnicotinate

¹H-NMR(DMSO-d₆): δ 1.25-1.48 (6H, m), 1.68-1.78 (2H, m), 1.98-2.05 (2H, m), 4.08-4.19 (1H, m), 5.27 (2H, s), 6.30 (1H, d, J=7.9 Hz), 7.22-7.42 (5H, m), 7.92 (1H, d, J=7.5 Hz), 8.00 (1H, d, J=7.9 Hz)

Preparation 153

The following compound was obtained in substantially the same manner as in Preparation 91.

2-(Cyclohexylamino)-6-methylnicotinic acid

¹H-NMR(DMSO-d₆): δ 1.05-1.41 (6H, m), 1.55-1.69 (2H, m), 1.89-1.99 (2H, m), 2.31 (3H, s), 3.98-4.09 (1H, m), 6.40 (1H, d, J=7.9 Hz), 7.92 (1H, d, J=7.9 Hz), 8.17 (1H, d, J=7.2 Hz)

### EXAMPLE 230

The following compound was obtained in substantially the same manner as in Preparation 52.

tert-Butyl [4-({[6-methyl-2-(methylamino)-3-pyridinyl]carbonyl}amino)phenyl][2-(2-pyridinyl)ethyl]carbamate

¹H-NMR(DMSO-d₆): δ 1.33 (9H, s), 2.36 (3H, s), 2.87-2.93 (5H, m), 3.88-3.95 (2H, m), 6.50 (1H, d, J=7.8 Hz), 7.14 -7.26 (4H, m), 7.63-7.73 (3H, m), 7.96-8.00 (2H, m), 8.45-8.47 (1H, m), 10.09 (1H, s)

### EXAMPLE 231

The following compound was obtained in substantially the same manner as in Example 44.

6-Methyl-2-(methylamino)-N-(4-{[2-(2-pyridinyl)ethyl]amino}phenyl)nicotinamide

¹H-NMR(DMSO-d₆): δ 2.34 (3H, s), 2.90 (3H, d, J=4.7 Hz), 2.91-3.02 (2H, m), 3.32-3.42 (2H, m), 5.58 (1H, t, J=5.7 Hz), 6.46 (1H, d, J=7.8 Hz), 6.57 (2H, d, J=8.9 Hz), 7.19-7.30 (2H, m), 7.36 (2H, d, J=8.9 Hz), 7.67-7.75 (1H, m), 7.93 (1H, d, J=7.8 Hz), 8.06-8.09(1H, m), 8.51-8.53 (1H, m), 9.74 (1H, s) ESI-MS(m/z): 384(M+Na)⁺, 362(M+H)⁺

### EXAMPLE 232

The following compound was obtained in substantially the same manner as in Example 91.

2-(Cyclohexylamino)-N-(4-{formyl[2-(2-pyridinyl)ethyl]amino}phenyl)-6-methylnicotinamide

¹H-NMR(DMSO-d₆): δ 1.27-1.46 (6H, m), 1.55-1.64 (2H, m), 1.89-1.99 (2H, m), 2.34 (3H, s), 2.74-2.95 (2H, m), 4.01-4.15 (3H, m), 6.49 (1H, d, J=7.9 Hz), 7.18-7.31 (4H, m), 7.64-7.73 (3H, m), 7.96-8.03 (1H, m), 8.22 (1H, d, J=7.6 Hz), 8.35 (1H, s), 8.46-8.49 (1H, m), 10.12 (1H, s)

### EXAMPLE 233

The following compound was obtained in substantially the same manner as in Example 92.

2-(Cyclohexylamino)-6-methyl-N-(4-{[2-(2-pyridinyl)ethyl]amino}phenyl)nicotinamide

¹H-NMR(DMSO-d₆): δ 1.40-1.64 (8H, m), 1.88-1.99 (2H, m), 2.32 (3H, s), 2.99 (2H, t, J=7.4 Hz), 3.32-3.42 (2H, m), 3.98-4.01 (1H, m), 5.59 (1H, t, J=5.7 Hz), 6.43 (1H, d, J=7.8 Hz), 6.58 (2H, d, J=8.8 Hz), 7.20-7.35 (4H, m), 7.67-7.75 (1H, m), 7.94 (1H, d, J=7.8 Hz), 8.30 (1H, d, J=7.6 Hz), 8.51-8.53 (1H, m), 9.73 (1H, s) ESI-MS(m/z): 452(M+Na)⁺, 430(M+H)⁺

### EXAMPLE 234

The following compound was obtained in substantially the same manner as in Example 43.

tert-Butyl [4-({[2-(isopropylamino)-6-methyl-3-pyridinyl]carbonyl}amino)phenyl][2-(2-pyridinyl)ethyl]carbamate

¹H-NMR(DMSO-d₆): δ 1.24 (6H, d, J=6.7 Hz), 1.33 (9H, s), 2.35 (3H, s), 2.90 (2H, t, J=7.4 Hz), 3.92 (2H, t, J=7.4 Hz), 4.22-4.32 (1H, m), 6.49 (1H, d, J=7.9 Hz), 7.14-7.26 (4H, m), 7.61-7.74 (3H, m), 7.98-8.07 (2H, m), 8.45-8.48 (1H, m), 10.08 (1H, s)

### EXAMPLE 235

The following compound was obtained in substantially the same manner as in Example 44.

2-(Isopropylamino)-6-methyl-N-(4-{[2-(2-pyridinyl)ethyl]amino}phenyl)nicotinamide

¹H-NMR(DMSO-d₆): δ 1.16 (6H, d, J=6.4 Hz), 2.33 (3H, s), 2.99 (2H, t, J=7.4 Hz), 3.33-3.41 (2H, m), 4.15-4.32 (1H, m), 5.62 (1H, br.s), 6.44 (1H, d, J=7.7 Hz), 6.57 (2H, d, J=8.8 Hz), 7.19-7.36(4H, m), 7.67-7.77 (1H, m), 7.94 (1H, d, J=7.9 Hz), 8.14 (1H, d, J=7.3 Hz), 8.51-8.53 (1H, m), 9.73 (1H, s) ESI-MS(m/z): 412(M+Na)⁺, 390(M+H)⁺

### EXAMPLE 236

The following compound was obtained in substantially the same manner as in Example 43.

tert-Butyl [4-({[6-methyl-2-(1,3-thiazolidin-3-yl)-3-pyridinyl]carbonyl}amino)phenyl][2-(2-pyridinyl)ethyl]carbamate

¹H-NMR(DMSO-d₆): δ 1.36 (9H, s), 2.40 (3H, s), 2.69-3.10 (4H, m), 3.75 (2H, t, J=6.2 Hz), 3.87-3.94 (2H, m), 4.56 (2H, s), 6.79 (1H, d, J=7.6 Hz), 7.15-7.26 (4H, m), 7.64-7.74 (4H, m), 8.45-8.48 (1H, m), 10.40 (1H, s)

### EXAMPLE 237

The following compound was obtained in substantially the same manner as in Example 44.

6-Methyl-N-(4-{[2-(2-pyridinyl)ethyl]amino}phenyl)-2-(1,3-thiazolidin-3-yl)nicotinamide

¹H-NMR(DMSO-d₆): δ 2.78 (3H, s), 2.95-3.02 (4H, m), 3.34-3.40 (2H, m), 3.72-3.78 (2H, m), 4.55 (2H, s), 5.57 (1H, br.s), 6.57 (2H, d, J=8.7 Hz), 6.76 (1H, d, J=7.6 Hz), 7.18-7.25 (1H, m), 7.31 (1H, d, J=7.8 Hz), 7.40 (2H, d, J=8.7 Hz), 7.62-7.75 (2H, m), 8.51 (1H, d, J=4.2 Hz), 9.97 (1H, s) ESI-MS(m/z):442(M+Na)⁺, 420(M+H)⁺

### EXAMPLE 238

A mixture of 1-methyl-1,2,3,4-tetrahydro-8-quinolinecarboxylic acid (230 mg), 2-(4-aminophenyl)-N-(2-pyridinyl)acetamide (284 mg),1-hydroxybenzotriazole (170 mg) and 1-[3-(dimethylamino)propyl]-3-ethylcarbodiimide (196 mg) in N,N-dimethylformamide (10 ml) was stirred at ambient temperature for 15 hours. The reaction mixture was poured into a mixture of ethyl acetate and water and the organic layer was washed with brine and dried over magnesium sulfate. The solvent was evaporated in vacuo, and the residue was chromatographed on silica gel eluting with ethyl acetate: n-hexane (7:3 v/v). The eluted fractions containing the desired product were collected and the solvent was evaporated in vacuo and the residue was crystallized from a mixture of ethyl acetate and diisopropyl ether to give 1-methyl-N-{4-[2-oxo-2-(2-pyridinylamino)ethyl]phenyl}-1,2,3,4-tetrahydro-8-quinolinecarboxamide (153 mg).

¹H-NMR(DMSO-d₆): δ 1.82-1.87 (2H, m), 2.70-2.76 (2H, m), 2.76 (3H, s), 3.15-3.20 (2H, m), 3.68 (2H, s), 6.71-6.79 (1H, m), 7.05-7.12 (2H, m), 7.24 (1H, d, J=1.2 Hz), 7.30 (2H, d, J=8.4 Hz), 7.66 (2H, d, J=8.4 Hz), 7.71-7.80 (1H, m), 8.05 (1H, d, J=8.4 Hz), 8.31-8.33 (1H, m), 10.46 (1H, s), 10.67 (1H, s) ESI-MS(m/z): 423(M+Na)⁺, 401(M+H)⁺

### EXAMPLE 239

The following compound was obtained in substantially the same manner as in Example 238.

1-Methyl-N-(4-{[(2-pyridinylcarbonyl)amino]methyl}phenyl)-1,2,3,4-tetrahydro-8-quinolinecarboxamide

¹H-NMR(DMSO-d₆): δ 1.81-1.90 (2H, m), 2.73 (3H, s), 2.70-2.75 (2H, m), 3.14-3.19 (2H, m), 4.45 (2H, d, J=6.3 Hz), 6.71-6.78 (1H, d, J=6.6 Hz), 7.23-7.32 (3H, m), 7.78-7.67 (3H, m), 7.96-8.08 (2H, m), 8.65 (1H, d, J=4.7 Hz), 9.30 (1H, t, J=6.3 Hz), 10.40 (1H, s) ESI-MS(m/z):423(M+Na)⁺, 401(M+H)⁺

### EXAMPLE 240

The following compound was obtained in substantially the same manner as in Example 120.

1-Methyl-N-{4-[2-(1-pyrazol-1-yl)ethoxy]phenyl}-1,2,3,4-tetrahydro-8-quinoline carboxamide

¹H-NMR(DMSO-d₆): δ 1.78-1.90 (2H, m), 2.69-2.76 (2H, m), 2.76 (3H, s), 3.16 (2H, t, J=5.4 Hz), 4.30 (2H, t, J=5.3 Hz), 4.48 (2H, t, J=5.3 Hz), 6.24-6.26 (1H, m), 6.70-6.78 (1H m), 6.85 (2H, d, J=9.0 Hz), 7.03-7.07 (1H, m),7.23-7.27 (1H, m), 7.46 (1H, d, J=2.0 Hz), 7.61 (2H, d, J=9.0 Hz), 7.78 (1H, d, J=2.0 Hz), 10.28 (1H, s) ESI-MS(m/z): 377(M+H)⁺

### EXAMPLE 241

The following compound was obtained in substantially the same manner as in Example 120.

2-Isopropoxy-4-methyl-N-{4-[2-(1H-pyrazol-1-yl)ethoxy]phenyl}benzamide

¹H-NMR(DMSO-d₆): δ 1.38 (6H, d, J=6.0 Hz), 2.35 (3H, s), 4.31 (2H, t, J=5.3 Hz), 4.49 (2H, t, J=5.3 Hz), 4.73-4.85 (1H, m), 6.24-6.26 (1H, m), 6.86-6.94 (3H, m), 7.03 (1H, s), 7.46 (1H, d, J=1.9 Hz), 7.60 (2H, d, J=9.0 Hz), 7.71 (1H, d, J=7.9 Hz), 7.78 (1H, d, J=1.9 Hz), 9.99 (1H, s) ESI-MS(m/z): 380(M+H)⁺

### EXAMPLE 242

The following compound was obtained in substantially the same manner as in Example 123.

2-Isopropoxy-4-methyl-N-{4-([2-(1H-1,2,4-triazol-1-yl)ethyl]amino}phenyl)benzamide

¹H-NMR(DMSO-d₆): δ 1.39 (6H, d, J=6.0 Hz), 2.35 (3H, s), 3.41-3.50 (2H, m),4.33 (2H, t, J=6.2 Hz), 4.75-4.84 (1H, m), 5.63-5.66 (1H, m), 6.58 (2H, d, J=8.8 Hz), 6.88 (1H, d, J=8.2 Hz), 7.03 (1H, s), 7.42 (2H, d, J=8.8 Hz), 7.74 (2H, d, J=8.2 Hz), 7.99 (1H, s), 8.47 (1H, s), 9.84 (1H, s) ESI-MS(m/z): 402(M+Na)⁺, 380(M+H)⁺

### EXAMPLE 243

A mixture of tert-butyl 4-{[(2-chloro-6-methyl-3-pyridinyl)carbonyl]amino}phenyl[2-(2-pyridinyl)ethyl]carbamate (467 mg) and sodium isopropoxide (328 mg) in isopropanol (10 ml) was refluxed under stirring for 4 hours. The reaction mixture was evaporated in vacuo and the residue was dissolved in a mixture of ethyl acetate and water, and the organic layer was washed with brine and dried over magnesium sulfate. The solvent was evaporated in vacuo and the residue was chromatographed on silica gel eluting with ethyl acetate: n-hexane (7:3 v/v). The eluted fractions containing the desired product were collected and evaporated in vacuo to give tert-butyl 4-{[(2-isopropoxy-6-methyl-3-pyridinyl)carbonyl]amino}phenyl[2-(2-pyridinyl)ethyl]carbamate (345 mg).

¹H-NMR(DMSO-d₆): δ 1.33 (9H, s), 1.42 (2H, d, J=6.2 Hz), 2.46 (3H, s), 2.90 (2H, t, J=7.4 Hz), 3.92 (2H, t, J=7.4 Hz), 5.37-5.49 (1H, m), 6.99 (1H, d, J=7.7 Hz), 7.17-7.26 (4H, m), 7.64-7.74 (3H, m), 8.07 (1H, d, J=7.6 Hz), 8.45-8.48 (1H, m), 10.09 (1H, s)

### EXAMPLE 244

The following compound was obtained in substantially the same manner as in Example 44.

2-Isopropoxy-6-methyl-N-(4-{[2-(2-pyridinyl)ethyl]amino}phenyl)nicotinamide

¹H-NMR(DMSO-d₆): δ 1.42 (6H, d, J=7.3 Hz), 2.45 (3H, s), 2.99 (2H, t, J=7.4 Hz), 3.34-3.42 (2H, m), 5.38-5.50 (1H, m), 5.64 (1H, br.s), 6.61 (2H, d, J=8.8 Hz), 6.98 (1H, d, J=7.6 Hz), 7.39-7.23 (1H, m), 7.32 (1H, d, J=7.8 Hz), 7.43 (2H, d, J=8.8 Hz), 7.67-7.76 (1H, m), 8.11 (1H, d, J=7.6 Hz), 8.51-8.53 (1H, m), 9.77 (1H, s) ESI-MS(m/z):413(M+Na)⁺, 391(M+H)⁺

### EXAMPLE 245

A mixture of 2,6-dichloro-N-(4-{formyl[2-(2-pyridinyl)ethyl]amino}phenyl)nicotinamide (623 mg) and sodium isopropoxide (984 mg) in isopropanol (20 ml) was refluxed under stirring for 9 hours. The reaction mixture was evaporated in vacuo and the residue was dissolved in a mixture of ethyl acetate and water, and the organic layer was washed with brine and dried over magnesium sulfate. The solvent was evaporated in vacuo and the residue was chromatographed on silica gel eluting with ethyl acetate: n-hexane (7:3 v/v). The eluted fractions containing the desired product were collected and evaporated in vacuo. The residue was crystallized from a mixture of diisopropyl ether and n-hexane to give 2,6-diisopropoxy-N-(4-{[2-(2-pyridinyl)ethyl]amino}phenyl)nicotinamide (310 mg).

¹H-NMR(DMSO-d₆): δ 1.35 (6H, d, J=6.2 Hz), 1.47 (6H, d, J=6.2 Hz), 3.00 (2H, t, J=7.3 Hz), 3.34-3.44 (2H, m), 5.21-5.59 (2H, m), 5.62 (1H, t, J=5.8 Hz), 6.45 (1H, d, J=8.3 Hz), 6.63 (2H, d, J=8.8 Hz), 7.19-7.26 (1H, m), 7.32 (1H, d, J=7.7 Hz), 7.43 (2H, d, J=8.8 Hz), 7.67-7.76 (1H, m), 8.19 (1H, d, J=8.3 Hz), 8.52-8.55 (1H, m), 9.63 (1H, s) ESI-MS(m/z):457(M+Na)⁺, 435(M+H)⁺

### EXAMPLE 246

A mixture of 2-chloro-N-(4-{formyl[2-(2-pyridinyl)ethyl]amino}phenyl)-6-methylnicotinamide (632 mg), 2-propanethiol (366 mg) and potassium tert-butoxide (539 mg) in N,N-dimethylformamide (15 ml) was stirred at ambient temperature for 8 hours. The reaction mixture was poured into a mixture of ethyl acetate and water, and the organic layer was washed with brine and dried over magnesium sulfate. The solvent was evaporated in vacuo and the residue was chromatographed on silica gel eluting with ethyl acetate. The eluted fractions containing the desired product was collected and evaporated in vacuo to give N-(4-{formyl[2-(2-pyridinyl)ethyl]amino}phenyl)-2-(isopropylthio)-6-methylnicotinamide (510 mg).

¹H-NMR(DMSO-d₆): δ 1.33 (6H, d, J=6.9 Hz), 2.51 (3H, s), 2.92 (2H, t, J=7.3 Hz), 3.95-4.16 (3H, m), 7.09 (1H, d, J=7.8 Hz), 7.21-7.23 (4H, m), 7.64-7.80 (4H, m), 7.96 (1H, s), 8.35 (1H, s), 8.46-8.49 (1H, m), 10.44 (1H, s)

### EXAMPLE 247

The following compound was obtained in substantially the same manner as in Example 92.

2-(Isopropylthio)-6-methyl-N-(4-{[2-(2-pyridinyl)ethyl]amino}phenyl)nicotinamide

¹H-NMR(DMSO-d₆): δ 1.31 (6H, d, J=6.8 Hz), 2.49 (3H, s), 2.99 (2H, t, J=7.3 Hz), 3.32-3.42 (2H, m), 3.93-4.08 (1H, m), 5.58 (1H, t, J=5.7 Hz), 6.58 (2H, d, J=8.8 Hz), 7.04 (1H, d, J=7.8 Hz), 7.21-7.25 (1H, m), 7.31 (1H, d, J=7.8 Hz), 7.42 (2H, d, J=8.8 Hz), 7.66-7.75 (2H, m), 8.52 (1H, d, J=4.6 Hz), 9.94 (1H, s) ESI-MS(m/z): 429(M+Na)⁺, 407(M+H)⁺

### EXAMPLE 248

The following compound was obtained in substantially the same manner as in Example 246.

N-(4-{Formyl[2-(2-pyridinyl)ethyl]amino}phenyl)-6-methyl-2-(propylthio)nicotinamide

¹H-NMR(DMSO-d₆): δ 0.97 (3H, t, J=7.3 Hz), 1.60-1.70 (2H, m), 2.51 (3H, s), 2.92 (2H, t, J=7.7 Hz), 3.12 (2H, t, J=7.0 Hz), 4.13 (2H, t, J=7.3 Hz), 7.10 (1H, d, J=7.8 Hz), 7.21-7.31 (4H, m), 7.68-7.82 (4H, m), 7.96 (1H, s), 8.47-8.50 (1H, m), 10.45 (1H, s)

### EXAMPLE 249

The following compound was obtained in substantially the same manner as in Example 92.

6-Methyl-2-(propylthio)-N-(4-{[2-(2-pyridinyl)ethyl]amino}phenyl)nicotinamide

¹H-NMR(DMSO-d₆): δ 0.96 (3H, t, J=7.2 Hz), 1.54-1.72 (2H, m), 2.48 (3H, s), 2.99 (2H, t, J=7.4 Hz), 3.07 (2H, t, J=7.0 Hz), 3.32-3.42 (2H, m), 5.59 (1H, t, J=5.7 Hz), 6.58 (2H, , J=8.8 Hz), 7.05 (1H, d, J=7.8 Hz), 7.19-7.26 (1H, m), 7.31 (1H, d, J=7.7 Hz), 7.42 (2H, d, J=8.8 Hz), 7.66-7.75 (2H, m), 8.51 (1H, d, J=4.5 Hz), 9.95 (1H, s) ESI-MS(m/z): 429(M+Na)⁺, 407(M+H)⁺

### EXAMPLE 250

A mixture of tert-butyl 4-{[(2-chloro-6-methyl-3-pyridinyl)carbonyl]amino}phenyl[2-(2-pyridinyl)ethyl]carbamate (560 mg) and sodium thiomethoxide (252 mg) in N,N-dimethylformamide (15 ml) was stirred at ambient temperature for 15 hours. The reaction mixture was poured into a mixture of ethyl acetate and water, and the organic layer was washed with brine and dried over magnesium sulfate. The solvent was evaporated in vacuo to give tert-butyl 4-({[6-methyl-2-(methylthio)-3-pyridinyl]carbonyl)amino)phenyl[2-(2-pyridinyl)ethyl]carbamate (550 mg).

¹H-NMR(DMSO-d₆): δ 1.30 (9H, s), 2.46 (3H, s), 2.89 (3H, s), 2.84-2.94 (2H, m), 3.92 (2H, t, J=7.3 Hz), 7.11 (1H, d, J=7.8 Hz), 7.16-7.26 (3H, m), 7.65-7.74 (3H, m), 7.83 (1H, d, J=7.8 Hz), 7.96 (1H, s), 8.46-8.48 (1H, m), 10.39 (1H, s)

### EXAMPLE 251

The following compound was obtained in substantially the same manner as in Example 44.

6-Methyl-2-(methylthio)-N-(4-{[2-(2-pyridinyl)ethyl]amino}phenyl)nicotinamide

¹H-NMR(DMSO-d₆): δ 2.44 (3H, s), 2.50 (3H, s), 2.99 (2H, t, J=7.3 Hz), 3.34-3.42 (2H, m), 5.59 (1H, t, J=5.7 Hz), 6.58 (2H, d, J=8.8 Hz), 7.06 (1H, d, J=7.7 Hz), 7.19-7.26 (1H, m), 7.31 (1H, , J=7.7 Hz), 7.41 (2H, d, J=8.8 Hz), 7.66-7.79 (2H, m), 8.52 (1H, d, J=4.2 Hz), 9.96 (1H, s) negative ESI-MS(m/z): 377 (M−H)⁻

### EXAMPLE 252

The following compound was obtained in substantially the same manner as in Example 250.

N-(4-{Formyl[2-(2-pyridinyl)ethyl]amino}phenyl)-2,6-bis(methylthio)nicotinamide

¹H-NMR(DMSO-d₆): δ 2.51 (6H, s), 2.91 (2H, t, J=7.3 Hz), 4.11 (2H, t, J=7.3 Hz), 7.14-7.32 (5H, m), 7.64-7.76 (3H, m), 7.83 (1H, d, J=8.1 Hz), 8.34 (1H, s), 8.46-8.49 (1H, m), 10.42 (1H, s)

### EXAMPLE 253

The following compound was obtained in substantially the same manner as in Example 92.

2,6-Bis(methylthio)-N-(4-{[2-(2-pyridinyl)ethyl]amino}phenyl)nicotinamide

¹H-NMR(DMSO-d₆): δ 2.50 (3H, s), 2.60 (3H, s), 2.99 (2H, t, J=7.3 Hz), 3.37 (2H, t, J=7.3 Hz), 5.62 (1H, s), 6.58 (2H, J=8.7 Hz), 7.11 (1H, d, J=8.1 Hz), 7.22-7.36 (2H, m), 7.40 (2H, d, J=8.7 Hz), 7.67-7.79 (1H, m), 7.94 (1H, d, J=8.0 Hz), 8.50-8.53 (1H, m), 9.94 (1H, s) ESI-MS(m/z): 433(M+Na)⁺, 411(M+H)⁺

### EXAMPLE 254

The following compound was obtained in substantially the same manner as in Example 91.

N-(4-{Formyl[2-(2-pyridinyl)ethyl]amino}phenyl)-2-isopropoxy-4-methylbenzamide

¹H-NMR(DMSO-d₆): δ 1.32 (6H, d, J=6.0 Hz), 2.32 (3H, s), 2.91 (2H, t, J=7.3 Hz), 4.05-4.15 (2H, m), 4.75-4.87 (1H, m), 6.90 (1H, d, J=7.9 Hz), 7.05 (1H, s), 7.20-7.33 (4H, m), 7.64-7.76 (4H, m), 8.34 (1H, s), 8.46-8.48 (1H, m), 10.17 (1H, s)

### EXAMPLE 255

The following compound was obtained in substantially the same manner as in Example 92.

2-Isopropoxy-4-methyl-N-(4-{[2-(2-pyridinyl)ethyl]amino}phenyl)benzamide

¹H-NMR(DMSO-d₆): δ 1.39 (6H, d, J=6.0 Hz), 2.35 (3H, s), 2.99 (2H, t, J=7.3 Hz), 3.34-3.41 (2H, m), 4.75-4.88 (1H, m), 5.61 (1H, s), 6.60 (2H, d, J=8.8 Hz), 6.88 (1H, d, J=7.9 Hz), 7.03 (1H, s), 7.19-7.26 (1H, m), 7.32 (1H, d, J=7.7 Hz), 7.42 (2H, d, J=8.8 Hz), 7.67-7.79 (2H, m), 8.52 (1H, d, J=4.3 Hz), 9.84 (1H, s) ESI-MS(m/z):412(M+Na)⁺, 390(M+H)⁺

### EXAMPLE 256

The following compound was obtained in substantially the same manner as in Example 91.

4-Chloro-N-(4-{formyl[2-(2-pyridinyl)ethyl]amino}phenyl)-2-isopropoxybenzamide

¹H-NMR(DMSO-d₆) δ 1.36 (6H, d, J=6.0 Hz), 2.91 (2H, t J=7.3 Hz), 3.98-4.15 (2H, m), 4.77-4.89 (1H, m), 7.10-7.31 (6H, m), 7.64-7.76 (4H, m), 8.35 (1H, s), 8.46-8.48 (1H, s), 10.16 (1H, s)

### EXAMPLE 257

The following compound was obtained in substantially the same manner as in Example 92.

4-Chloro-2-isopropoxy-N-(4-{[2-(2-pyridinyl)ethyl]amino}phenyl)benzamide

¹H-NMR(DMSO-d₆): δ 1.36 (6H, d, J=6.0 Hz), 2.99 (2H, t, J=7.4 Hz), 3.34-3.41 (2H, m), 4.78-4.90 (1H, m), 5.63 (1H, br.s), 6.60 (2H, d, J=8.8 Hz), 7.09-7.34 (4H, m), 7.42 (2H, d, J=8.8 Hz), 7.67-7.78 (2H, m) 8.51-8.53 (1H, m), 9.76 (1H, s) ESI-MS(m/z):432(M+Na)⁺, 410(M+H)⁺

### EXAMPLE 258

To a solution of tert-butyl 6-[2-(4-aminophenoxy)ethyl]-2-pyridinylcarbamate (448 mg), 2-isopropoxy-4-methylbenzoic acid (291 mg) and 1-hydroxybenzotriazole (250 mg) in N,N-dimethylformamide (30 ml) was added 1-[3-(dimethylamino)propyl]-3-ethylcarbodiimide hydrochloride (WSC.HCl) (313 mg), followed by triethylamine (0.29 ml) at ambient temperature. The reaction mixture was stirred for 15 hours at the same temperature and concentrated in vacuo. The residue was dissolved in ethyl acetate and water, and extracted with ethyl acetate. The organic layer was washed with water and brine, dried over magnesium sulfate, filtered and concentrated in vacuo. The residue was purified by column chromatography on silica gel eluting with hexane: ethyl acetate (2:1) to give tert-butyl 6-(2-{4-[(2-isopropoxy-4-methylbenzoyl)amino]phenoxy}ethyl)-2-pyridinylcarbamate (495 mg) as a yellow foam.

¹H-NMR(CDCl₃): δ 1.48(6H, d, J=5.9 Hz), 1.52(9H, s), 2.38(3H, s), 3.12(2H, t, J=6.7 Hz), 4.30(2H, t, J=6.7 Hz), 4.73-4.87(1H, m), 6.80(1H, s, J=s Hz), 6.85-6.92(4H, m), 7.54-7.60(3H, m), 7.77(1H, d, J=8.2 Hz), 8.17(1H, d, J=8.2 Hz), 10.07(1H, s) ESI-MS(m/z): 506(M+H)⁺

### EXAMPLE 259

To a solution of tert-butyl 6-(2-{4-[(2-isopropoxy-4-methylbenzoyl)amino]phenoxy}ethyl)-2-pyridinylcarbamate (485 mg) in dichloromethane (6 ml) was added trifluoroacetic acid (1.48 ml). The reaction mixture was stirred for 19 hours at ambient temperature, quenched with 10% aqueous potassium carbonate solution, and extracted with dichloromethane. The organic layer was washed with brine, dried over magnesium sulfate, filtered and concentrated in vacuo. The residue was recrystallized from ethyl acetate-diisopropyl ether to give N-{4-[2-(6-amino-2-pyridinyl)ethoxy]phenyl}-2-isopropoxy-4-methylbenzamide (327 mg) as a pale yellow solid.

¹H-NMR(DMSO-d₆): δ 1.37(6H, d, J=5.9 Hz), 2.35(3H, s), 2.92(2H, t, J=6.9 Hz), 4.24(2H, t, J=6.9 Hz), 4.73-4.86(1H, m), 5.83(2H, s), 6.29(2H, d, J=7.6 Hz), 6.45(1H, d, J=6.6 Hz), 6.86-6.93(3H, m), 7.03(1H, s), 7.29(1H, dd, J=8.2,7.2 Hz), 7.59(2H, d, J=8.9 Hz), 7.72(1H, d, J=7.9 Hz), 9.97(1H, s) ESI-MS(m/z): 406(M+H)⁺

### EXAMPLE 260

The following compound was obtained in substantially the same manner as in Example 258.

tert-Butyl (2-{6-[(tert-butoxycarbonyl)amino]-2-pyridinyl)ethyl){4-[(2-isopropoxy-4-methylbenzoyl)amino]phenyl carbamate

¹H-NMR(CDCl₃): δ 1.41(18H, s), 1.51(6H, d, J=5.9 Hz), 2.39(3H, s), 3.04(2H, t, J=5.3 Hz), 3.93(2H, t, J=5.3 Hz), 4.77-4.86(1H, m), 6.82(1H, s), 6.92(1H, d, J=7.9 Hz), 7.08(2H, d, J=7.9 Hz), 7.14(2H, d, J=8.6 Hz), 7.58-7.65(3H, m), 8.17(1H, d, J=7.9 Hz), 10.21(1H, s) ESI-MS(m/z): 605(M+H)⁺

### EXAMPLE 261

The following compound was obtained in substantially the same manner as in Example 259.

N-(4-{[2-(6-Amino-2-pyridinyl)ethyl]amino}phenyl)-2-isopropoxy-4-methylbenzamide

¹H-NMR(DMSO-d₆): δ 1.38(6H, d, J=5.9 Hz), 2.35(3H, s), 2.73(2H, t, J=7.2 Hz), 3.24-3.31(2H, m), 4.76-4.85(1H, m), 5.56(1H, t, J=5.6 Hz), 5.83(2H, s), 6.28(1H, d, J=7.9 Hz), 6.40(1H, d, J=7.2 Hz), 6.59(2H, d, J=8.6 Hz), 6.88(1H, d, J=7.9 Hz), 7.02(1H, s), 7.27(1H, d, J=7.6 Hz), 7.40(2H, d, J=8.9 Hz), 7.75(1H, d, J=7.9 Hz), 9.82(1H, s) ESI-MS(m/z): 405(M+H)⁺

### EXAMPLE 262

The following compound was obtained in substantially the same manner as in Example 258.

tert-Butyl 2-{2-[(tert-butoxycarbonyl)amino]-1,3-thiazol-4-yl}ethyl{4-[(2-isopropoxy-4-methylbenzoyl)amino]phenyl}carbamate

¹H-NMR(CDCl₃): δ 1.49(18H, s), 1.51(6H, d, J=5.9 Hz), 2.39(3H, s), 2.94(2H, t, J=7.9 Hz), 3.91(2H, t, J=7.9 Hz), 4.77-4.86(1H, m), 6.80(2H, d, J=9.6 Hz), 6.92(1H, dd, J=7.2,0.6 Hz), 7.13(2H, d, J=8.6 Hz), 7.63(2H, d, J=8.6 Hz), 8.17(1H, d, J=7.9 Hz), 10.21(1H, s) ESI-MS(m/z): 611(M+H)⁺

### EXAMPLE 263

The following compound was obtained in substantially the same manner as in Example 259.

N-(4-{[2-(2-Amino-1,3-thiazol-4-yl)ethyl]amino}phenyl)-2-isopropoxy-4-methylbenzamide

¹H-NMR(DMSO-d₆): δ 1.39(6H, d, J=5.9 Hz), 2.35(3H, s), 2.66(2H, t, J=7.3 Hz), 3.23(2H, t, J=7.3 Hz), 4.74-4.88(1H, m), 5.52(1H, s), 6.21(1H, s), 6.57(2H, s, J=8.9 Hz), 6.85(2H, s), 6.87(1H, dd, J=7.9,0.6 Hz), 7.02(1H, s), 7.40(2H, d, J=8.9 Hz), 7.75(1H, d, J=7.9 Hz), 9.82(1H, s) ESI-MS(m/z): 411(M+H)⁺

### EXAMPLE 264

To a solution of tert-butyl 4-[2-(4-aminophenoxy)ethyl]-1,3-thiazol-2-ylcarbamate (252 mg), 2-isopropoxy-4-methylbenzoic acid (153 mg) and 1-hydroxybenzotriazole (126 mg) in N,N-dimethylformamide (2.5 ml) was added 1-[3-(dimethylamino)propyl]-3-ethylcarbodiimide hydrochloride (WSC.HCl) (158 mg), followed by triethylamine (91 mg) at ambient temperature. The reaction mixture was stirred for 19 hours at ambient temperature and concentrated in vacuo. The residue was dissolved in ethyl acetate and water, and extracted with ethyl acetate. The organic layer was washed with water and brine, dried over magnesium sulfate, filtered and concentrated in vacuo. The residue was purified by column chromatography on silica gel eluting with hexane: ethyl acetate (4:1→2:1) to give tert-butyl [4-(2-{4-[(2-isopropoxy-4-methylbenzoyl)amino]phenoxy}ethyl)-1,3-thiazol-2-yl]carbamate (0.316 g) as a pale yellow foam.

¹H-NMR(CDCl₃): δ 1.50(6H, d, J=5.9 Hz), 1.54(9H, s), 2.39(3H, s), 3.12(2H, t, J=6.8 Hz), 4.24(2H, t, J=7.0 Hz), 4.81(1H, sept, J=5.9 Hz), 6.80-6.93(4H, m), 7.57(2H, d, J=8.9 Hz), 8.17(2H, d, J=8.1 Hz), 10.08(1H, s) ESI-MS(m/z): 534(M+Na)⁺

### EXAMPLE 265

To a solution of tert-butyl 4-(2-{4-[(2-isopropoxy-4-methylbenzoyl)amino]phenoxy}ethyl)-1,3-thiazol-2-ylcarbamate (312 mg) in dichloromethane (3.1 ml) was added trifluoroacetic acid (0.705 ml). The mixture was stirred for 12 hours, quenched with 10% aqueous potassium carbonate solution and extracted with dichloromethane. The organic layer was washed with brine, dried over magnesium sulfate, filtered and concentrated in vacuo. The residue was recrystallized from hexane-ethyl acetate to give N-{4-[2-(2-Amino-1,3-thiazol-4-yl)ethoxy]phenyl}-2-isopropoxy-4-methylbenzamide (0.182 g) as pale brown powder.

¹H-NMR(CDCl₃): δ 1.51(6H, d, J=6.2 Hz), 2.39(3H, s), 3.02(2H, t, J=6.5 Hz), 4.24(2H, t, J=7.0 Hz), 4.81(1H, sept, J=6.5 Hz), 4.98(2H, br s), 6.27(1H, s), 6.81(1H, s), 6.89-6.92(3H, m), 7.58(2H, d, J=8.9 Hz), 8.17(1H, d, J=8.4 Hz), 10.08(1H, s) ESI-MS(m/z): 412(M+H)⁺

Preparation 154

To a solution of tert-butyl 6-nitro-3,4-dihydro-2(1H)-isoquinolinecarboxylate (495 mg), 2-chloro-6-methylnicotinic acid (359 mg) and 1-hydroxybenzotriazole (320 mg) in N,N-dimethylformamide (5 ml) was added 1-[3-(dimethylamino)propyl]-3-ethylcarbodiimide hydrochloride (WSC.HCl) (401 mg), followed by N,N-dimethylaminopyridine (4.86 mg) at ambient temperature. The reaction mixture was stirred for 12 hours at ambient temperature and concentrated in vacuo. The residue was dissolved in ethyl acetate and water, and extracted with ethyl acetate. The organic layer was washed with water and brine, dried over magnesium sulfate, filtered and concentrated in vacuo. The residue was purified by column chromatography on silica gel eluting with hexane: ethyl acetate (6:1→2:1) to give tert-butyl 6-{[(2-chloro-6-methyl-3-pyridinyl)carbonyl]amino)-3,4-dihydro-2(1H)-isoquinolinecarboxylate (0.768 g) as a pale yellow foam.

¹H-NMR(CDCl₃): δ 1.49(9H, s), 2.54(3H, s), 2.83(2H, t, J=5.9 Hz), 3.63(2H, t, J=5.9 Hz), 4.53(2H, s), 7.08(1H, d, J=8.4 Hz), 7.16(1H, d, J=7.8 Hz), 7.41-7.70(2H, m), 8.03(1H, d, J=7.6 Hz), 8.63(1H, br s) ESI-MS(m/z): 402(M+H)⁺

Preparation 155

To a solution of tert-butyl 6-{[(2-chloro-6-methyl-3-pyridinyl)carbonyl]amino}-3,4-dihydro-2(1H)-isoquinolinecarboxylate (1.23 g) in tetrahydrofuran (17 ml) was added 4-methylpiperidine (911 mg). The reaction mixture was stirred for 4 hours at 60° C. The mixture was poured into water and extracted with ethyl acetate. The organic layer was washed with water and brine, dried over magnesium sulfate, filtered and concentrated in vacuo. The residue was purified by column chromatography on silica gel eluting with hexane: ethyl acetate (2:1) to give tert-butyl 6-({[6-methyl-2-(4-methyl-1-piperidinyl)-3-pyridinyl]carbonyl}amino)-3,4-dihydro-2(1H)-isoquinolinecarboxylate (0.758 g) as a white solid.

¹H-NMR(CDCl₃): δ 1.03(3H, d, J=6.5 Hz), 1.35-1.50(12H, m), 1.84(2H, br d, J=12.7 Hz), 2.52(3H, s), 2.86(2H, t, J=5.4 Hz), 3.00(2H, td, J=12.2, 4.6 Hz), 3.34(2H, d, J=12.7 Hz), 3.65(2H, t, J=5.7 Hz), 4.56(2H, s), 7.02(1H, d, J=7.7 Hz), 7.10(1H, d, J=8.1 Hz), 7.20-7.54(1H, m), 7.56-7.89(1H, m), 8.34(1H, d, J=7.8 Hz), 11.76(1H, s) ESI-MS(m/z): 465(M+H)⁺

Preparation 156

To a solution of tert-butyl 6-({[6-methyl-2-(4-methyl-1-piperidinyl)-3-pyridinyl]carbonyl}amino)-3,4-dihydro-2(1H)-isoquinolinecarboxylate (742.9 mg) in dichloromethane (7.4 ml) was added trifluoroacetic acid (0.62 ml). The mixture was stirred for 48 hours, quenched with 10% aqueous potassium carbonate solution, and extracted with dichloromethane. The organic layer was washed with brine, dried over magnesium sulfate, filtered and concentrated in vacuo. The residue was recrystallized from hexane-ethyl acetate to give 6-methyl-2-(4-methyl-1-piperidinyl)-N-(1,2,3,4-tetrahydro-6-isoquinolinyl)nicotinamide (0.338 g) as a pale yellow solid.

¹H-NMR(CDCl₃): δ 1.03(3H, d, J=6.2 Hz), 1.42(2H, td, J=11.1, 2.2 Hz), 1.53-1.70(1H, m), 1.86(2H, dd, J=12.4, 2.4 Hz), 2.52(3H, s), 3.00(2H, br t, J=11.9 Hz), 3.15(2H, br t, J=5.9 Hz), 3.33(2H, br d, J=19.2 Hz), 3.44(2H, br t, J=5.9 Hz), 4.30(2H, s), 7.03(1H, d, J=7.8 Hz), 7.10(1H, d, J=8.4 Hz), 7.39(1H, d, J=7.8 Hz), 7.80(1H, s), 8.33(1H, d, J=7.6 Hz), 11.90(1H, s) ESI-MS(m/z): 365(M+H)⁺

### EXAMPLE 266

To a solution of 6-methyl-2-(4-methyl-1-piperidinyl)-N-(1,2,3,4-tetrahydro-6-isoquinolinyl)nicotinamide (200 mg) in tetrahydrofuran (4 ml) was added triethylamine (83 mg) and (1-trityl-1H-1,2,4-triazol-3-yl)methyl methanesulfonate (276 mg). The mixture was stirred at ambient temperature for 17 hours. The reaction mixture was poured into water and extracted with ethyl acetate. The organic layer was washed with brine, dried over magnesium sulfate and evaporated in vacuo. The residue was purified by column chromatography on silica gel eluting with hexane: ethyl acetate (2:1→1:1) to give 6-methyl-2-(4-methyl-1-piperidinyl)-N-{2-[(1-trityl-1H-1,2,4-triazol-3-yl)methyl]-1,2,3,4-tetrahydro-6-isoquinolinyl}nicotinamide (196 mg) as a pale yellow foam.

¹H-NMR(CDCl₃): δ 1.02(3H, d, J=6.2 Hz), 1.34-1.51(2H, m), 1.52-1.72(1H, m), 1.84(2H, br d, J=11.3 Hz), 2.51(3H, s), 2.80-3.10(6H, m), 3.35(2H, br d, J=13.0 Hz), 3.68(2H, s), 3.91(2H, s), 6.94(1H, d, J=8.1 Hz), 7.00(1H, d, J=8.1 Hz), 7.09-7.25(6H, m), 7.28-7.43(11H, m), 7.55(1H, s), 7.94(1H, s), 8.33(1H, d, J=7.8 Hz), 11.61(1H, s) ESI-MS(m/z): 688(M+H)⁺

### EXAMPLE 267

To a solution of 6-methyl-2-(4-methyl-1-piperidinyl)-N-{2-[(1-trityl-1H-1,2,4-triazol-3-yl)methyl]-1,2,3,4-tetrahydro-6-isoquinolinyl}nicotinamide (196 mg) in methanol (2.0 ml) was added 35% hydrochloric acid (0.12 ml). The mixture was stirred at ambient temperature for 12 hours. The mixture was poured into water and saturated sodium bicarbonate solution and extracted with ethyl acetate. The organic layer was washed with brine, dried over magnesium sulfate and evaporated in vacuo. The residue was recrystallized from ethyl acetate-hexane to give 6-methyl-2-(4-methyl-1-piperidinyl)-N-[2-(1H-1,2,4-triazol-3-ylmethyl)-1,2,3,4-tetrahydro-6-isoquinolinyl]nicotinamide (92 mg) as white powder.

¹H-NMR(DMSO-d₆): δ 0.88(3H, d, J=6.2 Hz), 1.12-1.25(2H, m), 1.35-1.58(1H, m), 1.62(2H, br d, J=12.4 Hz), 2.39(3H, s), 2.72-2.90(6H, m), 3.57(2H, s), 3.63(2H, br d, J=12.7 Hz), 3.78(2H, s), 6.83(1H, d, J=7.6 Hz), 7.00(1H, d, J=8.1 Hz), 7.39(1H, dd, J=8.1, 1.6 Hz), 7.55(1H, s), 7.75(1H, d, J=7.6 Hz), 8.16(1H, m), 10.46(1H, s) ESI-MS(m/z): 446(M+H)⁺

### EXAMPLE 268

To a solution of 6-methyl-2-(4-methyl-1-piperidinyl)-N-(1,2,3,4-tetrahydro-6-isoquinolinyl)nicotinamide (128 mg) in dichloromethane (1.3 ml) were added 4-formylbenzonitrile (92.1 mg) and sodium triacetoxyborohydride (223 mg). The mixture was stirred at ambient temperature for 2.5 hours. The reaction mixture was quenched with 10% aqueous potassium carbonate solution and extracted with ethyl acetate. The organic layer was washed with brine, dried over magnesium sulfate, filtered and concentrated in vacuo. The residue was recrystallized from ethyl acetate-hexane to give N-[2-(4-cyanobenzyl)-1,2,3,4-tetrahydro-6-isoquinolinyl]-6-methyl-2-(4-methyl-1-piperidinyl)nicotinamide (117 mg) as pale yellow powder.

¹H-NMR(DMSO-d₆): δ 0.89(3H, d, J=6.2 Hz), 1.12-1.25(2H, m), 1.48-1.58(1H, m), 1.62(2H, br d, J=12.7 Hz), 2.39(3H, s), 2.67-2.90(6H, m), 3.51(2H, s), 3.63(2H, br d, J=12.7 Hz), 3.74(2H, s), 6.82(1H, d, J=7.6 Hz), 6.98(1H, d, J=8.4 Hz), 7.38(1H, d, J=8.4 Hz), 7.71(3H, d, J=7.8 Hz), 7.74(1H, d, J=7.6 Hz), 7.81(2H, d, J=7.8 Hz), 10.45(1H, s) ESI-MS(m/z): 480(M+H)⁺

Preparation 157

The following compound was obtained in substantially the same manner as in Preparation 155.

tert-Butyl 6-({[2-(dimethylamino)-6-methyl-3-pyridinyl]carbonyl}amino)-3,4-dihydro-2(1H)-isoquinolinecarboxylate

¹H-NMR(CDCl₃): δ 1.50(9H, s), 2.52(3H, s), 2.84-2.92(8H, m), 3.65(2H, br t, J=5.1 Hz), 4.56(2H, s), 6.97(1H, d, J=7.6 Hz), 7.10(1H, d, J=8.4 Hz), 7.20-7.80(2H, m), 8.26(1H, d, J=7.8 Hz), 10.80(1H, s) ESI-MS(m/z): 433(M+Na)⁺

Preparation 158

The following compound was obtained in substantially the same manner as in Preparation 156.

2-(Dimethylamino)-6-methyl-N-(1,2,3,4-tetrahydro-6-isoquinolinyl)nicotinamide

¹H-NMR(CDCl₃): δ 2.52(3H, s), 2.79(1H, br s), 2.80-3.10(8H, m), 3.21(2H, t, J=5.9 Hz), 4.06(2H, s), 6.96(1H, d, J=7.8 Hz), 7.01(1H, d, J=8.1 Hz), 7.34(1H, d, J=8.4 Hz), 7.54(1H, s), 8.24(1H, d, J=7.6 Hz), 10.78(1H, s) ESI-MS(m/z): 311(M+H)⁺

### EXAMPLE 269

The following compound was obtained in substantially the same manner as in Example 266.

2-(Dimethylamino)-6-methyl-N-{2-[(1-trityl-1H-1,2,4-triazol-3-yl)methyl]-1,2,3,4-tetrahydro-6-isoquinolinyl}nicotinamide

¹H-NMR(CDCl₃): δ 2.51(3H, s), 2.75-3.00(10H, m), 3.68(2H, s), 3.91(2H, s), 6.94(2H, dd, J=8.1, 3.0 Hz), 7.14-1.24(6H, m), 7.25-7.35(9H, m), 7.47(1H, s), 7.54(1H, d, J=6.8 Hz), 7.97(1H, s), 8.25(1H, d, J=7.8 Hz), 10.67(1H, s) ESI-MS(m/z): 634(M+H)⁺

### EXAMPLE 270

The following compound was obtained in substantially the same manner as in Example 267.

2-(Dimethylamino)-6-methyl-N-[2-(1H-1,2,4-triazol-3-ylmethyl)-1,2,3,4-tetrahydro-6-isoquinolinyl}nicotinamide

¹H-NMR(CDCl₃): δ 2.50(3H, s), 2.72-2.90(8H, s), 3.55(2H, s), 3.60(2H, d, J=12.5 Hz), 3.78(2H, s), 6.92(2H, br d, J=7.3 Hz), 7.02(1H, br s), 7.36(1H, br s), 7.70(1H, br s), 8.00-8.20(2H, m) ESI-MS(m/z): 392(M+H)⁺

Preparation 159

To a solution of tert-butyl 6-amino-3,4-dihydro-2(1H)-isoquinolinecarboxylate (261.6 mg), 2-isopropoxy-4-methylbenzoic acid (225 mg) and 1-hydroxybenzotriazole (178 mg) in N,N-dimethylformamide (2 ml) was added 1-[3-(dimethylamino)propyl]-3-ethylcarbodiimide hydrochloride (WSC.HCl) (222 mg), followed by N,N-dimethylaminopyridine (6.44 mg) at ambient temperature. The reaction mixture was stirred for 14 hours at ambient temperature and concentrated in vacuo. The residue was dissolved in ethyl acetate and water, and extracted with ethyl acetate. The organic layer was washed with water and brine, dried over magnesium sulfate, filtered and concentrated in vacuo. The residue was purified by column chromatography on silica gel eluting with hexane: ethyl acetate (6:1) to give tert-butyl 6-[(2-isopropoxy-4-methylbenzoyl)amino]-3,4-dihydro-2(1H)-isoquinolinecarboxylate (0.267 g) as yellow oil.

¹H-NMR(CDCl₃): δ 1.47-1.52(15H, m), 2.40(3H, s), 2.85(2H, t, J=5.7 Hz), 3.64(2H, t, J=5.7 Hz), 4.55(2H, s), 4.82(1H, sept, J=5.9 Hz), 6.82(1H, S), 6.92(1H, d, J=7.8 Hz), 7.08(1H, d, J=8.1 Hz), 7.15-7.85(1H, m), 8.17(1H, d, J=7.8 Hz), 10.16(1H, s) ESI-MS(m/z): 447(M+Na)⁺

Preparation 160

To a solution of tert-butyl 6-[(2-isopropoxy-4-methylbenzoyl)amino]-3,4-dihydro-2(1H)-isoquinolinecarboxylate (260 mg) in dichloromethane (2.6 ml) was added trifluoroacetic acid (0.472 ml). The mixture was stirred for 14 hours, quenched with 10% aqueous potassium carbonate solution, and extracted with ethyl acetate—tetrahydrofuran. The organic layer was washed with brine, dried over magnesium sulfate, filtered and concentrated in vacuo. The residue was purified by column chromatography on silica gel eluting with chloroform: methanol (6:1) to give 2-isopropoxy-4-methyl-N-(1,2,3,4-tetrahydro-6-isoquinolinyl)benzamide (0.141 g) as a pale yellow solid.

¹H-NMR(CDCl₃): δ 1.51(6H, d, J=5.9 Hz), 2.39(3H, s), 3.07(2H, t, J=5.9 Hz), 3.37(2H, t, J=5.9 Hz), 4.22(2H, s), 4.81(1H, sept, J=6.5 Hz), 6.81(1H, s), 6.91(1H, d, J=8.4 Hz), 7.05(1H, d, J=8.6 Hz), 7.32(1H, dd, J=8.6, 2.2 Hz), 7.69(1H, s), 8.14(1H, d, J=8.4 Hz), 10.18(1H, s) ESI-MS(m/z): 325(M+H)⁺

### EXAMPLE 271

To a solution of 2-isopropoxy-4-methyl-N-(1,2,3,4-tetrahydro-6-isoquinolinyl)benzamide (131.2 mg) in tetrahydrofuran (1.3 ml) was added triethylamine (61.4 mg) and (1-trityl-1H-1,2,4-triazol-3-yl)methyl methanesulfonate (187 mg). The mixture was stirred at ambient temperature for 16 hours. The reaction mixture was poured into water and extracted with ethyl acetate. The organic layer was washed with brine, dried over magnesium sulfate, filtered and evaporated in vacuo. The residue was purified by column chromatography on silica gel eluting with hexane: ethyl acetate (1:6→1:8) to give 2-isopropoxy-4-methyl-N-{2-[(1-trityl-1H-1,2,4-triazol-3-yl)methyl]-1,2,3,4-tetrahydro-6-isoquinolinyl}benzamide (0.121 g) as a pale yellow foam.

¹H-NMR(CDCl₃): δ 1.50(6H, d, J=5.9 Hz), 2.39(3H, s), 2.82(2H, t, J=5.7 Hz), 2.91(2H, t, J=5.1 Hz), 3.68(2H, s), 3.91(2H, s), 4.80(1H, sept, J=5.9 Hz), 6.81(1H, s), 6.92(2H, d, J=8.6 Hz), 7.14-7.19(6H, m), 7.29-7.55(10H, m), 7.55(1H, s), 7.95(1H, s), 8.17(1H, d, J=8.1 Hz), 10.10(1H, s) ESI-MS(m/z): 670(M+Na)⁺

### EXAMPLE 272

To a solution of 2-isopropoxy-4-methyl-N-{2-[(1-trityl-1H-1,2,4-triazol-3-yl)methyl]-1,2,3,4-tetrahydro-6-isoquinolinyl}benzamide (114.3 mg) in methanol (2.0 ml) was added 35% hydrochloric acid (27.4 μl). The mixture was stirred at ambient temperature for 18 hours. The mixture was poured into water and saturated sodium bicarbonate solution, and extracted with ethyl acetate. The organic layer was washed with brine, dried over magnesium sulfate, filtered and evaporated in vacuo. The residue was recrystallized from ethyl acetate-hexane to give 2-isopropoxy-4-methyl-N-[2-(1H-1,2,4-triazol-3-ylmethyl)-1,2,3,4-tetrahydro-6-isoquinolinyl]benzamide (0.056 g) as pale yellow powder.

¹H-NMR(DMSO-d₆): δ 1.51(6H, d, J=6.2 Hz), 2.40(3H, s), 2.87(2H, t, J=5.1 Hz), 2.98(2H, t, J=5.1 Hz), 3.74(2H, s), 3.96(2H, s), 4.81(1H, sept, J=6.2 Hz), 6.82(1H, s), 6.92(1H, d, J=7.8 Hz), 6.97(1H, d, J=8.3 Hz), 7.27(1H, d, J=8.3 Hz), 7.65(1H, s), 8.00(1H, s), 8.17(1H, d, J=8.4 Hz), 10.15(1H, s) ESI-MS(m/z): 406(M+H)⁺

Preparation 161

The following compound was obtained in substantially the same manner as in Preparation 159.

tert-Butyl 6-{[2-(dimethylamino)benzoyl]amino}-3,4-dihydro-2(1H)-isoquinolinecarboxylate

¹H-NMR(CDCl₃): δ 1.50(9H, s), 2.83-2.89(8H, m), 3.65(2H, t, J=5.4 Hz), 4.55(2H, s), 7.09(1H, d, J=8.4 Hz), 7.24-7.80(5H, m), 8.26(1H, dd, J=7.8, 1.4 Hz), 12.11(1H, s) ESI-MS(m/z): 418(M+Na)⁺

Preparation 162

The following compound was obtained in substantially the same manner as in Preparation 160.

2-(Dimethylamino)-N-(1,2,3,4-tetrahydro-6-isoquinolinyl)benzamide

¹H-NMR(CDCl₃): δ 2.83(6H, s), 3.13(2H, t, J=5.9 Hz), 3.43(2H, t, J=5.9 Hz), 4.28(2H, s), 7.08(1H, d, J=8.4 Hz), 7.24-7.27(1H, m), 7.31(1H, d, J=7.0 Hz), 7.39(1H, dd, J=8.6, 2.4 Hz), 7.46-7.52(1H, m), 7.69(1H, s), 8.23(1H, dd, J=7.8, 1.4 Hz), 12.30(1H, s) ESI-MS(m/z): 296(M+H)⁺

### EXAMPLE 273

The following compound was obtained in substantially the same manner as in Example 271.

2-(Dimethylamino)-N-{2-[(1-trityl-1H-1,2,4-triazol-3-yl)methyl]-1,2,3,4-tetrahydro-6-isoquinolinyl}benzamide

¹H-NMR(CDCl₃): δ 2.80-2.89(8H, m), 2.92(2H, t, J=5.1 Hz), 3.69(2H, s), 3.91(2H, s), 6.93(1H, d, J=8.1 Hz), 7.14-7.44(18H, m), 7.47(1H, dd, J=2.7, 1.1 Hz), 7.50(1H, d, J=1.6 Hz), 7.95(1H, s), 8.29(1H, dt, J=7.8, 1.4 Hz), 12.00(1H, s) ESI-MS(m/z): 619(M+H)⁺

### EXAMPLE 274

The following compound was obtained in substantially the same manner as in Example 272.

2-(Dimethylamino)-N-[2-(1H-1,2,4-triazol-3-ylmethyl)-1,2,3,4-tetrahydro-6-isoquinolinyl]benzamide

¹H-NMR(CDCl₃): δ 2.79-2.85(8H, m), 2.97(2H, t, J=5.4 Hz), 3.72(2H, s), 3.93(2H, s), 6.98(1H, d, J=8.4 Hz), 7.23-7.40(3H, m), 7.45-7.52(1H, m), 7.61(1H, s), 8.26(1H, dd, J=7.6, 1.1 Hz), 12.11(1H, s) ESI-MS(m/z): 377(M+H)⁺

### EXAMPLE 275

To a solution of tert-butyl 4-aminophenyl[2-(1H-pyrazol-1-yl)ethyl]carbamate (363 mg), 2-(4-methylphenyl)-1-cyclohexene-1-carboxylic acid (286 mg) and 1-hydroxybenzotriazole hydrate (221 mg) in N,N-dimethylformamide (5 ml) was added 1-[3-(dimethylamino)propyl]-3-ethylcarbodiimide hydrochloride (276 mg) at ambient temperature. The reaction mixture was stirred for 21 hours at the same temperature and concentrated in vacuo. The residue was dissolved in ethyl acetate and water, and extracted with ethyl acetate. The organic layer was washed with water and brine, dried over magnesium sulfate, filtered and concentrated in vacuo. The residue was purified by column chromatography on silica gel eluting with hexane: ethyl acetate (1:1) to give tert-butyl [4-({[2-(4-methylphenyl)-1-cyclohexen-1-yl]carbonyl}amino)phenyl][2-(1H-pyrazol-1-yl)ethyl]carbamate (297 mg) as a yellow foam.

¹H-NMR(CDCl₃): δ 1.37(9H, s), 1.76(4H, br s), 2.32(3H, s), 2.41(2H, br s), 2.50(2H, br s), 3.94(2H, t, J=6.2 Hz), 4.29(2H, t, J=6.1 Hz), 6.20(1H, t, J=2.0 Hz), 6.61(1H, s), 6.75(2H, br s), 6.89(2H, d, J=8.6 Hz), 7.15(4H, br s), 7.34(1H, d, J=2.0 Hz), 7.45(1H, d, J=1.6 Hz) ESI-MS(m/z): 523(M+Na)⁺

### EXAMPLE 276

To a solution of tert-butyl 4-({[2-(4-methylphenyl)-1-cyclohexen-1-yl]carbonyl}amino)phenyl[2-(1H-pyrazol-1-yl)ethyl]carbamate (292 mg) in dichloromethane (10 ml) was added trifluoroacetic acid (0.674 ml). The reaction mixture was stirred for 20 hours at ambient temperature, quenched with 10% aqueous potassium carbonate solution and extracted with dichloromethane. The organic layer was washed with brine, dried over magnesium sulfate, filtered and concentrated in vacuo. The residue was recrystallized from ethyl acetate-diisopropyl ether to give 2-(4-methylphenyl)-N-(4-{[2-(1H-pyrazol-1-yl)ethyl]amino}phenyl)-1-cyclohexene-1-carboxamide (200 mg) as a white solid.

¹H-NMR(DMSO-d₆): δ 1.69(4H, br s), 2.21(3H, s), 2.35(4H, br s), 3.37(2H, t, J=6.4 Hz), 4.22(2H, t, J=6.4 Hz), 6.20(1H, t, J=2.0 Hz), 6.47(2H, d, J=8.9 Hz), 7.04(1H, d, J=7.9 Hz), 7.08(1H, d, J=8.9 Hz), 7.17(1H, d, J=7.9 Hz), 7.44(1H, d, J=1.3 Hz), 7.69(1H, d, J=2.0 Hz), 9.11(1H, s) ESI-MS(m/z): 401(M+H)⁺

### EXAMPLE 277

To a solution of 4′-(trifluoromethyl)[1,1′-biphenyl]-2-carboxylic acid (384 mg) in toluene (5 ml) were added thionyl chloride (342 mg) and N,N-dimethylformamide (1 drop) and the mixture was stirred at 50° C. for an hour. The mixture was evaporated in vacuo and the residue was dissolved in tetrahydrofuran (2 ml). The acid chloride in tetrahydrofuran was added to a solution of tert-butyl 4-aminophenyl[2-(1H-pyrazol-1-yl)ethyl]carbamate (363 mg) and triethylamine (0.25 ml) in tetrahydrofuran (8 ml) at ambient temperature and the mixture was stirred at the same temperature for an hour. The mixture was poured into water and extracted with ethyl acetate. The organic layer was washed with brine, dried over magnesium sulfate and evaporated in vacuo. The residue was purified by column chromatography on silica gel eluting with hexane: ethyl acetate (1:1) to give tert-butyl 2-(1H-pyrazol-1-yl)ethyl[4-({[4′-(trifluoromethyl)-1,1′-biphenyl-2-yl]carbonyl}amino)phenyl]carbamate (660 mg) as a yellow foam.

¹H-NMR(CDCl₃): δ 1.38(9H, s), 3.97(2H, t, J=6.1 Hz), 4.31(2H, t, J=6.1 Hz), 6.21(1H, t, J=2.0 Hz), 6.81(2H, br s), 7.01(1H, s), 7.08(2H, d, J=8.6 Hz), 7.35(1H, d, J=2.0 Hz), 7.41-7.59(6H, m), 7.67(2H, d, J=7.9 Hz), 7.78(1H, dd, J=7.6, 1.3 Hz) ESI-MS(m/z): 573(M+Na)⁺

### EXAMPLE 278

To a solution of tert-butyl 2-(1H-pyrazol-1-yl)ethyl[4-({[4′-(trifluoromethyl)-1,1′-biphenyl-2-yl]carbonyl}amino)phenyl]carbamate (660 mg) in dichloromethane (10 ml) was added trifluoroacetic acid (0.674 ml). The reaction mixture was stirred for 20 hours at ambient temperature, quenched with 10% aqueous potassium carbonate solution and extracted with dichloromethane. The organic layer was washed with brine, dried over magnesium sulfate, filtered and concentrated in vacuo. The residue was recrystallized from ethyl acetate-diisopropyl ether to give N-(4-{[2-(1H-pyrazol-1-yl)ethyl]amino}phenyl)-4′-(trifluoromethyl)-1,1′-biphenyl-2-carboxamide (464 mg) as a white solid.

¹H-NMR(DMSO-d₆): δ 3.42(2H, t, J=6.2 Hz), 4.26(2H, t, J=6.2 Hz), 6.22(1H, t, J=2.0 Hz), 6.59(2H, d, J=8.6 Hz), 7.25(2H, d, J=8.9 Hz), 7.45-7.64(7H, m), 7.71-7.26(3H, m), 10.01(1H, s) ESI-MS(m/z): 451(M+H)⁺

### EXAMPLE 279

The following compound was obtained in substantially the same manner as in Example 277.

tert-Butyl 4-{[(4′-methyl-1,1′-biphenyl-2-yl)carbonyl]amino}phenyl[2-(1H-pyrazol-1-yl)ethyl]carbamate

¹H-NMR(CDCl₃): δ 1.39(9H, s), 2.40(3H, s), 3.97(2H, t, J=6.1 Hz), 4.31(2H, t, J=6.1 Hz), 6.22(1H, t, J=2.0 Hz), 6.82(2H, br s), 6.91(1H, s), 7.04(2H, d, J=8.9 Hz), 7.25(2H, d, J=8.9 Hz), 7.33-7.55(7H, m), 7.87(1H, dd, J=8.2, 1.3 Hz) ESI-MS(m/z): 519(M+Na)⁺

### EXAMPLE 280

The following compound was obtained in substantially the same manner as in Example 278.

4′-Methyl-N-(4-{[2-(1H-pyrazol-1-yl)ethyl]amino}phenyl)-1,1′-biphenyl-2-carboxamide

¹H-NMR(DMSO-d₆): δ 2.29(3H, s), 3.38(2H, q, J=6.2 Hz), 4.23(2H, t, J=6.4 Hz), 5.53(1H, t, J=6.1 Hz), 6.21(1H, t, J=2.0 Hz), 6.48(2H, d, J=8.9 Hz), 7.17(2H, d, J=7.9 Hz), 7.23(2H, d, J=8.9 Hz), 7.34(2H, d, J=7.9 Hz), 7.39-7.54(5H, m), 7.71(1H, d, J=2.0 Hz), 9.81(1H, s) ESI-MS(m/z): 419(M+Na)⁺

Preparation 163

A solution of N-(4-nitrophenyl)-N-[2-(1H-pyrazol-1-yl)ethyl]amine (300 mg) in methanol (8 ml) was hydrogenated over 10% palladium on carbon (60 mg, 50% wet) at ambient temperature under atmospheric pressure of hydrogen for an hour. The reaction mixture was filtered with pad of Celite, and the filtrate was concentrated in vacuo to give N-[2-(1H-pyrazol-1-yl)ethyl]-1,4-benzenediamine (261 mg) as a yellow oil. The product was used at the next step without purification.

¹H-NMR(CDCl₃): δ 3.41(2H, br s), 3.51-3.55(3H, m), 4.29-4.33(2H, m), 6.24(1H, t, J=2.0 Hz), 6.49(2H, d, J=8.6 Hz), 6.60(2H, d, J=8.6 Hz), 7.35(1H, d, J=2.3 Hz), 7.54(1H, d, J=1.6 Hz) ESI-MS(m/z): 525(M+Na)⁺

### EXAMPLE 281

To a solution of N-[2-(1H-pyrazol-1-yl)ethyl]-1,4-benzenediamine (251 mg), 4′-ethyl-1,1′-biphenyl-2-carboxylic acid (309 mg) and 1-hydroxybenzotriazole hydrate (228 mg) in N,N-dimethylformamide (8 ml) was added 1-[3-(dimethylamino)propyl]-3-ethylcarbodiimide hydrochloride (285 mg) at ambient temperature. The reaction mixture was stirred for 4 hours at the same temperature and concentrated in vacuo. The residue was dissolved in ethyl acetate and water, and extracted with ethyl acetate. The organic layer was washed with water and brine, dried over magnesium sulfate, filtered and concentrated in vacuo. The residue was recrystallized from ethyl acetate-diisopropyl ether to give 4′-ethyl-N-(4-{[2-(1H-pyrazol-1-yl)ethyl]amino}phenyl)-1,1′-biphenyl-2-carboxamide (282 mg) as a white solid.

¹H-NMR(DMSO-d₆): δ 1.17(3H, t, J=7.6 Hz), 2.60(2H, q, J=7.6 Hz), 3.39(2H, q, J=6.3 Hz), 4.23(2H, t, J=6.3 Hz), 5.53(1H, t, J=6.1 Hz), 6.21(1H, t, J=2.0 Hz), 6.48(2H, d, J=8.9 Hz), 7.19-7.22(4H, m), 7.34-7.54(7H, m), 7.71(1H, d, J=2.0 Hz), 9.80(1H, s) ESI-MS(m/z): 433(M+Na)⁺

### EXAMPLE 282

To a solution of 2-isopropoxy-4-methylbenzoic acid (266 mg) in toluene (5 ml) were added thionyl chloride (245 mg) and N,N-dimethylformamide (1 drop), and the mixture was stirred at 50° C. for 30 minutes. The mixture was evaporated in vacuo and the residue was dissolved in tetrahydrofuran (4 ml). The acid chloride in tetrahydrofuran was added to a solution of tert-butyl 4-aminophenyl[2-(1H-pyrazol-1-yl)ethyl]carbamate (413 mg) and triethylamine (152 mg) in tetrahydrofuran (10 ml) at ambient temperature, and the mixture was stirred at the same temperature for 13 hours. The mixture was poured into water and extracted with ethyl acetate. The organic layer was washed with brine, dried over magnesium sulfate, and concentrated in vacuo. The residue was purified by column chromatography on silica gel eluting with hexane:ethyl acetate (1:1) to give tert-butyl 4-[(2-isopropoxy-4-methylbenzoyl)amino]phenyl[2-(1H-pyrazol-1-yl)ethyl]carbamate (516 mg) as a colorless oil.

¹H-NMR(CDCl₃): δ 1.40(9H, s), 1.51(6H, d, J=6.0 Hz), 2.40(3H, s), 4.03(2H, t, J=6.1 Hz), 4.36(2H, t, J=6.1 Hz), 4.82(1H, sept, J=6.0 Hz), 6.24(1H, t, J=2.0 Hz), 6.82(1H, s), 6.85-7.03(3H, m), 7.39(1H, d, J=2.0 Hz), 7.49(1H, d, J=2.0 Hz), 7.59(2H, d, J=8.2 Hz), 8.17(1H, d, J=7.9 Hz), 10.20(1H, s) (+)ESI-MS(m/z): 501(M+Na)⁺

### EXAMPLE 283

The following compound was obtained in substantially the same manner as in Example 179.

2-Isopropoxy-4-methyl-N-(4-{[2-(1H-pyrazol-1-yl)ethyl]amino}phenyl)benzamide

¹H-NMR(CDCl₃): δ 1.49(6H, d, J=5.9 Hz), 2.38(3H, s), 3.60(2H, t, J=5.6 Hz), 3.94(1H, brs), 4.34(2H, t, J=5.6 Hz), 4.80(1H, sep, J=5.9 Hz), 6.25(1H, t, J=2.0 Hz), 6.60(2H, d, J=8.9 Hz), 6.80(1H, s), 6.90(1H, d, J=7.9 Hz), 7.35(1H, d, J=2.0 Hz), 7.49(2H, d, J=8.9 Hz), 7.55(1H, d, J=1.6 Hz), 8.17(1H, d, J=8.2 Hz), 9.99(1H, s) (+)ESI-MS(m/z): 401(M+Na)⁺

### EXAMPLE 284

To a solution of 2-isopropoxy-4-methylbenzoic acid (443 mg) in toluene (3.5 ml) were added thionyl chloride (0.331 ml) and N,N-dimethylformamide (8.4 mg) and the mixture was stirred at 80° C. for an hour. The mixture was evaporated in vacuo and the residue was dissolved in tetrahydrofuran (1.3 ml). The acid chloride solution was added to a solution of N-[2-(6-amino-3,4-dihydroisoquinolin-2(1H)-yl)ethyl]acetamide (409.6 mg) and triethylamine (0.367 ml) in tetrahydrofuran (2.0 ml) at ambient temperature and the mixture was stirred at the same temperature for 2 hours. The mixture was poured into water and extracted with ethyl acetate. The extract was washed with brine, dried over magnesium sulfate and evaporated in vacuo. The residue was purified by column chromatography on silica gel eluting with chloroform: methanol (95:5) to give N-{2-[2-(acetylamino)ethyl]-1,2,3,4-tetrahydro-6-isoquinolinyl}-2-isopropoxy-4-methylbenzamide (0.399 g) as pale yellow powder.

¹H-NMR(CDCl₃): δ 1.51(6H, d, J=5.9 Hz), 1.97(3H, s), 2.40(3H, s), 2.67(2H, t, J=5.9 Hz), 2.77(2H, t, J=5.9 Hz), 2.94(2H, t, J=5.9 Hz), 3.45(2H, q, J=5.9 Hz), 3.63(2H, s), 4.82(1H, sept, J=5.9 Hz), 6.17(1H, br s), 6.82(1H, s), 6.92(1H, d, J=7.8 Hz), 7.01(1H, d, J=8.1 Hz), 7.28(1H, dd, J=8.1, 2.4 Hz), 7.66(1H, d, J=1.9 Hz), 8.17(1H, d, J=7.8 Hz), 10.14(1H, s) ESI-MS (m/z): 410(M+H)⁺

### EXAMPLE 285

To a solution of N-[2-(6-amino-3,4-dihydroisoquinolin-2(1H)-yl)ethyl]acetamide (331.6 mg), 2-(dimethylamino)benzoic acid (280 mg) and 1-hydroxybenzotriazole (261 mg) in N,N-dimethylformamide (3.3 ml) was added 1-[3-(dimethylamino)propyl]-3 -ethylcarbodiimide hydrochloride (WSC.HCl) (327 mg), followed by triethylamine (0.296 ml) at ambient temperature. The reaction mixture was stirred for an hour at ambient temperature and concentrated in vacuo. The residue was dissolved in ethyl acetate and water, and extracted with ethyl acetate. The organic layer was washed with water and brine, dried over magnesium sulfate, filtered and concentrated in vacuo. The residue was purified by column chromatography on silica gel eluting with chloroform: methanol (95:5) to give N-{2-[2-(acetylamino)ethyl]-1,2,3,4-tetrahydro-6-isoquinolinyl}-2-(dimethylamino)benzamide (0.505 g) as a pale yellow foam.

¹H-NMR(CDCl₃): δ 1.97(3H, s), 2.66(2H, t, J=5.9 Hz), 2.76(2H, t, J=5.7 Hz), 2.83(6H, s), 2.94(2H, t, J=5.7 Hz), 3.44(2H, q, J=5.9 Hz), 3.62(2H, s), 6.17(1H, br s), 7.02(1H, d, J=8.1 Hz), 7.23-7.35(3H, m), 7.48(1H, ddd, J=7.8, 5.1, 1.6 Hz), 7.61(1H, d, J=1.9 Hz), 8.25(1H, dd, J=7.6, 1.1 Hz), 12.08(1H, s) ESI-MS(m/z): 381(M+H)⁺

### EXAMPLE 286

The following compound was obtained in substantially the same manner as in Example 182.

tert-Butyl 4-({[2-(4-methylphenyl)-3-pyridinyl]carbonyl}amino)phenyl[2-(1H-pyrazol-1-yl)ethyl]carbamate

¹H-NMR(CDCl₃): δ 1.39(9H, s), 2.40(3H, s), 3.98(2H, t, J=6.1 Hz), 4.32(2H, t, J=6.1 Hz), 6.23(1H, t, J=2.0 Hz), 6.83(2H, d, J=7.2 Hz), 7.09(2H, d, J=8.6 Hz), 7.14(1H, s), 7.27(2H, d, J=7.6 Hz), 7.34-7.39(2H, m), 7.46(1H, d, J=1.6 Hz), 7.58(2H, d, J=7.9 Hz), 8.15(1H, dd, J=7.9, 1.7 Hz), 8.76(1H, dd, J=4.8, 1.8 Hz) ESI-MS(m/z): 520(M+Na)⁺

### EXAMPLE 287

The following compound was obtained in substantially the same manner as in Example 183.

2-(4-Methylphenyl)-N-(4-{[2-(1H-pyrazol-1-yl)ethyl]amino}phenyl)nicotinamide

¹H-NMR(DMSO-d₆): δ 2.31(3H, s), 3.40(2H, q, J=6.3 Hz), 4.24(2H, t, J=6.3 Hz), 5.60(1H, t, J=6.0 Hz), 6.22(1H, t, J=2.0 Hz), 6.52(2H, d, J=8.9 Hz), 7.21(2H, d, J=8.0 Hz), 7.26(2H, d, J=8.9 Hz), 7.41-7.46(2H, m), 7.62(2H, d, J=8.2 Hz), 7.72(1H, d, J=1.7 Hz), 7.90(1H, dd, J=7.6, 1.7 Hz), 8.71(1H, dd, J=4.8, 1.8 Hz), 10.02(1H, s) ESI-MS(m/z): 398(M+H)⁺

### EXAMPLE 288

The following compound was obtained in substantially the same manner as in Example 182.

tert-Butyl 4-({[2-(4-ethylphenyl)-3-pyridinyl]carbonyl}amino)phenyl[2-(1H-pyrazol-1-yl)ethyl]carbamate

¹H-NMR(CDCl₃): δ 1.26(3H, t, J=7.6 Hz), 1.39(9H, s), 2.71(2H, q, J=7.6 Hz), 3.97(2H, t, J=6.3 Hz), 4.32(2H, t, J=6.3 Hz), 6.22(1H, t, J=2.0 Hz), 6.81(2H, d, J=7.3 Hz), 7.05(2H, d, J=8.9 Hz), 7.11(1H, s), 7.31(2H, d, J=8.2 Hz), 7.35-7.40(2H, m), 7.46(1H, d, J=1.6 Hz), 7.61(2H, d, J=8.2 Hz), 8.18(1H, dd, J=7.9, 1.0 Hz), 8.77(1H, dd, J=4.7, 1.8 Hz) ESI-MS(m/z): 534(M+Na)⁺

### EXAMPLE 289

The following compound was obtained in substantially the same manner as in Example 183.

2-(4-Ethylphenyl)-N-(4-{[2-(1H-pyrazol-1-yl)ethyl]amino}phenyl)nicotinamide

¹H-NMR(DMSO-d₆): δ 1.18(3H, t, J=7.6 Hz), 2.62(2H, q, J=7.6 Hz), 3.40(2H, q, J=0.3 Hz), 4.24(2H, t, J=6.3 Hz), 5.60(1H, t, J=6.0 Hz), 6.22(1H, t, J=2.0 Hz), 6.52(2H, d, J=8.9 Hz), 7.22-7.27(4H, m), 7.41-7.46(2H, m), 7.64(2H, d, J=8.2 Hz), 7.72(1H, d, J=2.3 Hz), 7.90(1H, dd, J=7.6, 1.7 Hz), 8.72(1H, dd, J=4.6, 1.7 Hz), 10.38(1H, s) ESI-MS(m/z): 412(M+H)⁺

### EXAMPLE 290

To a solution of 2-(4-methylphenyl)-1-cyclohexene-1-carboxylic acid (291 mg) in toluene (2.9 ml) were added thionyl chloride (0.195 ml) and N,N-dimethylformamide (1 drop) and the mixture was stirred at 80° C. for an hour. The mixture was evaporated in vacuo, and the residue was dissolved in tetrahydrofuran (1.0 ml). The acid chloride was added to a solution of tert-butyl 5-amino-2-pyridinyl[2-(1H-pyrazol-1-yl) ethyl]carbamate (314 mg) and triethylamine (0.22 ml) in tetrahydrofuran (2.14 ml) at ambient temperature and the mixture was stirred at the same temperature for 16 hours. The mixture was poured into water and extracted with ethyl acetate. The organic layer was washed with brine, dried over magnesium sulfate and evaporated in vacuo. The residue was purified by column chromatography on silica gel eluting with hexane:ethyl acetate (4:1→3:1→1:1) to give tert-butyl 5-({[2-(4-methylphenyl)-1-cyclohexen-1-yl]carbonyl}amino)-2-pyridinyl[2-(1H-pyrazol-1-yl)ethyl]carbamate (311 mg) as a pale yellow foam.

¹H-NMR(CDCl₃): δ 1.42(9H, s), 1.77(4H, m), 2.35(3H, s), 2.43(2H, br s), 2.53(2H, br s), 4.25(2H, t, J=5.4 Hz), 4.36(2H, t, J=5.4 Hz), 6.17(1H, t, J=2.2 Hz), 6.61(1H, br s), 7.17(4H, s), 7.30-7.31(2H, m), 7.42(1H, d, J=1.9 Hz), 7.55(1H, dd, J=8.4, 2.4 Hz), 7.73(1H, d, J=2.7 Hz) ESI-MS(m/z): 524(M+Na)⁺

### EXAMPLE 291

To a solution of tert-butyl 5-({[2-(4-methylphenyl)-1-cyclohexen-1-yl]carbonyl}amino)-2-pyridinyl[2-(1H-pyrazol-1-yl)ethyl]carbamate (304 mg) in dichloromethane (3 ml) was added trifluoroacetic acid (0.7 ml). The reaction mixture was stirred for 18 hours, quenched with 10% aqueous potassium carbonate solution, and extracted with dichloromethane. The organic layer was washed with brine, dried over magnesium sulfate, filtered and concentrated in vacuo. The residue was recrystallized from ethyl acetate-hexane to give 2-(4-methylphenyl)-N-(6-{[2-(1H-pyrazol-1-yl)ethyl]amino)-3-pyridinyl)-1-cyclohexene-1-carboxamide (182 mg) as pale yellow powder.

¹H-NMR(CDCl₃): δ 1.75-1.78(4H, m), 2.34(3H, s), 2.41(2H, br s), 2.51(2H, br s), 3.72(2H, q, J=5.7 Hz), 4.31(2H, t, J=5.7 Hz), 4.62(1H, br t, J=5.9 Hz), 6.21(1H, d, J=2.4 Hz), 6.24(1H, d, J=7.6 Hz), 6.41(1H, br s), 7.13-7.20(4H, m), 7.31-7.37(2H, m), 7.40(1H, br d, J=2.4 Hz), 7.52(1H, d, J=1.4 Hz) ESI-MS(m/z): 402(M+H)⁺

### EXAMPLE 292

The following compound was obtained in substantially the same manner as in Example 290.

tert-Butyl {5-[(2-isopropoxy-4-methylbenzoyl)amino]-2-pyridinyl}[2-(1H-pyrazol-1-yl)ethyl]carbamate

¹H-NMR(CDCl₃): δ 1.46(9H, s), 1.53(6H, d, J=6.5 Hz), 2.41(3H, s), 4.33(2H, t, J=5.1 Hz), 4.44(2H, t, J=5.7 Hz), 4.85(1H, sept, J=6.2 Hz), 6.20(1H, t, J=1.9 Hz), 6.84(1H, s), 6.93(1H, d, J=8.9 Hz), 7.37(1H, dd, J=2.4, 0.5 Hz), 7.45-7.49(2H, m), 8.17(1H, d, J=7.8 Hz), 8.27(1H, dd, J=8.9, 2.7 Hz), 8.44(1H, d, J=2.4 Hz), 10.28(1H, s) ESI-MS(m/z): 502(M+Na)⁺

### EXAMPLE 293

The following compound was obtained in substantially the same manner as in Example 291.

2-Isopropoxy-4-methyl-N-(6-{[2-(1H-pyrazol-1-yl)ethyl]amino}-3-pyridinyl)benzamide

¹H-NMR(CDCl₃): δ 1.50(6H, d, J=5.9 Hz), 2.39(3H, s), 3.81(2H, q, J=5.7 Hz), 4.38(2H, t, J=5.1 Hz), 4.70(1H, br t, J=5.9 Hz), 4.81(1H, sept, J=5.9 Hz), 6.24(1H, t, J=2.2 Hz), 6.41(1H, d, J=8.9 Hz), 6.81(1H, s), 6.91(1H, d, J=7.8 Hz), 7.36(1H, d, J=1.6 Hz), 7.55(1H, d, J=1.1 Hz), 8.07(1H, dd, J=8.9, 2.7 Hz), 8.14(2H, m), 10.01(1H, s) ESI-MS(m/z): 380(M+H)⁺

### EXAMPLE 294

A mixture of 2-(4-methylphenyl)-1-cyclohexene-1-carboxylic acid (325 mg), 4-[2-(1H-pyrazol-1-yl)ethoxy]phenylamine (321 mg), 1-hydroxybenzotriazole hydrate (242 mg) and 1-[3-(dimethylamino)propyl]-3-ethylcarbodiimide (245 mg) in N,N-dimethylformamide (20 ml) was stirred at ambient temperature for 15 hours. The reaction mixture was poured into a mixture of ethyl acetate and water, and the organic layer was washed with brine and dried over magnesium sulfate. The solvent was evaporated in vacuo and the residue was chromatographed on silica gel eluting with ethyl acetate: n-hexane (6:4 v/v). The eluting fraction was concentrated in vacuo and the precipitate was collected by filtration to give 2-(4-methylphenyl)-N-{4-[2-(1H-pyrazol-1-yl)ethoxy]phenyl}-1-cyclohexene-1-carboxamide (398 mg).

¹H-NMR(DMSO-d₆): δ 1.70(4H, br.s), 2.30(3H, s), 2.34(4H, br.s), 4.23(2H, t, J=5.3 Hz), 4.44(2H, t, J=5.3 Hz), 6.22-6.23(1H, m), 6.74(2H, d, J=9.3 Hz), 7.03(1H, d, J=8.1 Hz), 7.18(1H, d, J=8.1 Hz), 7.25(2H, d, J=9.3 Hz), 7.44(1H, d, J=1.4 Hz), 7.75(1H, d, J=2.0 Hz), 9.37(1H, s) ESI-MS(m/z): 424(M+Na)⁺, 402(M+H)⁺

### EXAMPLE 295

The following compound was obtained in substantially the same manner as in Example 294.

N-{4-[2-(1H-Pyrazol-1-yl)ethoxy]phenyl}-2-[4-(trifluoromethyl)phenyl]-1-cyclohexene-1-carboxamide

¹H-NMR(DMSO-d₆): δ 1.73(4H, br.s), 2.39(4H, br.s), 4.23(2H, t, J=5.2 Hz), 4.44(2H, t, J=5.2 Hz), 6.21-6.23(1H, m), 6.74 (2H, d, J=9.9 Hz), 7.21(2H, d, J=9.0 Hz),7.44(1H, d, J=1.7 Hz), 7.47(2H, d, J=8.3 Hz), 7.62 (2H, d, J=8.3 Hz), 7.74 (1H, d, J=2.2 Hz), 9.50 (1H, s) ESI-MS(m/z): 478(M+Na)⁺, 456(M+H)⁺

### EXAMPLE 296

The following compound was obtained in substantially the same manner as in Example 294.

2-[4-(Dimethylamino)phenyl]-N-{4-[2-(1H-pyrazol-1-yl)ethoxy]phenyl}-1-cyclohexene-1-carboxamide

¹H-NMR(DMSO-d₆): δ 1.68 (4H, br.s), 2.32 (4H, br.s), 2.81 (6H, s), 4.23 (2H, t, J=5.3 Hz), 4.44 (2H, t, J=5.3 Hz), 6.22-6.23 (1H, m), 6.65 (2H, d, J=8.8 Hz), 6.74 (2H, d, J=9.0 Hz), 7.14 (2H, d, J=8.8 Hz), 7.23 (2H, d, J=9.0 Hz), 7.44 (1H, d, J=1.5 Hz), 7.74 (1H, d, J=2.2 Hz), 9.30 (1H, s) ESI-MS(m/z): 453(M+Na)⁺, 431(M+H)⁺

### EXAMPLE 297

The following compound was obtained in substantially the same manner as in Example 294.

2-(4-Methylphenyl)-N-{6-[2-(1H-pyrazol-1-yl)ethoxy]-3-pyridinyl}-1-cyclohexene-1-carboxamide

¹H-NMR(DMSO-d₆): δ 1.71 (4H, br.s), 2.21 (3H, s), 2.36 (4H, br.s), 4.42-4.52 (4H, m), 6.21-6.23 (1H, m), 6.65 (1H, d, J=8.4 Hz), 7.05 (2H, d, J=8.0 Hz), 7.18 (2H, d, J=8.0 Hz),7.43 (1H, d, J=1.4 Hz), 7.64 (1H, dd, J=2.7, 8.8 Hz), 7.72 (1H, d, J=2.1 Hz), 8.09 (1H, d, J=2.7 Hz), 9.53 (1H, s) ESI-MS(m/z): 425(M+Na)⁺, 403(M+H)⁺

### EXAMPLE 298

The following compound was obtained in substantially the same manner as in Example 294.

N-{6-[2-(1H-Pyrazol-1-yl)ethoxy]-3-pyridinyl)-2-[4-(trifluoromethyl)phenyl]-1-cyclohexene-1-carboxamide

¹H-NMR(DMSO-d₆): δ 1.74 (4H, br.s), 2.40 (2H, br.s), 4.04-4.54 (4H, m), 6.20-6.22 (1H, m), 6.65 (1H, d, J=9.0 Hz), 7.42-7.76 (7H, m), 8.06 (1H, d, J=2.5 Hz), 9.67 (1H, s) ESI-MS(m/z): 479(M+Na)⁺, 457(M+H)⁺

### EXAMPLE 299

The following compound was obtained in substantially the same manner as in Example 294.

2-(4-Methylphenyl)-N-{4-[2-(1H-1,2,4-triazol-1-yl)ethoxy]phenyl}-1-cyclohexene-1-carboxamide

¹H-NMR(DMSO-d₆): δ 1.70(4H, br.s), 2.20(3H, s), 2.34(4H, br.s), 4.25(2H, t, J=5.0 Hz), 4.53(2H, t, J=5.0 Hz), 6.74(2H, d, J=9.0 Hz), 6.84(2H, d, J=9.0 Hz), 7.03(1H, d, J=8.0 Hz), 7.17(2H, d, J=8.0 Hz), 7.98(1H, s), 8.54(1H, s), 9.37(1H, s)

### EXAMPLE 300

The following compound was obtained in substantially the same manner as in Example 294.

N-{4-[2-(1H-1,2,4-Triazol-1-yl)ethoxy]phenyl}-2-[4-(trifluoromethyl)phenyl]-1-cyclohexene-1-carboxamide

¹H-NMR(DMSO-d₆): δ 1.73(4H, br.s), 2.39(4H, br.s), 4.25(2H, t, J=5.0 Hz), 4.53(2H, t, J=5.0 Hz), 6.75(2H, d, J=9.0 Hz), 7.22(2H, d, J=9.0 Hz), 7.48(2H, d, J=8.3 Hz), 7.62(2H, d, J=8.3 Hz), 7.93(1H, s), 8.54(1H, s), 9.51(1H, s)

### EXAMPLE 301

The following compound was obtained in substantially the same manner as in Example 294.

2-(4-Methylphenyl)-N-(4-{[2-(1H-1,2,4-triazol-1-yl)ethyl]amino}phenyl)-1-cyclohexene-1-carboxamide

¹H-NMR(DMSO-d₆): δ 1.69(4H, br.s), 2.22(3H, s), 2.33(4H, br.s), 3.24-3.43(2H, m), 4.27(2H, t, J=6.0 Hz), 5.52(1H, t, J=6.0 Hz), 6.41(2H, d, J=8.8 Hz), 7.02-7.08(4H, m), 7.18(1H, d, J=8.1 Hz), 7.97(1H, s), 8.44(1H, s), 9.10(1H, s) ESI-MS(m/z): 424(M+Na)⁺, 402(M+H)⁺

### EXAMPLE 302

The following compound was obtained in substantially the same manner as in Example 294.

N-(4-{[2-(1H-1,2,4-Triazol-1-yl)ethyl]amino}phenyl)-2-[4-(trifluoromethyl)phenyl]-1-cyclohexene-1-carboxamide

¹H-NMR(DMSO-d₆): δ 1.72(4H, br.s), 2.38(4H, br.s), 3.35-3.41(2H, m), 4.28(2H, t, J=6.1 Hz), 5.52(1H, t, J=6.1 Hz), 6.42(2H, d, J=8.8 Hz), 7.01(2H, d, J=8.8 Hz), 7.48(1H, d, J=8.2 Hz), 7.63(2H, d, J=8.2 Hz), 7.97(1H, s), 8.44(1H, s), 9.24(1H, s) ESI-MS(m/z): 477(M+Na)⁺, 456(M+H)⁺

### EXAMPLE 303

The following compound was obtained in substantially the same manner as in Example 294.

N-{4-[3-(1H-1,2,4-Triazol-1-yl)propyl]phenyl}-2-[4-(trifluoromethyl)phenyl]-1-cyclohexene-1-carboxamide

¹H-NMR(DMSO-d₆): δ 1.74 (4H, br.s), 1.87-2.16 (2H, m), 2.40 (4H, br.s), 2.04-2.48 (2H, m), 4.14 (2H, t, J=7.0 Hz), 7.02 (2H, d, J=8.4 Hz), 7.26 (2H, d, J=8.4 Hz), 7.49 (2H, d, J=8.3 Hz), 7.62 (2H, d, J=8.3 Hz), 7.96 (1H, s), 8.50 (1H, s), 9.61 (1H, s) ESI-MS(m/z): 477(M+Na)⁺, 455(M+H)⁺

### EXAMPLE 304

A mixture of 4′-(dimethylamino)-1,1′-biphenyl-2-carboxylic acid (242 mg), 4-[2-(1H-1,2,4-triazol-1-yl)ethoxy]aniline (215 mg), 1-hydroxybenzotriazole (142 mg) and 1-[3-(dimethylamino)propyl]-3-ethylcarbodiimide (163 mg) in N,N-dimethylformamide (20 ml) was stirred at ambient temperature for 15 hours. The reaction mixture was poured into a mixture of ethyl acetate and water, and the organic layer was washed with brine and dried over magnesium sulfate. The solvent was evaporated in vacuo and the residue was chromatographed on silica gel eluting with ethyl acetate:methanol (94:6 v/v). The eluted fractions were concentrated in vacuo and the precipitate was collected by filtration to give 4′-(dimethylamino)-N-{4-[2-(1H-1,2,4-triazol-1-yl)ethoxy]phenyl}-1,1′-biphenyl-2-carboxamide (300 mg).

¹H-NMR(DMSO-d₆): δ 2.88(6H, s), 4.29(2H, t, J=4.9 Hz), 4.55(2H, t, J=4.9 Hz), 6.70(2H, d, J=8.8 Hz), 6.83(2H, d, J=8.9 Hz), 7.29(2H, d, J=8.8 Hz), 7.36-7.53(6H, m), 7.99(1H, s), 8.56(1H,s), 10.06(1H,s) ESI-MS(m/z): 450(M+Na)⁺, 428(M+H)⁺

### EXAMPLE 305

The following compound was obtained in substantially the same manner as in Example 304.

N-(4-{[2-(1H-1,2,4-Triazol-1-yl)ethyl]amino}phenyl)-4′-(trifluoromethyl)-1,1′-biphenyl-2-carboxamide

¹H-NMR(DMSO-d₆): δ 3.38-3.47(2H, m), 4.30(2H, t, J=6.1 Hz), 5.61(1H, t, J=6.1 Hz), 6.49(2H, d, J=8.8 Hz), 7.22(2H, d, J=8.8 Hz), 7.46-7.65(6H, m), 7.76(2H, d, J=8.4 Hz), 7.98(1H, s), 8.46(1H, s), 9.54(1H, s) ESI-MS (m/z): 474 (M+Na)⁺, 452 (M+H)⁺

### EXAMPLE 306

The following compound was obtained in substantially the same manner as in Example 304.

4′-Methyl-N-(4-{[2-(1H-1,2,4-triazol-1-yl)ethyl]amino}phenyl)-1,1′-biphenyl-2-carboxamide

¹H-NMR(DMSO-d₆): δ 2.29(3H, s), 3.38-3.47(2H, m), 4.30(2H, t, J=6.1 Hz), 5.61(1H, t, J=6.1 Hz), 6.49(2H, d, J=8.8 Hz), 7.15-7.56(10H, m), 7.98(1H, s), 8.46(1H, s), 9.84(1H, s) ESI-MS(m/z): 420(M+Na)⁺, 398(M+H)⁺

### EXAMPLE 307

The following compound was obtained in substantially the same manner as in Example 304.

5-Methyl-N-(4-{[2-(1H-1,2,4-triazol-1-yl)ethyl]amino}phenyl)-4′-(trifluoromethyl)-1,1′-biphenyl-2-carboxamide

¹H-NMR(DMSO-d₆): δ 2.41(3H, s), 3.38-3.47(2H, m), 4.30(2H, t, J=6.1 Hz), 5.61(1H, t, J=6.0 Hz), 6.49(2H, d, J=8.8 Hz), 7.21(2H, d, J=8.8 Hz), 7.33(1H, d, J=7.6 Hz), 7.35(1H, s), 7.49(1H, d, J=7.6 Hz), 7.61(2H, d, J=8.3 Hz), 7.75(2H, d, J=8.3 Hz), 7.98(1H, s), 8.45(1H, s), 9.87(1H, s) ESI-MS(m/z): 488(M+Na)⁺, 466(M+H)⁺

### EXAMPLE 308

The following compound was obtained in substantially the same manner as in Example 304.

4′,6-Dimethyl-N-{4-[2-(1H-1,2,4-triazol-1-yl)ethoxy]phenyl}-1,1′-biphenyl-2-carboxamide

¹H-NMR(DMSO-d₆): δ 2.08(3H, s), 2.78(3H, s), 4.27(2H, t, J=5.0 Hz), 4.54(2H, t, J=5.0 Hz), 6.77(2H, d, J=9.0 Hz), 7.14(3H, s), 7.29-7.42(6H, m), 7.98(1H, s), 8.55(1H, s), 9.87(1H, s) ESI-MS(m/z): 435(M+Na)⁺, 413(M+H)⁺

### EXAMPLE 309

The following compound was obtained in substantially the same manner as in Example 304.

4′,5-Dimethyl-N-{4-[2-(1H-1,2,4-triazol-1-yl)ethoxy]phenyl}-1,1′-biphenyl-2-carboxamide

¹H-NMR(DMSO-d₆): δ 2.28(3H, s), 2.38(3H,s), 4.29(2H, t, J=5.0 Hz), 4.55(2H, t, J=5.0 Hz), 6.82(2H, d, J=9.0 Hz), 7.15(2H, d, J=8.0 Hz), 7.24-7.33(5H, m), 7.41(2H, d, J=8.8 Hz), 7.99(1H, s), 8.56(1H, s), 9.99(1H, s) ESI-MS (m/z): 435 (M+Na)⁺, 413 (M+H)⁺

### EXAMPLE 310

The following compound was obtained in substantially the same manner as in Example 294.

N-{4-[2-(1H-Pyrazol-1-yl)ethoxy]phenyl}-4′-(trifluoromethyl)-1,1′-biphenyl-2-carboxamide

¹H-NMR(DMSO-d₆): δ 4.30(2H, t, J=5.2 Hz), 4.46(2H, t, J=5.2 Hz), 6.23-6.25(1H, m), 6.83(2H, d, J=9.0 Hz), 7.39-7.64(9H, m), 7.73-7.77(3H, m), 10.22(1H, s)

### EXAMPLE 311

The following compound was obtained in substantially the same manner as in Example 294.

4′-(Dimethylamino)-N-{4-[2-(1H-pyrazol-1-yl)ethoxy]phenyl}-1,1′-biphenyl-2-carboxamide

¹H-NMR(DMSO-d₆): δ 2.87(6H, s), 4.27(2H, t, J=5.3 Hz), 4.46(2H, t, J=5.3 Hz), 6.23-6.25(1H, m), 6.70(2H, d, J=8.8 Hz), 6.83(2H, d, J=9.0 Hz), 7.86(2H, d, J=8.7 Hz), 7.31-7.53(7H, m), 7.70(1H, d, J=2.0 Hz), 10.06(1H, s) ESI-MS(m/z): 449(M+Na)⁺, 427(M+H)⁺

### EXAMPLE 312

The following compound was obtained in substantially the same manner as in Example 294.

N-{6-[2-(1H-Pyrazol-1-yl)ethoxy]-3-pyridinyl}-4′-(trifluoromethyl)-1,1′-biphenyl-2-carboxamide

¹H-NMR(DMSO-d₆): δ 4.44-4.59(4H, m), 6.22-6.24(1H, m), 6.75(1H, d J=9.0 Hz), 7.44(1H, d, J=1.5 Hz), 7.51-7.82(10H, m), 8.28(1H, d, J=2.4 Hz),10.39(1H, S) ESI-MS(m/z): 475(M+Na)⁺, 453(M+H)⁺

### EXAMPLE 313

The following compound was obtained in substantially the same manner as in Example 294.

4′,5-Dimethyl-N-{6-[2-(1H-pyrazol-1-yl)ethoxy]-3-pyridinyl}-1,1′-biphenyl-2-carboxamide

¹H-NMR(DMSO-d₆): δ 2.29(3H, s), 2.40(3H, s), 4.43-4.58(4H, m), 6.22-6.24(1H, m), 6.74(1H, d, J=8.9 Hz), 7.17(2H, d, J=8.0 Hz), 7.26-7.33(4H, m), 7.43-7.47(2H, m), 7.73-7.82(2H, m), 8.27(1H, d, J=2.5 Hz), 10.16(1H, s) ESI-MS(m/z): 435(M+Na)⁺, 413(M+H)⁺

### EXAMPLE 314

The following compound was obtained in substantially the same manner as in Example 294.

4′,5-Dimethyl-N-{4-[2-(1H-pyrazol-1-yl)ethoxy]phenyl}-1,1′-biphenyl-2-carboxamide

¹H-NMR(DMSO-d₆): δ 2.28(3H, s), 2.39(3H, s), 4.27(2H, d, J=5.3 Hz), 4.46(2H, d, J=5.3 Hz), 6.23-6.25(1H, m), 6.81(2H, d, J=8.1 Hz), 7.23-7.46(8H, m), 7.76(1H, d, J=2.1 Hz), 9.99(1H, s) ESI-MS(m/z): 434(M+Na)⁺, 412(M+H)⁺

### EXAMPLE 315

The following compound was obtained in substantially the same manner as in Example 294.

4′-Methoxy-5-methyl-N-{4-[2-(1H-pyrazol-1-yl)ethoxy]phenyl}-1,1′-biphenyl-2-carboxamide

¹H-NMR(DMSO-d₆): δ 2.39(3H, s), 3.73(3H, s), 4.27(2H, t, J=5.3 Hz), 4.46(2H, t, J=5.3 Hz), 6.23-6.25(1H, m), 6.81(2H, d, J=9.0 Hz), 6.92(2H, d, J=7.1 Hz), 7.21-7.24(2H, m), 7.32-7.46(6H, m), 7.76(1H, d, J=2.2 Hz), 9.97(1H, s) ESI-MS (m/z): 450 (M+Na)⁺, 428 (M+H)⁺

### EXAMPLE 316

The following compound was obtained in substantially the same manner as in Example 294.

4′-Chloro-5-methyl-N-{4-[2-(1H-pyrazol-1-yl)ethoxy]phenyl}-1,1′-biphenyl-2-carboxamide

¹H-NMR(DMSO-d₆): δ 2.40(3H, s), 4.27(2H, t, J=5.3 Hz), 4.46(2H, t, J=5.3 Hz), 6.23-6.25(1H, m), 6.82(2H, d, J=9.0 Hz), 7.29(2H, d, J=8.4 Hz), 7.38-7.48(8H, m), 7.76(1H, d, J=2.2 Hz), 10.05(1H, s) ESI-MS(m/z): 454(M+Na)⁺, 432(M+H)⁺

### EXAMPLE 317

The following compound was obtained in substantially the same manner as in Example 294.

4′-(Dimethylamino)-5-methyl-N-{4-[2-(1H-pyrazol-1-yl)ethoxy]phenyl}-1,1′-biphenyl-2-carboxamide

¹H-NMR(DMSO-d₆): δ 2.37(3H, s), 2.88(6H, s), 4.27(2H, t, J=5.2 Hz), 4.46(2H, t, J=5.2 Hz), 6.23-6.25(1H, m), 6.69(2H, d, J=8.7 Hz), 6.82(2H, d, J=9.0 Hz), 7.14-7.46(8H, m), 7.76(1H, d, J=2.0 Hz), 9.95(1H, s) ESI-MS(m/z): 463(M+Na)⁺, 441(M+H)⁺

### EXAMPLE 318

The following compound was obtained in substantially the same manner as in Example 304.

N-{4-[3-(1H-1,2,4-Triazol-1-yl)propyl]phenyl}-4′-(trifluoromethyl)-1,1′-biphenyl-2-carboxamide

¹H-NMR(DMSO-d₆): δ 1.99-2.12(2H, m), 2.37-2.49(2H, m), 4.16(2H, t, J=7.0 Hz), 7.11(2H, d, J=8.8 Hz), 7.45(2H, d, J=8.4 Hz), 7.49-7.66(6H, m), 7.76(2H, d, J=8.3 Hz), 7.97(1H, s), 8.52(1H, s), 10.32(1H,s) ESI-MS(m/z): 473(M+Na)⁺, 451(M+H)⁺

### EXAMPLE 319

The following compound was obtained in substantially the same manner as in Example 304.

4′-(Dimethylamino)-N-{4-[3-(1H-1,2,4-triazol-1-yl)propyl]phenyl}-1,1′-biphenyl-2-carboxamide

¹H-NMR(DMSO-d₆): δ 1.99-2.18(2H, m), 2.40-2.50(2H, m), 2.88(6H, s), 4.16(2H, t, J=6.9 Hz), 6.70(2H, d, J=8.7 Hz), 7.10(2H, d, J=8.3 Hz), 7.29(2H, d, J=8.7 Hz), 7.32-7.51(6H, m), 7.97(1H, s), 8.50(1H, s), 10.16(1H,s) ESI-MS(m/z): 448(M+Na)⁺, 425(M+H)⁺

Preparation 164

The following compound was obtained in substantially the same manner as in Preparation 96.

1-[2-(4-Nitrophenoxy)ethyl]-1H-pyrrole

¹H-NMR(DMSO-d₆): δ 4.27-4.46(4H, m), 6.00-6.01(2H, m), 6.83-6.85(2H, m), 7.09-7.17(2H, m), 8.15-8.23(2H, m)

Preparation 165

The following compound was obtained in substantially the same manner as in Preparation 97.

4-[2-(1H-Pyrrol-1-yl)ethoxy]aniline

¹H-NMR(DMSO-d₆): δ 3.97-4.07(2H, m), 4.14-4.19(2H, m), 4.62(2H, s), 5.91-5.99(2H, m), 6.45-6.52(2H, m), 6.56-6.68(2H, m), 6.77-6.81(2H, m)

### EXAMPLE 320

The following compound was obtained in substantially the same manner as in Example 304.

N-{4-[2-(1H-Pyrrol-1-yl)ethoxy]phenyl}-2-[4-(trifluoromethyl)phenyl]-1-cyclohexene-1-carboxamide

¹H-NMR(DMSO-d₆): δ 1.74(4H, br.s), 2.39(4H, br.s), 4.01-4.22(4H, m), 5.96-5.98(2H, m), 6.73-6.80(4H, m), 7.22(2H, d, J=9.0 Hz), 7.48(2H, d, J=8.1 Hz), 7.62(2H, d, J=8.1 Hz), 9.49(1H, s) ESI-MS(m/z): 477(M+Na)⁺, 455(M+H)⁺

### EXAMPLE 321

The following compound was obtained in substantially the same manner as in Example 304.

N-{4-[2-(1H-Pyrrol-1-yl)ethoxy]phenyl}-4′-(trifluoromethyl)-1,1′-biphenyl-2-carboxamide

¹H-NMR(DMSO-d₆): δ 4.16-4.24(4H, m), 5.98-6.00(2H, m), 6.80-6.96(3H, m), 7.42(2H, d, J=9.0 Hz), 7.48-7.65(5H, m), 7.75(2H, d, J=8.3 Hz), 10.21(1H, s) ESI-MS(m/z): 473(M+Na)⁺, 451(M+H)⁺

Preparation 166

A solution of chloroacetylchoride (357 mg) in tetrahydrofuran (5 ml) was dropwise added to a mixture of N-(2,3-dihydro-1H-indol-5-yl)-4′-(trifluoromethyl)-1,1′-biphenyl-2-carboxamide (1.15 g) and triethylamine (670 mg) in tetrahydrofuran (30 ml) at 5-20° C. under stirring and the resultant mixture was stirred at ambient temperature for 6 hours. The reaction mixture was poured into a mixture of ethyl acetate and water, and the organic layer was washed with brine and dried over magnesium sulfate. The solvent was evaporated in vacuo and the residue was chromatographed on silica gel eluting with ethyl acetate and n-hexane (6:4). The fraction was concentrated in vacuo and the precipitate was collected by filtration to give N-[1-(chloroacetyl)-2,3-dihydro-1H-indol-5-yl]-4′-(trifluoromethyl)-1,1′-biphenyl-2-carboxamide (1.09 g).

¹H-NMR(DMSO-d₆): δ 3.13(2H, t, J=8.3 Hz), 4.11(2H, t, J=8.3 Hz), 4.51(2H, s), 7.27(1H, dd, J=1.8, 8.6 Hz), 7.50-7.65(7H, m), 7.76(2H, d, J=8.4 Hz), 7.93(1H, d, J=8.6 Hz), 10.33(1H, s)

### EXAMPLE 322

A mixture of imidazole (136 mg) and potassium tert-butoxide (225 mg) in N,N-dimethylformamide (10 ml) was stirred at ambient temperature for 30 minutes. N-[1-(chloroacetyl)-2,3-dihydro-1H-indol-5-yl]-4′-(trifluoromethyl)-1,1′-biphenyl-2-carboxamide (460 mg) was added to an above mixture and the resultant mixture was stirred at 65-70° C. for 6 hours. The reaction mixture was poured into a mixture of ethyl acetate and water, and the organic layer was washed with brine and dried over magnesium sulfate. The solvent was concentrated in vacuo and the precipitate was collected by filtration to give N-[1-(1H-imidazol-1-ylacetyl)-2,3-dihydro-1H-indol-5-yl]-4′-(trifluoromethyl)-1,1′-biphenyl-2-carboxamide (340 mg).

¹H-NMR(DMSO-d₆): δ 3.17(2H, t, J=8.3 Hz), 4.15(2H, t, J=8.3 Hz), 5.09(2H, s), 6.89(1H, s), 7.11(1H, s), 7.22(1H, dd, J=1.6, 8.7 Hz), 7.49-7.65(8H, m), 7.76(2H, d, J=8.3 Hz), 7.88(1H, d, J=8.7 Hz), 10.32(1H, s) ESI-MS(m/z): 513(M+Na)⁺, 491(M+H)⁺

### EXAMPLE 323

A mixture of N-[1-(chloroacetyl)-2,3-dihydro-1H-indol-5-yl]-4′-(trifluoromethyl)-1,1′-biphenyl-2-carboxamide (460 mg) and 1,2,4-triazole sodium salt (128 mg) in N,N-dimethylformamide (10 ml) was stirred at 65-70° C. for 4.5 hours. The reaction mixture was poured into a mixture of ethyl acetate and water, and the organic layer was washed with brine and dried over magnesium sulfate. The solvent was evaporated in vacuo and the residue was chromatographed on silica gel eluting with ethyl acetate: methanol (95:5-90:10 v/v). The eluted fractions were concentrated in vacuo and the precipitate was collected by filtration to give N-[1-(1H-1,2,4-triazol-1-ylacetyl)-2,3-dihydro-1H-indol-5-yl]-4′-(trifluoromethyl)-1,1′-biphenyl-2-carboxamide (370 mg).

¹H-NMR(DMSO-d₆): δ 3.18(2H, t, J=8.3 Hz), 4.19(2H, t, J=8.3 Hz), 5.37(2H, s), 7.22-7.25(1H, m), 7.51-7.64(7H, m), 7.77(2H, d, J=8.3 Hz), 7.86(1H, d, J=8.7 Hz), 8.00(1H, s), 8.50(1H, s), 10.33(1H, s) negative ESI-MS(m/z): 490(M−H)⁻

### EXAMPLE 324

The following compound was obtained in substantially the same manner as in Example 186.

2-Isopropoxy-4-methyl-N-[2-(2-pyridinylacetyl)-2,3-dihydro-1H-isoindol-5-yl]benzamide

¹H-NMR(DMSO-d₆): δ 1.37(6H, t, J=6.0 Hz), 2.36(3H, s), 3.4-3.8(6H, m), 4.81(1H, septet, J=6.0 Hz.), 6.8-7.8(8H, m), 8.51(1H, d, J=4.5 Hz), 10.03(1H, s) ESI-MS(m/z): 452(M+Na)⁺, 430(M+H)⁺

Preparation 167

To a solution of ethyl 2-methyl-6-oxo-1,6-dihydro-5-pyrimidinecarboxylate (9.109 g) and diisopropylethylamine (7.75 g) in 1,2-dichloroethane (200 ml) was added dropwise trifluoromethanesulfonic anhydride (15.5 g) at 5° C. and the mixture was stirred at ambient temperature for 20 hours. The mixture was poured into iced water (100 ml) and the separated organic layer was washed with water and brine, dried over magnesium sulfate and dried in vacuo. The residue was purified by flash chromatography on silica gel eluting with ethyl acetate to give crude ethyl 2-methyl-4-{[(trifluoromethyl)sulfonyl]oxy}-5-pyrimidinecarboxylate (14.69 g) as a dark-brown oil.

¹H-NMR(DMSO-d₆): δ 1.26(3H, t, J=7.1 Hz), 2.35(3H, s), 4.21(2H, q, J=7.1 Hz), 8.44(1H, s) ESI-MS(m/z): 337(M+H)⁺

Preparation 168

To a solution of ethyl 2-methyl-4-{[(trifluoromethyl)sulfonyl]oxy}-5-pyrimidinecarboxylate (14.67 g) in acetonitrile (70 ml) was added 4-methylpiperidine (13.9 g) and the mixture was refluxed for 16 hours. The mixture was evaporated in vacuo and the residue was purified by column chromatography on silica gel eluting with ethyl acetate to give ethyl 2-methyl-4-(4-methyl-1-piperidinyl)-5-pyrimidinecarboxylate (10.23 g) as a yellow oil.

¹H-NMR(DMSO-d₆): δ 0.92(3H, d, J=6.1 Hz), 1.0-1.3(3H, m), 1.32(3H, t, J=7.2 Hz), 1.6-1.8(2H, m), 2.41(3H, s), 2.85-3.05(2H, m), 3.9-4.05(2H, m), 4.26(2H, q, J=7.2 Hz), 8.42(1H, s) ESI-MS(m/z): 286(M+Na)⁺, 264(M+H)⁺

Preparation 169

To a solution of ethyl 2-methyl-4-(4-methyl-1-piperidinyl)-5-pyrimidinecarboxylate (10.20 g) in ethanol (50 ml) was added 5N aqueous sodium hydroxide solution (15.5 ml) and the mixture was refluxed for 5 hours. The mixture was cooled to 5° C., adjusted to pH 7 by addition of 6N hydrochloric acid and evaporated in vacuo to remove ethanol. The residue was adjusted to pH 5 by addition of 6N hydrochloric acid and extracted with ethyl acetate. The separated organic layer was washed with brine, dried over magnesium sulfate and dried in vacuo. The residue was triturated with diisopropyl ether and collected by filtration to give 2-methyl-4-(4-methyl-1-piperidinyl)-5-pyrimidinecarboxylic acid (4.74 g) as a white crystal.

¹H-NMR(DMSO-d₆): δ 0.91(3H, d, J=6.0 Hz), 1.0-1.3(2H, m), 1.55-1.7(3H, m), 2.39(3H, s), 2.9-3.1(2H, m), 4.0-4.2(2H, m), 8.39(1H, s) ESI-MS(m/z): 258(M+Na)⁺, 236(M+H)⁺

### EXAMPLE 325

To a solution of 4-aminophenyl[2-(2-pyridinyl)ethyl]formamide (724 mg), 2-methyl-4-(4-methyl-1-piperidinyl)-5-pyrimidinecarboxylic acid (706 mg) and benzotriazol-1-yl-oxytripyrrolidinophosphonium hexafluorophosphate (PyBOP) (1.87 g) in N,N-dimethylformamide (30 ml) was added diisopropylethylamine (776 mg) at ambient temperature and the mixture was stirred at the same temperature for 20 hours. The mixture was poured into a mixture of ethyl acetate, water and 6N hydrochloric acid. The separated organic layer was washed with water and brine, dried over magnesium sulfate and evaporated in vacuo. The residue was purified by column chromatography on silica gel eluting with ethyl acetate to give N-(4-{formyl[2-(2-pyridinyl)ethyl]amino}phenyl)-2-methyl-4-(4-methyl-1-piperidinyl)-5-pyrimidinecarboxamide (926 mg) as a pale brown powder.

¹H-NMR(DMSO-d₆): δ 0.89(3H, t, J=5.9 Hz), 1.0-1.25(2H, m), 1.55-1.8(3H, m), 2.42(3H, s), 2.8-3.1(4H, m), 3.9-4.2(4H, m), 7.1-7.3(4H, m), 7.6-7.75(3H, m), 8.23(1H, s), 8.34(1H, s), 8.45-8.5(1H, m), 10.55(1H, s) negative ESI-MS(m/z): 457(M−H)⁻

### EXAMPLE 326

To a suspension of N-(4-{formyl[2-(2-pyridinyl)ethyl]amino}phenyl)-2-methyl-4-(4-methyl-1-piperidinyl)-5-pyrimidinecarboxamide (910 mg) in methanol (10 ml) was added concentrated hydrochloric acid (0.83 ml) at ambient temperature and the resultant solution was stirred at the same temperature for 20 hours. The solution was poured into a mixture of ethyl acetate and water and adjusted to pH 9 by addition of 50% potassium carbonate aqueous solution. The separated organic layer was washed with water and brine, dried over magnesium sulfate and evaporated in vacuo. The residue was purified by column chromatography on silica gel eluting with ethyl acetate to give 2-methyl-4-(4-methyl-1-piperidinyl)-N-(4-{[2-(2-pyridinyl)ethyl]amino}phenyl)-5-pyrimidinecarboxamide (436 mg) as a pale brown powder.

¹H-NMR(DMSO-d₆): δ 0.88(3H, d, J=6.0 Hz), 1.0-1.3(2H, m), 1.5-1.7(3H, m), 2.41(3H, s), 2.8-3.05(4H, m), 3.36(2H, t, J=7.0 Hz), 4.05-4.25(2H, m), 5.85(1H, br), 6.57(2H, d, J=8.9 Hz), 7.2-7.3(1H, m), 7.31(1H, d, J=7.9 Hz), 7.38(2H, d, J=8.9 Hz), 7.65-7.75(1H, m), 8.17(1H, s), 8.5-8.55(1H, m), 10.07(1H, s) negative ESI-MS(m/z): 429(M−H)⁻

Preparation 170

The following compound was obtained in substantially the same manner as in Preparation 168.

Ethyl 4-(4-methyl-1-piperidinyl)-2-(trifluoromethyl)-5-pyrimidinecarboxylate

¹H-NMR(DMSO-d6): δ 0.90(3H, t, J=6.1 Hz), 1.1-1.4(2H, m), 1.31(3H, t, J=7.1 Hz), 1.6-1.9(3H, m), 3.0-3.2(2H, m), 3.9-4.1(2H, m), 4.32(2H, q, J=7.1 Hz), 8.64(1H, s) ESI-MS(m/z): 340(M+Na)⁺, 318(M+H)⁺

Preparation 171

The following compound was obtained in substantially the same manner as in Preparation 169.

4-(4-Methyl-1-piperidinyl)-2-(trifluoromethyl)-5-pyrimidinecarboxylic acid

¹H-NMR(DMSO-d₆): δ 0.92(3H, d, J=6.1 Hz), 1.05-1.3(2H, m), 1.6-1.8(3H, m), 2.95-3.2(2H, m), 3.95-4.15(2H, m), 8.62(1H, s), 13.65(1H, brs) negative ESI-MS(m/z): 288(M−H)⁻

### EXAMPLE 327

The following compound was obtained in substantially the same manner as in Example 325.

N-(4-{Formyl[2-(2-pyridinyl)ethyl]amino}phenyl)-4-(4-methyl-1-piperidinyl)-2-(trifluoromethyl)-5-pyrimidinecarboxamide

¹H-NMR(DMSO-d₆): δ 0.90(3H, d, J=6.0 Hz), 1.0-1.3(2H, m), 1.5-1.8(3H, m), 2.85-2.95(2H, m), 4.05-4.3(4H, m), 7.15-7.4(4H, m), 7.65-7.75(3H, m), 8.36(1H, s), 8.45-8.5(1H, m), 8.50(1H, s), 10.78(1H, s) ESI-MS(m/z): 535(M+Na)⁺, 513(M+H)⁺

### EXAMPLE 328

The following compound was obtained in substantially the same manner as in Example 326.

4-(4-Methyl-1-piperidinyl)-N-(4-{[2-(2-pyridinyl)ethyl]amino}phenyl)-2-(trifluoromethyl)-5-pyrimidinecarboxamide

¹H-NMR(DMSO-d₆): δ 0.89(3H, t, J=6.0 Hz), 1.0-1.3(2H, m), 1.6-1.8(3H, m), 2.98(2H, t, J=6.8 Hz), 2.95-3.15(2H, m), 3.35(2H, dd, J=6.8 and 5.7 Hz), 4.1-4.3(2H, m), 5.66(1H, t, J=5.7 Hz), 6.59(2H, d, J=8.9 Hz), 7.15-7.25(1H, m), 7.32(1H, d, J=7.8 Hz), 7.38(2H, d, J=8.9 Hz), 7.65-7.75(1H, m), 8.41(1H, s), 8.5-8.55(1H, m), 10.30(1H, s) ESI-MS(m/z): 507(M+Na)⁺, 485(M+H)⁺

Preparation 172

The following compound was obtained in substantially the same manner as in Preparation 168.

Ethyl 4-(4-methyl-1-piperidinyl)-2-(methylthio)-5-pyrimidinecarboxylate

¹H-NMR(DMSO-d₆): δ 0.92(3H, d, J=6.0 Hz), 1.0-1.3(2H, m), 1.6-1.8(3H, m), 2.46(3H, s), 2.9-3.1(2H, m), 3.9-4.05(2H, m), 4.25(2H, q, J=7.1 Hz), 8.37(1H, s) ESI-MS(m/z): 318(M+Na)⁺, 296(M+H)⁺

Preparation 173

4-(4-Methyl-1-piperidinyl)-2-(methylthio)-5-pyrimidinecarboxylic acid was obtained in substantially the same manner as in Preparation 169. This compound was used in Example 329 without purification.

### EXAMPLE 329

The following compound was obtained in substantially the same manner as in Example 325.

N-(4-{Formyl[2-(2-pyridinyl)ethyl]amino}phenyl)-4-(4-methyl-1-piperidinyl)-2-(methylthio)-5-pyrimidinecarboxamide

¹H-NMR(DMSO-d₆): δ 0.89(3H, d, J=5.9 Hz), 1.0-1.3(2H, m), 1.6-1.8(3H, m), 2.54(3H, s), 2.8-3.1(4H, m), 3.85-4.0(2H, m), 4.0-4.2(2H, m), 6.56(2H, d, J=8.6 Hz), 6.90(2H, d, J=8.6 Hz), 7.15-7.3(4H, m), 7.65-7.75(3H, m), 8.18(1H, s), 8.33(1H, s), 8.45-8.5(1H, m), 10.52(1H, s)

### EXAMPLE 330

The following compound was obtained in substantially the same manner as in Example 326.

4-(4-Methyl-1-piperidinyl)-2-(methylthio)-N-(4-{[2-(2-pyridinyl)ethyl]amino}phenyl)-5-pyrimidinecarboxamide

¹H-NMR(DMSO-d₆): δ 0.88(3H, t, J=6.0 Hz), 1.0-1.3(2H, m), 1.55-1.75(3H, m), 2.46(3H, s), 2.85-3.05(2H, m), 2.98(2H, t, J=7.2 Hz), 3.35(2H, td, J=7.2, 5.7 Hz), 4.1-4.3(2H, m), 5.59(1H, s), 6.57(2H, d, J=8.8 Hz), 7.15-7.25(1H, m), 7.31(1H, d, J=7.7 Hz), 7.37(1H, d, J=8.8 Hz), 7.65-7.8(1H, m), 8.10(1H, s), 8.5-8.55(1H, m), 10.04(1H, s)

### EXAMPLE 331

To a solution of 4-{2-[3-(2,5-dimethyl-1H-pyrrol-1-yl)-1H-pyrazol-1-yl]ethoxy}aniline (1.48 g) in dichloromethane (40 ml) was added triethylamine, followed by dropwise addition of a solution of 4′-(trifluoromethyl)-1,1′-biphenyl-2-carbonyl chloride (1.42 g) in dichloromethane (10 ml) at ambient temperature and the mixture was stirred for 5 hours at the same temperature. The mixture was poured into water and the separated organic layer was washed with brine, dried over magnesium sulfate and evaporated in vacuo. The residue was purified by column chromatography on silica gel eluting with hexane: ethyl acetate (2:1) to give N-(4-{2-[3-(2,5-dimethyl-1H-pyrrol-1-yl)-1H-pyrazol-1-yl]ethoxy}phenyl)-4′-(trifluoromethyl)-1,1′-biphenyl-2-carboxamide (2.31 g) as white powder.

¹H-NMR(DMSO-d₆): δ 1.94(6H, s), 4.21(4H, s), 5.45(1H, d, J=2.3 Hz), 5.73(2H, s), 6.27(1H, d, J=2.3 Hz), 7.3-7.8(12H, m), 10.21(1H, s) negative ESI-MS(m/z): 543(M−H)⁻

### EXAMPLE 332

To a suspension of N-(4-{2-[3-(2,5-dimethyl-1H-pyrrol-1-yl)-1H-pyrazol-1-yl]ethoxy}phenyl)-4′-(trifluoromethyl)-1,1′-biphenyl-2-carboxamide (2.29 g) in a mixture of ethanol (40 ml) and water (10 ml) were added hydroxylamine hydrochloride (2.92 g) and triethylamine (851 mg) at ambient temperature. The mixture was refluxed for 6 hours and evaporated to dryness. The residue was extracted from ethyl acetate and the organic layer was washed with brine, dried over magnesium sulfate and evaporated in vacuo. The residue was purified by column chromatography on silica gel eluting with ethyl acetate to give N-{4-[2-(3-amino-1H-pyrazol-1-yl)ethoxy]phenyl}-4′-(trifluoromethyl)-1,1′-biphenyl-2-carboxamide (890 mg) as a white crystal.

¹H-NMR(DMSO-d₆): δ 4.17(4H, s), 4.56(2H, brs), 5.37(1H, d, J=2.1 Hz), 6.82(2H, d, J=9.0 Hz), 7.34(1H, d, J=2.1 Hz), 7.41(2H, d, J=9.0 Hz), 7.5-7.7(7H, m), 7.75(2H, d, J=8.3 Hz), 10.21(1H, s)

Preparation 174

The following compound was obtained in substantially the same manner as in Preparation 124.

2-[5-(2,5-Dimethyl-1H-pyrrol-1-yl)-1H-pyrazol-1-yl]ethanol

¹H-NMR(DMSO-d₆): δ 1.91(6H, s), 3.6-3.7(4H, m), 4.83(1H, t, J=5.3 Hz), 5.85(2H, s), 6.33(1H, d, J=1.7 Hz), 7.62(1H, d, J=1.7 Hz)

Preparation 175

The following compound was obtained in substantially the same manner as in Preparation 125.

5-(2,5-Dimethyl-1H-pyrrol-1-yl)-1-[2-(4-nitrophenoxy)ethyl]-1H-pyrazole

¹H-NMR(DMSO-d₆): δ 1.96(6H, s), 4.04(2H, t, J=5.0 Hz), 4.48(2H, t, J=5.0 Hz), 5.89(2H, s), 6.41(1H, d, J=2.0 Hz), 7.0-7.1(2H, m), 7.67(1H, d, J=2.0 Hz), 8.15-8.25(2H, m) ESI-MS(m/z): 349(M+Na)⁺

Preparation 176

The following compound was obtained in substantially the same manner as in Preparation 126.

4-{2-[5-(2,5-Dimethyl-1H-pyrrol-1-yl)-1H-pyrazol-1-yl]ethoxy}aniline

¹H-NMR(DMSO-d₆): δ 1.91(6H, s), 3.92(2H, t, J=5.1 Hz), 4.12(2H, t, J=5.1 Hz), 4.61(2H, brs), 5.88(2H, s), 6.37(1H, d, J=1.9 Hz), 6.4-6.6(4H, m), 7.65(1H, d, J=1.9 Hz)

### EXAMPLE 333

The following compound was obtained in substantially the same manner as in Example 331.

N-(4-{2-[5-(2,5-Dimethyl-1H-pyrrol-1-yl)-1H-pyrazol-1-yl]ethoxy}phenyl)-4′-(trifluoromethyl)-1,1′-biphenyl-2-carboxamide

¹H-NMR(DMSO-d₆): δ 1.95(6H, s), 3.98(2H, t, J=4.9 Hz), 4.24(2H, t, J=4.9 Hz), 5.89(2H, s), 6.39(1H, d, J=1.9 Hz), 6.73(2H, d, J=9.0 Hz), 7.38(2H, d, J=9.0 Hz), 7.45-7.7(7H, m), 7.75(2H, d, J=8.3 Hz), 10.20(1H, s) ESI-MS(m/z): 567(M+Na)⁺

### EXAMPLE 334

The following compound was obtained in substantially the same manner as in Example 332.

N-{4-[2-(5-Amino-1H-pyrazol-1-yl)ethoxy]phenyl}-4′-(trifluoromethyl)-1,1′-biphenyl-2-carboxamide

¹H-NMR(DMSO-d₆): δ 4.19(4H, s), 5.16(2H, brs), 5.27(1H, d, J=1.7 Hz), 6.84(2H, d, J=9.0 Hz), 7.06(1H, d, J=1.7 Hz), 7.41(2H, d, J=9.0 Hz), 7.3-7.8(8H, m), 10.29(1H, s) ESI-MS(m/z): 489(M+Na)⁺, 467(M+H)⁺

### EXAMPLE 335

The following compound was obtained in substantially the same manner as in Example 331.

N-{4-[(1H-Pyrazol-1-ylacetyl)amino]phenyl}-4′-(trifluoromethyl)-1,1′-biphenyl-2-carboxamide

¹H-NMR(DMSO-d₆): δ 4.99(2H, s), 6.25-6.3(1H, m), 7.4-7.8(4H, m), 10.26(1H, s), 10.32(1H, s) ESI-MS(m/z): 487(M+Na)⁺

### EXAMPLE 336

To a solution of N-(4-aminophenyl)-2-(1H-pyrazol-1-yl)acetamide (432 mg), 2-(4-methylphenyl)-1-cyclohexene-1-carboxylic acid (432 mg) and benzotriazol-1-yl-oxytripyrrolidinophosphonium hexafluorophosphate (PyBOP) (1.25 g) in N,N-dimethylformamide (40 ml) was added diisopropylethylamine (516 mg) at ambient temperature and the mixture was stirred at the same temperature for 24 hours. The mixture was poured into a mixture of ethyl acetate, water and 6N hydrochloric acid, and the separated organic layer was washed with water and brine, dried over magnesium sulfate and evaporated in vacuo. The residue was purified by column chromatography on silica gel eluting with ethyl acetate to give 2-(4-methylphenyl)-N-{4-[(1H-pyrazol-1-ylacetyl)amino]phenyl}-1-cyclohexene-1-carboxamide (625 mg) as a pale brown powder.

¹H-NMR(DMSO-d₆): δ 1.6-1.8(4H, m), 2.20(3H, s), 2.3-2.45(4H, m), 4.96(2H, s), 6.26(1H, dd, J=2.3 and 1.8 Hz), 7.03(2H, d, J=8.1 Hz), 7.18(2H, d, J=8.1 Hz), 7.31(2H, d, J=9.1 Hz), 7.39(2H, d, J=9.1 Hz), 7.44(1H, d, J=1.8 Hz), 7.73(1H, d, J=2.3 Hz), 9.48(1H, s), 10.18(1H, s) ESI-MS(m/z): 437(M+Na)⁺

### EXAMPLE 337

The following compound was obtained in substantially the same manner as in Example 331.

N-[1-(1H-Pyrazol-1-ylacetyl)-2,3-dihydro-1H-indol-5-yl]-4′-(trifluoromethyl)-1,1′-biphenyl-2-carboxamide

¹H-NMR(DMSO-d₆): δ 3.16(2H, t, J=8.3 Hz), 4.17(2H, t, J=8.3 Hz), 5.22(2H, s), 6.29(1H, dd, J=2.2 and 1.7 Hz), 7.22(1H, dd, J=8.7 and 1.7 Hz), 7.46(1H, d, J=1.7 Hz), 7.5-7.7(6H, m), 7.71(1H, d, J=2.2 Hz), 7.76(2H, d, J=8.6 Hz), 7.86(2H, d, J=8.6 Hz), 10.32(1H, s)

### EXAMPLE 338

The following compound was obtained in substantially the same manner as in Example 331.

4′-Methyl-N-[1-(1H-pyrazol-1-ylacetyl)-2,3-dihydro-1H-indol-5-yl]-1,1′-biphenyl-2-carboxamide

¹H-NMR(DMSO-d₆): δ 2.29(3H, s), 3.16(2H, t, J=7.6 Hz), 4.17(2H, t, J=7.6 Hz), 5.22(2H, s), 6.29(1H, dd, J=2.1 and 1.4 Hz), 7.17(2H, d, J=8.0 Hz), 7.2-7.3(1H, m), 7.32(2H, d, J=8.0 Hz), 7.4-7.55(5H, m), 7.55(1H, d, J=1.4 Hz), 7.71(1H, d, J=2.1 Hz), 7.86(1H, d, J=8.7 Hz), 10.21(1H, s) ESI-MS(m/z): 459(M+Na)⁺, 437(M+H)⁺

### EXAMPLE 339

To a solution of 1-(1H-pyrazol-1-ylacetyl)-5-indolinamine (905 mg), 4′-(dimethylamino)-1,1′-biphenyl-2-carboxylic acid (901 mg) and benzotriazol-1-yl-oxytripyrrolidinophosphonium hexafluorophosphate (PyBOP) (2.33 g) in N,N-dimethylformamide (30 ml) was added dropwise diisopropylethylamine (966 mg) at ambient temperature and the mixture was stirred at the same temperature for 20 hours. The mixture was poured into a mixture of ethyl acetate and water and the separated organic layer was washed with water and brine, dried over magnesium sulfate and evaporated in vacuo. The residue was purified by column chromatography on silica gel eluting with ethyl acetate to give 4′-(dimethylamino)-N-[1-(1H-pyrazol-1-ylacetyl)-2,3-dihydro-1H-indol-5-yl]-1,1′-biphenyl-2-carboxamide (954 mg) as a pale yellow powder.

¹H-NMR(DMSO-d₆): δ 2.88(6H, s), 3.17(2H, t, J=8.3 Hz), 4.17(2H, t, J=8.3 Hz), 5.22(2H, s), 6.29(1H, dd, J=2.3 and 1.4 Hz), 6.70(2H, d, J=8.8 Hz), 7.29(2H, d, J=8.8 Hz), 7.25-7.55(5H, m), 7.46(1H, d, J=1.4 Hz), 7.56(1H, s), 7.71(1H, d, J=2.3 Hz), 7.86(1H, d, J=8.6 Hz), 10.14(1H, s) ESI-MS(m/z): 488(M+Na)⁺

### EXAMPLE 340

The following compound was obtained in substantially the same manner as in Example 339.

2-(4-Methylphenyl)-N-[1-(1H-pyrazol-1-ylacetyl)-2,3-dihydro-1H-indol-5-yl]-1-cyclohexene-1-carboxamide

¹H-NMR(DMSO-d₆): δ 1.6-1.8(4H, m), 2.21(3H, s), 2.25-2.4(4H, m), 3.11(2H, t, J=8.5 Hz), 4.13(2H, t, J=8.5 Hz), 5.20(2H, s), 6.28(1H, dd, J=2.0 and 1.7 Hz), 7.04(2H, d, J=8.1 Hz), 7.0-7.1(1H, m), 7.17(2H, d, J=8.1 Hz), 7.40(1H, s), 7.45(1H, d, J=1.7 Hz), 7.69(1H, d, J=2.0 Hz), 7.77(1H, d, J=8.6 Hz), 9.48(1H, s)

### EXAMPLE 341

The following compound was obtained in substantially the same manner as in Example 339.

2-[4-(Dimethylamino)phenyl]-N-[1-(1H-pyrazol-1-ylacetyl)-2,3-dihydro-1H-indol-5-yl]-1-cyclohexene-1-carboxamide

¹H-NMR(DMSO-d₆): δ 1.6-1.8(4H, m), 2.3-2.45(4H, m), 2.82(6H, s), 3.12(2H, t, J=8.6 Hz), 4.13(2H, t, J=8.6 Hz), 5.20(2H, s), 6.28(1H, dd, J=2.2 and 1.6 Hz), 6.58(2H, d, J=8.8 Hz), 7.09(1H, dd, J=8.6 and 1.3 Hz), 7.13(2H, d, J=8.8 Hz), 7.42(1H, d, J=1.6 Hz), 7.69(1H, d, J=2.2 Hz), 7.78(1H, d, J=8.6 Hz), 9.41(1H, s) negative ESI-MS(m/z): 468(M−H)⁻

Preparation 177

To a solution of 5-nitroindoline (11.72 g) and triethylamine (8.67 g) in N,N-dimethylformamide (150 ml) was added dropwise chloroacetyl chloride (8.06 g) at 5° C. and the mixture was stirred at ambient temperature for 20 hours. The mixture was poured into a mixture of ethyl acetate and water and the separated organic layer was washed with water and brine, dried over magnesium sulfate and evaporated in vacuo. The residue was triturated with ethyl acetate and collected by filtration to give 1-(chloroacetyl)-5-nitroindoline (14.66 g) as a yellow crystal.

¹H-NMR(DMSO-d₆): δ 3.28(2H, t, J=8.6 Hz), 4.25(2H, t, J=8.6 Hz), 4.64(2H, s), 8.1-8.2(3H, m) ESI-MS(m/z): 263(M+Na)⁺

Preparation 178

To a solution of 1-(chloroacetyl)-5-nitroindoline (4.81 g) in N,N-dimethylformamide (80 ml) was added 1,2,4-triazole sodium derivative (purity 90%)(2.18 g) at ambient temperature and the mixture was stirred at 50° C. for 6 hours. The mixture was poured into a mixture of ethyl acetate and water and the separated organic layer was washed with water and brine, dried over magnesium sulfate and evaporated in vacuo. The residue was purified by column chromatography on silica gel eluting with hexane: ethyl acetate (1:2) to give 5-nitro-1-(1H-1,2,4-triazol-1-ylacetyl)indoline (2.63 g) as a yellow powder.

¹H-NMR(DMSO-d₆): δ 3.33(2H, t, J=8.7 Hz), 4.34(2H, t, J=8.7 Hz), 5.47(2H, s), 8.03(1H, s), 8.1-8.2(3H, m), 8.51(1H, s) negative ESI-MS(m/z): 272(M−H)⁻

Preparation 179

To a solution of 5-nitro-1-(1H-1,2,4-triazol-1-ylacetyl)indoline (2.62 g) in N,N-dimethylformamide (50 ml) was added 5% palladium on carbon (50% wet) (1 g) and the mixture was hydrogenated for 4 hours at 45° C. The catalyst was removed by filtration and washed with N,N-dimethylformamide (10 ml). The filtrate containing 1-(1H-1,2,4-triazol-1-ylacetyl)-5-indolinamine was used to next step without further purification.

### EXAMPLE 342

The following compound was obtained in substantially the same manner as in Example 339.

2-(4-Methylphenyl)-N-[1-(1H-1,2,4-triazol-1-ylacetyl)-2,3-dihydro-1H-indol-5-yl]-1-cyclohexene-1-carboxamide

¹H-NMR(DMSO-d₆): δ 1.6-1.85(4H, m), 2.21(3H, s), 2.3-2.45(4H, m), 3.13(2H, t, J=8.4 Hz), 4.15(2H, t, J=8.4 Hz), 5.33(2H, s), 7.02(2H, d, J=8.1 Hz), 7.04(1H, d, J=8.6 Hz), 7.17(2H, d, J=8.1 Hz), 7.41(1H, s), 7.76(1H, d, J=8.6 Hz), 7.99(1H, s), 8.48(1H, s), 9.49(1H, s) ESI-MS(m/z): 464(M+Na)⁺

### EXAMPLE 343

To a solution of N-(2,3-dihydro-1H-indol-5-yl)-4′-(trifluoromethyl)-1,1′-biphenyl-2-carboxamide (765 mg), 1H-tetrazol-1-ylacetic acid (256 mg) and benzotriazol-1-yl-oxytripyrrolidinophosphonium hexafluorophosphate (PyBOP) (1.25 g) in N,N-dimethylformamide (40 ml) was added dropwise diisopropylethylamine (966 mg) at ambient temperature and the mixture was stirred at the same temperature for 20 hours. The mixture was poured into a mixture of ethyl acetate and water and the separated organic layer was washed with water and brine, dried over magnesium sulfate and evaporated in vacuo. The residue was purified by column chromatography on silica gel eluting with hexane:ethyl acetate (1:2) to give N-[1-(1H-tetrazol-1-ylacetyl)-2,3-dihydro-1H-indol-5-yl]-4′-(trifluoromethyl)-1,1′-biphenyl-2-carboxamide (810 mg) as a white crystal.

¹H-NMR(DMSO-d₆): δ 3.20(2H, t, J=8.3 Hz), 4.23(2H, t, J=8.3 Hz), 5.72(2H, s), 7.23(1H, dd, J=8.7 and 1.7 Hz), 7.5-7.9(10H, m), 9.37(1H, s), 10.33(1H, s) ESI-MS(m/z): 515(M+Na)⁺

Preparation 180

The following compound was obtained in substantially the same manner as in Preparation 130.

5-Nitro-1-(1H-tetrazol-1-ylacetyl)indoline

¹H-NMR(DMSO-d₆): δ 3.35(2H, t, J=8.5 Hz), 4.37(2H, t, J=8.5 Hz), 5.82(2H, s), 8.05-8.2(3H, m), 9.38(1H, s)

Preparation 181

1-(1H-Tetrazol-1-ylacetyl)-5-indolinamine was obtained in substantially the same manner as in Preparation 179. This compound was used in Example 344 without purification.

### EXAMPLE 344

The following compound was obtained in substantially the same manner as in Example 339.

4′-(Dimethylamino)-N-[1-(1H-tetrazol-1-ylacetyl)-2,3-dihydro-1H-indol-5-yl]-1,1′-biphenyl-2-carboxamide

¹H-NMR(DMSO-d₆): δ 2.88(6H, s), 3.21(2H, t, J=8.5 Hz), 4.23(2H, t, J=8.5 Hz), 5.75(2H, s), 6.70(2H, d, J=8.7 Hz), 7.28(2H, t, J=8.7 Hz), 7.3-7.6(6H, m), 7.83(1H, d, J=8.7 Hz), 9.90(1H, s), 10.22(1H, s) ESI-MS(m/z): 490(M+Na)⁺, 468(M+H)⁺

### EXAMPLE 345

The following compound was obtained in substantially the same manner as in Example 339.

2-(4-Methylphenyl)-N-[1-(1H-tetrazol-1-ylacetyl)-2,3-dihydro-1H-indol-5-yl]-1-cyclohexene-1-carboxamide

¹H-NMR(DMSO-d₆): δ 1.6-1.8(4H, m), 2.21(3H, s), 2.3-2.4(4H, m), 3.15(2H, t, J=8.4 Hz), 4.19(2H, t, J=8.4 Hz), 5.70(2H, s), 7.04(2H, d, J=8.1 Hz), 7.0-7.1(1H, m), 7.17(2H, d, J=8.1 Hz), 7.43(1H, s), 7.75(1H, d, J=8.7 Hz), 9.35(1H, s), 9.2(1H, s)

### EXAMPLE 346

The following compound was obtained in substantially the same manner as in Example 200.

2-(4-Methylphenyl)-N-[2-(1H-pyrazol-1-ylacetyl)-2,3-dihydro-1H-isoindol-5-yl]-1-cyclohexene-1-carboxamide

¹H-NMR(DMSO-d₆): δ 1.6-1.8(4H, m), 2.27(3H, s), 2.3-2.45(4H, m), 4.5-4.6(2H, m), 4.7-4.8(2H, m), 5.13(2H, s), 6.25-6.3(1H, dd, J=2.2 and 1.6 Hz), 7.05-7.3(7H, m), 7.44(1H, d, J=1.6 Hz), 7.68(1H, d, J=2.2 Hz), 9.60(1H, s) ESI-MS(m/z): 463(M+Na)⁺, 441(M+H)⁺

### EXAMPLE 347

The following compound was obtained in substantially the same manner as in Example 200.

N-[2-(1H-Pyrazol-1-ylacetyl)-2,3-dihydro-1H-isoindol-5-yl]-2-[4-(trifluoromethyl)phenyl]-1-cyclohexene-1-carboxamide

¹H-NMR(DMSO-d₆): δ 1.6-1.8(4H, m), 2.3-2.45(4H, m), 4.5-4.6(2H, m), 4.8-4.9(2H, m), 5.13(2H, s), 6.27(1H, dd, J=2.3 and 1.7 Hz), 7.18(2H, s), 7.44(1H, d, J=1.7 Hz), 7.48(2H, d, J=8.5 Hz), 7.63(2H, d, J=8.5 Hz), 7.68(1H, d, J=2.3 Hz), 9.73 and 9.75(total 1H, s) ESI-MS(m/z): 517(M+Na)⁺, 495(M+H)⁺

### EXAMPLE 348

The following compound was obtained in substantially the same manner as in Example 200.

2-[4-(Dimethylamino)phenyl]-N-[2-(1H-pyrazol-1-ylacetyl)-2,3-dihydro-1H-isoindol-5-yl]-1-cyclohexene-1-carboxamide

¹H-NMR(DMSO-d₆): δ 1.6-1.8(4H, m), 2.3-2.45(4H, m), 2.84(6H, s), 4.5-4.6(2H, m), 4.8-4.9(2H, m), 5.13(2H, s), 6.27(1H, dd, J=2.0 and 1.7 Hz), 6.58(2H, d, J=8.7 Hz), 7.14(2H, d, J=8.7 Hz), 7.1-7.3(2H, m), 7.44(1H, d, J=1.7 Hz), 7.52(1H, s), 7.68(1H, d, J=2.0 Hz), 9.53(1H, s) negative ESI-MS(m/z): 468(M−H)⁻

### EXAMPLE 349

A mixture of 6-methyl-2-(4-methyl-1-piperidinyl)nicotinic acid (7.4 g), 1-acetyl-2,3-dihydro-1H-indol-5-ylamine (5.3 g), 1-hydroxybenzotriazole hydrate (4.84 g) and 1-[3-(dimethylamino)propyl]-3-ethylcarbodiimide (4.9 g) in N,N-dimethylformamide (50 ml) was stirred at ambient temperature for 15 hours. The reaction mixture was poured into a mixture of ethyl acetate and water, and the organic layer was washed with brine and dried over magnesium sulfate. The solvent was concentrated in vacuo and the precipitate was collected by filtration to give N-(1-acetyl-2,3-dihydro-1H-indol-5-yl)-6-methyl-2-(4-methyl-1-piperidinyl)nicotinamide (10.15 g).

¹H-NMR(DMSO-d₆): δ 0.89(3H, d J=6.18 Hz), 1.11-1.28(2H, m), 1.42-1.65(3H, m), 2.14(3H, s), 2.39(3H, s), 2.74-2.86(2H, m), 3.14(2H, t J=8.32 Hz), 3.14(2H, t J=8.38 Hz), 3.61-3.68(2H, m), 4.08(2H, t J=8.32 Hz), 6.82(1H, d J=7.60 Hz), 7.39(1H, dd J=1.74 Hz, 8.62 Hz), 7.72(1H, s), 7.74(1H, d J=7.60 Hz), 7.99(1H, d J=8.62 Hz), 10.48(1H, s)

Preparation 182

A mixture of N-(1-acetyl-2,3-dihydro-1H-indol-5-yl)-6-methyl-2-(4-methyl-1-piperidinyl)nicotinamide (10.1 g) and 6N hydrochloric acid (28 ml) in methanol (40 ml) and tetrahydrofuran (40 ml) was refluxed under stirring for 9 hours. The reaction mixture was evaporated in vacuo and the residue was dissolved in a mixture of ethyl acetate and water and adjusted to pH 8.0 with 20% potassium carbonate solution. The organic layer was washed with brine and dried over magnesium sulfate. The solvent was concentrated in vacuo and the precipitate was collected by filtration to give N-(2,3-dihydro-1H-indol-5-yl)-6-methyl-2-(4-methyl-1-piperidinyl)nicotinamide (7.9 g).

¹H-NMR(DMSO-d₆): δ 0.93(3H, d J=6.22 Hz), 1.17-1.30(2H, m), 1.46-1.64(3H, m), 2.38(3H, s), 2.74-2.94(4H, m), 3.34-3.44(2H, m), 3.60-3.67(2H, m), 5.35(1H, s), 6.46(1H, d J=8.24 Hz), 6.82(1H, d J=7.64 Hz), 7.20(1H, dd J=2.04 Hz, 8.24 Hz), 7.46(1H, s), 7.74(1H, d J=7.64 Hz), 10.24(1H, s)

### EXAMPLE 350

The following compound was obtained in substantially the same manner as in Example 31.

tert-Butyl 6-{2-[5-({[6-methyl-2-(4-methyl-1-piperidinyl)-3-pyridinyl]carbonyl}amino)-2,3-dihydro-1H-indol-1-yl]-2-oxoethyl}-2-pyridinylcarbamate

¹H-NMR(DMSO-d₆): δ 0.89(3H, d J=6.14 Hz), 1.14-1.26(2H, m), 1.42-1.65(2H, m), 1.46(9H, s), 2.39(3H, s), 2.75-2.86(2H, m), 3.18(2H, t J=8.26 Hz), 3.61-3.63(2H, m), 3.86(2H, s), 4.28(2H, t J=8.26 Hz), 6.82(1H, d J=7.70 Hz), 6.96-7.00(1H, m), 7.38(1H, dd J=1.76 Hz, 8.66 Hz), 7.64-7.76(4H, m), 7.91(1H, d J=8.66 Hz), 9.66(1H, s), 10.48(1H, s)

### EXAMPLE 351

The following compound was obtained in substantially the same manner as in Example 32.

N-{1-[(6-Amino-2-pyridinyl)acetyl]-2,3-dihydro-1H-indol-5-yl}-6-methyl-2-(4-methyl-1-piperidinyl)nicotinamide

¹H-NMR(DMSO-d₆): δ 0.89(3H, d J=6.12 Hz), 1.13-1.26(2H, m), 1.48-1.65(3H, m), 2.39(3H, s), 2.74-2.86(2H, m), 3.15(2H, t J=8.26 Hz), 3.60-3.63(2H, m), 3.68(2H, s), 4.20(2H, t J=8.26 Hz), 5.87(2H, s), 6.31(1H, d J=8.02 Hz), 6.43(1H, d J=7.14 Hz), 6.82(1H, d J=7.64 Hz), 7.28-7.41(2H, m), 7.72-7.76(2H, m), 8.00(1H, d J=8.66 Hz), 10.48(1H, s) ESI-MS(m/z): 507(M+Na)⁺, 485(M+1)⁺

### EXAMPLE 352

The following compound was obtained in substantially the same manner as in Example 349.

2-(Isopropylamino)-6-methyl-N-[1-(2-pyridinylacetyl)-2,3-dihydro-1H-indol-5-yl]nicotinamide

¹H-NMR(DMSO-d₆): δ 1.17(6H, d J=6.46 Hz), 2.34(3H, s), 3.16(2H, t J=8.30 Hz), 3.98(2H, s), 4.08-4.34(3H, m), 6.47(1H, d J=7.86 Hz), 7.25-7.31(1H, m), 7.36(2H, d J=7.94 Hz), 7.63(1H, s), 7.72-7.80(1H, m), 7.98(2H, d J=7.94 Hz), 8.09(1H, d J=7.36 Hz), 8.49-8.51(1H, s), 9.99(1H, s) ESI-MS(m/z): 452(M+Na)⁺, 430(M+1)⁺

### EXAMPLE 353

The following compound was obtained in substantially the same manner as in Example 349.

2-(Cyclohexylamino)-6-methyl-N-[1-(2-pyridinylacetyl)-2,3-dihydro-1H-indol-5-yl]nicotinamide

¹H-NMR(DMSO-d₆): δ 1.47-1.75(8H, m), 1.89-1.99(2H, m), 2.33(3H, s), 3.16(2H, t J=8.26 Hz), 4.01(2H, s), 3.90-4.09(1H, m), 4.22(2H, t J=8.26 Hz), 6.46(1H, d J=7.88 Hz), 7.2-7.31(1H, m), 7.37(2H, d J=7.78 Hz), 7.62(1H, d J=1.20 Hz), 7.72-7.77(1H, m), 7.98(2H, d J=7.78 Hz), 8.25(1H, d J=7.62 Hz), 8.49-8.51(1H, m), 9.98(1H, s) ESI-MS(m/z): 492(M+Na)⁺, 470(M+1)⁺

### EXAMPLE 354

The following compound was obtained in substantially the same manner as in Example 349.

2-(Ethylmethylamino)-6-methyl-N-[1-(2-pyridinylacetyl)-2,3-dihydro-1H-indol-5-yl]nicotinamide

¹H-NMR(DMSO-d₆): δ 1.06(3H, t J=6.96 Hz), 2.35(3H, s), 2.86(3H, s), 3.16(2H, t J=8.28 Hz), 3.43(2H, q J=6.96 Hz), 4.01(2H, s), 4.21(2H, t J=8.28 Hz), 6.62(1H, d J=7.56 Hz), 7.28-7.42(3H, m), 7.56(1H, d J=7.50 Hz), 7.67(1H, s), 7.74-7.78(1H, m), 7.97(1H, d J=8.68 Hz), 8.49-8.51(1H, m), 10.31(1H, s) ESI-MS(m/z): 452(M+Na)⁺, 430(M+1)⁺

### EXAMPLE 355

The following compound was obtained in substantially the same manner as in Example 349.

2-(Diethylamino)-6-methyl-N-[1-(2-pyridinylacetyl)-2,3-dihydro-1H-indol-5-yl]nicotinamide

¹H-NMR(DMSO-d₆): δ 1.05(6H, t J=6.88 Hz), 2.37(3H, s), 3.16(2H, t J=8.22 Hz), 3.25-3.35(4H, m), 4.00(2H, s), 4.21(2H, t J=8.22 Hz), 6.70(1H, d J=7.62 Hz), 7.26-7.43(3H, m), 7.64-7.77(3H, m), 7.99(1H, dd J=8.70 Hz), 8.49-8.51(1H, m), 10.71(1H, s) ESI-MS (m/z): 466 (M+Na)⁺, 444 (M+1)⁺

### EXAMPLE 356

The following compound was obtained in substantially the same manner as in Example 358 as mentioned below.

N-(1-{[6-(Acetylamino)-2-pyridinyl]methyl}-2,3-dihydro-1H-indol-5-yl)-6-methyl-2-(4-methyl-1-piperidinyl)nicotinamide

¹H-NMR(DMSO-d₆): δ 0.90(3H, d J=6.20 Hz), 1.13-1.30(2H, m), 1.49-1.66(3H, m), 1.99(3H, s), 2.39(3H, s), 2.75-2.86(2H, m), 2.94(2H, t J=7.86 Hz), 3.37(2H, t J=7.86 Hz), 3.61-3.67(2H, m), 4.26(2H, s), 6.51(1H, d J=8.40 Hz), 6.82(1H, d J=7.66 Hz), 7.11(1H, d J=7.36 Hz), 7.26(1H, dd J=1.80 Hz, 8.40 Hz), 7.51(1H, d J=1.38 Hz), 7.70-7.78(2H, m), 7.98(1H, d J=8.18 Hz), 10.30(1H, s), 10.52(1H, s) ESI-MS(m/z): 521 (M+Na)⁺, 499 (M+1)⁺

### EXAMPLE 357

A mixture of N-(1-{[6-(acetylamino)-2-pyridinyl]methyl}-2,3-dihydro-1H-indol-5-yl)-6-methyl-2-(4-methyl-1-piperidinyl)nicotinamide (485 mg) and 6N hydrochloric acid (1 ml) in methanol (10 ml) and tetrahydrofuran (10 ml) was refluxed under stirring for 5 hours. The reaction mixture was evaporated in vacuo and the residue was dissolved in a mixture of ethyl acetate and water and adjusted to PH 8.0 with 20% potassium carbonate solution. The organic layer was washed with brine and dried over magnesium sulfate. The solvent was concentrated in vacuo and the precipitate was collected by filtration to give N-{1-[(6-amino-2-pyridinyl)methyl]-2,3-dihydro-1H-indol-5-yl}-6-methyl-2-(4-methyl-1-piperidinyl)nicotinamide (370 mg).

¹H-NMR(DMSO-d₆): δ 0.90(3H, d J=6.20 Hz), 1.17-1.30(2H, m), 1.43-1.67(3H, m), 2.38(3H, s), 2.52-2.80(2H, m), 2.92(2H, t J=7.98 Hz), 3.32-3.41(2H, m), 3.61-3.67(2H, m), 4.08(2H, s), 5.90(2H, s), 6.32(1H, d J=8.08 Hz), 6.41(1H, dd J=4.34 Hz, 7.66 Hz), 6.81(1H, d J=7.56 Hz), 7.22-7.36(2H, m), 7.48(1H, d J=1.84 Hz), 7.74(1H, d J=7.56 Hz), 10.28(1H, s) ESI-MS(m/z): 579(M+Na)⁺, 457(M+1)⁺

### EXAMPLE 358

A mixture of N-(2,3-dihydro-1H-indol-5-yl)-6-methyl-2-(4-methyl-1-piperidinyl)nicotinamide (525 mg), 2-pyridinecarboxaldehyde (193 mg) and sodium triacetoxyborohydride (952 mg) in chloroform (20 ml) was stirred at ambient temperature for 15 hours. A water (10 ml) was added to a reaction mixture and adjusted to PH 8.5 with 10% potassium carbonate solution and stirred at ambient temperature for 30 minutes. The organic layer was washed with brine and dried over magnesium sulfate. The solvent was evaporated in vacuo and the residue was chromatographed on silica gel eluting with ethyl acetate:n-hexane (6:4 v/v). The eluted fractions containing the desired product were collected and the solvent was evaporated in vacuo and the residue was recrystallized from a mixture of diisopropyl ether and n-hexane to give 6-methyl-2-(4-methyl-1-piperidinyl)-N-[1-(2-pyridinylmethyl)-2,3-dihydro-1H-indol-5-yl]nicotinamide (295 mg).

¹H-NMR(DMSO-d₆): δ 0.89(3H, d J=6.16 Hz), 1.18-1.29(2H, m), 1.43-1.66(3H, m), 2.39(3H, s), 2.75-2.97(4H, m), 3.34-3.42(2H, m), 3.61-3.67(2H, m), 4.35(2H, s), 6.51(1H, d J=8.42 Hz), 6.82(1H, d J=7.66 Hz), 7.25-7.34(2H, m), 7.41(1H, d J=7.80 Hz), 7.51(1H, s), 7.79-7.81(2H, m), 8.53-8.55(1H, m), 10.31(1H, s) ESI-MS(m/z): 464(M+Na)⁺, 442(M+1)⁺

Preparation 183

A solution of chloroacetylchoride (967 mg) in tetrahydrofuran (5 ml) was dropwise added to a mixture of N-(2,3-dihydro-1H-indol-5-yl)-6-methyl-2-(4-methyl-1-piperidinyl)nicotinamide (2.5 g) and triethylamine (1.73 mg) in tetrahydrofuran (50 ml) at 5-20° C. with stirring and the resultant mixture was stirred at ambient temperature for 15 hours. The reaction mixture was poured into a mixture of ethyl acetate and water, and the organic layer was washed with brine and dried over magnesium sulfate. The solvent was evaporated in vacuo and the residue was chromatographed on silica gel eluting with ethyl acetate and n-hexane (6:4). The fraction was concentrated in vacuo and the precipitate was collected by filtration to give N-(1-chloroacetyl-2,3-dihydro-1H-indol-5-yl)-6-methyl-2-(4-methyl-1-piperidinyl)nicotinamide.

¹H-NMR(DMSO-d₆): δ 0.88 (3H, d J=6.08 Hz), 1.16-1.23 (2H, m), 1.47-1.51 (1H, m), 1.60-1.63 (2H, m), 2.39 (3H, s), 2.78-2.83 (2H, m), 3.19 (2H, t J=8.36 Hz), 3.64-3.67 (2H, m), 4.14 (2H, t J=8.36 Hz), 4.52 (2H, s), 6.82 (1H, d J=7.60 Hz), 7.43 (1H, d J=8.68 Hz), 7.74 (1H, d J=7.60 Hz), 7.76 (1H, s), 7.99 (1H, d J=8.68 Hz), 10.51 (1H, s)

### EXAMPLE 359

A mixture of imidazole (150 mg) and potassium tert-butoxide (247 mg) in N,N-dimethylformamide (10 ml) was stirred at ambient temperature for 30 minutes. A N-(1-chloroacetyl-2,3-dihydro-1H-indol-5-yl)-6-methyl-2-(4-methyl-1-piperidinyl)nicotinamide (470 mg) was added to a above mixture and the resultant mixture was stirred at 65-70° C. for 6 hours. The reaction mixture was poured into a mixture of ethyl acetate and water, and the organic layer was washed with brine and dried over magnesium sulfate. The solvent was concentrated in vacuo and the precipitate was collected by filtration to give N-[1-(1H-imidazol-1-ylacetyl)-2,3-dihydro-1H-indol-5-yl]-6-methyl-2-(4-methyl-1-piperidinyl)nicotinamide (270 mg).

¹H-NMR(DMSO-d₆): δ 0.88(3H, d J=6.00 Hz), 1.17-1.19(2H, m), 1.48(1H, m), 1.60-1.63(2H, m), 2.78-2.83(2H, m), 3.32(2H, t J=7.36 Hz), 3.64-3.67(2H, m), 4.18(2H, t J=7.36 Hz), 5.10(2H, s), 6.81(1H, d J=7.24 Hz), 6.90(1H, s), 7.01(1H, s), 7.40(1H, d J=7.60 Hz), 7.58(1H, s), 7.73(1H, d J=7.24 Hz), 7.78(1H, s), 7.95(1H, d J=7.60 Hz), 10.50(1H, s) ESI-MS (m/z): 481(M+Na)⁺, 459 (M+1)⁺

### EXAMPLE 360

A mixture of N-(1-acetyl-2,3-dihydro-1H-indol-5-yl)-6-methyl-2-(4-methyl-1-piperidinyl)nicotinamide (470 mg) and 1,2,4-triazole sodium salt (140 mg) in N,N-dimethylformamide (10 ml) was stirred at 65-70° C. for 7 hours. The reaction mixture was poured into a mixture of ethyl acetate and water, and the organic layer was washed with brine and dried over magnesium sulfate. The solvent was concentrated in vacuo and the precipitate was collected by filtration to give 6-methyl-2-(4-methyl-1-piperidinyl)-N-[1-(1H-1,2,4-triazol-1-ylacetyl)-2,3-dihydro-1H-indol-5-yl]nicotinamide (336 mg).

¹H-NMR(DMSO-d₆): δ 0.88(3H, d J=6.10 Hz), 1.10-1.27(2H, m), 1.45-1.64(3H, m), 2.39(3H, s), 2.73-2.89(2H, m), 3.23(2H, t J=8.24 Hz), 3.62-3.69(2H, m), 4.22(2H, t J=8.24 Hz), 5.38(2H, s), 6.81(1H, d J=7.64 Hz), 7.41(1H, dd J=1.70 Hz, 8.70 Hz), 7.73(1H, d J=7.64 Hz), 7.77(1H, s), 7.93(1H, d J=8.70 Hz), 8.01(1H, s), 8.51(1H, s), 10.50(1H, s) ESI-MS(m/z): 482(M+Na)⁺, 460(M+1)⁺

### EXAMPLE 361

The following compound was obtained in substantially the same manner as in Example 349.

N-(1-Acetyl-2,3-dihydro-1H-indol-5-yl)-2-isopropoxy-4-methylbenzamide

¹H-NMR(DMSO-d₆): δ 1.39 (6H, d J=6.00 Hz), 2.14 (3H, s), 2.36 (3H, s), 3.15 (2H, t J=8.40 Hz), 4.08 (2H, t J=8.40 Hz), 4.78-4.84 (1H, m), 6.89 (1H, d J=7.68 Hz), 7.04 (1H, s), 7.37 (1H, dd J=1.04 Hz, 8.68 Hz), 7.67 (1H, d J=1.04 Hz), 7.72 (1H, d J=7.68 Hz), 7.99 (1H, d J=8.68 Hz), 10.06 (1H, s)

Preparation 184

The following compound was obtained in substantially the same manner as in Preparation 182.

N-(2,3-Dihydro-1H-indol-5-yl)-2-isopropoxy-4-methylbenzamide

¹H-NMR(DMSO-d₆): δ 1.39 (6H, d J=6.00 Hz), 2.35 (3H, s), 2.91 (2H, t J=8.26 Hz), 4.75-4.87 (1H, m), 5.36 (1H, s), 6.49 (1H, d J=8.24 Hz), 6.88 (1H, d J=7.92 Hz),7.02 (1H, s), 7.20 (1H, dd J=1.92 Hz, 8.24 Hz), 7.43 (1H, s), 7.77 (1H, d J=7.92 Hz), 9.84 (1H, s)

Preparation 185

The following compound was obtained in substantially the same manner as in Preparation 183.

N-[1-(Chloroacetyl)-2,3-dihydro-1H-indol-5-yl]-2-isopropoxy-4-methylbenzamide

¹H-NMR(DMSO-d₆): δ 1.39 (6H, d J=6.00 Hz), 2.36 (3H, s), 3.19 (2H, t J=8.22 Hz), 4.15 (2H, t J=8.22 Hz), 4.52 (2H, s), 4.74-4.86 (1H, m), 6.89 (1H, d J=7.74 Hz), 7.04 (1H, s), 7.42 (1H, dd J=1.54 Hz, 8.62 Hz), 7.70-7.73 (2H, m), 7.99 (1H, d J=8.62 Hz), 10.08 (1H, s)

### EXAMPLE 362

The following compound was obtained in substantially the same manner as in Example 360.

2-Isopropoxy-4-methyl-N-[1-(1H-1,2,4-triazol-1-ylacetyl)-2,3-dihydro-1H-indol-5-yl]benzamide

¹H-NMR(DMSO-d₆): δ 1.39 (6H, d J=6.00 Hz), 2.36 (3H, s), 3.24 (2H, t J=8.28 Hz), 4.22 (2H, t J=8.28 Hz), 4.74-4.86 (1H, m), 5.38 (1H, s), 6.89 (1H, d J=7.74 Hz), 7.04 (1H, s), 7.40 (1H, dd J=1.70 Hz, 8.70 Hz), 7.71 (1H, d J=7.74 Hz), 7.73 (1H, s), 7.93 (1H, d J=8.70 Hz), 8.00 (1H, s), 8.51 (1H, s), 10.08 (1H, s)

Preparation 186

The following compound was obtained in substantially the same manner as in Preparation 40.

tert-Butyl 5-({[6-methyl-2-(4-thiomorpholinyl)-3-pyridinyl]carbonyl}amino)-1-indolinecarboxylate

¹H-NMR(DMSO-d₆): δ 1.51 (9H, s), 2.40 (3H, s), 2.62-2.66 (4H, m), 3.07 (2H, t J=8.36 Hz), 3.51-3.55 (4H, m), 3.91 (2H, t J=8.36 Hz), 6.84 (1H, d J=7.64 Hz), 7.42 (1H, d J=6.46 Hz), 7.66-7.72 (3H, m), 10.26 (1H, s)

Preparation 187

The following compound was obtained in substantially the same manner as in Preparation 41.

N-(2,3-Dihydro-1H-indol-5-yl)-6-methyl-2-(4-thiomorpholinyl)nicotinamide

¹H-NMR(DMSO-d₆): δ 2.39 (3H, s), 2.63-2.68 (4H, m), 2.90 (2H, t J=8.30 Hz), 3.33-3.44 (2H, m), 3.50-3.55 (4H, m), 5.35 (1H, s), 6.47 (1H, d J=8.26 Hz), 6.83 (1H, d J=7.60 Hz), 7.20 (1H, dd J=1.94 Hz, 8.26 Hz), 7.45 (1H, d J=1.94 Hz), 7.69 (1H, d J=7.60 Hz), 10.02 (1H, s)

### EXAMPLE 363

The following compound was obtained in substantially the same manner as in Example 26.

6-Methyl-N-[1-(2-pyridinylacetyl)-2,3-dihydro-1H-indol-5-yl]-2-(4-thiomorpholinyl)nicotinamide

¹H-NMR(DMSO-d₆): δ 2.40 (3H, s), 2.62-2.66 (4H, m), 3.17 (2H, t J=8.38 Hz), 3.51-3.56 (4H, m), 3.98 (2H, s), 4.22 (2H, t J=8.38 Hz), 6.84 (1H, d J=7.68 Hz), 7.25-7.45 (3H, m), 7.69-7.80 (3H, m), 7.99 (1H, d J=8.66 Hz), 10.32 (1H, s) negative ESI-MS(m/z): 472 (M−1)⁻

Preparation 188

A mixture of 2-chloro-6-methylnicotinic acid (1.72 g), 1-(2-(2-pyridinyl)ethyl)-5-indolinamine (2.4 g), 1-hydroxybenzotriazole hydrate (1.61 g) and 1-[3-(dimethylamino)propyl]-3-ethylcarbodiimide (1.63 g) in N,N-dimethylformamide (100 ml) was stirred at ambient temperature for 15 hours. The reaction mixture was poured into a mixture of ethyl acetate and water, and the organic layer was washed with brine and dried over magnesium sulfate. The solvent was evaporated in vacuo and the residue was chromatographed on silica gel eluting with ethyl acetate:n-hexane (8:2 v/v) The eluted fractions containing the desired product were collected and the solvent was concentrated in vacuo and the precipitate was collected by filtration to give 2-chloro-6-methyl-N-{1-[2-(2-pyridinyl)ethyl]-2,3-dihydro-1H-indol-5-yl}nicotinamide (2.81 g).

¹H-NMR(DMSO-d₆): δ 2.50 (3H, s), 2.73-3.02 (4H, m), 3.30-3.45 (4H, m), 6.49 (1H, d J=8.44 Hz), 7.21-7.41 (5H, m), 7.67-7.71 (1H, m), 7.88 (1H, d J=7.64 Hz), 8.50-8.53 (1H, m), 10.18 (1H, s)

### EXAMPLE 364

A mixture of 2-chloro-6-methyl-N-{1-[2-(2-pyridinyl)ethyl]-2,3-dihydro-1H-indol-5-yl}nicotinamide (590 mg) and 4-methylpiperidine(0.71 ml) in tetrahydrofuran (10 ml) was refluxed under stirring for 8 hours. The reaction mixture was poured into a mixture of ethyl acetate and water, and the organic layer was washed with brine and dried over magnesium sulfate. The solvent was evaporated in vacuo and the residue was chromatographed on silica gel eluting with ethyl acetate n-hexane (7:3 v/v). The eluted fractions containing the desired product were collected and the solvent was evaporated in vacuo and the residue was recrystallized from a mixture of ether and n-hexane to give 6-methyl-2-(4-methyl-1-piperidinyl)-N-{1-[2-(2-pyridinyl)ethyl]-2,3-dihydro-1H-indol-5-yl}nicotinamide (375 mg).

¹H-NMR(DMSO-d₆): δ 0.90 (3H, d J=6.10 Hz), 1.18-1.30 (2H, m) 1.48-1.66 (3H, m), 2.40 (3H, s), 2.75-3.02 (6H, m), 3.22-3.45 (4H, m), 3.60-3.67 (2H, m), 6.49 (1H, d J=8.42 Hz), 6.82 (1H, d J=7.60 Hz), 7.19-7.34 (3H, m), 7.46 (1H, s), 7.66-7.76 (2H, m), 8.51 (1H, d J=4.04 Hz), 10.29 (1H, s) ESI-MS(m/z): 478(M+Na)⁺, 459(M+1)⁺

Preparation 189

A mixture of tert-butyl 5-{[(2-chloro-6-methyl-3-pyridinyl)carbonyl]amino}-1-indolinecarboxylate (1.2 g) and sodium isopropoxide (1.02 g) in tetrahydrofuran (15 ml) was refluxed under stirring for 10 hours. The reaction mixture was evaporated in vacuo and the residue was dissolved in a mixture of ethyl acetate and water, and the organic layer was washed with brine and dried over magnesium sulfate. The solvent was evaporated in vacuo and the residue was chromatographed on silica gel eluting with ethyl acetate:n-hexane (7:3 v/v). The eluted fractions containing the desired product were collected and evaporated in vacuo to give tert-butyl 5-{[(2-isopropoxy-6-methyl-3-pyridinyl)carbonyl]amino}-1-indolinecarboxylate (770 mg).

¹H-NMR(DMSO-d₆): δ 1.35 (9H, s), 1.41 (6H, d J=6.24 Hz), 2.18 (3H, s), 3.07 (2H, m), 3.91 (2H, m), 5.32-5.46 (1H, m), 6.87 (1H, s), 6.99 (1H, d J=7.60 Hz), 7.32-7.41 (1H, m), 7.61 (1H, s), 8.07 (1H, d J=7.60 Hz), 9.96 (1H, s)

Preparation 190

The following compound was obtained in substantially the same manner as in Preparation 41.

N-(2,3-Dihydro-1H-indol-5-yl)-2-isopropoxy-6-methylnicotinamide

¹H-NMR(DMSO-d₆): δ 1.42 (6H, d J=6.26 Hz), 2.44 (3H, s), 2.92 (2H, t J=8.28 Hz), 3.41-3.45 (2H, m), 5.38-5.50 (2H, m), 6.48 (1H, d J=8.22 Hz), 6.98 (1H, d J=7.60 Hz), 7.20 (1H, dd J=1.94 Hz, 8.22 Hz), 7.41 (1H, d J=1.94 Hz), 8.10 (1H, d J=7.60 Hz), 9.76 (1H, s)

### EXAMPLE 365

The following compound was obtained in substantially the same manner as in Example 26.

2-Isopropoxy-6-methyl-N-[1-(2-pyridinylacetyl)-2,3-dihydro-1H-indol-5-yl]nicotinamide

¹H-NMR(DMSO-d₆): δ 1.41 (6H, d J=6.14 Hz), 2.45 (3H, s), 3.18 (2H, t J=8.26 Hz), 4.01 (2H, s), 4.23 (2H, t J=8.26 Hz), 5.40-5.46 (1H, m), 6.99 (1H, d J=7.68 Hz), 7.20-7.42 (3H, m), 7.67-7.77 (2H, m), 8.00 (1H, d J=8.70 Hz), 8.07 (1H, d J=7.62 Hz), 8.48-8.51 (1H, m), 10.00 (1H, s) ESI-MS(m/z): 453(M+Na)⁺, 431(M+1)⁺

Preparation 191

The following compound was obtained in substantially the same manner as in Example 349.

tert-Butyl 4-[4-({[6-methyl-2-(4-methyl-1-piperidinyl)-3-pyridinyl]carbonyl}amino)phenyl]-1-piperazinecarboxylate

¹H-NMR(DMSO-d₆): δ 0.90 (3H, d J=6.12 Hz), 1.17-1.21 (2H, m), 1.42-1.50 (1H, m), 1.63-1.64 (2H, m), 2.39 (3H, s), 2.77-2.83 (2H, m), 3.03-3.06 (4H, m), 3.44-3.45 (4H, m), 3.63-3.66 (2H, m), 6.82 (1H, d J=7.60 Hz), 6.95 (2H, d J=9.00 Hz), 7.58 (2H, d J=9.00 Hz), 7.74 (1H, d J=7.60 Hz), 10.39 (1H, s) ESI-MS(m/z): 495(M+Na)⁺, 473(M+1)⁺

Preparation 192

The following compound was obtained in substantially the same manner as in Preparation 41.

6-Methyl-2-(4-methyl-1-piperidinyl)-N-[4-(1-piperazinyl)phenyl]nicotinamide

¹H-NMR(DMSO-d₆): δ 0.90 (3H, d J=6.14 Hz), 1.17-1.22 (2H, m), 1.40-1.53 (1H, m), 1.61-1.64 (2H, m), 2.39 (3H, s), 2.77-2.83 (2H, m), 2.91-2.94 (4H, m), 3.05-3.08 (4H, m), 3.58-3.66 (2H, m), 6.82 (1H, d J=7.64 Hz), 6.93 (2H, d J=9.08 Hz), 7.58 (1H, d J=9.08 Hz), 7.75 (1H, d J=7.64 Hz), 10.39 (1H, s)

### EXAMPLE 366

A mixture of 6-methyl-2-(4-methyl-1-piperidinyl)-N-[4-(1-piperazinyl)phenyl]nicotinamide (512 mg), pyrrole-2-carboxaldehyde (148 mg) and sodium triacetoxyborohydride (827 mg) in chloroform (20 ml) was stirred at ambient temperature for 15 hours. A water (10 ml) was added to a reaction mixture and adjusted to PH 8.5 with 10% potassium carbonate solution and stirred at ambient temperature for 30 minutes. The organic layer was washed with brine and dried over magnesium sulfate. The solvent was evaporated in vacuo and the residue was chromatographed on silica gel eluting with ethyl acetate:n-hexane (6:4 v/v). The eluted fractions containing the desired product were collected and the solvent was evaporated in vacuo and the residue was recrystallized from a mixture of diisopropyl ether and n-hexane to give 6-methyl-2-(4-methyl-1-piperidinyl)-N-{4-[4-(1H-pyrrol-2-ylmethyl)-1-piperazinyl]phenyl}nicotinamide (260 mg).

¹H-NMR(DMSO-d₆): δ 0.90 (3H, d J=6.20 Hz), 1.11-1.29 (2H, m), 1.46-1.66 (3H, m), 2.39 (3H, s), 2.48-2.51 (4H, m), 2.74-2.86 (2H, m), 3.34-3.44 (4H, m), 3.77 (2H, s), 4.01-4.05 (2H, m), 5.88-5.95 (2H, m), 6.63-6.66 (1H, m), 6.82 (1H, d J=7.56 Hz), 6.91 (2H, d J=8.94 Hz), 7.56 (2H, d J=8.94 Hz), 7.75 (1H, d J=7.56 Hz), 10.38 (1H, s)

### EXAMPLE 367

The following compound was obtained in substantially the same manner as in Example 366.

6-Methyl-2-(4-methyl-1-piperidinyl)-N-{4-[4-(2-thienylmethyl)-1-piperazinyl]phenyl}nicotinamide

¹H-NMR(DMSO-d₆): δ 0.90 (3H, d J=6.14 Hz), 1.17-1.29 (2H, m), 1.41-1.66 (3H, m), 2.39 (3H, s), 2.50-2.57 (4H, m), 2.74-2.86 (2H, m), 3.07-3.12 (4H, m), 3.73 (2H, s), 3.60-3.67 (2H, m), 6.82 (1H, d J=7.68 Hz), 6.89-6.99 (4H, m), 7.43-7.46 (1H, m), 7.57 (2H, d J=8.94 Hz), 7.75 (1H, d J=7.58 Hz), 10.39 (1H, s) ESI-MS(m/z): 512(M+Na)⁺, 490(M+1)⁺

### EXAMPLE 368

The following compound was obtained in substantially the same manner as in Example 366.

N-{4-[4-(2-Furylmethyl)-1-piperazinyl]phenyl}-6-methyl-2-(4-methyl-1-piperidinyl)nicotinamide

¹H-NMR(DMSO-d₆): δ 0.90 (3H, d J=6.16 Hz), 1.11-1.29 (2H, m), 1.46-1.66 (3H, m), 2.39 (3H, s), 2.49-2.54 (4H, m), 2.74-2.86 (2H, m), 3.06-3.11 (4H, m), 3.54 (2H, s), 3.60-3.67 (2H, m), 6.31 (1H, d J=3.18 Hz), 6.41-6.43 (1H, m), 6.82 (1H, d J=7.56 Hz), 6.91 (2H, d J=9.00 hz), 7.56 (2H, d J=9.00 Hz), 7.60-7.61 (1H, m), 7.75 (1H, d J=7.56 Hz), 10.38 (1H, s) ESI-MS(m/z): 496(M+Na)⁺, 474(M+1)⁺

Preparation 193

The following compound was obtained in substantially the same manner as in Example 349.

tert-Butyl 4-{4-[(2-isopropoxy-4-methylbenzoyl)amino]phenyl}-1-piperazinecarboxylate

¹H-NMR(DMSO-d₆): δ 1.38 (6H, d J=6.00 Hz), 1.42 (9H, s), 2.36 (3H, s), 3.03-3.07 (4H, m), 3.45-3.49 (4H, m), 4.75-4.87 (1H, m), 6.86-7.03 (3H, m), 7.03 (1H, s), 7.56 (2H, d J=8.90 Hz), 7.74 (1H, d J=7.80 Hz), 9.96 (1H, s)

Preparation 194

The following compound was obtained in substantially the same manner as in Preparation 41.

2-Isopropoxy-4-methyl-N-[4-(1-piperazinyl)phenyl]benzamide

¹H-NMR(DMSO-d₆): δ 1.39 (6H, d J=6.00 Hz), 2.36 (3H, s), 3.25-3.30 (8H, m), 4.75-4.87 (1H, m), 6.88 (1H, d J=7.70 Hz), 6.97-7.04 (3H, m), 7.59 (2H, d J=8.94 Hz), 7.72 (1H, d J=7.88 Hz), 8.76 (1H, m), 9.98 (1H, s)

### EXAMPLE 369

The following compound was obtained in substantially the same manner as in Example 366.

2-Isopropoxy-4-methyl-N-{4-[4-(1H-pyrrol-2-ylmethyl)-1-piperazinyl]phenyl}benzamide

¹H-NMR(DMSO-d₆): δ 1.38 (6H, d J=6.02 Hz), 2.35 (3H, s), 2.49-2.50 (4H, m), 3.06-3.08 (4H, m), 3.47 (2H, s), 4.75-4.87 (1H, m), 5.90-5.96 (2H, m), 6.63-6.66 (1H, m), 6.86-6.94 (2H, m), 7.03 (1H, s), 7.54 (2H, d J=8.92 Hz), 7.73 (1H, d J=7.90 hz), 9.94 (1H, s), 10.70 (1H, s) ESI-MS(m/z): 455(M+Na)⁺, 433(M+1)⁺

### EXAMPLE 370

The following compound was obtained in substantially the same manner as in Example 366.

N-{4-[4-(3-Cyanobenzyl)-1-piperazinyl]phenyl}-2-isopropoxy-4-methylbenzamide

¹H-NMR(DMSO-d₆): δ 1.39 (6H, d J=6.00 Hz), 2.36 (3H, s), 2.50-2.52 (4H, m), 3.08-3.11 (4H, m), 3.59 (2H, s), 4.75-4.87 (1H, m), 6.86-6.94 (3H, m), 7.03 (1H, s), 7.52-7.60 (3H, m), 7.68-7.77 (4H, m), 9.95 (1H, s)

Preparation 195

A solution of 2-chloro-6-methylnicotinoyl chloride (1.91 g) in tetrahydrofuran (10 ml) was added to a mixture of 6-amino-2-[2-(2-pyridinyl)ethyl]-1-isoindolinone (2.58 g) and triethylamine (4.06 g) in tetrahydrofuran (50 ml) at ambient temperature with stirring. The mixture was stirred at ambient temperature for 5 hours. The resultant mixture was poured into a mixture of ethyl acetate and water and the organic layer was washed with 5% potassium carbonate solution and brine and dried over magnesium sulfate. The solvent was evaporated in vacuo and the residue was chromatographed on silica gel eluting with chloroform: methanol (95:5 v/v). The eluted fractions containing the desired product were collected and the solvent was concentrated in vacuo and the precipitate was collected by filtration to give 2-chloro-6-methyl-N-{3-oxo-2-[2-(2-pyridinyl)ethyl]-2,3-dihydro-1H-isoindol-5-yl}nicotinamide (2.86 g).

¹H-NMR(DMSO-d₆): δ 2.53 (3H, s), 3.09 (2H, t J=7.28 Hz), 3.91 (2H, t J=7.28 Hz), 4.40 (2H, s), 7.21-7.25 (1H, m), 7.30 (1H, d J=7.86 Hz), 7.43 (1H, d J=7.76 Hz), 7.55 (1H, d J=8.24 Hz), 7.65-7.80 (2H, m), 7.99 (1H, d J=7.76 Hz), 8.08 (1H, d J=1.70 Hz), 8.47-8.50 (1H, m), 10.76 (1H, s)

### EXAMPLE 373

The following compound was obtained in substantially the same manner as in Example 364.

6-Methyl-2-(4-methyl-1-piperidinyl)-N-{3-oxo-2-[2-(2-pyridinyl)ethyl]-2,3-dihydro-1H-isoindol-5-yl}nicotinamide

¹H-NMR(DMSO-d₆): δ 0.87 (3H, d J=6.20 Hz), 1.14-1.25 (2H, m), 1.47-1.64 (3H, m), 2.40 (3H, s), 2.75-2.87 (2H, m), 3.08 (2H, t J=7.34 Hz), 3.65-3.71 (2H, m), 3.90 (2H, t J=7.34 Hz), 4.39 (2H, s), 6.82 (1H, d J=7.66 Hz), 7.21-7.25 (1H, m), 7.31 (1H, d J=7.88 Hz), 7.53 (1H, d J=8.20 Hz), 7.66-7.82 (3H, m), 8.13 (1H, d J=1.54 Hz), 8.48-8.50 (1H, m), 10.56 (1H, s) negative ESI-MS(m/z): 468 (M−1)⁻

Preparation 196

The mixture of 2-fluoro-3-(trifluoromethyl)benzonitrile (2.8 g) and 2 moL/L tetrahydrofuran solution of dimethylamine (22.2 ml) was heated at 80° C. in sealed tube for 7 hours. To the reaction mixture was added a mixture of ethyl acetate and water. The separated organic layer was washed with water, dried over magnesium sulfate and evaporated in vacuo to give 2-(dimethylamino)-3-(trifluoromethyl)benzonitrile (3.04 g).

¹H-NMR(DMSO-d₆): δ 2.88(6H, s), 7.52-7.58(1H, m), 8.02(1H, dd, J=1.3 Hz, 8.0 Hz), 8.11(1H, dd, J=1.3 Hz, 7.8 Hz)

Preparation 197

The mixture of 2-(dimethylamino)-3-(trifluoromethyl)benzonitrile (3.0 g) and sodium hydroxide (1.1 g) in ethylene glycol (12 mL) was stirred at 180° C. for 8 hours. After the mixture was added a water (22 mL) at 80° C. and the mixture was stirred at same temperature for 1 hour. To the mixture was added saturated aqueous sodium chloride and adjusted to pH 4 with 6N hydrochloric acid. The mixture was extracted with a mixture of ethyl acetate and tetrahydrofuran. The extract layer was dried over magnesium sulfate and evaporated in vacuo. The residue was purified by column chromatography on silica gel using a mixture of ethyl acetate and isopropyl ether (1:1 v/v) as an eluant. The eluted fractions containing the desired product were collected and evaporated in vacuo to give 2-(dimethylamino)-3-(trifluoromethyl)benzoic acid (1.01 g).

¹H-NMR(DMSO-d₆): δ 2.76(6H, s), 7.42(1H, t, J=7.7 Hz), 7.75-7.92(2H, m), 13.55(1H, s) (+)ESI-MS(m/z): 234(M+H)⁺, 256(M+Na)⁺

### EXAMPLE 374

1-[3-(Dimethylamino)propyl]-3-ethylcarbodiimide (0.19 g) was added to the solution of 1-(2-pyridinylacetyl)-5-indolinamine (0.25 g), 2-(dimethylamino)-3-(trifluoromethyl)benzoic acid (0.28 g), 1-hydroxybenzotriazole (0.16 g) and 4-dimethylaminopyridine (6 mg) in dimethylformamide (5 ml) under ice-cooling and the mixture was stirred at ambient temperature for 18 hours. The reaction mixture was poured into a mixture of ethyl acetate and water. The separated organic layer was washed with water, dried over magnesium sulfate and evaporated in vacuo. The residue was triturated with a mixture of ethyl acetate and isopropyl ether to give 2-(dimethylamino)-N-[1-(2-pyridinylacetyl)-2,3-dihydro-1H-indol-5-yl]-3-(trifluoromethyl)benzamide (0.19 g).

¹H-NMR(DMSO-d₆): δ 2.74(6H, s), 3.18(2H, t, J=8.3 Hz), 4.01(2H, s), 4.22(2H, t, J=8.3 Hz), 7.28(1H, dd, J=5.2 Hz,6.8 Hz), 7.33-7.50(3H, m), 7.63-7.86(4H, m), 8.01(1H, d, J=8.7 Hz), 8.48-8.54(1H, m), 10.46(1H, s) (+) ESI-MS (m/z): 469 (M+H)⁺, 491 (M+Na)⁺

### EXAMPLE 375

The following compound was obtained in substantially the same manner as in Example 374.

2-(Dimethylamino)-3-methyl-N-[1-(2-pyridinylacetyl)-2,3-dihydro-1H-indol-5-yl]benzamide

¹H-NMR(DMSO-d₆): δ 2.31(3H, s), 2.75(6H, s), 3.17(2H, t, J=8.3 Hz), 4.01(2H, s), 4.22(2H, t, J=8.3 Hz), 7.08(1H, t, J=7.5 Hz), 7.23-7.48(5H, m), 7.69-7.83(2H, m), 7.98(1H, dd, J=8.7 Hz), 8.47-8.54(1H, m), 10.78(1H, s) (+)ESI-MS(m/z): 415(M+H)⁺, 437(M+Na)⁺

### EXAMPLE 376

The following compound was obtained in substantially the same manner as in Example 374.

2-(Dimethylamino)-N-[1-(2-pyridinylacetyl)-2,3-dihydro-1H-indol-5-yl]-4-(trifluoromethyl)benzamide

¹H-NMR(DMSO-d₆): δ 2.85(6H, s), 3.17(2H, t, J=8.3 Hz), 4.01(2H, s), 4.22(2H, t, J=8.3 Hz), 7.20-7.47(5H, m), 7.62(1H, d, J=8.0 Hz), 7.69(1H, s), 7.77(1H, dt, J=1.8 Hz,7.6 Hz), 7.99(1H, d, J=8.7 Hz), 8.47-8.53(1H, m), 10.57(1H, s) (+)ESI-MS(m/z): 469(M+H)⁺, 491(M+Na)⁺

### EXAMPLE 377

The following compound was obtained in substantially the same manner as in Example 374.

4-Chloro-2-(dimethylamino)-N-[1-(2-pyridinylacetyl)-2,3-dihydro-1H-indol-5-yl]benzamide

¹H-NMR(DMSO-d₆): δ 2.80(6H, s), 3.16(2H, t, J=8.3 Hz), 4.01(2H, s), 4.22(2H, t, J=8.3 Hz), 7.02(1H, dd, J=1.8 Hz,8.2 Hz), 7.10(1H, d, J=1.8 Hz), 7.27(1H, dd, J=5.4 Hz, 7.1 Hz), 7.33-7.47(2H, m), 7.53(1H, d, J=8.2 Hz), 7.69(1H, s), 7.71-7.82(1H, m), 7.99(1H, d, J=8.7 Hz), 8.48-8.54(1H, m), 10.71(1H, s) (+)ESI-MS(m/z): 435(M+H)⁺, 457(M+Na)⁺

### EXAMPLE 378

The following compound was obtained in substantially the same manner as in Example 374.

2-(Dimethylamino)-4-fluoro-N-[1-(2-pyridinylacetyl)-2,3-dihydro-1H-indol-5-yl]benzamide

¹H-NMR(DMSO-d₆): δ 2.79(6H, s), 3.17(2H, t, J=8.3 Hz), 4.01(2H, s), 4.22(2H, t, J=8.3 Hz), 6.73-6.85(1H, m), 6.90(1H, dd, J=2.4 Hz, 12.1 Hz), 7.24-7.32(1H, m), 7.33-7.46(2H, m), 7.52-7.62(1H, m), 7.69(1H, s), 7.72-7.82(1H, m), 7.98(1H, d, J=8.6 Hz), 8.48-8.53(1H, m), 10.68(1H, s) (+) ESI-MS (m/z): 419 (M+H)⁺, 441 (M+Na)⁺

### EXAMPLE 379

The following compound was obtained in substantially the same manner as in Example 374.

2-(Dimethylamino)-4-ethyl-N-[1-(2-pyridinylacetyl)-2,3-dihydro-1H-indol-5-yl]benzamide

¹H-NMR(DMSO-d₆): δ 1.20(3H, t, J=7.5 Hz), 2.63(2H, q, J=7.5 Hz), 2.76(6H, s), 3.17(2H, t, J=8.3 Hz), 4.01(2H, s), 4.22(2H, t, J=8.3 Hz), 6.94-7.01(1H, m), 7.10(1H, s), 7.23-7.32(1H, m), 7.34-7.47(2H, m), 7.63-7.82(3H, m), 8.00(1H, d, J=8.6 Hz), 8.48-8.53(1H, m), 11.43(1H, s) (+)ESI-MS(m/z): 429 (M+H)⁺, 451 (M+Na)⁺

### EXAMPLE 380

The following compound was obtained in substantially the same manner as in Example 374.

2-(Dimethylamino)-4-isopropyl-N-[1-(2-pyridinylacetyl)-2,3-dihydro-1H-indol-5-yl]benzamide

¹H-NMR(DMSO-d₆): δ 1.22(6H, d, J=6.8 Hz), 2.77(6H, s), 2.83-3.01(1H, m), 3.17(2H, t, J=8.3 Hz), 4.01(2H, s), 4.22(2H, t, J=8.3 Hz), 7.01(1H, d, J=8.1 Hz), 7.10(1H, s), 7.23-7.33(1H, m), 7.33-7.48(2H, m), 7.63-7.82(3H, m), 7.99(1H, d, J=8.6 Hz), 8.48-8.53(1H, m), 11.36(1H, s) (+)ESI-MS(m/z): 443(M+H)⁺, 465(M+Na)⁺

### EXAMPLE 381

The following compound was obtained in substantially the same manner as in Example 374.

4-tert-Butyl-2-(dimethylamino)-N-[1-(2-pyridinylacetyl)-2,3-dihydro-1H-indol-5-yl]benzamide

¹H-NMR(DMSO-d₆): δ 1.31(9H, s), 2.78(6H, s), 3.17(2H, t, J=8.3 Hz), 4.01(2H, s), 4.22(2H, t, J=8.3 Hz), 7.15(1H, dd, J=1.6 Hz, 8.2 Hz), 7.20-7.33(2H, m), 7.34-7.47(2H, m), 7.64-7.83(3H, m), 7.99(1H, d, J=8.6 Hz), 8.48-8.54(1H, m), 11.41(1H, s) (+)ESI-MS(m/z): 457 (M+H)⁺, 479 (M+Na)⁺

### EXAMPLE 382

The following compound was obtained in substantially the same manner as in Example 374.

2-(Dimethylamino)-4-methoxy-N-[1-(2-pyridinylacetyl)-2,3-dihydro-1H-indol-5-yl]benzamide

¹H-NMR(DMSO-d₆): δ 2.76(6H, s), 3.17(2H, t, J=8.3 Hz), 3.81(3H, s), 4.01(2H, s), 4.23(2H, t, J=8.3 Hz), 6.68-6.78(2H, m), 7.24-7.45(3H, m), 7.69-7.82(3H, m), 7.98(1H, d, J=8.5 Hz), 8.48-8.52(1H, m), 11.41(1H, s) (+)ESI-MS(m/z): 431(M+H)⁺, 453(M+Na)⁺

### EXAMPLE 383

The following compound was obtained in substantially the same manner as in Example 374.

4-Acetyl-2-(dimethylamino)-N-[1-(2-pyridinylacetyl)-2,3-dihydro-1H-indol-5-yl]benzamide

¹H-NMR(DMSO-d₆): δ 2.61(3H, s), 2.83(6H, s), 3.17(2H, t, J=8.4 Hz), 4.01(2H, s), 4.23(2H, t, J=8.4 Hz), 7.24-7.32(1H, m), 7.37(1H, d, J=7.7 Hz), 7.39-7.48(1H, m), 7.56-7.82(5H, m), 8.00(1H, d, J=8.7 Hz), 8.48-8.53(1H, m), 10.86(1H, s) (+)ESI-MS(m/z): 443(M+H)⁺, 465(M+Na)⁺

### EXAMPLE 384

The following compound was obtained in substantially the same manner as in Example 374.

2-(Dimethylamino)-5-methyl-N-[1-(2-pyridinylacetyl)-2,3-dihydro-1H-indol-5-yl]benzamide

¹H-NMR(DMSO-d₆): δ 2.30(3H, s), 2.73(6H, s), 3.18(2H, t, J=8.4 Hz), 4.01(2H, s), 4.22(2H, t, J=8.4 Hz), 7.21(1H, d, J=8.2 Hz), 7.26-7.32(2H, m), 7.37(1H, d, J=7.8 Hz), 7.43(1H, d, J=8.6 Hz), 7.60(1H, s), 7.71(1H, s), 7.74-7.80(1H, m), 8.00(1H, d, J=8.6 Hz), 8.49-8.53(1H, m), 11.71(1H, s) (+)ESI-MS(m/z): 415(M+H)⁺, 437(M+Na)⁺

### EXAMPLE 385

The following compound was obtained in substantially the same manner as in Example 374.

5-Chloro-2-(dimethylamino)-N-[1-(2-pyridinylacetyl)-2,3-dihydro-1H-indol-5-yl]benzamide

¹H-NMR(DMSO-d₆): δ 2.77(6H, s), 3.17(2H, t, J=8.3 Hz), 4.01(2H, s), 4.22(2H, t, J=8.3 Hz), 7.17(1H, d, J=8.8 Hz), 7.23-7.32(1H, m), 7.34-7.49(3H, m), 7.56(1H, d, J=2.5 Hz), 7.70(1H, s), 7.72-7.82(1H, m), 8.00(1H, d, J=8.6 Hz), 8.47-8.53(1H, m), 11.00(1H, s) (+)ESI-MS(m/z): 435(M+H)⁺, 457(M+Na)⁺

### EXAMPLE 386

The following compound was obtained in substantially the same manner as in Example 374.

2-(Dimethylamino)-4,5-dimethoxy-N-[1-(2-pyridinylacetyl)-2,3-dihydro-1H-indol-5-yl]benzamide

¹H-NMR(DMSO-d₆): δ 2.75(6H, s), 3.18(2H, t, J=8.4 Hz), 3.79(3H, s), 3.86(3H, s), 4.01(2H, s), 4.23(2H, t, J=8.4 Hz), 7.05(1H, s), 7.24-7.33(1H, m), 7.33-7.47(2H, m), 7.56(1H, s), 7.68-7.83(2H, m), 8.00(1H, d, J=8.6 Hz), 8.47-8.53(1H, m), 12.71(1H, s) (+)ESI-MS(m/z): 461(M+H)⁺, 483(M+Na)⁺

### EXAMPLE 387

The following compound was obtained in substantially the same manner as in Example 374.

2-(Diethylamino)-4-methyl-N-[1-(2-pyridinylacetyl)-2,3-dihydro-1H-indol-5-yl]benzamide ¹H-NMR(DMSO-d₆): δ 0.96(6H, t, J=7.1 Hz), 2.37(3H, s), 3.02-3.27(6H, m), 4.01(2H, s), 4.23(2H, t, J=8.3 Hz), 7.15(1H, d, J=8.1 Hz), 7.22-7.47(4H, m), 7.67-7.83(2H, m), 7.97-8.07(2H, m), 8.47-8.55(1H, m), 13.13(1H, s) (+) ESI-MS (m/z): 443 (M+H)⁺, 465 (M+Na)⁺

### EXAMPLE 388

The following compound was obtained in substantially the same manner as in Example 374.

tert-Butyl [5-methyl-2-({[1-(2-pyridinylacetyl)-2,3-dihydro-1H-indol-5-yl]amino}carbonyl)phenyl]carbamate

¹H-NMR(DMSO-d₆): δ 1.46(9H, s), 2.35(3H, s), 3.18(2H, t, J=8.3 Hz), 4.02(2H, s), 4.23(2H, t, J=8.3 Hz), 6.96(1H, d, J=7.3 Hz), 7.24-7.32(1H, m), 7.34-7.45(2H, m), 7.65(1H, s), 7.71-7.82(2H, m), 7.97-8.06(2H, m), 8.48-8.54(1H, m), 10.26(1H, s), 10.30(1H, s) (+) ESI-MS (m/z): 487 (M+H)⁺, 509 (M+Na)⁺

### EXAMPLE 389

The mixture of tert-butyl 5-methyl-2-({[1-(2-pyridinylacetyl)-2,3-dihydro-1H-indol-5-yl]amino}carbonyl)phenylcarbamate (1.5 g) and trifluoroacetic acid (1.9 mL) in dichloromethane (3.0 mL) was stirred for 20 hours at ambient temperature. The reaction mixture was poured into a mixture of ethyl acetate and water and the mixture was adjusted to pH 9 with potassium carbonate. The isolated precipitate was collected by filtration to give 2-amino-4-methyl-N-[1-(2-pyridinylacetyl)-2,3-dihydro-1H-indol-5-yl]benzamide (1.14 g).

¹H-NMR(DMSO-d₆): δ 2.20(3H, s), 3.15(2H, t, J=8.3 Hz), 4.00(2H, s), 4.22(2H, t, J=8.3 Hz), 4.27-4.46(3H, m), 6.54(1H, s), 6.23-6.33(1H, m), 6.33-6.47(2H, m), 6.54(1H, d, J=8.0 Hz), 7.65(1H, s), 7.71-7.82(1H, m), 7.97(1H, d, J=8.7 Hz), 8.47-8.54(1H, m), 9.83(1H, s) (−)ESI-MS(m/z): 385(M−H)⁻

### EXAMPLE 390

The following compound was obtained in substantially the same manner as in Example 374.

4-Methoxy-2-(4-methyl-1-piperidinyl)-N-[1-(2-pyridinylacetyl)-2,3-dihydro-1H-indol-5-yl]benzamide

¹H-NMR(DMSO-d₆): δ 0.95(3H, d, J=5.9 Hz), 1.20-1.63(3H, m), 1.65-1.82(2H, m), 2.70-2.86(2H, m), 3.03-3.25(4H, m), 3.82(3H, s), 4.01(2H, s), 4.23(2H, t, J=8.3 Hz), 6.77-6.86(2H, m), 7.24-7.33(1H, m), 7.33-7.44(2H, m), 7.72-7.84(2H, m), 7.88(1H, d, J=8.7 Hz), 8.02(1H, d, J=8.7 Hz), 8.47-8.55(1H, m), 11.76(1H, s) (+)ESI-MS(m/z): 485(M+H)⁺, 507 (M+Na)⁺

### EXAMPLE 391

The following compound was obtained in substantially the same manner as in Example 374.

1-Methyl-N-[1-(2-pyridinylacetyl)-2,3-dihydro-1H-indol-5-yl]-1,2,3,4-tetrahydro-8-quinolinecarboxamide

¹H-NMR(DMSO-d₆): δ 1.18-1.89(2H, m), 2.73(2H, t, J=6.3 Hz), 2.76(3H, s), 3.13-3.22(4H, m), 4.00(2H, s), 4.21(2H, t, J=8.4 Hz), 6.76(1H, t, J=7.5 Hz), 7.06(1H, d, J=7.5 Hz), 7.25-7.31(2H, m), 7.37(1H, d, J=7.5 Hz), 7.42(1H, d, J=8.7 Hz), 7.69(1H, s), 7.74-7.80(1H, m), 7.98(1H, d, J=8.7 Hz), 8.49-8.53(1H, m), 10.39(1H, s) (+) ESI-MS (m/z): 427 (M+H)⁺, 449 (M+Na)⁺

### EXAMPLE 392

The following compound was obtained in substantially the same manner as in Example 374.

1-Ethyl-N-[1-(2-pyridinylacetyl)-2,3-dihydro-1H-indol-5-yl]-1,2,3,4-tetrahydro-8-quinolinecarboxamide

¹H-NMR(DMSO-d₆): δ 0.97(3H, t, J=7.0 Hz), 1.71-1.88(2H, m), 2.76(2H, t, J=6.2 Hz), 3.01(2H, q, J=7.0 Hz), 3.08-3.24(4H, m), 4.00(2H, s), 4.21(2H, t, J=8.4 Hz), 6.83(1H, t, J=7.5 Hz), 7.03-7.13(1H, m), 7.22-7.33(2H, m), 7.36(1H, d, J=7.8 Hz), 7.43(1H, dd, J=2.0 Hz, 8.5 Hz), 7.69-7.83(2H, m), 7.97(1H, d, J=8.7 Hz), 8.46-8.54(1H, m), 10.40(1H, s) (+)ESI-MS(m/z): 441(M+H)⁺, 463(M+Na)⁺

### EXAMPLE 393

The following compound was obtained in substantially the same manner as in Example 374.

5-Chloro-1-methyl-N-[1-(2-pyridinylacetyl)-2,3-dihydro-1H-indol-5-yl]-1,2,3,4-tetrahydro-8-quinolinecarboxamide

¹H-NMR(DMSO-d₆): δ 1.79-1.95(2H, m), 2.74(2H, t, J=6.3 Hz), 2.78(3H, s), 3.09-3.23(4H, m), 4.00(2H, s), 4.22(2H, t, J=8.3 Hz), 6.89(1H, d, J=8.2 Hz), 7.22-7.33(2H, m), 7.33-7.45(2H, m), 7.67(1H, s), 7.76(1H, dt, J=1.8Hz, 7.7 Hz), 7.98(1H, d, J=8.7 Hz), 8.47-8.53(1H, m), 10.29(1H, s) (+)ESI-MS(m/z): 461(M+H)⁺, 483(M+Na)⁺

Preparation 198

A mixture of methyl 3-amino-4-methyl-2-thiophenecarboxylate (5.0 g), sodium hydrogencarbonate (14.7 g) and dimethyl sulfate (8.3 mL) in 2-butanone (50 mL) was heated under reflux for 17 hours. The solvent was removed by concentration. The residue was diluted with water and extracted with ethyl acetate. The extract layer was washed with water, dried over magnesium sulfate and evaporated in vacuo to give methyl 3-(dimethylamino)-4-methyl-2-thiophenecarboxylate (5.65 g).

¹H-NMR(DMSO-d₆): δ 2.13(3H, s), 2.83(6H, s), 3.73(3H, s), 7.36(1H, s)

Preparation 199

A mixture of methyl 3-(dimethylamino)-4-methyl-2-thiophenecarboxylate (5.6 g) and lithium hydroxide monohydrate (2.4 g) in a mixture of methanol (56 ml) and water (12 mL) was stirred for 6 days at ambient temperature. To the reaction mixture was added conc. hydrochloric acid (4.7 mL) and the mixture was evaporated in vacuo. The residue was purified by column chromatography on silica gel using a mixture of chloroform and methanol (19:1 v/v) as an eluant. The eluted fractions containing the desired product were collected and evaporated in vacuo to give 3-(dimethylamino)-4-methyl-2-thiophenecarboxylic acid (0.55 g).

¹H-NMR(DMSO-d₆): δ 2.24(3H, d, J=0.8 Hz), 2.84(6H, s), 7.43(1H, d, J=0.8 Hz), 14.50(1H, s) (−)ESI-MS(m/z): 184(M−H)⁻

### EXAMPLE 394

The following compound was obtained in substantially the same manner as in Example 374.

3-(Dimethylamino)-4-methyl-N-[1-(2-pyridinylacetyl)-2,3-dihydro-1H-indol-5-yl]-2-thiophenecarboxamide

¹H-NMR(DMSO-d₆): δ 2.31(3H, s), 2.87(6H, s), 3.18(2H, t, J=8.3 Hz), 4.01(2H, s), 4.23(2H, t, J=8.3 Hz), 7.23-7.33(1H, m), 7.34-7.45(3H, m), 7.65(1H, s), 7.77(1H, dt, J=1.7 Hz, 7.6 Hz), 8.02(1H, d, J=8.6 Hz), 8.48-8.54(1H, m), 11.90(1H, s) (+)ESI-MS(m/z): 421(M+H)⁺, 443 (M+Na)⁺

### EXAMPLE 395

The following compound was obtained in substantially the same manner as in Example 374.

2-Isopropyl-4-methyl-N-[1-(2-pyridinylacetyl)-2,3-dihydro-1H-indol-5-yl]benzamide

¹H-NMR(DMSO-d₆): δ 1.20(6H, d, J=6.9 Hz), 2.34(3H, s), 3.07-3.36(3H, m), 4.00(2H, s), 4.21(2H, t, J=8.4 Hz), 7.07(1H, d, J=8.1 Hz), 7.20-7.48(5H, m), 7.69-7.82(2H, m), 7.97(1H, d, J=8.7 Hz), 8.47-8.54(1H, m), 10.21(1H, s) (+)ESI-MS(m/z): 414(M+H)⁺, 436(M+Na)⁺

### EXAMPLE 396

The following compound was obtained in substantially the same manner as in Example 374.

2-Isopropenyl-4-methyl-N-[1-(2-pyridinylacetyl)-2,3-dihydro-1H-indol-5-yl]benzamide

¹H-NMR(DMSO-d₆): δ 2.03(3H, s), 2.34(3H, s), 3.15(2H, t, J=8.3 Hz), 4.00(2H, s), 4.21(2H, t, J=8.3 Hz), 4.94(1H, s), 5.05(1H, s), 7.13-7.42(6H, m), 7.64(1H, s), 7.76(1H, dt, J=1.8 Hz, 7.6 Hz), 7.96(1H, d, J=8.7 Hz), 8.47-8.54(1H, m), 10.08(1H, s) (+)ESI-MS(m/z): 412(M+H)⁺, 434(M+Na)⁺

### EXAMPLE 397

The following compound was obtained in substantially the same manner as in Example 374.

2-tert-Butyl-4-methyl-N-[1-(2-pyridinylacetyl)-2,3-dihydro-1H-indol-5-yl]benzamide

¹H-NMR(DMSO-d₆): δ 1.36(9H, s), 2.33(3H, s), 3.16(2H, t, J=8.3 Hz), 4.00(2H, s), 4.21(2H, t, J=8.3 Hz), 7.05-7.11(1H, m), 7.14(1H, d, J=7.6 Hz), 7.23-7.44(4H, m), 7.68(1H, s), 7.71-7.81(1H, m), 7.96(1H, d, J=8.7 Hz), 8.48-8.53(1H, m), 10.23(1H, s) (+)ESI-MS(m/z): 428(M+H)⁺, 450(M+Na)⁺

### EXAMPLE 398

The following compound was obtained in substantially the same manner as in Example 374.

4-Chloro-2-cyclohexyl-N-[1-(2-pyridinylacetyl)-2,3-dihydro-1H-indol-5-yl]benzamide

¹H-NMR(DMSO-d₆): δ 1.08-1.58(5H, m), 1.58-1.94(5H, m), 2.74-2.96(1H, m), 3.17(2H, t, J=8.2 Hz), 4.01(2H, s), 4.22(2H, t, J=8.2 Hz), 7.20-7.51(6H, m), 7.64-7.84(2H, m), 7.99(1H, d, J=8.7 Hz), 8.44-8.56(1H, m), 10.33(1H, s) (+)ESI-MS(m/z): 474(M+H)⁺, 496(M+Na)⁺

### EXAMPLE 399

The following compound was obtained in substantially the same manner as in Example 374.

2-Cyclohexyl-N-[1-(2-pyridinylacetyl)-2,3-dihydro-1H-indol-5-yl]benzamide

¹H-NMR(DMSO-d₆): δ 1.16-1.31(3H, m), 1.37-1.51(2H, m), 1.62-1.87(5H, m), 2.78-2.89(1H, m), 3.17(2H, t, J=8.3 Hz), 4.01(2H, s), 4.22(2H, t, J=8.3 Hz), 7.23-7.47(7H, m), 7.71(1H, s), 7.77(1H, dt, J=1.7 Hz, 7.6 Hz), 7.98(1H, d, J=8.7 Hz), 8.48-8.54(1H, m), 10.28(1H, s) (+)ESI-MS(m/z): 440(M+H)⁺, 462(M+Na)⁺

### EXAMPLE 400

The following compound was obtained in substantially the same manner as in Example 374.

2-(Methylthio)-N-[1-(2-pyridinylacetyl)-2,3-dihydro-1H-indol-5-yl]benzamide

¹H-NMR(DMSO-d₆): δ 2.43(3H, s), 3.16(2H, t, J=8.3 Hz), 4.01(2H, s), 4.22(2H, t, J=8.3 Hz), 7.19-7.54(7H, m), 7.66-7.84(2H, m), 7.98(1H, d, J=8.7 Hz), 8.45-8.56(1H, m), 10.26(1H, s) (+)ESI-MS(m/z): 404(M+H)⁺, 426(M+Na)⁺

### EXAMPLE 401

The following compound was obtained in substantially the same manner as in Example 374.

2-(Methylsulfonyl)-N-[1-(2-pyridinylacetyl)-2,3-dihydro-1H-indol-5-yl]benzamide

¹H-NMR(DMSO-d₆): δ 3.18(2H, t, J=8.3 Hz), 3.38(3H, s), 4.01(2H, s), 4.23(2H, t, J=8.3 Hz), 7.23-7.33(1H, m), 7.37(2H, d, J=7.9 Hz), 7.66-7.89(5H, m), 7.96-8.06(2H, m), 8.48-8.54(1H, m), 10.57(1H, s) (+)ESI-MS(m/z): 436(M+H)⁺, 458(M+Na)⁺

### EXAMPLE 402

The following compound was obtained in substantially the same manner as in Example 374.

4-Methyl-2-(methylthio)-N-[1-(2-pyridinylacetyl)-2,3-dihydro-1H-indol-5-yl]benzamide

¹H-NMR(DMSO-d₆): δ 2.36(3H, s), 2.42(3H, s), 3.16(2H, t, J=8.2 Hz), 4.00(2H, s), 4.21(2H, t, J=8.2 Hz), 7.05(1H, d, J=7.7 Hz), 7.21(1H, s), 7.23-7.33(1H, m), 7.33-7.48(3H, m), 7.66-7.82(2H, m), 7.98(1H, d, J=8.7 Hz), 8.47-8.54(1H, m), 10.17(1H, s) (+)ESI-MS(m/z): 418(M+H)⁺, 440(M+Na)⁺

### EXAMPLE 403

1-[3-(Dimethylamino)propyl]-3-ethylcarbodiimide (0.19 g) was added to a solution of tert-butyl 4-aminophenyl(2-(2-pyridinyl)ethyl)carbamate (0.31 g), 2-isopropyl-4-methylbenzoic acid (0.21 g), 1-hydroxybenzotriazole (0.16 g) and 4-dimethylaminopyridine (6 mg) in tetrahydrofuran (4 ml), and the mixture was stirred at ambient temperature for 18 hours. To the reaction mixture was added a solution of 4N solution of hydrogen chloride in dioxane (7.5 ml) and the mixture was stirred at same temperature for 30 hours. The reaction mixture was poured into a mixture of ethyl acetate and water, and the mixture was adjusted to pH 9 with potassium carbonate. The separated organic layer was washed with water, dried over magnesium sulfate and evaporated in vacuo. The residue was purified by column chromatography on silica gel using a mixture of ethyl acetate and isopropyl ether (1:1 v/v) as an eluant. The eluted fractions containing the desired product were collected and evaporated in vacuo to give 2-isopropyl-4-methyl-N-(4-{[2-(2-pyridinyl)ethyl]amino}phenyl)benzamide (0.27 g).

¹H-NMR(DMSO-d₆): δ 1.20(6H, d, J=6.8 Hz), 2.33(3H, s), 2.98(2H, t, J=7.3 Hz), 3.18-3.44(3H, m), 5.53(1H, t, J=5.7 Hz), 6.57(2H, d, J=8.7 Hz), 7.06(1H, d, J=7.7 Hz), 7.17-7.28(3H, m), 7.31(1H, d, J=7.7 Hz), 7.44(2H, d, J=8.7 Hz), 7.71(1H, dt, J=1.6 Hz, 7.6 Hz), 8.48-8.56(1H, m), 9.86(1H, s) (+)ESI-MS(m/z): 374(M+H)⁺, 396(M+Na)⁺

### EXAMPLE 404

The following compound was obtained in substantially the same manner as in Example 403.

2-(Methylthio)-N-(4-{[2-(2-pyridinyl)ethyl]amino}phenyl)benzamide

¹H-NMR(DMSO-d₆): δ 2.42(3H, s), 2.99(2H, t, J=7.2 Hz), 3.28-3.46(2H, m), 5.56(1H, s), 6.58(2H, d, J=8.8 Hz), 7.17-7.53(8H, m), 7.65-7.76(1H, m), 8.48-8.57(1H, m), 9.92(1H, s) (+)ESI-MS(m/z): 364(M+H)⁺, 386(M+Na)⁺

### EXAMPLE 405

The following compound was obtained in substantially the same manner as in Example 403.

4-Methyl-2-(methylthio)-N-(4-{[2-(2-pyridinyl)ethyl]amino}phenyl)benzamide

¹H-NMR(DMSO-d₆): δ 2.35(3H, s), 2.41(3H, s), 2.98(2H, t, J=7.2 Hz), 3.29-3.44(2H, m), 5.54(1H, t, J=5.7 Hz), 6.57(2H, d, J=8.8 Hz), 7.03(1H, d, J=7.7 Hz), 7.16-7.38(4H, m), 7.42(2H, d, J=8.8 Hz), 7.71(1H, dt, J=1.8 Hz, 7.7 Hz), 8.47-8.56(1H, m), 9.83(1H, s) (+)ESI-MS(m/z): 378(M+H)⁺, 400(M+Na)⁺

Preparation 200

1-[3-(Dimethylamino)propyl]-3-ethylcarbodiimide (2.3 g) was added to the solution of ethyl 4-aminobenzoate (2.0 g), 4-methyl-2-(4-methyl-1-piperidinyl)benzoic acid (3.1 g), 1-hydroxybenzotriazole (2.0 g) and 4-dimethylaminopyridine (74 mg) in tetrahydrofuran (30 ml) and the mixture was stirred at ambient temperature for 18 hours. The reaction mixture was poured into a mixture of ethyl acetate and water. The separated organic layer was washed with water, dried over magnesium sulfate and evaporated in vacuo. The residue was purified by column chromatography on silica gel using a mixture of hexane and ethyl acetate (9:1 v/v) as an eluant. The eluted fractions containing the desired product were collected and evaporated in vacuo to give ethyl 4-{[4-methyl-2-(4-methyl-1-piperidinyl)benzoyl]amino}benzoate (2.5 g).

¹H-NMR(DMSO-d₆): δ 0.96(3H, d, J=6.1 Hz), 1.20-1.62(3H, m), 1.32(3H, t, J=7.1 Hz), 1.65-1.81(2H, m), 2.36(3H, s), 2.70-2.88(2H, m), 3.05-3.19(2H, m), 4.30(2H, q, J=7.1 Hz), 7.06(1H, d, J=7.8 Hz), 7.18(1H, s), 7.80(1H, d, J=7.8 Hz), 7.88(2H, d, J=8.8 Hz), 7.99(2H, d, J=8.8 Hz), 12.17(1H, s)

Preparation 201

The mixture of ethyl 4-{[4-methyl-2-(4-methyl-1-piperidinyl)benzoyl]amino}benzoate (2.4 g) and sodium hydroxide (0.38 g) in a mixture of methanol (24 ml), tetrahydrofuran (20 mL) and water (8 mL) was stirred for 2 days at ambient temperature. The solvent was removed by concentration. The residue was diluted with water and adjusted to pH 5.5 with 6N hydrochloric acid. The mixture was extracted with ethyl acetate. The extract layer was washed with water, dried over magnesium sulfate and evaporated in vacuo to give 4-{[4-methyl-2-(4-methyl-1-piperidinyl)benzoyl]amino}benzoic acid (2.1 g).

¹H-NMR(DMSO-d₆): δ 0.96(3H, d, J=6.1 Hz), 1.20-1.63(3H, m), 1.65-1.82(2H, m), 2.36(3H, s), 2.70-2.87(2H, m), 3.05-3.20(2H, m), 7.05(1H, d, J=7.9 Hz), 7.18(1H, s), 7.79(1H, d, J=7.9 Hz), 7.85(2H, d, J=8.8 Hz), 7.96(2H, d, J=8.8 Hz), 12.10(1H, s), 12.74(1H, s) (+)ESI-MS(m/z): 353(M+H)⁺

### EXAMPLE 406

1-[3-(Dimethylamino)propyl]-3-ethylcarbodiimide (0.19 g) was added to the solution of 4-{[4-methyl-2-(4-methyl-1-piperidinyl)benzoyl]amino}benzoic acid (0.35 g), 2-aminopyridine (0.28 g), 1-hydroxybenzotriazole (0.16 g) and 4-dimethylaminopyridine (6 mg) in tetrahydrofuran (4 ml) and the mixture was stirred at ambient temperature for 40 hours. The reaction mixture was poured into a mixture of ethyl acetate and water and the mixture was adjusted to pH 9 with potassium carbonate. The separated organic layer was washed with water, dried over magnesium sulfate and evaporated in vacuo. The residue was triturated with hexane to give 4-methyl-2-(4-methyl-1-piperidinyl)-N-{4-[(2-pyridinylamino)carbonyl]phenyl}benzamide (80 mg).

¹H-NMR(DMSO-d₆): δ 0.97(3H, d, J=6.0 Hz), 1.22-1.62(3H, m), 1.67-1.83(2H, m), 2.36(3H, s), 2.70-2.88(2H, m), 3.05-3.21(2H, m), 7.06(1H, d, J=8.0 Hz), 7.12-7.22(2H, m), 7.76-7.92(4H, m), 8.10(2H, d, J=8.7 Hz), 8.21(1H, d, J=8.4 Hz), 8.36-8.43(1H, m), 10.68(1H, s), 12.11(1H, s) (+)ESI-MS(m/z): 429(M+H)⁺, 451(M+Na)⁺

### EXAMPLE 407

The following compound was obtained in substantially the same manner as in Example 406.

4-Methyl-2-(4-methyl-1-piperidinyl)-N-(4-{[(2-pyridinylmethyl)amino]carbonyl}phenyl)benzamide

¹H-NMR(DMSO-d₆): δ 0.96(3H, d, J=5.9 Hz), 1.20-1.63(3H, m), 1.64-1.83(2H, m), 2.36(3H, s), 2.70-2.87(2H, m), 3.04-3.21(2H, m), 4.58(2H, d, J=5.7 Hz), 7.06(1H, d, J=7.9 Hz), 7.19(1H, s), 7.27(1H, dd, J=5.4 Hz, 7.1 Hz), 7.33(1H, d, J=7.9 Hz), 7.69-8.03(6H, m), 8.48-8.56(1H, m), 9.04(1H, t, J=5.7 Hz), 12.08(1H, s) (+)ESI-MS(m/z): 443(M+H)⁺, 465(M+Na)⁺

### EXAMPLE 408

The following compound was obtained in substantially the same manner as in Example 406.

N-(4-{[(di-2-Pyridinylmethyl)amino]carbonyl}phenyl)-4-methyl-2-(4-methyl-1-piperidinyl)benzamide

¹H-NMR(DMSO-d₆): δ 0.96(3H, d, J=5.9 Hz), 1.23-1.63(3H, m), 1.68-1.81(2H, m), 2.36(3H, s), 2.71-2.88(2H, m), 3.06-3.19(2H, m), 6.46(1H, d, J=7.9 Hz), 7.06(1H, d, J=8.0 Hz), 7.19(1H, s), 7.26-7.35(2H, m), 7.55(2H, d, J=7.9 Hz), 7.74-7.90(5H, m), 8.00(2H, d, J=8.7 Hz), 8.50-8.57(2H, m), 9.13(1H, d, J=7.9 Hz), 12.13(1H, s) (+)ESI-MS(m/z): 520(M+H)⁺, 542(M+Na)⁺

### EXAMPLE 409

The following compound was obtained in substantially the same manner as in Example 406.

4-Methyl-2-(4-methyl-1-piperidinyl)-N-[4-({[2-(2-pyridinyl)ethyl]amino}carbonyl)phenyl]benzamide

¹H-NMR(DMSO-d₆): δ 0.96(3H, d, J=6.0 Hz), 1.20-1.63(3H, m), 1.65-1.84(2H, m), 2.36(3H, s), 2.70-2.87(2H, m), 3.00(2H, t, J=7.7 Hz), 3.06-3.19(2H, m), 3.54-3.70(2H, m), 7.06(1H, d, J=8.0 Hz), 7.15-7.33(3H, m), 7.65-7.93(6H, m), 8.45-8.58(2H, m), 12.07(1H, s) (+)ESI-MS(m/z): 457(M+H)⁺, 479(M+Na)⁺

### EXAMPLE 410

The following compound was obtained in substantially the same manner as in Example 406.

4-Methyl-2-(4-methyl-1-piperidinyl)-N-[4-({[1-(2-pyridinyl)ethyl]amino}carbonyl)phenyl]benzamide

¹H-NMR(DMSO-d₆): δ 0.96(3H, d, J=6.0 Hz), 1.20-1.63(3H, m), 1.51(3H, d, J=7.1 Hz), 1.67-1.82(2H, m), 2.36(3H, s), 2.71-2.88(2H, m), 3.06-3.19(2H, m), 5.11-5.29(1H, m), 7.06(1H, d, J=7.9 Hz), 7.19(1H, s), 7.21-7.30(1H, m), 7.41(1H, d, J=7.8 Hz), 7.77-7.88(4H, m), 7.96(2H, d, J=8.6 Hz), 8.50-8.56(1H, m), 8.74(1H, d, J=7.7 Hz), 12.11(1H, s) (+)ESI-MS(m/z): 457(M+H)⁺, 479(M+Na)⁺

Preparation 202

The following compound was obtained in substantially the same manner as in Preparation 200.

Ethyl 4-{[2-(dimethylamino)-4-methylbenzoyl]amino}benzoate

¹H-NMR(DMSO-d₆): δ 1.32(3H, t, J=7.1 Hz), 2.35(3H, s), 2.77(6H, s), 4.30(2H, q, J=7.1 Hz), 6.96(1H, d, J=7.7 Hz), 7.11(1H, s), 7.66(1H, d, J=7.7 Hz), 7.86(2H, d, J=8.8 Hz), 7.95(2H, d, J=8.8 Hz), 11.83(1H, s)

Preparation 203

The following compound was obtained in substantially the same manner as in Preparation 201.

4-{[2-(Dimethylamino)-4-methylbenzoyl]amino}benzoic acid

¹H-NMR(DMSO-d₆): δ 2.35(3H, s), 2.77(6H, s), 6.97(1H, d, J=7.9 Hz), 7.12(1H, s), 7.67(1H, d, J=7.9 Hz), 7.84(2H, d, J=8.8 Hz), 7.93(2H, d, J=8.8 Hz), 11.83(1H, s), 12.74(1H, s) (−)ESI-MS(m/z): 297(M−H)⁻

### EXAMPLE 411

The following compound was obtained in substantially the same manner as in Example 406.

2-(Dimethylamino)-4-methyl-N-[4-({[2-(2-pyridinyl)ethyl]amino}carbonyl)phenyl]benzamide

¹H-NMR(DMSO-d₆): δ 2.35(3H, s), 2.77(6H, s), 3.01(2H, t, J=7.4 Hz), 3.55-3.71(2H, m), 6.97(1H, d, J=7.8 Hz), 7.12(1H, s), 7.17-7.33(2H, m), 7.65-7.90(6H, m), 8.45-8.58(2H, m), 11.76(1H, s) (+)ESI-MS(m/z): 403(M+H)⁺, 425(M+Na)⁺

### EXAMPLE 412

The following compound was obtained in substantially the same manner as in Example 406.

2-(Dimethylamino)-4-methyl-N-[4-({[1-(2-pyridinyl)ethyl]amino}carbonyl)phenyl]benzamide

¹H-NMR(DMSO-d₆): δ 1.52(3H, d, J=7.0 Hz), 2.35(3H, s), 2.77(6H, s), 5.12-5.29(1H, m), 6.97(1H, d, J=7.9 Hz), 7.12(1H, s), 7.21-7.31(1H, m), 7.41(1H, d, J=7.9 Hz), 7.63-7.87(4H, m), 7.94(2H, d, J=8.6 Hz), 8.49-8.57(1H, m), 8.74(1H, d, J=7.7 Hz), 11.74(1H, s) (+)ESI-MS(m/z): 403(M+H)⁺, 425(M+Na)⁺

Preparation 204

4-Nitrobenzoyl chloride (1.0 g) was added dropwise to the mixture of [1-(2-pyridinyl)ethyl]amine (1.32 g) in acetone (13 mL) and water (8 mL) at 0-8° C. under keeping to pH 7-8 with 20% aqueous potassium carbonate and the mixture was stirred for 2 hours at same condition. The solvent was removed by concentration. The residue was diluted with water and adjusted to pH 9 with 20% aqueous potassium carbonate. The mixture was extracted with ethyl acetate. The extract layer was washed with water, dried over magnesium sulfate and evaporated in vacuo to give 4-nitro-N-[1-(2-pyridinyl)ethyl]benzamide (0.82 g).

¹H-NMR(DMSO-d₆): δ 1.54(3H, d, J=7.1 Hz), 5.14-5.32(1H, m), 7.23-7.32(1H, m), 7.44(1H, d, J=7.8 Hz), 7.72-7.84(1H, m), 8.11-8.21(2H, m), 8.28-8.38(2H, m), 8.51-8.58(1H, m), 9.11(1H, d, J=7.7 Hz)

Preparation 205

To a mixture of 4-nitro-N-[1-(2-pyridinyl)ethyl]benzamide (1.0 g) in methanol (15 ml) was added 10% palladium-on-charcoal (0.3 g, 50% wet). The reaction mixture was stirred at ambient temperature for 4 hours under hydrogen atmosphere. The catalyst was filtered off and the solvent was removed by concentration to give 4-amino-N-[1-(2-pyridinyl)ethyl]benzamide (0.86 g).

¹H-NMR(DMSO-d₆): δ 1.47(3H, d, J=7.1 Hz), 5.07-5.24(1H, m), 5.64(2H, s), 6.56(2H, d, J=8.6 Hz), 7.17-7.27(1H, m), 7.37(1H, d, J=7.9 Hz), 7.65(2H, d, J=8.6 Hz), 7.73(1H, dt, J=1.8 Hz, 7.6 Hz), 8.33(1H, d, J=7.8 Hz), 8.47-8.55(1H, m) (+)ESI-MS(m/z): 264(M+Na)⁺

### EXAMPLE 413

1-[3-(Dimethylamino)propyl]-3-ethylcarbodiimide (0.19 g) was added to the solution of 4-amino-N-[1-(2-pyridinyl)ethyl]benzamide (0.24 g), 6-methyl-2-(4-methyl-1-piperidinyl)nicotinic acid (0.28 g), 1-hydroxybenzotriazole (0.16 g) and 4-dimethylaminopyridine (6 mg) in dimethylformamide (4 ml) and the mixture was stirred at ambient temperature for 20 hours. The reaction mixture was poured into a mixture of ethyl acetate and water. The separated organic layer was washed with water, dried over magnesium sulfate and evaporated in vacuo. The residue was purified by column chromatography on silica gel using a mixture of ethyl acetate and isopropyl ether (1:1 v/v) as an eluant. The eluted fractions containing the desired product were collected and evaporated in vacuo to give 6-methyl-2-(4-methyl-1-piperidinyl)-N-[4-({[1-(2-pyridinyl)ethyl]amino}carbonyl)phenyl]nicotinamide (76.0 mg).

¹H-NMR(DMSO-d₆): δ 0.88(3H, d, J=6.1 Hz), 1.05-1.32(2H, m), 1.37-1.75(3H, m), 1.51(3H, d, J=7.1 Hz), 2.40(3H, s), 2.71-2.92(2H, m), 3.56-3.74(2H, m), 5.10-5.29(1H, m), 6.84(1H, d, J=7.6 Hz), 7.20-7.31(1H, m), 7.41(1H, d, J=7.9 Hz), 7.68-7.87(4H, m), 7.93(2H, d, J=8.8 Hz), 8.48-8.56(1H, m), 8.73(1H, d, J=7.8 Hz), 10.74(1H, s) (+)ESI-MS(m/z): 458(M+H)⁺, 480(M+Na)⁺

### EXAMPLE 414

The following compound was obtained in substantially the same manner as in Example 413.

4-Methyl-2-(4-methyl-1-piperidinyl)-N-(4-{[2-(2-pyridinyl)propanoyl]amino}phenyl)benzamide

¹H-NMR(DMSO-d₆): δ 0.95(3H, d, J=6.0 Hz), 1.20-1.63(3H, m), 1.48(3H, d, J=7.0 Hz), 1.66-1.81(2H, m), 2.34(3H, s), 2.69-2.87(2H, m), 3.03-3.17(2H, m), 4.01(1H, q, J=7.0 Hz), 7.04(1H, d, J=8.0 Hz), 7.16(1H, s), 7.23-7.32(1H, m), 7.45(1H, d, J=7.0 Hz), 7.56-7.73(4H, m), 7.73-7.85(2H, m), 8.49-8.56(1H, m), 10.14(1H, s), 11.85(1H, s) (+)ESI-MS(m/z): 457(M+H)⁺, 479(M+Na)⁺

### EXAMPLE 415

The following compound was obtained in substantially the same manner as in Example 413.

6-Methyl-2-(4-methyl-1-piperidinyl)-N-(4-{[2-(2-pyridinyl)propanoyl]amino}phenyl)nicotinamide

¹H-NMR(DMSO-d₆): δ 0.88(3H, d, J=6.1 Hz), 1.06-1.30(2H, m), 1.37-1.68(3H, m), 1.48(3H, d, J=7.0 Hz), 2.39(3H, s), 2.69-2.90(2H, m), 3.56-3.71(2H, m), 4.02(1H, q, J=7.0 Hz), 6.82(1H, d, J=7.7 Hz), 7.23-7.31(1H, m), 7.45(1H, d, J=7.9 Hz), 7.58(2H, d, J=9.2 Hz), 7.65(2H, d, J=9.2 Hz), 7.72-7.83(2H, m), 8.48-8.56(1H, m), 10.14(1H, s), 10.49(1H, s) (+)ESI-MS(m/z): 458(M+H)⁺, 480(M+Na)⁺

### EXAMPLE 416

The following compound was obtained in substantially the same manner as in Example 403.

3-(Dimethylamino)-4-methyl-N-(4-{[2-(2-pyridinyl)ethyl]amino}phenyl)-2-thiophenecarboxamide

¹H-NMR(DMSO-d₆): δ 2.31(3H, s), 2.86(6H, s), 2.98(2H, t, J=7.2 Hz), 3.30-3.44(2H, m), 5.60(1H, t, J=5.7 Hz), 6.60(2H, d, J=8.8 Hz), 7.18-7.27(1H, m), 7.29-7.44(4H, m), 7.71(1H, dt, J=1.8 Hz, 7.7 Hz), 8.49-8.54(1H, m), 11.54(1H, s) (+)ESI-MS(m/z): 381(M+H)⁺, 403(M+Na)⁺

### EXAMPLE 417

The following compound was obtained in substantially the same manner as in Example 403.

4-Ethyl-2-(methylamino)-N-(4-{[2-(2-pyridinyl)ethyl]amino}phenyl)benzamide

¹H-NMR(DMSO-d₆): δ 1.19(3H, t, J=7.5 Hz), 2.57(2H, q, J=7.5 Hz), 2.79(3H, d, J=5.0 Hz), 2.99(2H, t, J=7.0 Hz), 3.27-3.44(2H, m), 5.53(1H, t, J=5.7 Hz), 6.41-6.52(2H, m), 6.56(2H, d, J=8.8 Hz), 7.22(1H, dd, J=5.4 Hz, 7.0 Hz), 7.27-7.43(3H, m), 7.48(1H, q, J=5.0 Hz), 7.57(1H, d, J=8.1 Hz), 7.71(1H, dt, J=1.8 Hz, 7.6 Hz), 8.48-8.54(1H, m), 9.64(1H, s) (+)ESI-MS(m/z): 375(M+H)⁺, 397(M+Na)⁺

Preparation 206

To a mixture of 2-[5-(tritylamino)-1H-pyrazol-1-yl]ethanol (4.00 g) and 1-fluoro-4-nitrobenzene (1.906 g) in N,N-dimethylformamide (20 ml) was added potassium tert-butoxide (1.823 g) at ambient temperature. The reaction mixture was heated to 55° C. for 1 hour, cooled to room temperature, poured into water and extracted with ethyl acetate. The organic layer was washed with water and brine, dried over magnesium sulfate, filtered and concentrated in vacuo to yield a dark yellow solid. The residue was suspended in ethyl acetate—hexane (1:1), filtered and washed with diisopropyl ether, then hexane to give 1-[2-(4-nitrophenoxy)ethyl]-N-trityl-1H-pyrazol-5-amine (4.255 g) as pale yellow crystals.

¹H-NMR(CDCl₃): δ 4.36-4.41(2H, m), 4.44-4.49(2H, m), 4.63(1H, d, J=2.0 Hz), 5.68(1H, s), 6.45(2H, d, J=9.2 Hz), 7.03(1H, d, J=2.0 Hz), 7.27(15H, s), 8.07(2H, d, J=9.2 Hz) (+)ESI-MS(m/z): 491(M+H)⁺

Preparation 207

A solution of '1-[2-(4-nitrophenoxy)ethyl]-N-trityl-1H-pyrazol-5-amine (1.000 g) in methylalcohol—N,N-dimethylformamide (1:1) (40 ml) was hydrogenated over 10% Pd/C (50% wet, 0.200 g) at 40° C. under atmospheric pressure of hydrogen for 5 hours. The reaction mixture was filtered through a pad of celite, and the filtrate was concentrated in vacuo to give 1-[2-(4-aminophenoxy)ethyl]-N-trityl-1H-pyrazol-5-amine (0.767 g) as a pale orange powder.

¹H-NMR(CDCl₃): δ 3.38(2H, brs), 4.17-4.24(2H, m), 4.38-4.45(2H, m), 4.60(1H, d, J=2.0 Hz), 6.17(1H, brs), 6.27(2H, d, J=8.9 Hz), 6.50(2H, d, J=8.9 Hz), 7.00(1H, d, J=2.0 Hz), 7.19-7.38(15H, m) (+)ESI-MS(m/z): 461(M+H)⁺, 483(M+Na)⁺

### EXAMPLE 418

To a solution of 4-chloro-2-(dimethylamino)benzoic acid (184 mg), 1-[2-(4-aminophenoxy)ethyl]-N-trityl-1H-pyrazol-5-amine (386 mg) and 1-hydroxybenzotriazole monohydrate (167 mg) in N,N-dimethylformamide (10 ml) was added 1-[3-(dimethylamino)propyl]-3-ethylcarbodiimide hydrochloride (209 mg), followed by triethylamine (110 mg) at ambient temperature and the mixture was stirred at the same temperature for 4 days. The reaction mixture was poured into a mixture of ethyl acetate and water. The separated organic layer was washed with brine, dried over magnesium sulfate and concentrated in vacuo. The residue was suspended in ethyl acetate—hexane (1:1), filtered and washed with hexane to give 4-chloro-2-(dimethylamino)-N-(4-{2-[5-(tritylamino)-1H-pyrazol-1-yl]ethoxy}phenyl)benzamide (467 mg) as pale yellow crystals.

¹H-NMR(CDCl₃): δ 2.81(6H, s), 4.26-4.31(2H, s), 4.44-4.48(2H, m), 4.61(1H, d, J=2.0 Hz), 6.08(1H, s), 6.43(2H, d, J=8.9 Hz), 7.01(1H, d, J=2.0 Hz), 7.19-7.34(17H, m), 7.44(2H, d, J=8.9 Hz), 8.16(1H, d, J=8.9 Hz), 11.52(1H, s) (+)ESI-MS(m/z): 664(M+Na)⁺

### EXAMPLE 419

To a solution of 4-chloro-2-(dimethylamino)-N-(4-{2-[5-(tritylamino)-1H-pyrazol-1-yl]ethoxy}phenyl)benzamide (457 mg) in methanol (10 ml) was added concentrated hydrochloric acid (370 mg). The reaction mixture was stirred for 2 hours at 40° C., quenched with 10% potassium carbonate solution, and extracted with ethyl acetate. The organic layer was washed with brine, dried over magnesium sulfate, filtered and concentrated in vacuo. The residue was suspended in ethyl acetate—hexane (1:1), filtered and washed with hexane to give N-{4-[2-(5-amino-1H-pyrazol-1-yl)ethoxy]phenyl}-4-chloro-2-(dimethylamino)benzamide (257 mg) as faintly brown crystals.

¹H-NMR(CDCl₃): δ 2.81(6H, s), 3.97(2H, brs), 4.28-4.33(2H, m), 4.38-4.43(2H, m), 5.52(1H, d, J=2.0 Hz), 6.83(2H, d, J=8.9 Hz), 7.20-7.28(3H, m), 7.55(2H, d, J=9.2 Hz), 8.15(1H, d, J=8.9 Hz), 11.55(1H, brs) (+)ESI-MS(m/z): 400(M+H)⁺, 422(M+Na)⁺

### EXAMPLE 420

To a solution of 2-(4-methylphenyl)-1-cyclohexene-1-carboxylic acid (186 mg), 1-[2-(4-aminophenoxy)ethyl]-N-trityl-1H-pyrazol-5-amine (360 mg) and 1-hydroxybenzotriazole monohydrate (156 mg) in N,N-dimethylformamide (10 ml) was added 1-[3-(dimethylamino)propyl]-3-ethylcarbodiimide hydrochloride (195 mg), followed by triethylamine (103 mg) at ambient temperature and the mixture was stirred at 50° C. for 8 hours. The reaction mixture was poured into a mixture of ethyl acetate and water. The separated organic layer was washed with brine, dried over magnesium sulfate and concentrated in vacuo. The residue was purified by column chromatography on silica gel eluting with hexane: ethyl acetate (1:2) to give 2-(4-methylphenyl)-N-(4-{2-[5-(tritylamino)-1H-pyrazol-1-yl]ethoxy}phenyl)-1-cyclohexene-1-carboxamide (488 mg) as a colorless foamy solid.

¹H-NMR(CDCl₃): δ 1.76(4H, brs), 2.33(3H, s), 2.41(2H, brs), 2.51(2H, brs), 4.17-4.21(2H, m), 4.39-4.43(2H, m), 4.58(1H, d, J=2.0 Hz), 6.21(2H, d, J=8.9 Hz), 6.49(1H, brs), 6.73(2H, d, J=8.9 Hz), 6.98(1H, d, J=2.0 Hz), 7.10-7.31(19H, m) (+)ESI-MS(m/z): 681(M+Na)⁺

### EXAMPLE 421

To a solution of 2-(4-methylphenyl)-N-(4-{2-[5-(tritylamino)-1H-pyrazol-1-yl]ethoxy}phenyl)-1-cyclohexene-1-carboxamide (476 mg) in methanol (10 ml) was added concentrated hydrochloric acid (376 mg). The reaction mixture was stirred for 1 hour at 40° C., quenched with 10% potassium carbonate solution, and extracted with ethyl acetate. The organic layer was washed with brine, dried over magnesium sulfate, filtered and concentrated in vacuo. The residue was triturated with diisopropyl ether and filtered to give N-{4-[2-(5-amino-1H-pyrazol-1-yl)ethoxy]phenyl}-2-(4-methylphenyl)-1-cyclohexene-1-carboxamide (253 mg) as a colorless powder.

¹H-NMR(CDCl₃): δ 1.76(4H, brs), 2.32(3H, s), 2.41(2H, brs), 2.51(2H, brs), 3.93(2H, brs), 4.20(2H, t, J=4.6 Hz), 4.35(2H, t, J=4.6 Hz), 5.49(1H, d, J=2.0 Hz), 6.63(2H, d, J=8.9 Hz), 6.85(2H, d, J=8.9 Hz), 7.10-7.19(4H, m), 7.25(1H, d, J=2.0 Hz) (+)ESI-MS(m/z): 417(M+H)⁺, 439(M+Na)⁺

### EXAMPLE 422

To a solution of 1-[2-(4-aminophenoxy)ethyl]-N-trityl-1H-pyrazol-5-amine (400 mg), 4-methyl-2-(4-methyl-1-piperidinyl)benzoic acid (223 mg) and 1-hydroxybenzotriazole hydrate (160 mg) in N,N-dimethylformamide (5 ml) was added 1-[3-(dimethylamino)propyl]-3-ethylcarbodiimide hydrochloride (200 mg) and triethylamine (0.181 ml) at ambient temperature. The reaction mixture was stirred for 15 hours at 50° C. and concentrated in vacuo. The residue was dissolved in ethyl acetate and water, and extracted with ethyl acetate. The organic layer was washed with water and brine, dried over magnesium sulfate, filtered and concentrated in vacuo. The residue was purified by column chromatography on silica gel eluting with hexane:ethyl acetate (1:1) to give 4-methyl-2-(4-methyl-1-piperidinyl)-N-(4-{2-[5-(tritylamino)-1H-pyrazol-1-yl]ethoxy}phenyl)benzamide (552 mg) as a pale yellow foam.

¹H-NMR(CDCl₃): δ 1.05(3H, d, J=6.3 Hz), 1.35-1.45(2H, m), 1.48-1.62(1H, m), 1.85(2H, d, J=8.5 Hz), 2.38(3H, s), 2.78-2.86(2H, m), 3.06-3.18(2H, m), 4.29(2H, t, J=4.4 Hz), 4.47(2H, t, J=4.4 Hz), 4.60(1H, d, J=2.0 Hz), 6.17(1H, s), 6.38(2H, d, J=9.2 Hz), 7.01(1H, d, J=2.0 Hz), 7.07-7.09(2H, m), 7.18-7.34(12H, m), 7.55(2H, d, J=8.9 Hz), 8.16(1H, d, J=8.2 Hz), 12.48(1H, s) ESI-MS(m/z): 698(M+Na)⁺

### EXAMPLE 423

To a solution of 4-methyl-2-(4-methyl-1-piperidinyl)-N-(4-{2-[5-(tritylamino)-1H-pyrazol-1-yl]ethoxy}phenyl)benzamide (366 mg) in methanol (6 ml) was added concentrated hydrochloric acid (837 mg). The reaction mixture was stirred for 14 hours at room temperature, quenched with 10% potassium carbonate solution and extracted with ethyl acetate. The organic layer was washed with brine, dried over magnesium sulfate, filtered and concentrated in vacuo. The residue was recrystallized from ethyl acetate-diisopropyl ether to give N-(4-[2-(5-amino-1H-pyrazol-1-yl)ethoxy]phenyl}-4-methyl-2-(4-methyl-1-piperidinyl)benzamide (225 mg) as a white solid.

¹H-NMR(DMSO-d₆): δ 0.95(3H, d, J=6.3 Hz), 1.24-1.42(2H, m), 1.48-1.60(1H, m), 1.73(2H, d, J=11.5 Hz), 2.34(3H, s), 2.73-2.81(2H, m), 3.10(2H, d, J=11.5 Hz), 4.22(4H, s), 5.18(2H, s), 5.29(1H, d, J=2.0 Hz), 6.94(2H, d, J=9.2 Hz), 7.01-7.07(2H, m), 7.16(1H, s), 7.65(2H, d, J=8.9 Hz), 7.80(1H, d, J=7.9 Hz), 11.79(1H, s) ESI-MS(m/z): 434(M+H)⁺

### EXAMPLE 424

The following compound was obtained in substantially the same manner as in Example 422.

2-(Dimethylamino)-4-methyl-N-(4-{2-[5-(tritylamino)-1H-pyrazol-1-yl]ethoxy}phenyl)benzamide

¹H-NMR(CDCl₃): δ 2.39(3H, s), 2.79(6H, s), 4.28(2H, t, J=4.3 Hz), 4.46(2H, t, J=4.3 Hz), 4.61(1H, d, J=2.0 Hz), 6.11(1H, s), 6.41(2H, d, J=8.9 Hz), 7.01(1H, d, J=2.0 Hz), 7.07(1H, d, J=6.9 Hz), 7.08(1H, s), 7.22-7.34(15H, m), 7.46(2H, d, J=8.9 Hz), 8.14(1H, d, J=8.2 Hz), 12.08(1H, s) ESI-MS(m/z): 644(M+Na)⁺

### EXAMPLE 425

The following compound was obtained in substantially the same manner as in Example 423.

N-{4-[2-(5-Amino-1H-pyrazol-1-yl)ethoxy]phenyl}-2-(dimethylamino)-4-methylbenzamide

¹H-NMR(DMSO-d₆): δ 2.33(3H, s), 2.75(6H, s), 4.22(4H, br s), 5.19(2H, s), 5.29(1H, d, J=1.6 Hz), 6.89-6.94(3H, m), 7.07-7.08(2H, m), 7.60-7.65(3H, m), 11.34(1H, s) ESI-MS(m/z): 402(M+Na)⁺

### EXAMPLE 426

The following compound was obtained in substantially the same manner as in Example 422.

6-Methyl-2-(4-methyl-1-piperidinyl)-N-(4-{2-[5-(tritylamino)-1H-pyrazol-1-yl]ethoxy}phenyl)nicotinamide

¹H-NMR(CDCl₃): δ 1.03(3H, d, J=6.6 Hz), 1.34-1.4(2H, m), 1.55-1.65(1H, m), 1.80-1.86(2H, m), 2.51(3H, s), 2.94-3.04(2H, m), 3.29-3.34(2H, m), 4.29(2H, t, J=4.3 Hz), 4.46(2H, t, J=4.3 Hz), 4.60(1H, d, J=2.0 Hz), 6.12(1H, s), 6.40(2H, d, J=8.9 Hz), 7.00(1H, s), 7.02(1H, d, J=5.6 Hz), 7.20-7.33(18H, m), 7.52(2H, d, J=9.2 Hz), 8.34(1H, d, J=7.9 Hz), 11.72(1H, s) ESI-MS(m/z): 682(M+Na)⁺

### EXAMPLE 427

The following compound was obtained in substantially the same manner as in Example 423.

N-{4-[2-(5-Amino-1H-pyrazol-1-yl)ethoxy]phenyl}-6-methyl-2-(4-methyl-1-piperidinyl)nicotinamide

¹H-NMR(DMSO-d₆): δ 0.88(3H, d, J=6.3 Hz), 1.08-1.26(2H, m), 1.39-1.54(1H, m), 1.61(2H, d, J=12.2 Hz), 2.38(3H, s), 2.79(2H, t, J=11.7 Hz), 3.33-3.67(2H, m), 4.21(4H, s), 5.18(2H, s), 5.28(1H, s), 6.80(1H, d, J=7.6 Hz), 6.90(2H, d, J=8.6 Hz), 7.07(1H, s), 7.62(2H, d, J=8.6 Hz), 7.73(1H, d, J=7.6 Hz), 10.41(1H, s) ESI-MS(m/z): 457(M+Na)⁺

Preparation 208

To a mixture of 2-[5-(tritylamino)-1H-pyrazol-1-yl]ethanol (2.239 g) and 2-chloro-5-nitropyridine (1.059 g) in N,N-dimethylformamide (20 ml) was added potassium tert-butoxide (1.021 g) at ambient temperature. The reaction mixture was stirred at the same temperature for 2 hours, poured into water and extracted with ethyl acetate. The organic layer was washed with water and brine, dried over magnesium sulfate, filtered and concentrated in vacuo to yield dark yellow tar. The residue was recrystallized from ethyl acetate to give 1-{2-[(5-nitro-2-pyridinyl)oxy]ethyl}-N-trityl-1H-pyrazol-5-amine (2.291 g) as pale yellow crystals.

¹H-NMR(CDCl₃): δ 4.41(2H, t, J=5.5 Hz), 4.68(1H, d, J=1.6 Hz), 4.70(2H, t, J=5.5 Hz), 5.44(1H, s), 6.22(1H, d, J=9.6 Hz), 7.03(1H, d, J=2.0 Hz), 7.28(15H, s), 8.16-8.23(1H, m), 8.71-8.74(1H, m) (+)ESI-MS(m/z): 514(M+Na)⁺

Preparation 209

A solution of 1-{2-[(5-nitro-2-pyridinyl)oxy]ethyl}-N-trityl-1H-pyrazol-5-amine (400 mg) in methanol (6 ml) and tetrahydrofuran (6 ml) was hydrogenated over 10% Pd/C (50% wet, 80 mg) at 40° C. under atmospheric pressure of hydrogen at 40° C. for 2 hours. The reaction mixture was cooled to ambient temperature, added a chloroform, filtered with pad of Celite, and the filtrate was concentrated in vacuo. The residue was recrystallized from ethyl acetate-diisopropyl ether to give 6-{2-[5-(tritylamino)-1H-pyrazol-1-yl]ethoxy}-3-pyridinamine (328 mg) as a pale yellow solid.

¹H-NMR(CDCl₃): δ 3.28(2H, br s), 4.36-4.40(2H, m), 4.45-4.49(2H, m), 4.65(1H, d, J=2.0 Hz), 5.94(1H, dd, J=8.6, 0.7 Hz), 6.02(1H, s), 6.83(1H, dd, J=8.6, 3.0 Hz), 6.99(1H, d, J=2.0 Hz), 7.09(1H, d, J=2.6 Hz), 7.22-7.35(15H, m) ESI-MS(m/z): 482(M+Na)⁺

### EXAMPLE 428

To a solution of 6-{2-[5-(tritylamino)-1H-pyrazol-1-yl]ethoxy}-3-pyridinamine (318 mg), 4′-(trifluoromethyl)[1,1′-biphenyl]-2-carboxylic acid (202 mg) and 1-hydroxybenzotriazole hydrate (127 mg) in N,N-dimethylformamide (5 ml) was added 1-[3-(dimethylamino)propyl]-3-ethylcarbodiimide hydrochloride (158 mg) and triethylamine (0.144 ml) at ambient temperature. The reaction mixture was stirred for 17 hours at 50° C. and concentrated in vacuo. The residue was dissolved in ethyl acetate and water, and extracted with ethyl acetate. The organic layer was washed with water and brine, dried over magnesium sulfate, filtered and concentrated in vacuo. The residue was purified by column chromatography on silica gel eluting with hexane: ethyl acetate (3:7) to give 4′-(trifluoromethyl)-N-(6-{2-[5-(tritylamino)-1H-pyrazol-1-yl)ethoxy}-3-pyridinyl)-1,1′-biphenyl-2-carboxamide (430 mg) as a pale yellow foam.

¹H-NMR(CDCl₃): δ 4.37(2H, t, J=4.9 Hz), 4.50(2H, t, J=4.9 Hz), 4.61(1H, d, J=2.0 Hz), 5.89(1H, d, J=6.9 Hz), 5.93(1H, s), 6.90-6.96(2H, m), 7.18-7.32(16H, m), 7.43-7.61(7H, m), 7.68(2H, d, J=8.2 Hz), 7.77(1H, d, J=7.6 Hz) ESI-MS(m/z): 732(M+Na)⁺

### EXAMPLE 429

To a solution of 4′-(trifluoromethyl)-N-(6-{2-[5-(tritylamino)-1H-pyrazol-1-yl]ethoxy}-3-pyridinyl)-1,1′-biphenyl-2-carboxamide (420 mg) in methanol (6 ml) was added concentrated hydrochloric acid (617 mg). The reaction mixture was stirred for 14 hours at ambient temperature, quenched with 10% potassium carbonate solution and extracted with ethyl acetate. The organic layer was washed with brine, dried over magnesium sulfate, filtered and concentrated in vacuo. The residue was recrystallized from ethyl acetate-diisopropyl ether to give N-{6-[2-(5-amino-1H-pyrazol-1-yl)ethoxy]-3-pyridinyl}-4′-(trifluoromethyl)-1,1′-biphenyl-2-carboxamide (173 mg) as a white solid.

¹H-NMR(DMSO-d₆): δ 4.20(2H, t, J=5.9 Hz), 4.44(2H, t, J=5.9 Hz), 5.15(2H, s), 5.27(1H, d, J=2.0.Hz), 6.77(1H, d, J=8.9 Hz), 7.04(1H, d, J=1.7 Hz), 7.51-7.75(6H, m), 7.77(2H, d, J=9.2 Hz), 7.82(1H, d, J=2.9 Hz), 8.27(1H, d, J=2.7 Hz), 10.39(1H, s) ESI-MS(m/z): 468(M+H)⁺

### EXAMPLE 430

To a solution of 2-(4-methylphenyl)-1-cyclohexene-1-carboxylic acid (384 mg) in toluene (5 ml) were added thionyl chloride (342 mg) and N,N-dimethylformamide (1 drop) and the mixture was stirred at 50° C. for 1 hour. The mixture was evaporated in vacuo and the residue was dissolved in tetrahydrofuran (2 ml). The acid chloride in tetrahydrofuran was added to a solution of 6-{2-[5-(tritylamino)-1H-pyrazol-1-yl]ethoxy}-3-pyridinamine (363 mg) and triethylamine (0.25 ml) in tetrahydrofuran (8 ml) at ambient temperature and the mixture was stirred at the same temperature for 1 hour. The mixture was poured into water and extracted with ethyl acetate. The organic layer was washed with brine, dried over magnesium sulfate and evaporated in vacuo. The residue was purified by column chromatography on silica gel eluting with hexane:ethyl acetate (1:1) to give 2-(4-methylphenyl)-N-(6-{2-[5-(tritylamino)-1H-pyrazol-1-yl]ethoxy}-3-pyridinyl)-1-cyclohexene-1-carboxamide (443 mg) as a yellow foam.

¹H-NMR(CDCl₃): δ 1.77(4H, br s), 2.32(3H, s), 2.42(2H, br s), 2.50(2H, br s), 4.37(2H, t, J=4.6 Hz), 4.49(2H, t, J=4.6 Hz), 4.59(1H, d, J=2.0 Hz), 5.80(1H, d, J=8.9 Hz), 5.86(1H, s), 6.46(1H, s), 6.98(1H, d, J=2.0 Hz), 7.12-7.15(5H, m), 7.20-7.30(17H, m), 7.46(1H, dd, J=8.9, 2.6 Hz) ESI-MS (m/z): 682 (M+Na)⁺

### EXAMPLE 431

To a solution of 2-(4-methylphenyl)-N-(6-{2-[5-(tritylamino)-1H-pyrazol-1-yl]ethoxy}-3-pyridinyl)-1-cyclohexene-1-carboxamide (420 mg) in methanol (6 ml) was added concentrated hydrochloric acid (617 mg). The reaction mixture was stirred for 14 hours at ambient temperature, quenched with 10% potassium carbonate solution, and extracted with ethyl acetate. The organic layer was washed with brine, dried over magnesium sulfate, filtered and concentrated in vacuo. The residue was recrystallized from ethyl acetate-diisopropyl ether to give N-{6-[2-(5-amino-1H-pyrazol-1-yl)ethoxy]-3-pyridinyl}-2-(4-methylphenyl)-1-cyclohexene-1-carboxamide (204 mg) as a pale yellow solid.

¹H-NMR(DMSO-d₆): δ 1.71(4H, br s), 2.21(3H, s), 2.35(4H, br s), 4.17(2H, t, J=5.7 Hz), 4.40(2H, t, J=5.7 Hz), 5.15(2H, s), 5.26(1H, s), 6.68(1H, d, J=8.9 Hz), 7.03(1H, s), 7.05(2H, d, J=8.9 Hz), 7.17(1H, d, J=7.9 Hz), 7.64(1H, dd, J=8.9, 1.4 Hz), 8.08(1H, s), 9.52(1H, s) ESI-MS(m/z): 418(M+H)⁺

### EXAMPLE 432

To a solution of tert-butyl 4-aminophenyl[2-(1H-pyrazol-1-yl)ethyl]carbamate (264 mg), 4-chloro-2-(dimethylamino)benzoic acid (286 mg) and 1-hydroxybenzotriazole hydrate (221 mg) in N,N-dimethylformamide (7 ml) was added 1-[3-(dimethylamino)propyl]-3-ethylcarbodiimide hydrochloride (276 mg) at ambient temperature. The reaction mixture was stirred for 16 hours at 40° C., cooled and concentrated in vacuo. The residue was dissolved in ethyl acetate and water, and extracted with ethyl acetate. The organic layer was washed with water and brine, dried over magnesium sulfate, filtered and concentrated in vacuo. The residue was purified by column chromatography on silica gel eluting with hexane: ethyl acetate (1:1) to give tert-butyl 4-{[4-chloro-2-(dimethylamino)benzoyl]amino}phenyl[2-(1H-pyrazol-1-yl)ethyl]carbamate (451 mg) as a colorless foam.

¹H-NMR(CDCl₃): δ 1.41(9H, s), 2.82(6H, s), 4.03(2H, t, J=6.3 Hz), 4.34(2H, t, J=5.9 Hz), 6.24(1H, t, J=2.0 Hz), 6.93(2H, br s), 7.23(1H, dd, J=7.9, 2.0 Hz), 7.40(1H, d, J=2.0 Hz), 7.49(1H, d, J=1.3 Hz), 7.57(2H, d, J=8.6 Hz), 8.16(1H, dd, J=7.9, 1.3 Hz), 11.71(1H, s) ESI-MS(m/z): 506(M+Na)⁺

### EXAMPLE 433

To a solution of tert-butyl 4-{[4-chloro-2-(dimethylamino)benzoyl]amino}phenyl[2-(1H-pyrazol-1-yl)ethyl]carbamate (437 mg) in dichloromethane (15 ml) was added trifluoroacetic acid (1.04 ml). The reaction mixture was stirred for 18 hours at ambient temperature, quenched with 10% potassium carbonate solution and extracted with dichloromethane. The organic layer was washed with brine, dried over magnesium sulfate, filtered and concentrated in vacuo. The residue was recrystallized from ethyl acetate-diisopropyl ether to give 4-chloro-2-(dimethylamino)-N-(4-{[2-(1H-pyrazol-1-yl)ethyl]amino}phenyl)benzamide (200 mg) as yellow solids.

¹H-NMR(DMSO-d₆): δ 2.79(6H, s), 3.41(2H, q, J=6.3 Hz), 4.25(2H, t, J=6.3 Hz), 5.61(1H, t, J=6.1 Hz), 6.22(1H, t, J=2.0 Hz), 6.56(2H, d, J=8.9 Hz), 7.00(1H, dd, J=7.9, 2.0 Hz), 7.07(1H, d, J=2.0 Hz), 7.41(2H, d, J=8.9 Hz), 7.46(1H, d, J=1.3 Hz), 7.51(1H, d, J=8.2 Hz), 7.73(1H, d, J=2.3 Hz), 10.40(1H, s) ESI-MS(m/z): 406(M+Na)⁺

### EXAMPLE 434

The following compound was obtained in substantially the same manner as in Example 432.

tert-Butyl (2-{6-[(tert-butoxycarbonyl)amino]-2-pyridinyl}ethyl)(4-{[4-chloro-2-(dimethylamino)benzoyl]amino}phenyl)carbamate

¹H-NMR(CDCl₃): δ 1.42(18H, s), 2.83(6H, s), 3.04(2H, t, J=7.9 Hz), 3.94(2H, t, J=7.9 Hz), 7.08(2H, d, J=7.9 Hz), 7.16(2H, d, J=8.6 Hz), 7.24(1H, dd, J=7.9, 2.3 Hz), 7.58-7.64(3H, m), 8.17(1H, dd, J=7.9, 1.3 Hz), 11.69(1H, s) ESI-MS(m/z): 633(M+Na)⁺

### EXAMPLE 435

The following compound was obtained in substantially the same manner as in Example 433.

N-(4-{[2-(6-Amino-2-pyridinyl)ethyl]amino}phenyl)-4-chloro-2-(dimethylamino)benzamide

¹H-NMR(DMSO-d₆): δ 2.73(2H, t, J=7.3 Hz), 2.79(6H, s), 3.27(2H, t, J=7.3 Hz), 5.59(1H, br s), 5.85(2H, br s), 6.27(1H, d, J=7.9 Hz), 6.40(1H, d, J=0.9 Hz), 6.57(2H, d, J=8.9 Hz), 7.02(1H, dd, J=8.2, 2.0 Hz), 7.08(1H, d, J=2.0 Hz), 7.27(1H, dd, J=7.9, 7.3 Hz), 7.40(2H, d, J=8.9 Hz), 7.50(1H, d, J=7.3 Hz), 10.39(1H, s) ESI-MS(m/z): 410(M+H)⁺

### EXAMPLE 436

The following compound was obtained in substantially the same manner as in Example 432.

tert-Butyl {6-[2-(4-{[4-chloro-2-(dimethylamino)benzoyl]amino}phenoxy)ethyl]-2-pyridinyl}carbamate

¹H-NMR(CDCl₃): δ 1.51(9H, s), 2.82(3H, s), 3.13(2H, t, J=6.7 Hz), 4.30(2H, t, J=6.7 Hz), 6.86-6.92(3H, m), 7.21(1H, dd, J=7.9, 2.0 Hz), 7.23(1H, s), 7.54(2H, d, J=8.9 Hz), 7.60(1H, d, J=7.9 Hz), 7.77(1H, d, J=8.2 Hz), 8.14-8.17(1H, m), 11.48(1H, s) ESI-MS(m/z): 534(M+Na)⁺

### EXAMPLE 437

The following compound was obtained in substantially the same manner as in Example 433.

N-{4-[2-(6-Amino-2-pyridinyl)ethoxy]phenyl}-4-chloro-2-(dimethylamino)benzamide

¹H-NMR(DMSO-d₆): δ 2.79(6H, s), 2.91(2H, t, J=6.7 Hz), 4.23(2H, t, J=6.7 Hz), 5.85(2H, s), 6.28(1H, d, J=8.2 Hz), 6.44(1H, d, J=6.9 Hz), 6.91(2H, d, J=8.9 Hz), 6.99(1H, dd, J=8.2, 2.0 Hz), 7.08(1H, d, J=2.0 Hz), 7.28(1H, dd, J=7.3, 6.9 Hz), 7.49(1H, d, J=8.2 Hz), 7.60(2H, d, J=8.9 Hz), 10.56(1H, s) ESI-MS(m/z): 411(M+H)⁺

### EXAMPLE 438

The following compound was obtained in substantially the same manner as in Example 432.

tert-Butyl (2-{2-[(tert-butoxycarbonyl)amino]-1,3-thiazol-4-yl}ethyl)(4-{[4-chloro-2-(dimethylamino)benzoyl]amino}phenyl)carbamate

¹H-NMR(CDCl₃): δ 1.49(18H, s), 2.83(6H, s), 2.95(2H, t, J=7.2 Hz), 3.92(2H, t, J=7.2 Hz), 6.78(1H, s), 7.14(2H, d, J=8.2 Hz), 7.21-7.26(3H, m), 7.61(2H, d, J=8.9 Hz), 8.17(1H, dd, J=7.6, 1.0 Hz), 11.70(1H, s) ESI-MS(m/z): 739(M+Na)⁺

### EXAMPLE 439

The following compound was obtained in substantially the same manner as in Example 433.

N-(4-{[2-(2-Amino-1,3-thiazol-4-yl)ethyl]amino}phenyl)-4-chloro-2-(dimethylamino)benzamide

¹H-NMR(DMSO-d₆): δ 2.66(2H, t, J=7.3 Hz), 2.79(6H, s), 3.22(2H, t, J=7.3 Hz), 5.51(1H, br s), 6.21(1H, s), 6.55(2H, d, J=8.6 Hz), 6.87(2H, s), 7.08(1H, dd, J=8.2, 2.0 Hz), 7.41(2H, d, J=8.9 Hz), 7.52(1H, d, J=8.2 Hz), 10.40(1H, s) ESI-MS(m/z): 416(M+H)⁺

### EXAMPLE 440

To a solution of N-{2-[3-(tritylamino)-1H-pyrazol-1-yl]ethyl}-1,4-benzenediamine (131 mg), 4-chloro-2-(dimethylamino)benzoic acid (62.6 mg) and 1-hydroxybenzotriazole (48 mg) in N,N-dimethylformamide (1.3 ml) was added 1-[3-(dimethylamino)propyl]-3-ethylcarbodiimide hydrochloride (60.1 mg), followed by triethylamine (43.3 mg) at ambient temperature. The reaction mixture was stirred for 14 hours at 50° C. and concentrated in vacuo. The residue was dissolved in ethyl acetate and water, and extracted with ethyl acetate. The organic layer was washed with water and brine, dried over magnesium sulfate, filtered and concentrated in vacuo. The residue was purified by column chromatography on silica gel eluting with hexane:ethyl acetate (1:1) to give 4-chloro-2-(dimethylamino)-N-[4-({2-[3-(tritylamino)-1H-pyrazol-1-yl]ethyl}amino)phenyl]benzamide (0.157 g) as a yellow foam.

¹H-NMR(CDCl₃): δ 2.81(6H, s), 3.39(2H, t, J=5.7 Hz), 3.99(2H, t, J=5.4 Hz), 4.91(1H, d, J=2.4 Hz), 5.43(2H, br s), 6.48(2H, d, J=8.9 Hz), 6.76(1H, d, J=2.4 Hz), 7.16-7.32(11H, m), 7.36-7.43(8H, m), 8.14(1H, d, J=8.9 Hz), 11.37(1H, s) ESI-MS(m/z): 664(M+Na)⁺

### EXAMPLE 441

To a solution of 4-chloro-2-(dimethylamino)-N-[4-({2-[3-(tritylamino)-1H-pyrazol-1-yl]ethyl}amino)phenyl]benzamide (150 mg) in methanol (1.2 ml) was added 35% hydrochloric acid (0.104 ml). The mixture was stirred for 3 hours and concentrated in vacuo. The residue was dissolved in ethyl acetate and 10% potassium carbonate solution, and extracted with ethyl acetate. The organic layer was washed with brine, dried over magnesium sulfate, filtered and concentrated in vacuo. The residue was recrystallized from hexane-ethyl acetate to give N-(4-{[2-(3-amino-1H-pyrazol-1-yl)ethyl]amino}phenyl)-4-chloro-2-(dimethylamino)benzamide (0.073 g) as pale yellow powder.

¹H-NMR(CDCl₃): δ 2.82(6H, s), 3.53(2H, t, J=5.4 Hz), 4.12(2H, t, J=5.1 Hz), 5.58(1H, d, J=2.2 Hz), 6.62(2H, d, J=8.9 Hz), 7.09(1H, d, J=2.4 Hz), 7.19-7.23(2H, m), 7.47(2H, d, J=8.6 Hz), 8.15(1H, d, J=8.9 Hz), 11.35(1H, s) ESI-MS(m/z): 399(M+H)⁺

Preparation 210

The mixture of 1-(2-aminoethyl)-N-trityl-1H-pyrazol-3-amine (5.276 g), 2-chloro-5-nitropyridine (2.72 g) and triethylamine (3.99 ml) in dimethylformamide (26.4 ml) was heated to 50° C. for 18 hours. The reaction mixture was concentrated in vacuo. To the residue added water and extracted with ethyl acetate. The organic layer was washed with brine, dried over magnesium sulfate, filtered and concentrated in vacuo. The residue was recrystallized from ethyl acetate-hexane to give 5-nitro-N-{2-[3-(tritylamino)-1H-pyrazol-1-yl]ethyl}-2-pyridinamine (6.812 g) as pale yellow powder.

¹H-NMR(CDCl₃): δ 3.68(2H, br q, J=5.1 Hz), 3.97(2H, br t, J=5.1 Hz), 5.04(1H, d, J=2.2 Hz), 5.09(1H, s), 5.69(1H, br s), 6.06(1H, d, J=9.5 Hz), 6.81(1H, d, J=2.4 Hz), 7.16-7.30(10H, m), 7.36-7.47(6H, m), 8.08(1H, dd, J=9.2, 2.7 Hz), 8.98(1H, d, J=2.7 Hz) ESI-MS(m/z): 513(M+H)⁺

Preparation 211

A solution of 5-nitro-N-{2-[3-(tritylamino)-1H-pyrazol-1-yl]ethyl}-2-pyridinamine (2.5 g) in methanol (25 ml) was hydrogenated over 10% palladium on carbon (0.5 g 50% wet) at ambient temperature under atmospheric pressure of hydrogen for 11 hours. The reaction mixture was filtered through a short pad of celite, and the filtrate was concentrated in vacuo to give 5-amino-2-({2-[3-(tritylamino)-1H-pyrazol-1-yl]ethyl}amino)pyridine (2.3 g) as a dark purple foam.

¹H-NMR(CDCl₃): δ 3.50(2H, br q, J=5.1 Hz), 3.96(2H, br t, J=5.1 Hz), 4.05(1H, br s), 4.91(1H, d, J=2.2 Hz), 5.11(1H, s), 5.14(1H, dd, J=8.6, 0.8 Hz), 6.75(1H, d, J=2.4 Hz), 6.98(1H, dd, J=8.4, 3.0 Hz), 7.19-7.29(9H, m), 7.38-7.43(6H, m), 7.65(1H, d, J=2.7 Hz) ESI-MS(m/z): 483(M+Na)⁺

### EXAMPLE 442

To a solution of 5-amino-2-({2-[3-(tritylamino)-1H-pyrazol-1-yl]ethyl}amino)pyridine (218 mg), 4-chloro-2-(dimethylamino)benzoic acid (104 mg) and 1-hydroxybenzotriazole (79.7 mg) in N,N-dimethylformamide (2.2 ml) was added 1-[3-(dimethylamino)propyl]-3-ethylcarbodiimide hydrochloride (99.8 mg), followed by triethylamine (0.1 ml) at ambient temperature. The reaction mixture was stirred for 12 hours at 60° C. and concentrated in vacuo. The residue was dissolved in ethyl acetate and water, and extracted with ethyl acetate. The organic layer was washed with water and brine, dried over magnesium sulfate, filtered and concentrated in vacuo to give 4-chloro-2-(dimethylamino)-N-[6-({2-[3-(tritylamino)-1H-pyrazol-1-yl]ethyl}amino)-3-pyridinyl]benzamide (0.271 g) as a dark red foam.

¹H-NMR(CDCl₃): δ 2.81(6H, s), 3.61(2H, t, J=5.4 Hz), 4.04(2H, t, J=6.5 Hz), 4.88(1H, d, J=2.7 Hz), 6.33(1H, d, J=8.9 Hz), 6.82(1H, d, J=2.2 Hz), 7.16-7.45(18H, m), 7.98(1H, dd, J=8.9, 2.2 Hz), 8.14-8.17(2H, m), 11.70(1H, s) ESI-MS(m/z): 643(M+H)⁺

### EXAMPLE 443

To a solution of 4-chloro-2-(dimethylamino)-N-[6-({2-[3-(tritylamino)-1H-pyrazol-1-yl]ethyl}amino)-3-pyridinyl]benzamide (217.4 mg) in methanol (2.2 ml) was added 35% hydrochloric acid (0.185 ml). The mixture was stirred for 2 hours and concentrated in vacuo. The residue was dissolved in ethyl acetate and 10% potassium carbonate solution and extracted with ethyl acetate. The organic layer was washed with brine, dried over magnesium sulfate, filtered and concentrated in vacuo. The residue was purified by column chromatography on silica gel eluting with chloroform: methanol (19:1) to give N-(6-{[2-(3-amino-1H-pyrazol-1-yl)ethyl]amino}-3-pyridinyl)-4-chloro-2-(dimethylamino)benzamide (0.092 g) as pale brown powder.

¹H-NMR(CDCl₃): δ 2.82(6H, s), 3.73(2H, br q, J=5.9 Hz), 4.14(2H, t, J=5.1 Hz), 4.77(1H, br s), 5.57(1H, d, J=2.4 Hz), 6.42(1H, d, J=8.9 Hz), 7.09(1H, d, J=2.2 Hz), 7.21-7.27(2H, m), 7.99(1H, dd, J=8.9, 2.4 Hz), 8.14-8.18(2H, m), 11.56(1H, s) ESI-MS(m/z): 400(M+H)⁺

Preparation 212

To a solution of 2-(5-amino-1H-pyrazol-1-yl)ethanol (10 g) in 1,2-dichloroethane (100 ml) was added triethylamine (12.1 mL) and trityl chloride (24.1 g) at ambient temperature. The mixture was stirred at room temperature for 3 hours. The reaction mixture was poured into water, filtered, and washed with hexane. The solid was dissolved with ethyl acetate at 70° C., recrystallized from hexane to give 2-[5-(tritylamino)-1H-pyrazol-1-yl]ethanol (18.246 g) as white powder.

¹H-NMR(CDCl₃): δ 3.65(1H, br s), 3.81(2H, t, J=4.6 Hz), 3.95(2H, t, J=4.9 Hz), 4.77(1H, d, J=2.2 Hz), 5.76(1H, s), 6.96(1H, d, J=1.9 Hz), 7.18-7.33(15H, m) ESI-MS(m/z): 370(M+H)⁺

Preparation 213

To a solution of 2-[5-(tritylamino)-1H-pyrazol-1-yl]ethanol (12 g) in 1,2-dichloroethane (120 ml) was added triethylamine (6.79 ml) and 4-(N,N-dimethylamino)pyridine (0.4 g), followed by p-toluene sulfonylchloride (7.43 g). The mixture was stirred at ambient temperature for 16 hours. The reaction mixture was poured into water and extracted with 1,2-dichloroethane. The organic layer was washed with brine, dried over magnesium sulfate, filtered and concentrated in vacuo. The residue was recrystallized from ethyl acetate-hexane to give 2-[5-(tritylamino)-1H-pyrazol-1-yl]ethyl 4-methylbenzenesulfonate (13.055 g) as white powder.

¹H-NMR(CDCl₃): δ 2.41(3H, s), 4.16(2H, t, J=4.9 Hz), 4.29(2H, t, J=4.6 Hz), 4.82(1H, d, J=1.9 Hz), 5.01(1H, s), 6.98(1H, d, J=2.2 Hz), 7.19-7.30(17H, m), 7.55(2H, d, J=8.1 Hz) ESI-MS(m/z): 546(M+Na)⁺

Preparation 214

To a solution of 2-[5-(tritylamino)-1H-pyrazol-1-yl]ethyl 4-methylbenzenesulfonate (12.0 g) in N,N-dimethylformamide (120 ml) was added sodium azide (2.98 g). The mixture was stirred at ambient temperature for 12 hours. The reaction mixture was concentrated in vacuo, poured into water and extracted with ethyl acetate. The organic layer was washed with brine, dried over magnesium sulfate, filtered and concentrated in vacuo. The residue was recrystallized from ethyl acetate-hexane to give 1-(2-azidoethyl)-N-trityl-1H-pyrazol-5-amine (9.00 g) as pale yellow powder.

¹H-NMR(CDCl₃): δ 3.69(2H, t, J=5.4 Hz), 3.99(2H, t, J=5.7 Hz), 4.86(1H, d, J=1.6 Hz), 5.19(1H, s), 7.08(1H, d, J=1.9 Hz), 7.18-7.32(15H, m) ESI-MS(m/z): 395(M+H)⁺

Preparation 215

A solution of 1-(2-azidoethyl)-N-trityl-1H-pyrazol-5-amine (4.226 g) in methanol (42 ml) was hydrogenated over 10% palladium on carbon (0.845 g, 50% wet) at ambient temperature under atmospheric pressure of hydrogen for 1.5 hours. The reaction mixture was filtered through a short pad of celite, and the filtrate was concentrated in vacuo. The residue was purified by column chromatography on silica gel eluting with hexane: ethyl acetate (1:3) to give N-[1-(2-aminoethyl)-1H-pyrazol-5-yl]-N-tritylamine (2.139 g) as white powder.

¹H-NMR(CDCl₃): δ 3.07(2H, t, J=5.4 Hz), 4.01(2H, t, J=5.1 Hz), 4.74(1H, d, J=1.9 Hz), 6.82(1H, br s), 7.01(1H, d, J=2.2 Hz), 7.18-7.35(15H, m) ESI-MS(m/z): 369(M+H)⁺

Preparation 216

To a solution of N-[1-(2-aminoethyl)-1H-pyrazol-5-yl]-N-tritylamine (2.139 g) in 1,3-dimethyl-2-imidazolidinone (21 ml) was added to triethylamine (1.21 ml), followed by 1-fluoro-4-nitrobenzene (0.983 g) at ambient temperature. The mixture was stirred at 50° C. for 20 hours. The reaction mixture was cooled to ambient temperature, poured into water and extracted with ethyl acetate. The organic layer was washed with brine, dried over magnesium sulfate, filtered and concentrated in vacuo. The residue was recrystallized from ethyl acetate-hexane to give 1-{2-[(4-nitrophenyl)amino]ethyl}-N-trityl-1H-pyrazol-5-amine (2.119 g) as a yellow powder.

¹H-NMR(CDCl₃): δ 3.53(2H, br q, J=5.4 Hz), 4.06(2H, t, J=5.4 Hz), 4.42(1H, s), 4.91(1H, d, J=1.9 Hz), 5.18(1H, br t, J=5.1 Hz), 6.45(2H, d, J=9.2 Hz), 7.12-7.26(16H, m), 8.03(2H, d, J=8.9 Hz) ESI-MS(m/z): 512(M+Na)⁺

Preparation 217

To a solution of 1-{2-[(4-nitrophenyl)amino]ethyl}-N-trityl-1H-pyrazol-5-amine (2.114 g) and 4-(N,N-dimethylamino)pyridine (52.8 mg) in tetrahydrofuran (21 ml) was added di-t-butyl dicarbonate (1.13 g). The mixture was stirred at 40° C. for 15 hours. The reaction mixture was cooled to ambient temperature and concentrated in vacuo. The residue was dissolved in ethyl acetate and water, and extracted with ethyl acetate. The organic layer was washed with brine, dried over magnesium sulfate, filtered and concentrated in vacuo. The residue was recrystallized from ethyl acetate-hexane to give tert-butyl 4-nitrophenyl{2-[5-(tritylamino)-1H-pyrazol-1-yl]ethyl}carbamate (2.363 g) as pale yellow powder.

¹H-NMR(CDCl₃): δ 1.34(9H, s), 3.96(2H, t, J=5.9 Hz), 4.15(2H, t, J=6.2 Hz), 4.82(1H, d, J=2.2 Hz), 5.05(1H, br s), 7.00(1H, d, J=1.9 Hz), 7.04(2H, d, J=9.2 Hz), 7.18-7.33(15H, m), 8.07(2H, d, J=9.2 Hz) ESI-MS(m/z): 612(M+Na)⁺

Preparation 218

A solution of tert-butyl 4-nitrophenyl{2-[5-(tritylamino)-1H-pyrazol-1-yl]ethyl}carbamate (2.363 g) in ethyl acetate (24 ml) was hydrogenated over 10% palladium on carbon (0.473 g 50% wet) at room temperature under atmospheric pressure of hydrogen for 3 hours. The reaction mixture was filtered through a short pad of celite, and the filtrate was concentrated in vacuo. The residue was purified by column chromatography on silica gel eluting with hexane: ethyl acetate (4:1→1/1→1/2) to give tert-butyl 4-aminophenyl{2-[5-(tritylamino)-1H-pyrazol-1-yl]ethyl}carbamate (2.177 g) as a pale yellow foam.

¹H-NMR(CDCl₃): δ 1.26(9H, s), 3.64(2H, br s), 3.76(2H, t, J=7.3 Hz), 4.13(2H, t, J=7.3 Hz), 4.76(1H, br s), 6.53(2H, d, J=8.4 Hz), 6.73(2H, br d, J=8.4 Hz), 6.98(1H, d, J=2.2 Hz), 7.18-7.29(9H, m), 7.35-7.37(6H, m) ESI-MS(m/z): 582(M+Na)⁺

### EXAMPLE 444

To a solution of tert-butyl 4-aminophenyl{2-[5-(tritylamino)-1H-pyrazol-1-yl]ethyl}carbamate (400 mg), 4-chloro-2-(dimethylamino)benzoic acid (157 mg) and 1-hydroxybenzotriazole (120 mg) in N,N-dimethylformamide (4 ml) was added 1-[3-(dimethylamino)propyl]-3-ethylcarbodiimide hydrochloride (151 mg), followed by triethylamine (0.15 ml) at ambient temperature. The reaction mixture was stirred for 16 hours at 60° C. and concentrated in vacuo. The residue was dissolved in ethyl acetate and water and extracted with ethyl acetate. The organic layer was washed with water and brine, dried over magnesium sulfate, filtered and concentrated in vacuo. The residue was purified by column chromatography on silica gel eluting with hexane: ethyl acetate (3:1→1:1) to give tert-butyl 4-{[4-chloro-2-(dimethylamino)benzoyl]amino}phenyl{2-[5-(tritylamino)-1H-pyrazol-1-yl]ethyl}carbamate (424 mg) as a pale yellow foam.

¹H-NMR(CDCl₃): δ 1.29(9H, s), 3.82(2H, t, J=7.0 Hz), 4.23(2H, t, J=6.5 Hz), 4.76(1H, d, J=2.2 Hz), 6.92(2H, d, J=8.9 Hz), 6.97(1H, d, J=2.2 Hz), 7.20-7.39(17H, m), 7.54(2H, d, J=8.6 Hz), 8.17(1H, d, J=8.9 Hz), 11.74(1H, s) ESI-MS(m/z): 764(M+Na)⁺

### EXAMPLE 445

To a solution of tert-butyl 4-([4-chloro-2-(dimethylamino)benzoyl]amino}phenyl{2-[5-(tritylamino)-1H-pyrazol-1-yl]ethyl}carbamate (416 mg) in dichloromethane (4.2 ml) was added trifluoroacetic acid (0.648 ml). The reaction mixture was stirred for 7 hours, quenched with 10% potassium carbonate solution and extracted with dichloromethane. The organic layer was washed with brine, dried over magnesium sulfate, filtered and concentrated in vacuo. The residue was purified by column chromatography on silica gel eluting with hexane: ethyl acetate (1:1→1:3→1:5) to give N-(4-{[2-(5-amino-1H-pyrazol-1-yl)ethyl]amino}phenyl)-4-chloro-2-(dimethylamino)benzamide (148 mg) as pale yellow powder.

¹H-NMR(CDCl₃): δ 2.82(6H, s), 3.58(2H, dd, J=5.7, 4.3 Hz), 4.19(2H, t, J=5.1 Hz), 5.52(1H, d, J=2.2 Hz), 6.59(2H, d, J=8.9 Hz), 7.21(1H, dd, J=7.6, 1.9 Hz), 7.23(1H, s), 7.31(1H, d, J=1.9 Hz), 7.46(2H, d, J=8.9 Hz), 8.15(1H, d, J=9.2 Hz), 11.39(1H, s) ESI-MS(m/z): 339(M+H)⁺

### EXAMPLE 446

The following compound was obtained in substantially the same manner as in Example 444.

tert-Butyl (4-{[2-(dimethylamino)-4-methylbenzoyl]amino}phenyl){2-[5-(tritylamino)-1H-pyrazol-1-yl]ethyl}carbamate

¹H-NMR(CDCl₃): δ 1.28(9H, s), 2.40(3H, s), 2.81(6H, s), 3.82(2H, t, J=6.2 Hz), 4.21(2H, t, J=5.9 Hz), 4.76(1H, d, J=1.9 Hz), 6.91(2H, d, J=8.6 Hz), 6.98(1H, d, J=2.4 Hz), 7.09(1H, d, J=7.8 Hz), 7.10(1H, s), 7.17-7.37(15H, m), 7.56(2H, d, J=8.9 Hz), 8.15(1H, d, J=8.4 Hz), 12.31(1H, s) ESI-MS(m/z): 743(M+H)⁺

### EXAMPLE 447

The following compound was obtained in substantially the same manner as in Example 445.

N-(4-{[2-(5-Amino-1H-pyrazol-1-yl)ethyl]amino}phenyl)-2-(dimethylamino)-4-methylbenzamide

¹H-NMR(CDCl₃): δ 2.39(3H, s), 2.80(6H, s), 3.58(2H, t, J=5.4 Hz), 4.18(2H, t, J=5.1 Hz), 5.51(1H, d, J=1.9 Hz), 6.59(2H, d, J=8.9 Hz), 7.07(1H, d, J=7.0 Hz), 7.08(1H, s), 7.31(1H, d, J=1.9 Hz), 7.49(2H, d, J=8.9 Hz), 8.14(1H, d, J=8.4 Hz), 11.94(1H, br s) ESI-MS(m/z): 401(M+Na)⁺

### EXAMPLE 448

The following compound was obtained in substantially the same manner as in Example 444.

tert-Butyl [4-({[6-methyl-2-(4-methyl-1-piperidinyl)-3-pyridinyl]carbonyl}amino)phenyl]{2-[5-(tritylamino)-1H-pyrazol-1-yl]ethyl}carbamate

¹H-NMR(CDCl₃): δ 1.01(3H, d, J=6.2 Hz), 1.29(9H, s), 1.41(2H, qd, J=12.7, 4.3 Hz), 1.48-1.80(1H, m), 1.84(2H, br d, J=12.7 Hz), 2.65(3H, s), 3.00(2H, qd, J=12.4, 2.2 Hz), 3.33(2H, br d, J=12.7 Hz), 3.83(2H, t, J=7.8 Hz), 4.20(2H, t, J=7.8 Hz), 4.77(1H, d, J=2.4 Hz), 6.94(2H, d, J=8.6 Hz), 6.97(1H, d, J=2.4 Hz), 7.03(1H, d, J=7.8 Hz), 7.18-7.36(15H, m), 7.62(2H, d, J=8.9 Hz), 8.36(1H, d, J=8.4 Hz), 11.86(1H, s) ESI-MS(m/z): 798(M+Na)⁺

### EXAMPLE 449

The following compound was obtained in substantially the same manner as in Example 445.

N-(4-{[2-(5-Amino-1H-pyrazol-1-yl)ethyl]amino}phenyl)-6-methyl-2-(4-methyl-1-piperidinyl)nicotinamide

¹H-NMR(CDCl₃): δ 1.02(3H, d, J=6.2 Hz), 1.40(2H, qd, J=12.4, 3.8 Hz), 1.48-1.85(1H, m), 1.83(2H, br d, J=12.7 Hz), 2.18(3H, s), 2.52(3H, s), 2.98(2H, td, J=12.2, 2.2 Hz), 3.34(2H, br d, J=12.4 Hz), 3.59(2H, t, J=5.7 Hz), 4.20(2H, t, J=5.1 Hz), 5.53(1H, d, J=1.9 Hz), 6.61(2H, d, J=8.9 Hz), 7.01(1H, d, J=8.1 Hz), 7.32(1H, d, J=1.9 Hz), 7.55(2H, d, J=8.6 Hz), 8.35(1H, d, J=7.6 Hz), 11.56(1H, br s) ESI-MS(m/z): 434(M+H)⁺

### EXAMPLE 450

To a solution of 2-(4-methylphenyl)-1-cyclohexene-1-carboxylic acid (116 mg) in toluene (1.2 ml) were added thionyl chloride (0.078 ml) and N,N-dimethylformamide (1 drop) and the mixture was stirred at 80° C. for 1 hour. The mixture was evaporated in vacuo and the residue was dissolved in tetrahydrofuran (1.0 ml). The acid chloride in tetrahydrofuran was added to a solution of tert-butyl 4-aminophenyl{2-[5-(tritylamino)-1H-pyrazol-1-yl)ethyl}carbamate (250 mg) and triethylamine (0.093 ml) in tetrahydrofuran (1.5 ml) at ambient temperature and the mixture was stirred at the same temperature for 2 hours. The mixture was poured into water and extracted with ethyl acetate. The organic layer was washed with brine, dried over magnesium sulfate and evaporated in vacuo. The residue was recrystallized from ethyl acetate-hexane to give tert-butyl 4-({[2-(4-methylphenyl)-1-cyclohexen-1-yl]carbonyl}amino)phenyl{2-[5-(tritylamino)-1H-pyrazol-1-yl]ethyl}carbamate (155.8 mg) as pale yellow powder.

¹H-NMR(CDCl₃): δ 1.26(9H, s), 1.77(4H, br s), 2.31(3H, s), 2.42(2H, br s), 2.52(2H, br s), 3.74(2H, t, J=7.3 Hz), 4.08(2H, t, J=7.3 Hz), 4.76(1H, d, J=1.6 Hz), 6.61(1H, br s), 6.73(2H, d, J=8.4 Hz), 6.87(2H, d, J=8.9 Hz), 6.96(1H, d, J=2.2 Hz), 7.18-7.28(13H, m), 7.32-7.36(6H, m) ESI-MS(m/z): 780(M+Na)⁺

### EXAMPLE 451

The following compound was obtained in substantially the same manner as in Example 445.

N-(4-{[2-(5-Amino-1H-pyrazol-1-yl)ethyl]amino}phenyl)-2-(4-methylphenyl)-1-cyclohexene-1-carboxamide

¹H-NMR(CDCl₃): δ 1.76(4H, br s), 2.33(3H, s), 2.41(2H, br s), 2.51(2H, br s), 3.50(2H, t, J=5.4 Hz), 4.15(2H, t, J=5.4 Hz), 5.49(1H, d, J=1.9 Hz), 6.40(2H, d, J=8.6 Hz), 6.44(1H, s), 6.76(2H, d, J=8.9 Hz), 7.12-7.19(4H, m), 7.28(1H, d, J=1.6 Hz) ESI-MS(m/z): 416(M+H)⁺

### EXAMPLE 452

The following compound was obtained in substantially the same manner as in Example 450.

tert-Butyl (4-{[(4′-methyl-1,1′-biphenyl-2-yl)carbonyl]amino}phenyl){2-[5-(tritylamino)-1H-pyrazol-1-yl]ethyl}carbamate

¹H-NMR(CDCl₃): δ 1.28(9H, s), 2.39(3H, s), 3.77(2H, t, J=7.0 Hz), 4.11(2H, t, J=7.0 Hz), 4.77(1H, d, J=1.9 Hz), 6.79(2H, d, J=8.9 Hz), 6.94(1H, br s), 6.96(1H, d, J=2.2 Hz), 7.02(2H, d, J=8.9 Hz), 7.19-7.28(11H, m), 7.32-7.39(8H, m), 7.40-7.53(3H, m), 7.88(1H, dd, J=7.3, 1.1 Hz) ESI-MS(m/z): 776(M+Na)⁺

### EXAMPLE 453

The following compound was obtained in substantially the same manner as in Example 445.

N-(4-{[2-(5-Amino-1H-pyrazol-1-yl)ethyl]amino}phenyl)-4′-methyl-1,1′-biphenyl-2-carboxamide

¹H-NMR(CDCl₃): δ 2.40(3H, s), 3.52(2H, t, J=5.7 Hz), 4.14(2H, t, J=5.7 Hz), 5.51(1H, d, J=2.2 Hz), 6.45(2H, d, J=8.9 Hz), 6.77(1H, br s), 6.92(2H, d, J=7.8 Hz), 7.24(2H, d, J=7.8 Hz), 7.29(1H, d, J=1.9 Hz), 7.36(2H, d, J=6.2 Hz), 7.38(3H, m), 7.85(1H, dd, J=7.3, 1.1 Hz) ESI-MS(m/z): 412(M+H)⁺

Preparation 219

To a solution of N-(1-(2-aminoethyl)-1H-pyrazol-5-yl)-N-tritylamine (1.853 g) in N,N-dimethylformamide (18.5 ml) was added to triethylamine (1.05 ml), followed by 2-chloro-5-nitropyridine (0.956 g) at ambient temperature. The mixture was stirred at 50° C. for 14 hours. The reaction mixture was cooled to ambient temperature, poured into water and extracted with ethyl acetate. The organic layer was washed with brine, dried over magnesium sulfate, filtered and concentrated in vacuo. The residue was recrystallized from ethyl acetate-hexane to give 5-nitro-N-{2-[5-(tritylamino)-1H-pyrazol-1-yl]ethyl}-2-pyridinamine (2.23 g) as pale yellow powder.

¹H-NMR(CDCl₃): δ 3.73(2H, br q, J=6.2 Hz), 4.13(2H, t, J=6.5 Hz), 4.85(1H, d, J=1.9 Hz), 4.90(1H, br s), 5.80(1H, br t, J=5.7 Hz), 6.32(1H, d, J=9.5 Hz), 7.06(1H, d, J=2.2 Hz), 7.20-7.26(15H, m), 8.07(1H, dd, J=9.5, 2.7 Hz), 8.49(1H, d, J=2.4 Hz) ESI-MS(m/z): 513(M+Na)⁺

Preparation 220

To a solution of 5-nitro-N-{2-[5-(tritylamino)-1H-pyrazol-1-yl]ethyl}-2-pyridinamine (2.22 g) and 4-(N,N-dimethylamino)pyridine (55.3 mg) in tetrahydrofuran (22 ml) was added di-t-butyl dicarbonate (1.48 g). The mixture was stirred at 40° C. for 1.4 hours. The reaction mixture was cooled to ambient temperature and concentrated in vacuo. The residue was dissolved in ethyl acetate and water, and extracted with ethyl acetate. The organic layer was washed with brine, dried over magnesium sulfate, filtered and concentrated in vacuo. The residue was recrystallized from ethyl acetate-hexane to give tert-butyl 5-nitro-2-pyridinyl{2-[5-(tritylamino)-1H-pyrazol-1-yl]ethyl}carbamate (2.54 g) as dark yellow powder.

¹H-NMR(CDCl₃): δ 1.52(9H, s), 4.19-4.38(4H, m), 4.76(1H, d, J=1.9 Hz), 5.21(1H, s), 7.01(1H, d, J=2.2 Hz), 7.20-7.35(15H, m), 8.12-8.16(1H, m), 8.25-8.30(2H, m) ESI-MS(m/z): 613(M+Na)⁺

Preparation 221

A solution of tert-butyl 5-nitro-2-pyridinyl{2-[5-(tritylamino)-1H-pyrazol-1-yl]ethyl}carbamate (2.49 g) in methanol (25 ml) was hydrogenated over 10% palladium on carbon (0.50 g, 50% wet) at ambient temperature under atmospheric pressure of hydrogen for 2.5 hours. The reaction mixture was filtered through a short pad of celite, and the filtrate was concentrated in vacuo to give tert-butyl 5-amino-2-pyridinyl{2-[5-(tritylamino)-1H-pyrazol-1-yl]ethyl}carbamate (2.23 g) as a pale yellow foam.

¹H-NMR(CDCl₃): δ 1.40(9H, s), 3.36(2H, br s), 3.93(2H, t, J=7.6 Hz), 4.32(2H, t, J=7.3 Hz), 4.77(1H, d, J=2.2 Hz), 6.91(1H, dd, J=8.9, 3.0 Hz), 7.00(1H, d, J=1.9 Hz), 7.17-7.43(17H, m) ESI-MS (m/z): 583(M+Na)⁺

### EXAMPLE 454

To a solution of tert-butyl 5-amino-2-pyridinyl{2-[5-(tritylamino)-1H-pyrazol-1-yl]ethyl}carbamate (350 mg), 4-chloro-2-(dimethylamino)benzoic acid (137 mg) and 1-hydroxybenzotriazole (105 mg) in N,N-dimethylformamide (3.5 ml) was added 1-[3-(dimethylamino)propyl]-3-ethylcarbodiimide hydrochloride (132 mg), followed by triethylamine (0.1 ml) at ambient temperature. The reaction mixture was stirred for 16 hours at 60° C. and concentrated in vacuo. The residue was dissolved in ethyl acetate and water and extracted with ethyl acetate. The organic layer was washed with water and brine, dried over magnesium sulfate, filtered and concentrated in vacuo. The residue was purified by column chromatography on silica gel eluting with hexane: ethyl acetate (3:1→1:1) to give tert-butyl 5-{[4-chloro-2-(dimethylamino)benzoyl]amino}-2-pyridinyl{2-[5-(tritylamino)-1H-pyrazol-1-yl]ethyl}carbamate (299.5 mg) as a yellow foam.

¹H-NMR(CDCl₃): δ 1.42(9H, s), 2.83(6H, s), 4.07(2H, t, J=6.2 Hz), 4.31(2H, t, J=6.2 Hz), 4.77(1H, d, J=1.9 Hz), 5.81(1H, s), 7.01(1H, d, J=2.2 Hz), 7.17-7.39(17H, m), 7.56(1H, d, J=8.9 Hz), 7.63(1H, br d, J=2.7 Hz), 8.16(1H, dd, J=8.1, 1.1 Hz), 8.29(1H, dd, J=8.9, 3.0 Hz), 11.54(1H, s) ESI-MS(m/z): 743(M+H)⁺

### EXAMPLE 455

To a solution of tert-butyl 5-{[4-chloro-2-(dimethylamino)benzoyl]amino}-2-pyridinyl{2-[5-(tritylamino)-1H-pyrazol-1-yl]ethyl}carbamate (293.6 mg) in dichloromethane (3 ml) was added trifluoroacetic acid (0.914 ml). The reaction mixture was stirred for 12 hours, quenched with 10% potassium carbonate solution and extracted with dichloromethane. The organic layer was washed with brine, dried over magnesium sulfate, filtered and concentrated in vacuo. The residue was recrystallized from ethyl acetate-hexane to give N-(6-{[2-(5-amino-1H-pyrazol-1-yl)ethyl]amino}-3-pyridinyl)-4-chloro-2-(dimethylamino)benzamide (73.7 mg) as pale yellow green powder.

¹H-NMR(CDCl₃): δ 2.83(6H, s), 3.72(2H, br q, J=5.9 Hz), 4.02(2H, br s), 4.22(2H, t, J=6.5 Hz), 4.80(1H, br t, J=5.9 Hz), 5.51(1H, d, J=1.6 Hz), 6.44(1H, d, J=8.9 Hz), 7.23(1H, d, J=1.9 Hz), 7.26-7.29(2H, m), 7.93(1H, dd, J=8.9, 2.7 Hz), 8.16(1H, dd, J=7.8, 0.5 Hz), 8.23(1H, d, J=2.4 Hz), 11.59(1H, s) ESI-MS(m/z): 400(M+H)⁺

### EXAMPLE 456

To a solution of 2-(4-methylphenyl)-1-cyclohexene-1-carboxylic acid (127 mg) in toluene (1.3 ml) were added thionyl chloride (0.086 ml) and N,N-dimethylformamide (1 drop) and the mixture was stirred at 80° C. for 1 hour. The mixture was evaporated in vacuo and the residue was dissolved in tetrahydrofuran (1.0 ml). The acid chloride in tetrahydrofuran was added to a solution of tert-butyl 5-amino-2-pyridinyl{2-[5-(tritylamino)-1H-pyrazol-1-yl]ethyl}carbamate (300 mg) and triethylamine (0.09 mL) in tetrahydrofuran (2.0 ml) at ambient temperature and the mixture was stirred at the same temperature for 15 hours. The mixture was poured into water and extracted with ethyl acetate. The organic layer was washed with brine, dried over magnesium sulfate and evaporated in vacuo. The residue was purified by column chromatography on silica gel eluting with hexane: ethyl acetate (3:1→1:1) to give tert-butyl 5-({[2-(4-methylphenyl)-1-cyclohexen-1-yl]carbonyl}amino)-2-pyridinyl{2-[5-(tritylamino)-1H-pyrazol-1-yl]ethyl}carbamate (334.4 mg).

¹H-NMR(CDCl₃): δ 1.41(9H, s), 1.79(4H, br s), 2.32(3H, s), 2.45(2H, br s), 2.53(2H, br s), 3.96(2H, t, J=7.0 Hz), 4.23(2H, t, J=6.5 Hz), 4.75(1H, d, J=1.9 Hz), 5.75(1H, s), 6.81(1H, br d, J=2.7 Hz), 7.11-7.35(19H, m), 7.42(1H, d, J=8.9 Hz), 7.68(1H, dd, J=9.2, 2.7 Hz) ESI-MS(m/z): 781(M+Na)⁺

### EXAMPLE 457

The following compound was obtained in substantially the same manner as in Example 455.

N-(6-{[2-(5-Amino-1H-pyrazol-1-yl)ethyl]amino}-3-pyridinyl)-2-(4-methylphenyl)-1-cyclohexene-1-carboxamide

¹H-NMR(CDCl₃): δ 1.76(4H, br s), 2.34(3H, s), 2.41(2H, br s), 2.51(2H, br s), 3.63(2H, q, J=5.9 Hz), 3.93(2H, br s), 4.13(2H, t, J=6.5 Hz), 4.71(1H, br t, J=5.9 Hz), 5.46(1H, d, J=1.9 Hz), 6.23(1H, d, J=8.9 Hz), 6.40(1H, s), 7.05-7.10(4H, m), 7.21-7.33(2H, m), 7.48(1H, d, J=2.2 Hz) ESI-MS(m/z): 417(M+H)⁺

### EXAMPLE 458

The following compound was obtained in substantially the same manner as in Example 454.

tert-Butyl [5-({[6-methyl-2-(4-methyl-1-piperidinyl)-3-pyridinyl]carbonyl}amino)-2-pyridinyl]{2-[5-(tritylamino)-1H-pyrazol-1-yl]ethyl}carbamate

¹H-NMR(CDCl₃): δ 1.03(3H, d, J=6.5 Hz), 1.20-1.40(2H, m), 1.43(9H, s), 1.58-1.70(1H, m), 1.85(2H, br d, J=13.5 Hz), 2.55(3H, s), 3.02(2H, td, J=12.2, 2.4 Hz), 3.33(2H, br d, J=12.4 Hz), 4.09(2H, t, J=7.6 Hz), 4.30(2H, t, J=6.8 Hz), 4.79(1H, d, J=2.2 Hz), 5.66(1H, s), 7.01(1H, d, J=2.2 Hz), 7.06(1H, d, J=7.8 Hz), 7.12-7.41(15H, m), 7.57(1H, d, J=8.9 Hz), 7.86(1H, d, J=2.2 Hz), 8.27(1H, dd, J=9.2, 3.0 Hz), 8.37(1H, d, J=7.8 Hz), 11.76(1H, s) ESI-MS(m/z): 799(M+Na)⁺

### EXAMPLE 459

The following compound was obtained in substantially the same manner as in Example 455.

N-(6-{[2-(5-Amino-1H-pyrazol-1-yl)ethyl]amino}-3-pyridinyl)-6-methyl-2-(4-methyl-1-piperidinyl)nicotinamide

¹H-NMR(CDCl₃): δ 1.03(3H, d, J=6.8 Hz), 1.38(2H, qd, J=12.4, 3.2 Hz), 1.55-1.68(1H, m), 1.86(2H, br d, J=12.7 Hz), 2.52(3H, s), 3.01(2H, td, J=12.2, 2.2 Hz), 3.32(2H, d, J=12.4 Hz), 3.73(2H, q, J=5.9 Hz), 3.96(2H, br s), 4.22(2H, t, J=6.2 Hz), 4.82(1H, t, J=5.9 Hz), 5.50(1H, d, J=1.9 Hz), 6.46(1H, d, J=8.9 Hz), 7.03(1H, d, J=7.8 Hz), 7.28(1H, d, J=1.9 Hz), 7.98(1H, dd, J=8.9, 2.7 Hz), 8.33(1H, d, J=3.0 Hz), 8.35(1H, d, J=7.8 Hz), 11.75(1H, s) ESI-MS(m/z): 435(M+H)⁺

### EXAMPLE 460

The following compound was obtained in substantially the same manner as in Example 456.

tert-Butyl (5-{[(4′-methyl-1,1′-biphenyl-2-yl)carbonyl]amino}-2-pyridinyl){2-[5-(tritylamino)-1H-pyrazol-1-yl]ethyl}carbamate

¹H-NMR(CDCl₃): δ 1.43(9H, s), 2.39(3H, s), 3.99(2H, t, J=7.8 Hz), 4.27(2H, t, J=7.6 Hz), 4.76(1H, d, J=1.9 Hz), 5.79(1H, br s), 6.69(1H, br s), 6.98(1H, d, J=1.9 Hz), 7.13-7.59(24H, m), 7.84(2H, d, J=8.9 Hz) ESI-MS (m/z): 777 (M+Na)⁺

### EXAMPLE 461

The following compound was obtained in substantially the same manner as in Example 455.

N-(6-{[2-(5-Amino-1H-pyrazol-1-yl)ethyl]amino}-3-pyridinyl)-4′-methyl-1,1′-biphenyl-2-carboxamide

¹H-NMR(CDCl₃): δ 2.41(3H, s), 3.65(2H, q, J=6.2 Hz), 3.94(2H, br s), 4.15(2H, t, J=6.2 Hz), 4.79(1H, br t, J=5.9 Hz), 5.48(1H, d, J=1.9 Hz), 6.31(1H, d, J=8.9 Hz), 6.73(1H, s), 7.24-7.27(3H, m), 7.36(2H, d, J=7.8 Hz), 7.37-7.53(4H, m), 7.64(1H, d, J=2.2 Hz), 7.85(1H, dd, J=7.3, 1.4 Hz) ESI-MS(m/z): 413(M+H)⁺

### EXAMPLE 462

The following compound was obtained in substantially the same manner as in Example 454.

tert-Butyl (4-([2-(dimethylamino)-4-(trifluoromethyl)benzoyl]amino}phenyl){2-[5-(tritylamino)-1H-pyrazol-1-yl]ethyl}carbamate

¹H-NMR(CDCl₃): δ 1.28(9H, s), 2.87(6H, s), 3.83(2H, t, J=7.0 Hz), 4.27(2H, t, J=7.0 Hz), 4.76(1H, d, J=1.9 Hz), 6.94(2H, d, J=8.6 Hz), 6.97(1H, d, J=2.2 Hz), 7.19-7.42(15H, m), 7.48-7.50(2H, m), 7.55(2H, d, J=8.9 Hz), 8.32(1H, d, J=8.6 Hz), 11.67(1H, s) ESI-MS(m/z): 797(M+Na)⁺

### EXAMPLE 463

The following compound was obtained in substantially the same manner as in Example 455.

N-(4-{[2-(5-Amino-1H-pyrazol-1-yl)ethyl]amino}phenyl)-2-(dimethylamino)-4-(trifluoromethyl)benzamide

¹H-NMR(CDCl₃): δ 2.86(3H, s), 3.49(2H, br s), 3.57(2H, t, J=5.7 Hz), 4.20(2H, t, J=5.7 Hz), 5.53(1H, d, J=1.9 Hz), 6.60(2H, d, J=8.9 Hz), 7.32(1H, d, J=2.2 Hz), 7.46-7.49(4H, m), 7.30(1H, d, J=8.4 Hz), 11.31(1H, br s) ESI-MS (m/z): 433 (M+H)⁺

### EXAMPLE 464

To a solution of 6-{2-[5-(tritylamino)-1H-pyrazol-1-yl]ethoxy}-3-pyridinamine (320 mg), 4-chloro-2-(dimethylamino)benzoic acid (152 mg) and 1-hydroxybenzotriazole hydrate (127 mg) in N,N-dimethylformamide (5 ml) were added 1-[3-(dimethylamino)propyl]-3-ethylcarbodiimide hydrochloride (159 mg) and triethylamine (0.145 ml)at ambient temperature. The reaction mixture was stirred for 13 hours at 50° C. and concentrated in vacuo. The residue was dissolved in ethyl acetate and water and extracted with ethyl acetate. The organic layer was washed with water and brine, dried over magnesium sulfate, filtered and concentrated in vacuo. The residue was recrystallized from ethyl acetate-diisopropyl ether to give 4-chloro-2-(dimethylamino)-N-(6-{2-[5-(tritylamino)-1H-pyrazol-1-yl]ethoxy}-3-pyridinyl)benzamide (382 mg) as a pale yellow solid.

¹H-NMR(CDCl₃): δ 2.81(3H, s), 4.44(2H, t, J=4.7 Hz), 4.58(2H, t, J=4.7 Hz), 4.62(1H, d, J=2.0 Hz), 5.90(1H, s), 6.00(1H, d, J=8.9 Hz), 7.01(1H, d, J=2.0 Hz), 7.23-7.33(17H, m), 7.96(1H, d, J=2.6 Hz), 8.04(1H, dd, J=8.9, 2.6 Hz), 11.73(1H, s) ESI-MS(m/z): 665(M+Na)⁺

### EXAMPLE 465

To a solution of 4-chloro-2-(dimethylamino)-N-(6-{2-[5-(tritylamino)-1H-pyrazol-1-yl]ethoxy}-3-pyridinyl)benzamide (370 mg) in methanol (6 ml) was added concentrated hydrochloric acid (600 mg). The reaction mixture was stirred for 14 hours at ambient temperature, quenched with 10% potassium carbonate solution, and extracted with ethyl acetate. The organic layer was washed with brine, dried over magnesium sulfate, filtered and concentrated in vacuo. The residue was recrystallized from ethyl acetate-diisopropyl ether to give N-{6-[2-(5-amino-1H-pyrazol-1-yl)ethoxy]-3-pyridinyl}-4-chloro-2-(dimethylamino)benzamide (178 mg) as pale brown solids.

¹H-NMR(DMSO-d₆): δ 2.79(6H, s), 4.22(2H, t, J=6.0 Hz), 4.48(2H, t, J=6.0 Hz), 5.17(2H, s), 5.27(1H, d, J=1.6 Hz), 6.82(1H, d, J=8.9 Hz), 7.00(1H, dd, J=8.2, 2.0 Hz), 7.05(1H, d, J=2.0 Hz), 7.08(1H, d, J=2.0 Hz), 7.52(1H, d, J=8.2 Hz), 8.01(1H, dd, J=8.9, 2.0 Hz), 8.47(1H, d, J=2.0 Hz), 10.71(1H, s) ESI-MS(m/z): 423(M+Na)⁺

### EXAMPLE 466

To a solution of tert-butyl 5-amino-2-pyridinyl[2-(1H-pyrazol-1-yl)ethyl]carbamate (364 mg), 4-chloro-2-(dimethylamino)benzoic acid (263 mg) and 1-hydroxybenzotriazole hydrate (221 mg) in N,N-dimethylformamide (10 ml) were added 1-[3-(dimethylamino)propyl]-3-ethylcarbodiimide hydrochloride (276 mg) and triethylamine (0.145 ml) at ambient temperature. The reaction mixture was stirred for 13 hours at 50° C. and concentrated in vacuo. The residue was dissolved in ethyl acetate and water, and extracted with ethyl acetate. The organic layer was washed with water and brine, dried over magnesium sulfate, filtered and concentrated in vacuo. The residue was recrystallized from ethyl acetate-diisopropyl ether to give tert-butyl 5-{[4-chloro-2-(dimethylamino)benzoyl]amino}-2-pyridinyl[2-(1H-pyrazol-1-yl)ethyl]carbamate (357 mg) as a pale yellow solid.

¹H-NMR(CDCl₃): δ 1.46(9H, s), 2.84(6H, s), 4.30-4.35(2H, m), 4.42-4.46(2H, m), 6.20(1H, t, J=2.0 Hz), 7.25-7.29(4H, m), 7.37(1H, dd, J=1.6, 0.6 Hz), 7.46(1H, dd, J=1.6, 0.6 Hz), 7.49(1H, d, J=9.2 Hz), 8.17-8.21(2H, m), 8.45(1H, d, J=2.3 Hz), 12.02(1H, s) ESI-MS(m/z): 507(M+Na)⁺

### EXAMPLE 467

To a solution of tert-butyl 5-{[4-chloro-2-(dimethylamino)benzoyl]amino}-2-pyridinyl[2-(1H-pyrazol-1-yl)ethyl]carbamate (347 mg) in dichloromethane (10 ml) was added trifluoroacetic acid (0.674 ml). The reaction mixture was stirred for 20 hours at ambient temperature, quenched with 10% potassium carbonate solution and extracted with dichloromethane. The organic layer was washed with brine, dried over magnesium sulfate, filtered and concentrated in vacuo. The residue was recrystallized from ethyl acetate-diisopropyl ether to give 4-chloro-2-(dimethylamino)-N-(6-{[2-(1H-pyrazol-1-yl)ethyl]amino}-3-pyridinyl)benzamide (243 mg) as a pale yellow solid.

¹H-NMR(DMSO-d₆): δ 2.80(6H, s), 3.61(2H, q, J=6.3 Hz), 4.27(2H, t, J=6.3 Hz), 6.22(1H, t, J=2.3 Hz), 6.48(1H, d, J=8.6 Hz), 6.56(1H, t, J=5.7 Hz), 7.00(1H, dd, J=2.3, 0.6 Hz), 7.08(1H, d, J=2.0 Hz), 7.45(1H, dd, J=2.0, 0.6 Hz), 7.52(1H, d, J=8.2 Hz), 7.07(1H, dd, J=2.3, 0.6 Hz), 7.71(1H, dd, J=8.9, 2.6 Hz), 8.28(1H, d, J=2.6 Hz), 10.45(1H, s) ESI-MS(m/z): 385(M+H)⁺

### EXAMPLE 468

To a solution of tert-butyl 4-aminophenyl[2-(1H-pyrazol-1-yl)ethyl]carbamate (346 mg), 2-(dimethylamino)-4-(trifluoromethyl)benzoic acid (294 mg) and 1-hydroxybenzotriazole (193 mg) in N,N-dimethylformamide (3.5 ml) was added 1-[3-(dimethylamino)propyl]-3-ethylcarbodiimide hydrochloride (241 mg), followed by triethylamine (0.24 ml) at ambient temperature. The reaction mixture was stirred for 15 hours at 60° C. and concentrated in vacuo. The residue was dissolved in ethyl acetate and water, and extracted with ethyl acetate. The organic layer was washed with water and brine, dried over magnesium sulfate, filtered and concentrated in vacuo. The residue was purified by column chromatography on silica gel eluting with hexane: ethyl acetate (3:1) to give tert-butyl 4-{[2-(dimethylamino)-4-(trifluoromethyl)benzoyl]amino}phenyl[2-(1H-pyrazol-1-yl)ethyl]carbamate (440 mg) as pale yellow oil.

¹H-NMR(CDCl₃): δ 1.41(9H, s), 2.87(6H, s), 4.04(2H, t, J=5.9 Hz), 4.38(2H, t, J=5.9 Hz), 6.26(1H, t, J=1.9 Hz), 6.90-7.05(2H, m), 7.40-7.50(3H, m), 7.57(2H, d, J=8.6 Hz), 8.31(1H, dd, J=8.6, 0.5 Hz), 11.64(1H, s) ESI-MS (m/z): 540 (M+Na)⁺

### EXAMPLE 469

To a solution of tert-butyl 4-{[2-(dimethylamino)-4-(trifluoromethyl)benzoyl]amino}phenyl [2-(1H-pyrazol-1-yl)ethyl]carbamate (430 mg) in dichloromethane (3 ml) was added trifluoroacetic acid (0.96 ml). The reaction mixture was stirred for 14 hours, quenched with 10% potassium carbonate solution and extracted with dichloromethane. The organic layer was washed with brine, dried over magnesium sulfate, filtered and concentrated in vacuo. The residue was recrystallized from ethyl acetate-hexane to give 2-(dimethylamino)-N-(4-{([2-(1H-pyrazol-1-yl) ethyl]amino}phenyl)-4-(trifluoromethyl)benzamide (334 mg) as white powder.

¹H-NMR (CDCl₃): δ 2.86 (6H, s), 3.61 (2H, t, J=5.1 Hz), 4.03 (1H, br s), 4.36(2H, t, J=5.4 Hz), 6.26(1H, br s), 6.62(2H, d, J=8.6 Hz), 7.36-7.56(4H, m), 8.30(1H, d, J=8.1 Hz), 11.27(1H, br s) ESI-MS(m/z): 418(M+H)⁺

### EXAMPLE 470

The following compound was obtained in substantially the same manner as in Example 468.

tert-Butyl (4-{[4-methyl-2-(methylamino) benzoyl]amino}phenyl)[2-(1H-pyrazol-1-yl)ethyl]carbamate

¹H-NMR(CDCl₃): δ 1.39 (9H, s), 2.36 (3H, s), 2.87 (3H, s), 3.40 (1H, br q, J=6.2 Hz), 4.01(2H, t, J=5.9 Hz), 4.35(2H, t, J=6.2 Hz), 6.25(1H, t, J=2.2 Hz), 6.38-6.50(4H, m), 7.34-7.49(4H, m), 7.83(1H, br s) ESI-MS (m/z): 472 (M+Na)⁺

### EXAMPLE 471

The following compound was obtained in substantially the same manner as in Example 469.

4-Methyl-2-(methylamino)-N-(4-{[2-(1H-pyrazol-1-yl)ethyl]amino}phenyl)benzamide

¹H-NMR(CDCl₃): δ 2.34 (3H, s), 2.85 (3H, br s), 3.59 (2H, t, J=5.7 Hz), 4.34(2H, t, J=5.4 Hz), 6.25(1H, t, J=2.4 Hz), 6.45(1H, d, J=7.8 Hz), 6.49(1H, br s), 6.59(2H, d, J=8.6 Hz), 7.31(2H, d, J=8.6 Hz), 7.34(1H, d, J=7.8 Hz), 7.35(1H, d, J=2.2 Hz), 7.48(1H, br s), 7.54(1H, br s), 7.55(1H, d, J=1.9 Hz) ESI-MS(m/z): 350(M+H)⁺

### EXAMPLE 472

The following compound was obtained in substantially the same manner as in Example 468.

tert-Butyl (4-{[2-(dimethylamino)-4-ethylbenzoyl]amino}phenyl)[2-(1H-pyrazol-1-yl)ethyl]carbamate

¹H-NMR(CDCl₃): δ 1.27(3H, t, J=7.6 Hz), 1.41(9H, s), 2.70(2H, q, J=7.6 Hz), 2.82(6H, s), 4.03(2H, t, J=6.2 Hz), 4.36(2H, t, J=5.7 Hz), 6.25(1H, t, J=1.9 Hz), 6.90-7.01(2H, m), 7.11(1H, d, J=6.2 Hz), 7.12(1H, s), 7.40(1H, d, J=1.9 Hz), 7.50(1H, d, J=0.8 Hz), 7.59(2H, d, J=8.6 Hz), 8.18(1H, s, J=8.4 Hz), 12.29(1H, s) ESI-MS(m/z): 500(M+Na)⁺

### EXAMPLE 473

The following compound was obtained in substantially the same manner as in Example 469.

2-(Dimethylamino)-4-ethyl-N-(4-{[2-(1H-pyrazol-1-yl)ethyl]amino}phenyl)benzamide

¹H-NMR(CDCl₃): δ 1.26(3H, t, J=7.8 Hz), 2.68(2H, q, J=7.8 Hz), 2.81(6H, s), 3.60(2H, t, J=5.1 Hz), 3.95(1H, br s), 4.35(2H, t, J=5.4 Hz), 6.25(1H, t, J=1.9 Hz), 6.61(2H, d, J=8.6 Hz), 7.09(2H, br s), 7.36(1H, d, J=1.9 Hz), 7.49(2H, d, J=8.4 Hz), 7.56(1H, d, J=1.9 Hz), 8.17(1H, d, J=8.4 Hz), 11.89(1H, br s) ESI-MS(m/z): 378(M+H)⁺

### EXAMPLE 474

The following compound was obtained in substantially the same manner as in Example 468.

tert-Butyl (4-{[2-(dimethylamino)-4-fluorobenzoyl]amino}phenyl)[2-(1H-pyrazol-1-yl)ethyl]carbamate

¹H-NMR(CDCl₃): δ 1.40(9H, s), 2.81(6H, s), 4.03(2H, t, J=6.2 Hz), 4.37(2H, t, J=5.4 Hz), 6.25(1H, t, J=1.9 Hz), 6.86-6.99(2H, m), 7.26-7.56(3H, m), 7.70-7.79(3H, m), 8.17-8.24(1H, m), 11.64(1H, br s) ESI-MS(m/z): 490(M+Na)⁺

### EXAMPLE 475

The following compound was obtained in substantially the same manner as in Example 469.

2-(Dimethylamino)-4-fluoro-N-(4-{[2-(1H-pyrazol-1-yl)ethyl]amino}phenyl)benzamide

¹H-NMR(CDCl₃): δ 2.81(6H, s), 3.60(2H, t, J=5.4 Hz), 3.99(1H, br s), 4.35(2H, t, J=5.7 Hz), 6.25(1H, t, J=1.9 Hz), 6.61(2H, d, J=8.4 Hz), 6.86-6.96(2H, m), 7.36(1H, d, J=2.2 Hz), 7.47(2H, d, J=8.6 Hz), 7.56(1H, d, J=1.4 Hz), 8.17-8.24(1H, m), 11.24(1H, s) ESI-MS(m/z): 368(M+H)⁺

This application is based on applications Nos. 2002952331 and 2003902622, both of which were filed in Australia, and the contents of which are incorporated hereinto by reference.

1. A compound of the formula (I) 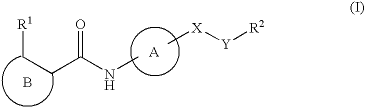
wherein
R¹ is hydrogen, lower alkyl, lower alkenyl, halo(lower)alkyl, cyclo(lower)alkyl, lower alkoxy, lower alkylthio, acyl, optionally substituted aryl or NR³R⁴, wherein
R³ and R⁴ are each independently hydrogen, lower alkyl, cyclo(lower)alkyl or acyl; or
R³, R⁴ and nitrogen atom to which they are attached form an optionally substituted, saturated or partially saturated N-containing heterocyclic group optionally having one or more oxygen or sulfur atom(s) and optionally having one or two lower alkyl(s);
R² is hydrogen; or aryl or heteroaryl in which imino group is optionally protected by amino protective group, each of which is optionally substituted by cyano, optionally protected amino, lower alkyl or heteroaryl substituted by one or more lower alkyl(s);
X is direct bond or bivalent residue derived from piperazine;
Y is -(A¹)ₙ-(A²)ₘ-
wherein
A¹ is —O—, —NH—, —N(R⁵)—, —CO—, —CH(OH)—, —NH—CO—, —CO—NH—, —CH₂—NH—CO—, —CH₂—CO—NH— or —(CH₂)₂—NH—CO—, wherein
R⁵ is amino protective group,
A² is lower alkylene optionally substituted with lower alkyl or heteroaryl, and
n and m are independently 0 or 1; 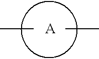
is bivalent residue derived from arene or heteroarene; and 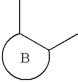
is bivalent residue derived from arene or heteroarene selected from 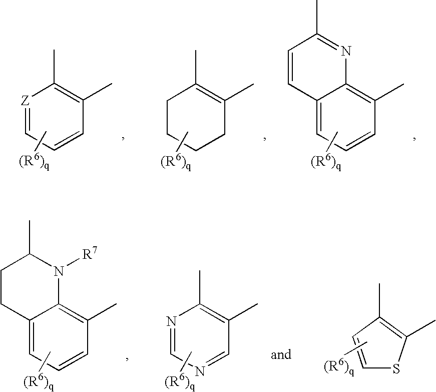
wherein
Z is N or C(R¹⁰)
R⁶ is hydrogen, halogen, lower alkyl, lower alkoxy, halo(lower)alkyl, lower alkanoyl, lower alkylthio or —NR⁸R⁹, wherein R⁸ and R⁹ are each independently lower alkyl, or R⁸, R⁹ and nitrogen atom to which they are attached form an optionally substituted, saturated or partially saturated N-containing heterocyclic group optionally having one or two lower alkyl(s);
R⁷ is lower alkyl;
R¹⁰ is the same as R⁶ defined above; and
q is 1 or 2,
or a salt thereof.

2. The compound of claim 1, wherein
R¹ is hydrogen, lower alkyl, lower alkenyl, halo(lower)alkyl, cyclo(lower)alkyl, lower alkoxy, lower alkylthio, lower alkylsulfonyl or NR³R⁴,
wherein R³ and R⁴ are each independently hydrogen, lower alkyl, cyclo(lower)alkyl, lower alkanoyl; or
R³, R⁴ and nitrogen atom to which they are attached form an optionally substituted, saturated or partially saturated N-containing heterocyclic group selected from 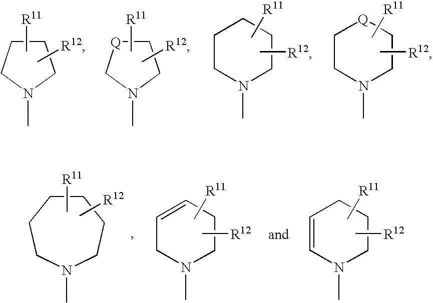
wherein R¹¹ and R¹² are each independently hydrogen or lower alkyl, and Q is —N(R¹³)—, —O—, —S—, —SO— or —SO₂—, wherein R¹³ is hydrogen or lower alkyl;
R² is hydrogen, phenyl, pyridinyl, pyrimidinyl, pyrazolyl, thiazolyl, pyrrolyl, triazolyl in which imino group is optionally protected by amino protective group, tetrazolyl, furanyl or thienyl, each of which is optionally substituted by cyano, optionally protected amino, lower alkyl or pyrrolyl substituted by one or more lower alkyl(s); 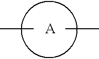
is phenylene, pyridinediyl, indolinediyl, isoindolynediyl, 3-oxo-2,3-dihydro-1H-indolediyl or 3,4-dihydro-2(1H)-isoquinolinediyl; and 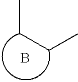
is bivalent residue derived from arene or heteroarene selected from 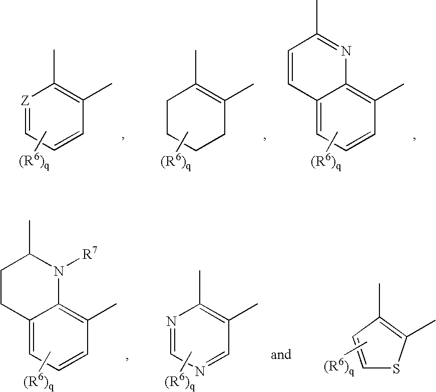
wherein
Z is N or C(R¹⁰)
R⁶ is hydrogen, halogen, lower alkyl, lower alkoxy, halo(lower)alkyl, lower alkanoyl, lower alkylthio or —NR⁸R⁹, wherein R⁸ and R⁹ are each independently lower alkyl, or
R⁸, R⁹ and nitrogen atom to which they are attached form an optionally substituted, saturated or partially saturated N-containing heterocyclic group selected from 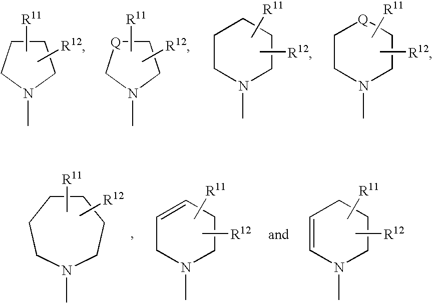
wherein R¹¹, R¹² and Q are as defined above;
R⁷ is as defined above; and
q is 1 or 2,
or a salt thereof.

3. A compound of the formula (I′) 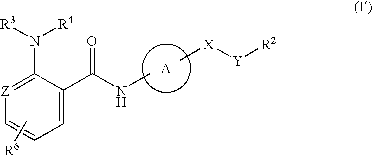
wherein
R² is aryl or heteroaryl, each of which is optionally substituted by cyano, optionally protected amino, lower alkyl or heteroaryl substituted by one or more lower alkyl(s);
R³ and R⁴ are each independently lower alkyl, or R³, R⁴ and nitrogen atom to which they are attached form an optionally substituted, saturated or partially saturated N-containing heterocyclic group;
R⁶ is hydrogen, halogen, lower alkyl, lower alkoxy, halo(lower)alkyl, lower alkanoyl or —NR⁸R⁹ (wherein R⁸ and R⁹ are each independently lower alkyl, or R⁸, R⁹ and nitrogen atom to which they are attached form an optionally substituted, saturated or partially saturated N-containing heterocyclic group); 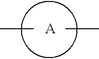
is bivalent residue derived from arene or heteroarene;
X is direct bond or bivalent residue derived from piperazine,
Y is -(A¹)ₙ-(A²)ₘ-
wherein A¹ is —O—, —NH—, —N(R⁵)—, —CO—, —CH(OH)—, —NH—CO—, —CH₂—NH—CO— or —CH₂—CO—NH—, wherein R⁵ is amino protective group,
A² is lower alkylene, and
n and m are independently 0 or 1;
Z is N or C(R¹⁰) (wherein R¹⁰ is the same as R⁶ defined above),
or a salt thereof.

4. The compound of claim 3, wherein
R² is phenyl, pyridinyl, pyrimidinyl, pyrazolyl, thiazolyl, pyrrolyl, triazolyl or tetrazolyl, each of which is optionally substituted by cyano, optionally protected amino, lower alkyl or pyrrolyl substituted by one or more lower alkyl(s),
R³ and R⁴ are each independently lower alkyl, or R³, R⁴ and nitrogen atom to which they are attached form a saturated or partially saturated N-containing heterocyclic group selected from 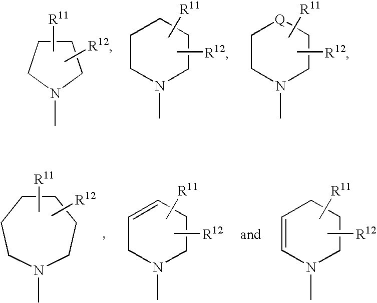
wherein R¹¹ and R¹² are each independently hydrogen or lower alkyl, and Q is —N(R¹³)—, —O—, —S—, —SO— or —SO₂— wherein R¹³ is hydrogen or lower alkyl;
R⁶ is hydrogen, halogen, lower alkyl, lower alkoxy, halo(lower)alkyl, lower alkanoyl or —NR⁸R⁹ (wherein R⁸ and R⁹ are each independently lower alkyl, or R¹¹, R¹² and nitrogen atom to which they are attached form a saturated or partially saturated N-containing heterocyclic group selected from 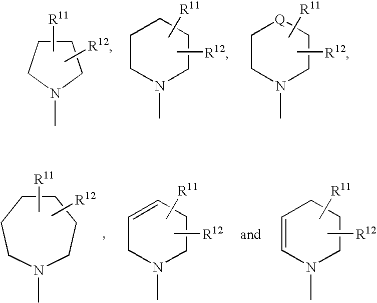
wherein R¹¹, R¹² and Q are as defined above); and 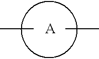
is phenylene, pyridinediyl, indolinediyl or isoindolinediyl, or a salt thereof.

5. The compound of claim 3, wherein
R² is phenyl, pyridinyl, pyrimidinyl, pyrazolyl, thiazolyl, pyrrolyl, triazolyl or tetrazolyl, each of which is optionally substituted by cyano, optionally protected amino, lower alkyl or pyrrolyl substituted by one or more lower alkyl(s);
R³ and R⁴ are each independently lower alkyl;
R⁶ is hydrogen, halogen, lower alkyl, lower alkoxy, lower alkanoyl or halo(lower)alkyl; and 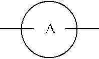
is phenylene, or a salt thereof.

6. The compound of claim 3, wherein
R² is phenyl, pyridinyl, pyrimidinyl, pyrazolyl, thiazolyl, pyrrolyl, triazolyl or tetrazolyl, each of which is optionally substituted by cyano, optionally protected amino, lower alkyl or pyrrolyl substituted by one or more lower alkyl(s);
R³ and R⁴ are each independently lower alkyl;
R⁶ is hydrogen, halogen, lower alkyl, lower alkoxy, lower alkanoyl or halo(lower)alkyl; and 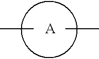
is indolinediyl or isoindolinediyl, or a salt thereof.

7. The compound of claim 3, wherein
R² is phenyl, pyridinyl, pyrimidinyl, pyrazolyl, thiazolyl, pyrrolyl, triazolyl or tetrazolyl, each of which is optionally substituted by cyano, optionally protected amino, lower alkyl or pyrrolyl substituted by one or more lower alkyl(s);
R³, R⁴ and nitrogen atom to which they are attached form a saturated N-containing heterocyclic group of the formula 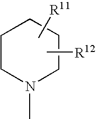
wherein R¹¹ and R¹² are each independently hydrogen or lower alkyl;
R⁶ is hydrogen, halogen, lower alkyl, lower alkoxy, lower alkanoyl or halo(lower)alkyl; and 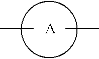
is phenylene, or a salt thereof.

8. The compound of claim 3, wherein
R² is phenyl, pyridinyl, pyrimidinyl, pyrazolyl, thiazolyl, pyrrolyl, triazolyl or tetrazolyl, each of which is optionally substituted by cyano, optionally protected amino, lower alkyl or pyrrolyl substituted by one or more lower alkyl(s);
R³, R⁴ and nitrogen atom to which they are attached form a saturated N-containing heterocyclic group of the formula 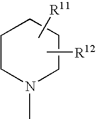
wherein R¹¹ and R¹² are each independently hydrogen or lower alkyl;
R⁶ is hydrogen, halogen, lower alkyl, lower alkoxy, lower alkanoyl or halo(lower)alkyl; and 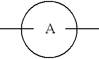
is indolinediyl or isoindolinediyl, or a salt thereof.

9. A compound of the formula (I″) 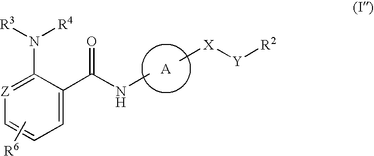
wherein
R² is aryl or heteroaryl, each of which is optionally substituted by cyano, amino, lower alkyl or heteroaryl substituted by one or more lower alkyl(s);
R³ and R⁴ are each independently lower alkyl, or R³, R⁴ and nitrogen atom to which they are attached form an optionally substituted, saturated or partially saturated N-containing heterocyclic group;
R⁶ is hydrogen, halogen, lower alkyl, lower alkoxy, halo(lower)alkyl or —NR⁸R⁹ (wherein R⁸ and R⁹ are each independently lower alkyl, or R⁸, R⁹ and nitrogen atom to which they are attached form an optionally substituted, saturated or partially saturated N-containing heterocyclic group); 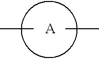
is bivalent residue derived from arene or heteroarene;
X is direct bond or bivalent residue derived from piperazine,
Y is -(A¹)ₙ-(A²)ₘ-
wherein A¹ is —O—, —NH—, —N(R⁵)—, —CO—or —NH—CO—,
wherein R⁵ is amino protective group,
A² is lower alkylene, and
n and m are independently 0 or 1; and
Z is N or C(R¹⁰) (wherein R¹⁰ is the same as R⁶ defined above),
or a salt thereof.

10. The compound of claim 9, wherein
R² is phenyl, pyridinyl, pyrimidinyl or thiazolyl, each of which is optionally substituted with cyano, amino, lower alkyl or pyrrolyl substituted with one or more lower alkyl;
R³ and R⁴ are each independently lower alkyl, or R³, R⁴ and nitrogen atom to which they are attached form a saturated or partially saturated N-containing heterocyclic group selected from 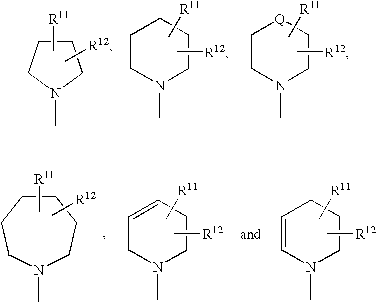
wherein R¹¹ and R¹² are each independently hydrogen or lower alkyl, and Q is —N(R¹³)—, —O—, —S—, —SO— or —SO₂— wherein R¹³ is hydrogen or lower alkyl;
R⁶ is hydrogen, halogen, lower alkyl, lower alkoxy, halo(lower)alkyl or —NR⁸R⁹ (wherein R⁸ and R⁹ are each independently lower alkyl, or R⁸, R⁹ and nitrogen atom to which they are attached form a saturated or partially saturated N-containing heterocyclic group selected from 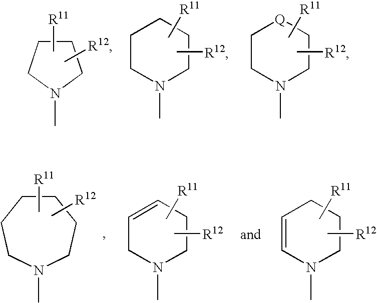
wherein R¹¹, R¹² and Q are as defined above); and 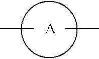
is phenylene, pyridinediyl or indolinediyl, or a salt thereof.

11. The compound of claim 9, wherein
R² is phenyl, pyridinyl, pyrimidinyl or thiazolyl, each of which is optionally substituted with cyano, amino, lower alkyl or pyrrolyl substituted with one or more lower alkyl;
R³ and R⁴ are each independently lower alkyl;
R⁶ is hydrogen, halogen, lower alkyl, lower alkoxy or halo(lower)alkyl; and 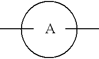
is phenylene, or a salt thereof.

12. The compound of claim 9, wherein
R² is phenyl, pyridinyl, pyrimidinyl or thiazolyl, each of which is optionally substituted with cyano, amino, lower alkyl or pyrrolyl substituted with one or more lower alkyl;
R³ and R⁴ are each independently lower alkyl;
R⁶ is hydrogen, halogen, lower alkyl, lower alkoxy or halo(lower)alkyl; and 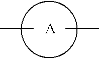
is indolinediyl, or a salt thereof.

13. The compound of claim 9, wherein
R² is phenyl, pyridinyl, pyrimidinyl or thiazolyl, each of which is optionally substituted with cyano, amino, lower alkyl or pyrrolyl substituted with one or more lower alkyl;
R³, R⁴ and nitrogen atom to which they are attached form a saturated N-containing heterocyclic group of the formula 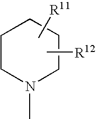
wherein R¹¹ and R¹² are each independently hydrogen or lower alkyl;
R⁶ is hydrogen, halogen, lower alkyl, lower alkoxy or halo(lower)alkyl; and 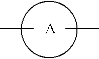
is phenylene, or a salt thereof.

14. The compound of claim 9, wherein
R² is phenyl, pyridinyl, pyrimidinyl or thiazolyl, each of which is optionally substituted with cyano, amino, lower alkyl or pyrrolyl substituted with one or more lower alkyl;
R³, R⁴ and nitrogen atom to which they are attached form a saturated N-containing heterocyclic group of the formula 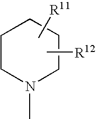
wherein R¹¹ and R¹² are each independently hydrogen or lower alkyl;
R⁶ is hydrogen, halogen, lower alkyl, lower alkoxy or halo(lower)alkyl; and 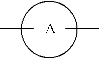
is indolinediyl, or a salt thereof.

15. The compound of claim 1 or a pharmaceutically acceptable salt thereof for use as a medicament.

16. A pharmaceutical composition comprising a compound of claim 1 or a pharmaceutically acceptable salt thereof in admixture with a pharmaceutically acceptable carrier.

17. A method for inhibiting or decreasing Apo B secretion in a mammal, which comprises administering an Apo B secretion inhibiting or decreasing amount of a compound of claim 1 or a pharmaceutically acceptable salt thereof to the mammal.

18. A method for preventing or treating a disease or condition resulting from elevated circulating levels of Apo B in a mammal, which comprises administering an effective amount of a compound of claim 1 or a pharmaceutically acceptable salt thereof to the mammal.

19. The method of claim 18, wherein the disease or condition resulting from the elevated circulating levels of Apo B is selected from the group consisting of hyperlipemia, hyperlipidemia, hyperlipoproteinemia, hypoalphalipoproteinemia, hypercholesterolemia, hypertriglyceridemia, atherosclerosis, pancreatitis, non-insulin dependent diabetes mellitus (NIDDM), obesity, coronary heart diseases, myocardial infarction, stroke, restenosis and Syndrome X.

20. The compound of claim 3 or a pharmaceutically acceptable salt thereof for use as a medicament.

21. A pharmaceutical composition comprising a compound of claim 3 or a pharmaceutically acceptable salt thereof in admixture with a pharmaceutically acceptable carrier.

22. A method for inhibiting or decreasing Apo B secretion in a mammal, which comprises administering an Apo B secretion inhibiting or decreasing amount of a compound of claim 3 or a pharmaceutically acceptable salt thereof to the mammal.

23. A method for preventing or treating a disease or condition resulting from elevated circulating levels of Apo B in a mammal, which comprises administering an effective amount of a compound of claim 3 or a pharmaceutically acceptable salt thereof to the mammal.

24. The method of claim 23, wherein the disease or condition resulting from the elevated circulating levels of Apo B is selected from the group consisting of hyperlipemia, hyperlipidemia, hyperlipoproteinemia, hypoalphalipoproteinemia, hypercholesterolemia, hypertriglyceridemia, atherosclerosis, pancreatitis, non-insulin dependent diabetes mellitus (NIDDM), obesity, coronary heart diseases, myocardial infarction, stroke, restenosis and Syndrome X.

25. The compound of claim 9 or a pharmaceutically acceptable salt thereof for use as a medicament.

26. A pharmaceutical composition comprising a compound of claim 9 or a pharmaceutically acceptable salt thereof in admixture with a pharmaceutically acceptable carrier.

27. A method for inhibiting or decreasing Apo B secretion in a mammal, which comprises administering an Apo B secretion inhibiting or decreasing amount of a compound of claim 9 or a pharmaceutically acceptable salt thereof to the mammal.

28. A method for preventing or treating a disease or condition resulting from elevated circulating levels of Apo B in a mammal, which comprises administering an effective amount of a compound of claim 9 or a pharmaceutically acceptable salt thereof to the mammal.

29. The method of claim 28, wherein the disease or condition resulting from the elevated circulating levels of Apo B is selected from the group consisting of hyperlipemia, hyperlipidemia, hyperlipoproteinemia, hypoalphalipoproteinemia, hypercholesterolemia, hypertriglyceridemia, atherosclerosis, pancreatitis, non-insulin dependent diabetes mellitus (NIDDM), obesity, coronary heart diseases, myocardial infarction, stroke, restenosis and Syndrome X.
